(19) United States
(12) Patent Application Publication
(10) Pub. No.: US 2026/0200890 A1
(43) Pub. Date: Jul. 16, 2026

(54) ADAR1 INHIBITORS AND METHODS OF USING THE SAME

(71) Applicants: AVAMMUNE THERAPEUTICS INC., Coatesville, PA (US); ATEN PORUS LIFESCIENCES PVT. LTD., Bangalore, Karnataka (IN)

(72) Inventors: Aditya KULKARNI, Bangalore, Karnataka (IN); Sandeep GOYAL, Bangalore, Karnataka (IN); Avijit GOSWAMI, Bangalore, Karnataka (IN); Kawaljit SINGH, Bangalore, Karnataka (IN); Princy KHURANA, Bangalore, Karnataka (IN)

(73) Assignees: AVAMMUNE THERAPEUTICS INC., Coatesville, PA (US); ATEN PORUS LIFESCIENCES PVT. LTD., Bangalore, Karnataka (IN)

(21) Appl. No.: 19/134,466

(22) PCT Filed: Nov. 29, 2023
(86) PCT No.: PCT/IN2023/051116

§ 371 Date: May 30, 2025

(30) Foreign Application Priority Data
Dec. 2, 2022 (IN) 202241069761

(51) Int. Cl. C07D 333/38; A61K 31/381; A61K 31/4025; A61K 31/4245; A61K 31/426; A61K 31/428; A61K 31/433; A61K 31/4418; A61K 31/4439; A61K 31/4535; A61K 31/5377; A61K 39/00; A61P 35/00; C07D 213/79; C07D 277/56; C07D 277/82; C07D 285/135; C07D 333/36; C07D 409/04; C07D 409/12; C07D 413/12; C07D 417/04; C07K 16/28

(52) U.S. Cl. CPC C07D 333/38; A61K 31/381; A61K 31/4025; A61K 31/4245; A61K 31/426; A61K 31/428; A61K 31/433; A61K 31/4418; A61K 31/4439; A61K 31/4535; A61K 31/5377; A61P 35/00; C07D 213/79; C07D 277/56; C07D 277/82; C07D 285/135; C07D 333/36; C07D 409/04; C07D 409/12; C07D 413/12; C07D 417/04; C07K 16/2818; A61K 2039/505

(57) ABSTRACT

Provided herein are compounds identified as inhibitors of ADAR1 activity, such as compounds having the formula:

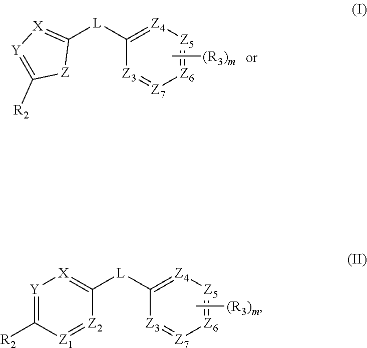

or a pharmaceutically acceptable salt or tautomer thereof, that are useful in treating cancer and other disorders, include those related to abnormal ADAR1 expression.

### CROSS-REFERENCE TO RELATED APPLICATIONS

This application claims the benefit of priority to Indian Provisional Application No. 202241069761 dated Dec. 2, 2022, which is incorporated herein by reference in its entirety for all purposes.

### BACKGROUND

Adenosine deaminase acting on RNA1 (ADAR1) is an RNA-binding protein that modifies double-stranded RNA (dsRNA) by catalyzing the posttranscriptional deamination of adenosine to inosine. Studies have found that ADAR1 is frequently amplified in many diverse types of cancers with elevated activity, including hepatocellular carcinoma, non-small cell lung cancer, thyroid cancer, pancreatic cancer, esophageal cancer, cervical cancer, and multiple myeloma. Moreover, depletion of ADAR1 in numerous cell lines caused reduced proliferation and increased apoptosis. Combined with evidence that suggests ADAR1 promotes progression and therapeutic resistance of a broad array of human malignancies, A-to-I editing of RNA catalyzed by ADAR1 has emerged as an important mechanism in cancer biology.

### BRIEF SUMMARY

Accordingly, described herein are various embodiments directed to compounds, compositions, and methods useful for treating diseases and conditions associated with aberrant ADAR1 expression. Consequently, in some embodiments, the compounds disclosed herein are selective inhibitors of ADAR1, e.g., inhibitors that target the p150 isoform.

In some aspects, the present disclosure provides a compound of Formula (I):

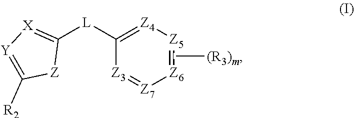

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, —S(O)₂NR_{c}—, or —C(O)—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

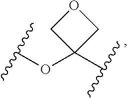 [SMILES: *OC1(*)COC1]

—OC(CF₃)—, —O—, —C(O)-alkylene-, —C(O)—, —NR_{d}S(O₂)—, —S(O₂)NR_{d}—, or heteroaryl;
R_{c} and R_{d} are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}—NR_{d}—, or S—S bond;
X is C—R₁ or N;
Y is C—R₄ or N;
Z is O, S, or N—R₅;
Z₃, Z₄, Z₅, Z₆, and Z₇ are each independently N or CH, wherein no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, alkyl, alkylenecycloalkyl, cycloalkyl, aryl, alkylenearyl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
   wherein, when present, at least one of R₁, R₂, and R₄ is not H; and wherein the compound is not:

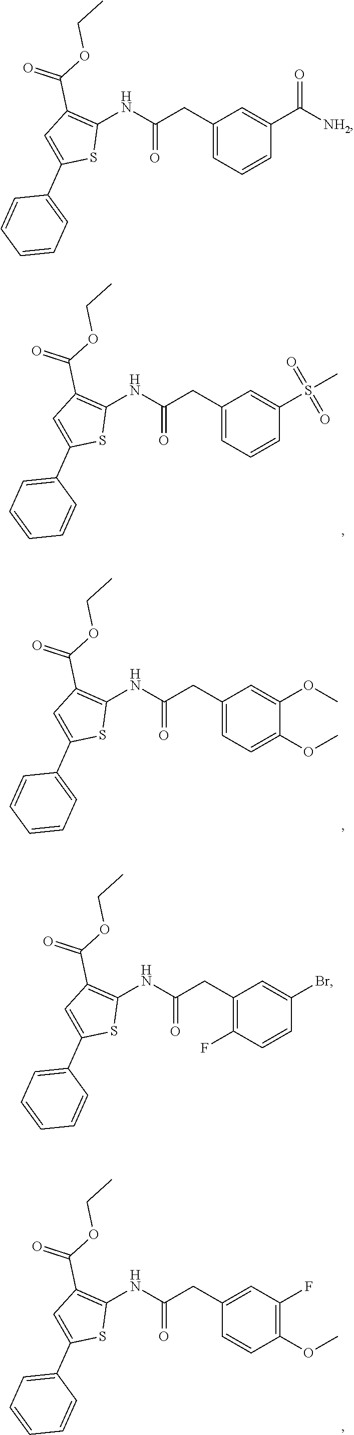 [SMILES: CCOC(=O)c1cc(-c2ccccc2)sc1NC(=O)Cc1cccc(C(N)=O)c1 ; CCOC(=O)c1cc(-c2ccccc2)sc1NC(=O)Cc1cccc(S(C)(=O)=O)c1 ; CCOC(=O)c1cc(-c2ccccc2)sc1NC(=O)Cc1ccc(OC)c(OC)c1 ; CCOC(=O)c1cc(-c2ccccc2)sc1NC(=O)Cc1cc(Br)ccc1F ; CCOC(=O)c1cc(-c2ccccc2)sc1NC(=O)Cc1ccc(OC)c(F)c1]

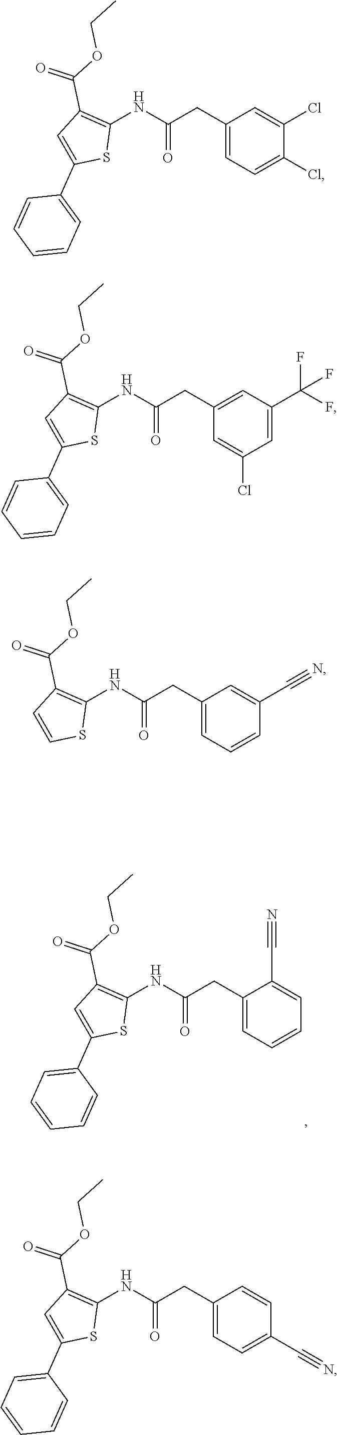 [SMILES: CCOC(=O)c1cc(-c2ccccc2)sc1NC(=O)Cc1ccc(Cl)c(Cl)c1 ; CCOC(=O)c1cc(-c2ccccc2)sc1NC(=O)Cc1cc(Cl)cc(C(F)(F)F)c1 ; CCOC(=O)c1ccsc1NC(=O)Cc1cccc(C#N)c1 ; CCOC(=O)c1cc(-c2ccccc2)sc1NC(=O)Cc1ccccc1C#N ; CCOC(=O)c1cc(-c2ccccc2)sc1NC(=O)Cc1ccc(C#N)cc1]

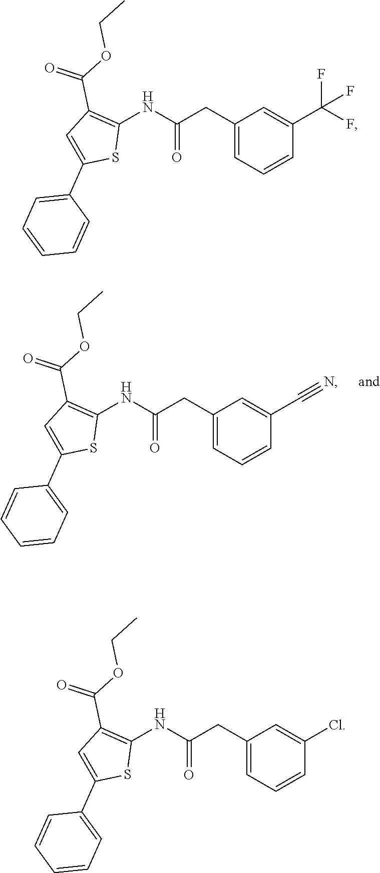 [SMILES: CCOC(=O)c1cc(-c2ccccc2)sc1NC(=O)Cc1cccc(C(F)(F)F)c1 ; CCOC(=O)c1cc(-c2ccccc2)sc1NC(=O)Cc1cccc(C#N)c1 ; "and" ; CCOC(=O)c1cc(-c2ccccc2)sc1NC(=O)Cc1cccc(Cl)c1]

In another aspect, the present disclosure provides a compound of Formula (II):

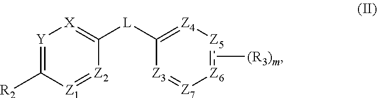

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, —S(O)₂NR_{c}—, or —C(O)—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

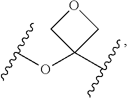 [SMILES: *OC1(*)COC1]

—OC(CF₃)—, —O—, —C(O)-alkylene-, —C(O)—, —NR_{d}S(O₂)—, —S(O₂)NR_{d}—, or heteroaryl; and
R_{c} and R_{d} are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}—NR_{d}—, or S—S bond;
X is C—R₁;
Y is C—R₄ or N;
Z₁ is N or C—R₅
Z₂ is N or CH;
Z₃, Z₄, Z₅, Z₆, and Z₇ are each independently N or CH, wherein no more than 2 of Y, Z₁, and Z₂ are N and no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, —NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, halogen, alkyl, alkoxy, alkylenecycloalkyl, cycloalkyl, aryl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
   wherein at least one of R₁, R₂, and R₄ is not H.

In a further aspect, the present disclosure provides a compound of Formula (A):

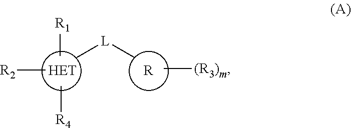

or a pharmaceutically acceptable salt or tautomer thereof,
wherein:

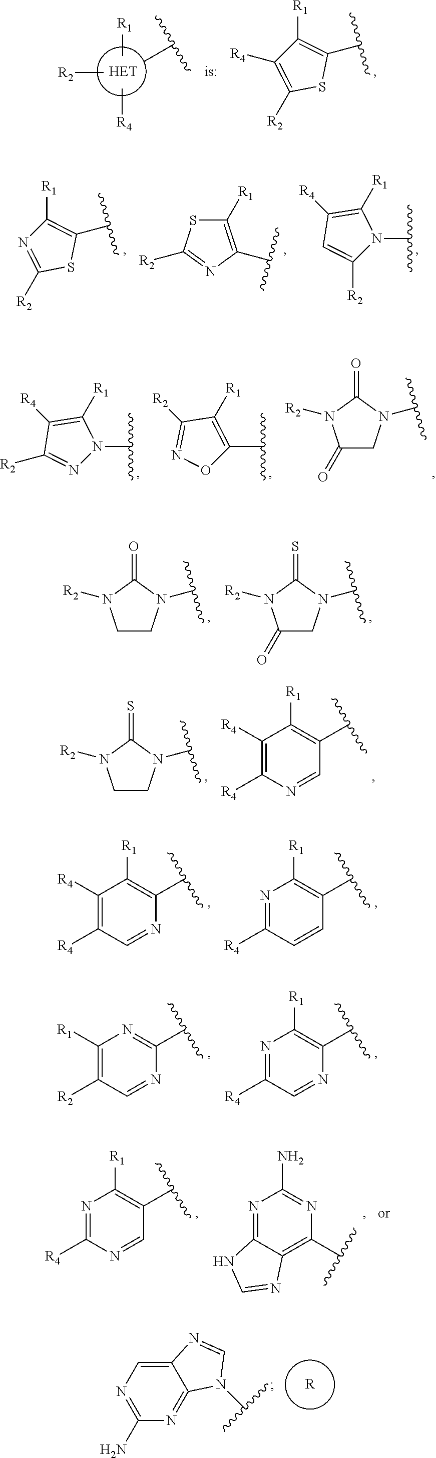

is aryl, heteroaryl, cycloalkyl, or heterocyclyl;
L is a linker having structure -A-L₁-B—,
wherein:
A is, —NR_{c}—, —NR_{c}C(O)—, —C(O)NR_{c}—, —NR_{c}S(O)₂—, —S(O)₂NR_{c}—, or —C(O)—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

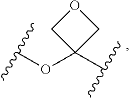 [SMILES: *OC1(*)COC1]

—OC(CF₃)—, —O—, —C(O)-alkylene-, —C(O)—, —NR_{d}S(O₂)—, —S(O₂)NR_{d}—, or heteroaryl; and
R_{c} and Rₐ are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}—NR_{d}—, or S—S bond;
R₁ is H, halogen, alkyl, —CN, —C(O)Rₐ, —C(O)ORₐ, —C(O)NHRₐ, —C(O)N(alkyl)(Rₐ), —C(O)NRₐRₐ, —NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
   wherein at least one of R₁, R₂, and R₄ is not H.

In some embodiments, the present disclosure provides a compound having the structure:

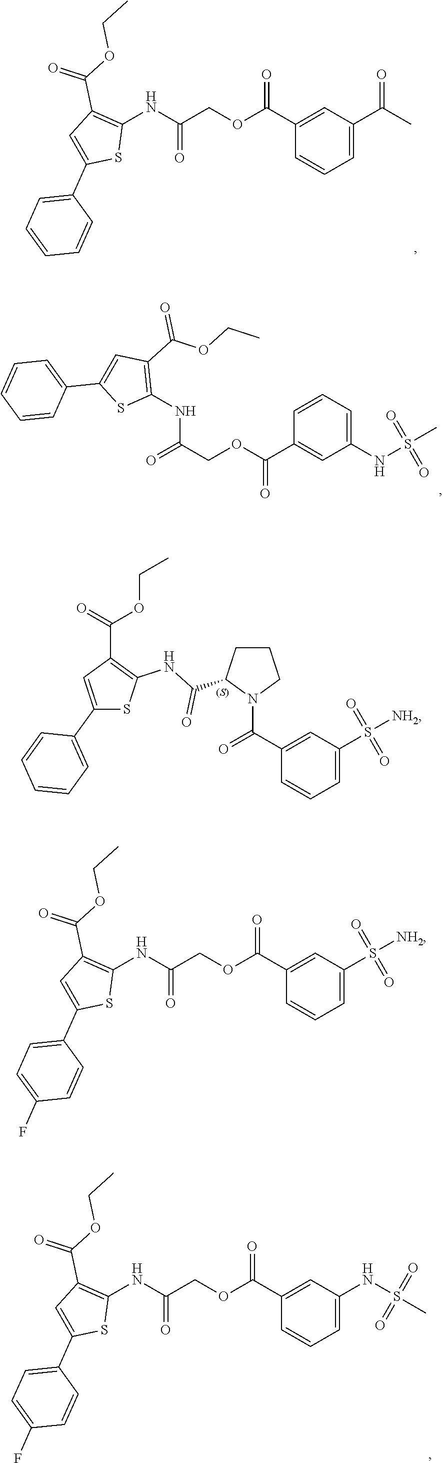

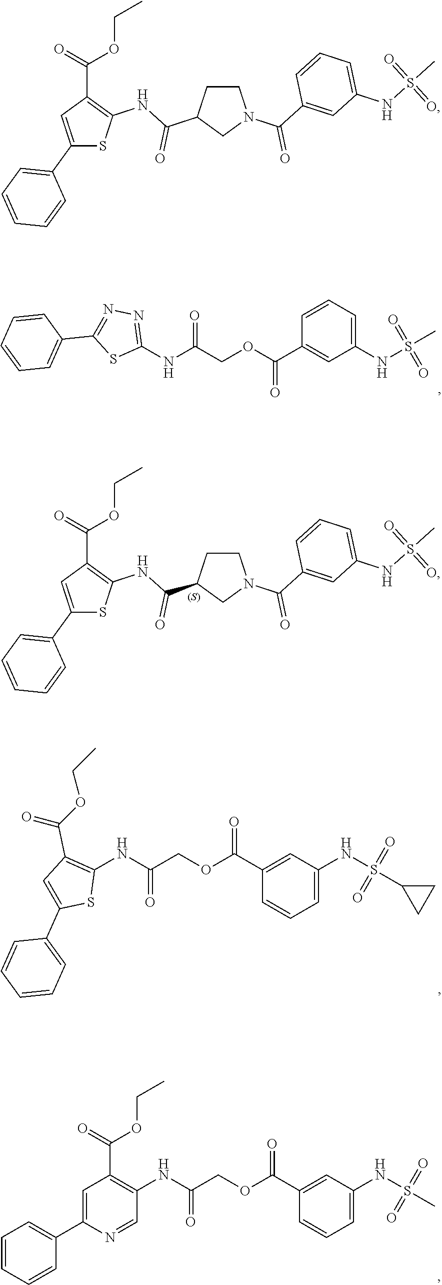

or a pharmaceutically acceptable salt thereof.

In some embodiments, the compounds of the present disclosure are adenosine deaminase acting on RNA1 (ADAR1) inhibitors. In some embodiments, the compounds of the present disclosure are inhibitors of ADAR1 p150. In some embodiments, the compounds of the present disclosure are selective inhibitors of ADAR1 p150.

In some embodiments, the present disclosure provides pharmaceutical compositions comprising (i) a compound disclosed herein; and (ii) one of more pharmaceutically acceptable carriers or excipients.

In some embodiments, the present disclosure provides methods of treating cancer in a subject in need thereof, comprising administering to the subject an effective amount of a compound disclosed herein or the pharmaceutical composition thereof.

In some embodiments, the cancer is a cancer characterized by overexpression of ADAR1. In some embodiments, the cancer is breast cancer, lung cancer, pancreatic cancer, melanoma, multiple myeloma, colon carcinoma, colorectal cancer, or glioblastoma.

In some embodiments, the present disclosure provides methods of treating an RNA viral infection in a subject in need thereof, comprising administering to the subject an effective amount of a compound disclosed herein or the pharmaceutical composition thereof.

In some embodiments, the RNA viral infection is HIV-1, HTLV-1, or SARS-CoV-2.

### BRIEF DESCRIPTION OF THE FIGURES

The skilled artisan will understand that the drawings primarily are for illustrative purposes and are not intended to limit the scope of the subject matter described herein.

FIG. 1 shows that treatment with Compound 1 induces IFN-β via inhibition of ADAR1. FIG. 1A demonstrates dose-dependent IFN-β induction in A549 p110 KO cells; FIG. 1B demonstrates that treatment of WT A549 cells with Compound 1 has lower IFN-β induction compared to FIG. 1A; FIG. 1C demonstrates IFN-β induction in A549 p110 KO cells treated with Compound 1 is superior compared to STING agonists cGAMP, ADU-S100, and MSA-002.

FIG. 2 provides a graph showing the relative MDA5 level in human cancer cell line p110 KO A549 treated with Compound 1.

FIG. 3 provides graphs showing the relative level of IFN-β (FIG. 3A), CXCL-10 (FIG. 3B), ISG15 (FIG. 3C), IFITM1 (FIG. 3D) in human cancer cell line p110 KO A549 treated with Compound 1.

FIG. 4 provides a graph showing the relative MDA5 level in human cancer cell line HCT116 treated with Compound 1.

FIG. 5 provides graphs showing the relative level of IFN-β (FIG. 5A), CXCL-10 (FIG. 5B), ISG15 (FIG. 5C), IFITM1 (FIG. 5D) in human cancer cell line HCT116 treated with Compound 1.

FIG. 6 provides a graph showing the relative MDA5 level in murine cancer cell line B16F10 treated with Compound 1.

FIG. 7 provides graphs showing the relative level of IFN-β (FIG. 7A) and CXCL-10 (FIG. 7B) in murine cancer cell line B16F10 treated with Compound 1.

FIG. 8 provides a graph showing the results of an efficacy study in B16F10 melanoma mice treated with vehicle control, anti-PD-1 therapy, Compound 1, or a combination of anti-PD-1 therapy and Compound 1. Anti-PD-1 was administered on Days 1, 4 and 7; Compound 1 was administered on Days 1, 5 and 9.

FIG. 9 provides a graph comparing tumor weight reduction in B16F10 melanoma mice treated with vehicle control, anti-PD-1 therapy, Compound 1, or a combination of anti-PD-1 therapy and Compound 1.

FIG. 10 provides a graph showing the relative MDA5 level in mice with B16F10 tumors treated with Compound 1.

FIG. 11 provides graphs showing the relative level of IFN-β (FIG. 11A) and CXCL-10 (FIG. 11B) in mice with B16F10 tumors treated with Compound 1.

### DETAILED DESCRIPTION

### Definitions

All definitions, as defined and used herein, should be understood to control over dictionary definitions, definitions in documents incorporated by reference, and/or ordinary meanings of the defined terms. Use of flow diagrams is not meant to be limiting with respect to the order of operations performed for all embodiments. The indefinite articles “a” and “an,” as used herein in the specification and in the claims, unless clearly indicated to the contrary, should be understood to mean “at least one.”

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

“Alkyl” or “alkyl group” refers to a fully saturated, straight or branched hydrocarbon chain radical, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical. Alkylenes comprising any number of carbon atoms from 1 to 12 are included. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Examples of C₁-C₃ alkyl includes methyl, ethyl, n-propyl, and i-propyl. Examples of C₁-C₄ alkyl includes methyl, ethyl, n-propyl, i-propyl, n-butyl, i-butyl, and sec-butyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl groups comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 5 to 18 carbon atoms and at least one aromatic ring. For purposes of this disclosure, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Alkylenearyl” refers to a radical of the formula —R_{b}—R_{c} where R_{b} is an alkylene, as defined above and R_{c} is one or more aryl radicals as defined above. Examples include benzyl, diphenylmethyl, and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, for example having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, for example having from three to ten carbon atoms, and which is attached to the rest of the molecule by a single bond. Monocyclic cycloalkenyl radicals include, for example, cyclopentenyl, cyclohexenyl, cycloheptenyl, cyclooctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, for example having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Alkylenecyclolalkyl” refers to a radical of the formula —R_{b}—Rₐ where R_{b} is an alkylene group as defined above and Rₐ is a cycloalkyl or cycloalkenyl radical as defined above. Unless stated otherwise specifically in the specification, an alkylenecycloalkyl group can be optionally substituted.

“Alkenylenecycloalkyl” refers to a radical of the formula —R_{b}—Rₐ where R_{b} is an alkenylene group as defined above and Rₐ is a cycloalkyl or cycloalkenyl radical as defined above. Unless stated otherwise specifically in the specification, an alkenylenecycloalkyl group can be optionally substituted.

“Alkynylenecycloalkyl” refers to a radical of the formula —R_{b}—Rₐ where R_{b} is an alkynylene group as defined above and Rₐ is a cycloalkyl or cycloalkenyl radical as defined above. Unless stated otherwise specifically in the specification, an alkynylenecycloalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Alkyleneheterocyclyl” refers to a radical of the formula —R_{b}—Rₑ where R_{b} is an alkylene as defined above and Rₑ is a heterocyclyl radical as defined above, and if the heterocyclyl is a nitrogen-containing heterocyclyl, the heterocyclyl can be attached to the alkylene radical at the nitrogen atom. Unless stated otherwise specifically in the specification, an alkyleneheterocyclyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in this disclosure, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Alkyleneheteroaryl” refers to a radical of the formula —R_{b}—R_{f} where R_{b} is an alkylene as defined above and R_{f} is a heteroaryl radical as defined above. Unless stated otherwise specifically in the specification, an alkyleneheteroaryl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —O SO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “ [symbol] ” infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Geminal” refers to any two substituents (e.g., those described herein such as alkyl, alkenyl, alkynyl, cycloalkyl, heterocycloalkyl, aryl, heteroaryl, etc.) that are attached to the same atom. In some embodiments, geminal substitution refers to substitution on the same carbon atom. The structure

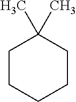

exemplifies geminal methyl substitution on cyclohexane. In some embodiments, the optional substitution is geminal substitution.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as I- or (S)- or, as (D)- or (L)- for amino acids. The present invention is meant to include all such possible isomers, as well as their racemic and optically pure forms whether or not they are specifically depicted herein. Optically active (+) and (−), I- and (S)-, or (D)- and (L)- isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. In some embodiments, inorganic salts include ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In particular embodiments, organic bases include isopropylamine, diethylamine, ethanolamine, trimethylamine, dicyclohexylamine, choline and caffeine.

Crystallization is a method commonly used to isolate a reaction product, for example one of the compounds disclosed herein, in purified form. Often, crystallization produces a solvate of the compound of the invention. As used herein, the term “solvate” refers to an aggregate that comprises one or more molecules of a compound of the invention with one or more molecules of solvent, typically in co-crystallized form. The solvent can be water, in which case the solvate can be a hydrate. Alternatively, the solvent can be an organic solvent. Thus, the compounds of the present invention can exist as a hydrate, including a monohydrate, dihydrate, hemihydrate, sesquihydrate, trihydrate, tetrahydrate and the like, as well as the corresponding solvated forms. The compound of the invention can be true solvates, while in other cases, the compound of the invention can merely retain adventitious water or be a mixture of water plus some adventitious solvent.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 and/or ChemDraw Professional 16.0.0.82 software naming program (CambridgeSoft), or the like. For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

The invention disclosed herein is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabeled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

As used herein, a “subject” can be a human, non-human primate, mammal, rat, mouse, cow, horse, pig, sheep, goat, dog, cat, insect and the like. The subject can be suspected of having or at risk for having a cancer, such as a blood cancer, or another disease or condition. Diagnostic methods for various cancers, and the clinical delineation of cancer, are known to those of ordinary skill in the art. The subject can also be suspected of having an infection or abnormal cardiovascular function.

“Mammal” includes humans and both domestic animals such as laboratory animals and household pets (e.g., cats, dogs, swine, cattle, sheep, goats, horses, rabbits), and non-domestic animals such as wildlife and the like.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of a cancer to a resistant form. Typically, a prophylactic dose is used in subjects prior to or at an earlier stage of disease, so that a prophylactically effective amount can be less than a therapeutically effective amount.

“Treating” or “treatment” as used herein covers the treatment of the disease or condition of interest in a mammal, for example in a human, having the disease or condition of interest, and includes (but is not limited to):
1. preventing the disease or condition from occurring in a mammal, in particular, when such mammal is predisposed to the condition but has not yet been diagnosed as having it;
2. inhibiting the disease or condition, i.e., arresting its development;
3. relieving the disease or condition, i.e., causing regression of the disease or condition (ranging from reducing the severity of the disease or condition to curing the disease of condition); or
4. relieving the symptoms resulting from the disease or condition, i.e., relieving pain without addressing the underlying disease or condition. As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

Throughout the present specification, the terms “about” and/or “approximately” can be used in conjunction with numerical values and/or ranges. The term “about” is understood to mean those values near to a recited value. For example, “about 40 [units]” can mean within ±25% of 40 (e.g., from 30 to 50), within ±20%, ±15%, ±10%, ±9%, ±8%, ±7%, ±6%, +5%, ±4%, ±3%, ±2%, ±1%, less than ±1%, or any other value or range of values herein. Furthermore, the phrases “less than about [a value]” or “greater than about [a value]” should be understood in view of the definition of the term “about” provided herein. The terms “about” and “approximately” can be used interchangeably.

Numerical ranges may be provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

Following below are more detailed descriptions of various concepts related to, and embodiments of inventive compounds and methods for the treatment of liver diseases and abnormal conditions of the liver. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Adenosine deaminase acting on RNA (ADAR1) is an RNA-binding protein that modifies double stranded RNA (dsRNA) through deamination of adenosine (A) to inosine (I). ADAR1 is overexpressed in various cancers such as breast, lung, pancreatic, and others. Thus, identification of inhibitors or this enzyme may provide therapeutic benefits.

Provided herein are compounds and compositions that are useful for inhibiting ADAR1 activity and treating cancer.

### Compounds and Compositions

In some embodiments of the present disclosure provides compounds of Formula (I):

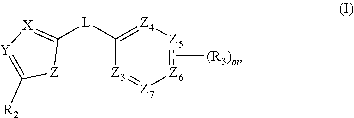

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, —S(O)₂NR_{c}—, or —C(O)—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

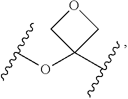 [SMILES: *OC1(*)COC1]

—OC(CF₃)—, —O—, —C(O)-alkylene-, —C(O)—, —NR_{d}S(O₂)—, —S(O₂)NR_{d}—, or heteroaryl;
R_{c} and R_{d} are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}— —NR_{d}—, or S—S bond;
X is C—R₁ or N;
Y is C—R₄ or N;
Z is O, S, N—R₅, or N(R₅)—CH₂—;
Z₃, Z₄, Z₅, Z₆, and Z₇ are each independently N or CH, wherein no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl) (Rₐ), —C(O)N(Rₐ)(Rₐ), —C(O)NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, NR_{b}R_{b}, —CN, or —P(O)(OR_{b})₂, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, alkyl, alkylenecycloalkyl, cycloalkyl, aryl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
   wherein the compound is not:

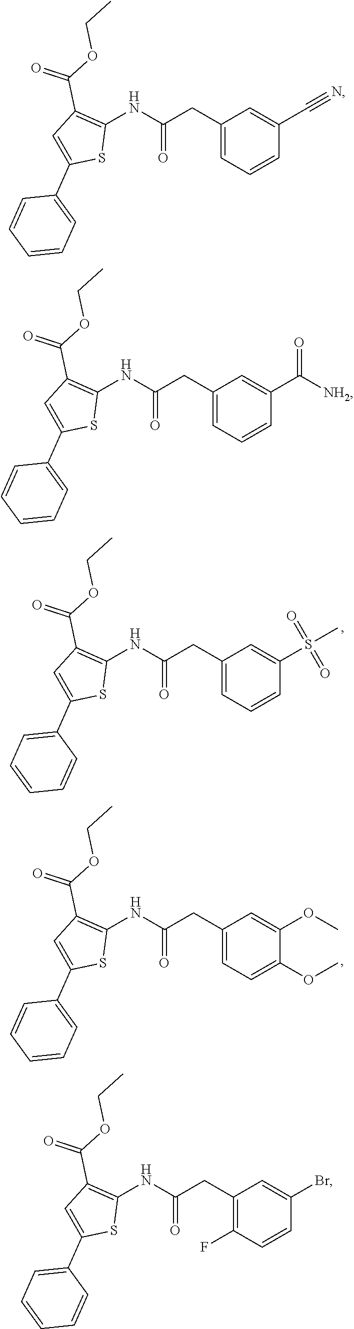

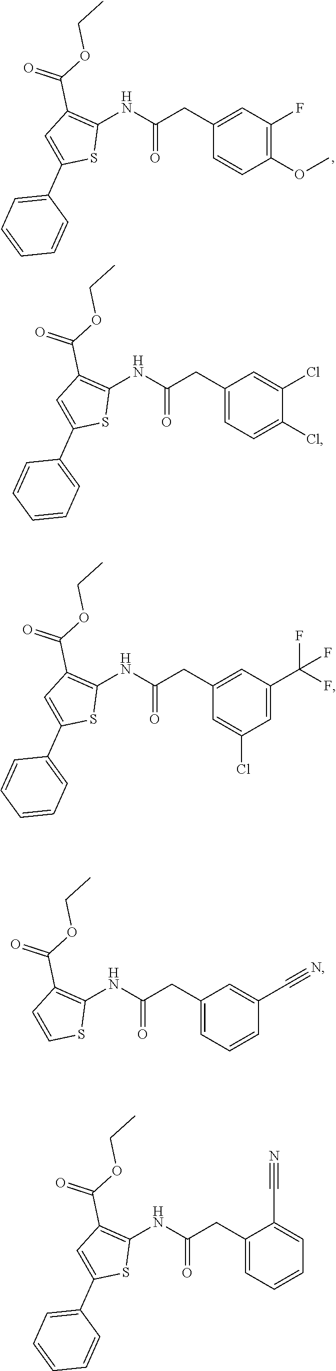 [SMILES: CCOC(=O)c1cc(-c2ccccc2)sc1NC(=O)Cc1ccc(OC)c(F)c1 ; CCOC(=O)c1cc(-c2ccccc2)sc1NC(=O)Cc1ccc(Cl)c(Cl)c1 ; CCOC(=O)c1cc(-c2ccccc2)sc1NC(=O)Cc1cc(Cl)cc(C(F)(F)F)c1 ; CCOC(=O)c1ccsc1NC(=O)Cc1cccc(C#N)c1 ; CCOC(=O)c1cc(-c2ccccc2)sc1NC(=O)Cc1ccccc1C#N]

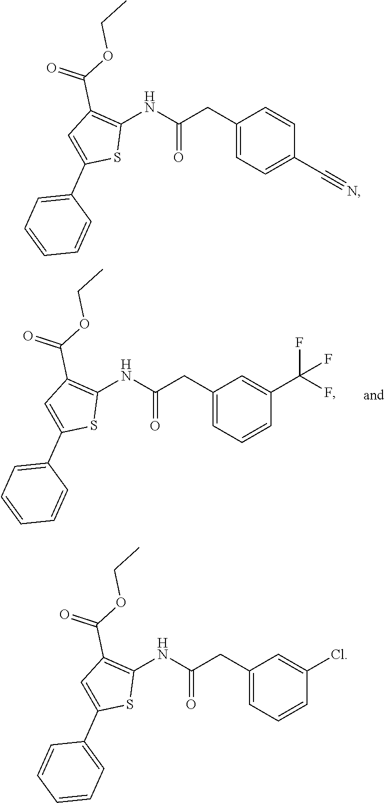

In some embodiments of the present disclosure provides a compound of Formula (I):

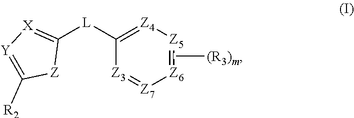

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, —S(O)₂NR_{c}—, or —C(O)—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

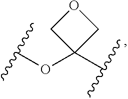 [SMILES: *OC1(*)COC1]

—OC(CF₃)—, —O—, —C(O)-alkylene-, —C(O)—, —NR_{d}S(O₂)—, —S(O₂)NR_{d}—, or heteroaryl;
R_{c} and R_{d} are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}— —NR_{d}—, or S—S bond;
X is C—R₁ or N;
Y is C—R₄ or N;
Z is O, S, N—R₅, or N(R₅)—CH₂—;
Z₃, Z₄, Z₅, Z₆, and Z₇ are each independently N or CH, wherein no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl) (Rₐ), —C(O)N(Rₐ)(Rₐ), —C(O)NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, NR_{b}R_{b}, —CN, or —P(O)(OR_{b})₂, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, alkyl, alkylenecycloalkyl, cycloalkyl, aryl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
wherein when X is C—R₁, Y is C—R₅, and Z is S; -A-L₁-B— is not

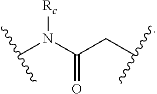

In some embodiments, X is C—R₁. In some embodiments, X is N.

In some embodiments, Y is C—R₄. In some embodiments, Y is N.

In some embodiments, Z is O, S, or N—R₅. In some embodiments, Z is O or S. In some embodiments, Z is S. In some embodiments, Z is O. In some embodiments, Z is N—R₅.

X, Y, and Z can be combined in any manner that provides a stable heteroaryl ring. In some embodiments, X is C—R₁ or N, Y is C—R₄ or N, and Z is S. In some embodiments, X is C—R₁, Y is C—R₄, and Z is S. In some embodiments, X is C—R₁, Y is N, and Z is S. In some embodiments, X and Y are N and Z is S. In some embodiments, X is N, Y is C—R₄, and Z is S.

In some embodiments, Z₃, Z₄, Z₅, Z₆, and Z₇ is each H. In some embodiments, no more than one of Z₃, Z₄, Z₅, Z₆, and Z₇ is N. In some embodiments, one of Z₃, Z₄, Z₅, Z₆, and Z₇ is N. In some embodiments, Z₃ is N and Z₄, Z₅, Z₆, and Z₇ are each H. In some embodiments, Z₄ is N and Z₃, Z₅, Z₆, and Z₇ are each H. In some embodiments, Z₅ is N and Z₃, Z₄, Z₆, and Z₇ are each H. In some embodiments, Z₆ is N and Z₃, Z₄, Z₄, and Z₇ are each H. In some embodiments, Z₇ is N and Z₃, Z₄, Z₅, and Z₆ are each H. In some embodiments, no more than two of Z₃, Z₄, Z₅, Z₆, and Z₇ are each N. In some embodiments, two of Z₃, Z₄, Z₅, Z₆, and Z₇ are each N. In some embodiments, Z₃ and Z₄ are each N. In some embodiments, Z₃ and Z₅ are each N. In some embodiments, Z₃ and Z₆ are each N. In some embodiments, Z₄ and Z₆ are each N. In some embodiments, Z₄ and Z₇ are each N. In some embodiments, Z₅ and Z₇ are each N. In some embodiments, Z₃ and Z₇ are each N. In some embodiments, Z₆ and Z₇ are each N. In some embodiments, Z₅ and Z₆ are each N. In some embodiments, Z₄ and Z₅ are each N.

In some embodiments, A is —NR_{c}—, —NR_{c}C(O)—, —C(O)NR_{c}—, —NR_{c}S(O)₂—, or —S(O)₂NR_{c}—. In some embodiments, A is —NR_{c}C(O)—, —C(O)NR_{c}—, —NR_{c}S(O)₂—, or —S(O)₂NR_{c}—. In some embodiments, A is —NR_{c}C(O)—, —C(O)NR_{c}—, or —C(O)—. In some embodiments, A is —NR_{c}C(O)— or —NR_{c}S(O)₂—. In some embodiments, A is —NR_{c}C(O)— or —S(O)₂NR_{c}—. In some embodiments, A is —NR_{c}C(O)—. In some embodiments, R_{c} is H, C₁₋₅alkyl, C₃₋₆cycloalkyl, or —CH₂-phenyl. In some embodiments, R_{c} is H or C₁₋₅alkyl. In some embodiments, the C₁₋₅alkyl is methyl, ethyl, or isopropyl. In some embodiments, the C₁₋₅alkyl is methyl. In some embodiments, the C₃₋₆cycloalkyl is cyclopropyl. In some embodiments, R_{c} is H or methyl. In some embodiments, R_{c} is H.

In some embodiments, L₁ is absent, alkylene, cycloalkyl, heterocyclyl, or heteroaryl. In some embodiments, L₁ is absent, alkylene, cycloalkyl, or heterocyclyl. In some embodiments, L₁ is absent, alkylene, or heterocyclyl. In some embodiments, L₁ is alkylene or heterocyclyl. In some embodiments, the alkylene is a C₁₋₅alkylene. In some embodiments, the alkylene is a C₁₋₃alkylene. In some embodiments, the alkylene is methylene. In some embodiments, the alkylene is geminal substituted. In some embodiments, L₁ is alkylene, and the alkylene is a C₁₋₃alkylene. In some embodiments, the alkylene is an optionally substituted methylene. In some embodiments, the alkylene comprises the structure:

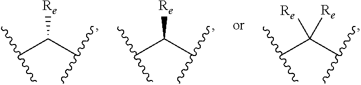

wherein Rₑ is each independently H, F, C₁₋₅ alkyl, —CH₂OH, —CH₂cycloalkyl, — or CH₂aryl, or two Rₑ taken together with the carbon atom to which they are attached form a C₃₋₆ cycloalkyl or a 4- to 6-membered heterocyclyl. In some embodiments, Rₑ is H or C₁₋₅ alkyl.

In some embodiments, L₁ comprises a heterocyclyl. In some embodiments, the heterocyclyl is a 3- to 12-membered heterocyclyl. In some embodiments, the heterocyclyl has the structure:

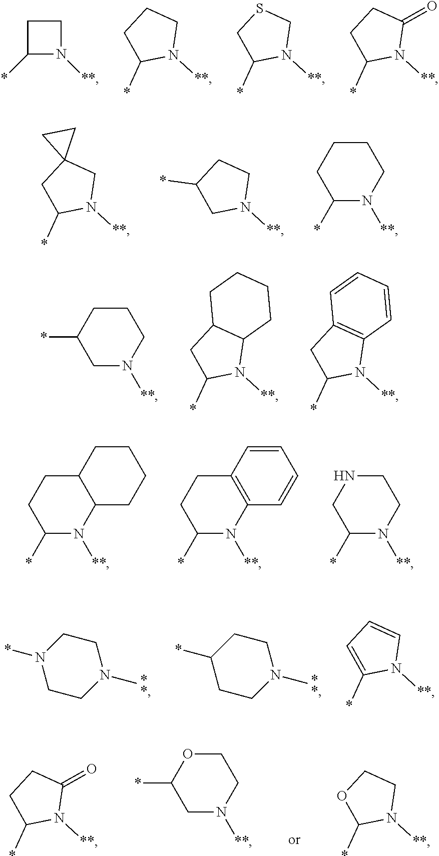

wherein * represents the attachment point to A and ** represents the attachment point to B.

In some embodiments, B is bond, —NR_{b}C(O)—, —NR_{b}S(O)₂—, —C(O)—, —OC(O)—, —C(O)—O—, or a heteroaryl. In some embodiments, B is a bond, —OC(O)—, or —C(O)—. In some embodiments, B is —OC(O)—. In some embodiments, B is —C(O)—. In some embodiments, B is a bond. In some embodiments, B is heteroaryl. In some embodiments, the heteroaryl is a 5- or 6-membered nitrogen-containing heteroaryl. In some embodiments, the heteroaryl is a triazole. In some embodiments, the heteroaryl is a 1,2,3-triazole or 1,2,4-triazole.

In some embodiments, L is a linker having the structure -A-L₁-B—, wherein A is —NR_{c}C(O)— or —C(O)NR_{c}; L₁ is absent, alkylene, cycloalkyl, or heterocyclyl; B is a bond, —NR_{d}—, —N(S(O)₂-alkyl)-, —NR_{d}C(O)—, —OC(O)—, —C(O)-alkylene-, —C(O)—, —NR_{d}S(O₂)—, or —S(O₂) NR_{d}—; and R_{c} and R_{d} are each independently H, alkyl, cycloalkyl, or alkylene-aryl, with the proviso that -A-L₁-B— does not contain an —O—O, —NR_{c}—O, —O—NR_{d}—, —NR_{c}—NR_{d}—, or S—S bond. In some embodiments, R_{c} and R_{d} are each independently H, alkyl, cycloalkyl, alkylene-aryl, acyl, or sulfonyl. In some embodiments, R_{c} and R_{d} are each independently H or alkyl. In some embodiments, R_{c} and R_{d} are each independently H or C₁₋₅alkyl. In some embodiments, R_{c} and R_{d} are each H.

In some embodiments, L is a linker having the structure -A-L₁-B—, wherein: A is —NR_{c}C(O)—; L₁ is alkylene or heterocyclyl; and B is —C(O)— or —OC(O)—. In some embodiments, A is —NR_{c}C(O)—; L₁ is C₁₋₃alkylene or 5- to 6-membered heterocyclyl; and B is —C(O)— or —OC(O)—. In some embodiments, A is —NR_{c}C(O)—; L₁ is methylene or pyrrolidine; and B is —C(O)— or —OC(O)—. In some embodiments, A is —NR_{c}C(O)—; L₁ is methylene or

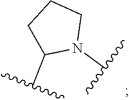 [SMILES: *C1CCCN1*]

and B is —C(O)— or —OC(O)—. In some embodiments, L is a linker having the structure -A-L₁-B—, wherein: A is —NR_{c}C(O)—; L₁ is absent; and B is a heteroaryl. In some embodiments, the heteroaryl is a 5-membered heteroaryl. In some embodiments, the heteroaryl is an oxadiazolyl, thiadiazolyl, triazolyl, oxazolyl, thiazolyl, or imidazolyl. In some embodiments, the heteroaryl has the structure of

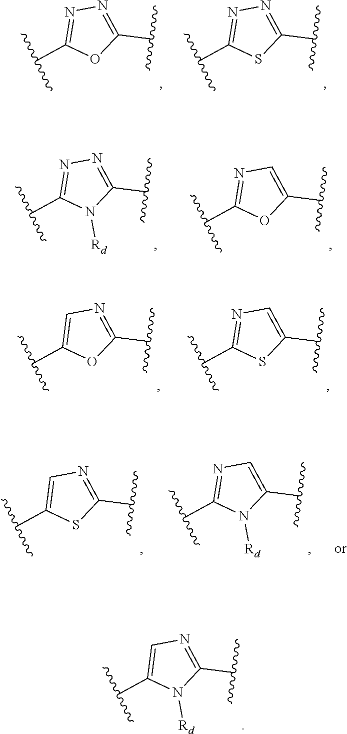

In some embodiments, -A-L₁-B— has the structure:

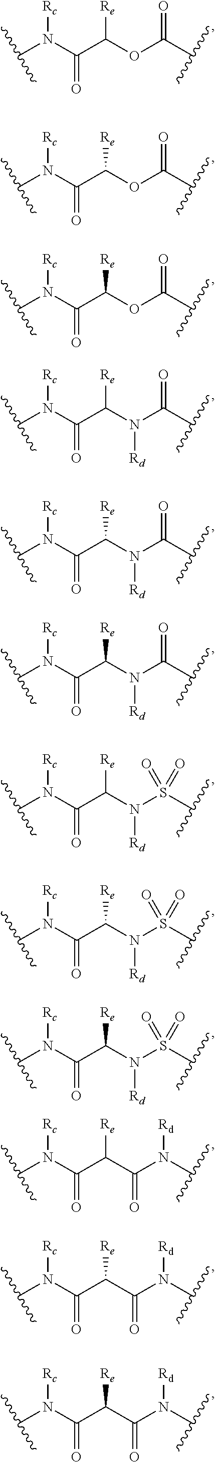

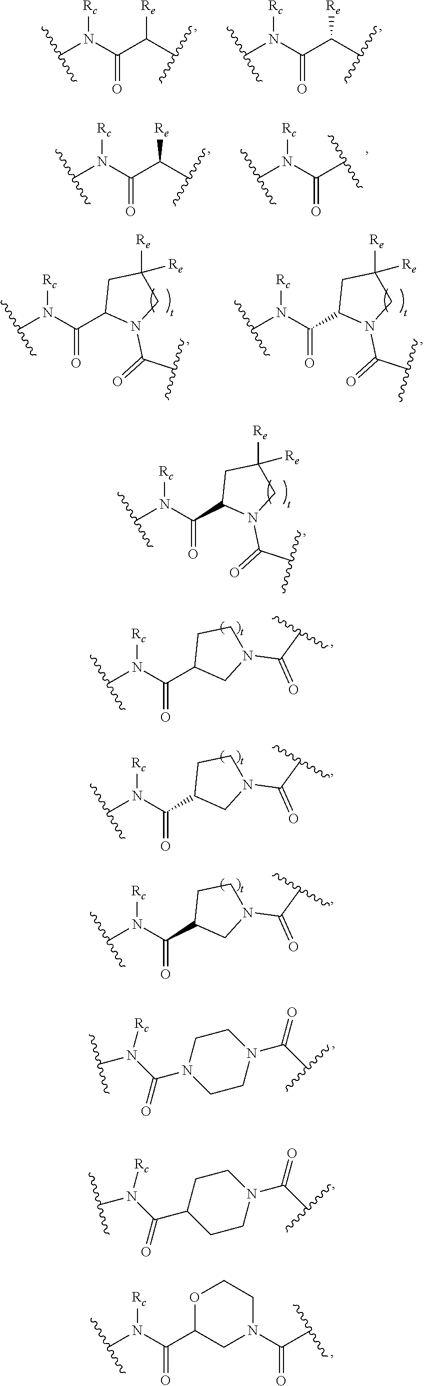

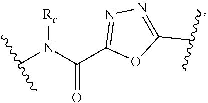

or
wherein R_{c} and R_{d} are each independently H, alkyl, cycloalkyl, alkylene-aryl, acyl, or sulfonyl; Rₑ is each independently H, F, C₁₋₅ alkyl, —CH₂cycloalkyl, — or CH₂aryl, or two Rₑ taken together with the carbon atom to which they are attached form a C₃₋₆ cycloalkyl or a 4- to 6-membered heterocyclyl; and t is 0, 1 or 2. In some embodiments, R_{c} is H or alkyl. In some embodiments, R_{c} is H or C₁₋₅alkyl. In some embodiments, R_{c} is H. In some embodiments, Rₑ is each independently H, F, or C₁₋₅ alkyl. In some embodiments, Rₑ is each independently H, F, Me, Et, or iPr. In some embodiments, Rₑ is H. In some embodiments, s is 1. In some embodiments, t is 1.

In some embodiments, -A-L₁-B— has the structure:

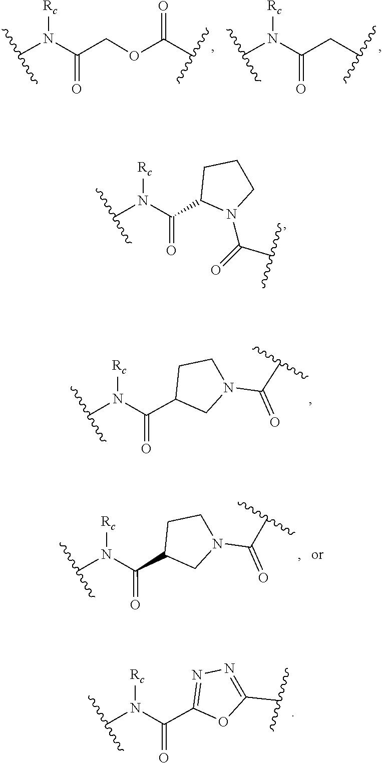

In some embodiments, R_{c} is H or alkyl. In some embodiments, R_{c} is H or C₁₋₅alkyl. In some embodiments, R_{c} is H.

In some embodiments, R₁ is alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ. In some embodiments, R₁ is alkyl, —C(O)ORₐ, —C(O)N(Rₐ)(Rₐ), or —C(O)NHRₐ. In some embodiments, R₁ is —C(O)ORₐ. In some embodiments, each Rₐ is independently H or C₁₋₅ alkyl (e.g., methyl, ethyl, or n-propyl, or i-propyl), or two Rₐ groups taken together with the nitrogen atom to which they are attached form a heterocyclyl. In some embodiments, the heterocyclyl is a 5- or 6-membered nitrogen-containing heterocyclyl. In some embodiments, the heterocyclyl has the structure:

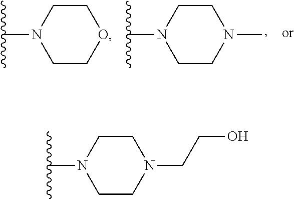

In some embodiments, Rₐ is C₁₋₅ alkyl. In some embodiments, Rₐ is methyl, ethyl, or n-propyl, or i-propyl. In some embodiments, Rₐ is ethyl.

In some embodiments, X is C—R₁, and R₁ and R_{c} taken together with the atoms to which they are attached form a heterocyclyl or heteroaryl. In some embodiments, the heterocyclyl is a 5- or 6-membered heterocyclyl. In some embodiments, the heteroaryl is a 5- or 6-membered heteroaryl.

In some embodiments, R₄ is H, alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ. In some embodiments, R₄ is H, alkyl, aryl, —C(O)ORₐ, or —C(O)NHRₐ. In some embodiments, R₄ is H, alkyl, —C(O)ORₐ, —C(O)N(Rₐ)(Rₐ), or —C(O)NHRₐ. In some embodiments, R₄ is H, halogen, or —C(O)ORₐ. In some embodiments, R₄ is H. In some embodiments, R₄ is halogen. In some embodiments, the halogen is F or Cl. In some embodiments the halogen is Cl. In some embodiments, R₄ is H or —C(O)ORₐ. In some embodiments, R₄ is H. In some embodiments, Rₐ is C₁₋₅ alkyl or —CH₂cycloalkyl. In some embodiments, Rₐ is C₁₋₅ alkyl. In some embodiments, Rₐ is methyl, ethyl, or n-propyl, or i-propyl. In some embodiments, each Rₐ is independently H or C₁₋₅ alkyl (e.g., methyl, ethyl, or n-propyl, or i-propyl), or two Rₐ groups taken together with the nitrogen atom to which they are attached form a heterocyclyl. In some embodiments, the heterocyclyl is a 5- or 6-membered nitrogen-containing heterocyclyl. In some embodiments, the heterocyclyl has the structure:

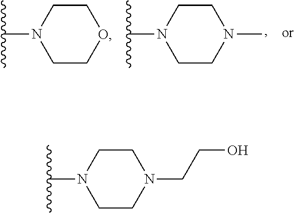

In some embodiments, Rₐ is C₁₋₅ alkyl. In some embodiments, Rₐ is methyl, ethyl, or n-propyl, or i-propyl. In some embodiments, Rₐ is ethyl.

In some embodiments, R₂ is aryl or heteroaryl. In some embodiments, the aryl is phenyl and the heteroaryl is 6-membered nitrogen-containing heteroaryl. In some embodiments, the aryl is phenyl and the heteroaryl is a pyridyl (e.g., 2-pyridyl, 3-pyridyl, or 4-pyridyl). In some embodiments, R₂ is phenyl or naphthyl. In some embodiments, R₂ is phenyl. In some embodiments, R₂ is

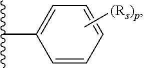

wherein Rₛ is each independently halogen, alkyl, haloalkyl, —OH, alkoxy, haloalkoxy, amino (e.g., —NH₂, —NHCH₃, —N(CH₃)₂, etc.), or heterocyclyl, or two Rₛ taken together with the atoms to which they are attached form an aryl, heteroaryl, cycloalkyl, or heterocycloalkyl; and p is 0, 1, 2, or 3. In some embodiments, R₂ is

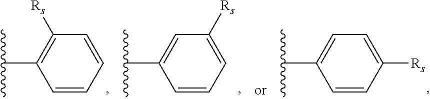

wherein R_{c} is each independently halogen, alkyl, haloalkyl, —OH, alkoxy, haloalkoxy, or amino. In some embodiments, R₂ is

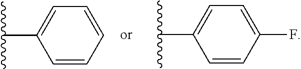 [SMILES: *c1ccccc1 ; *c1ccc(F)cc1]

In some embodiments, R₂ is a 6-membered nitrogen-containing heteroaryl. In some embodiments, R₂ is

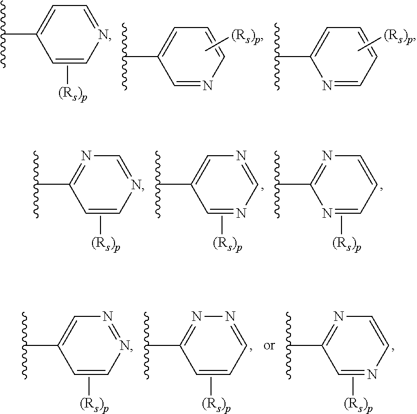

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, or 2. In some embodiments, R₂ is

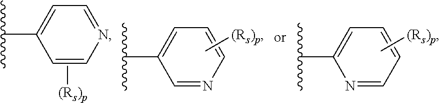

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, or 2. In some embodiments, Rₛ is each independently H, F, C₁₋₅alkyl, —OC₁₋₅alkyl, CF₃, —OCF₃, or —CN. In some embodiments, Rₛ is each independently H or F. In some embodiments, p is 0 or 1. In some embodiments, p is 1 or 2. In some embodiments, p is 0. In some embodiments, p is 1. In some embodiments, p is 2.

In some embodiments, R₂ and R₄ taken together with the carbon atoms to which they are attached form a cycloalkyl, heterocyclyl, aryl, or heteroaryl. In some embodiments, R₂ and R₄ taken together with the carbon atoms to which they are attached form a cycloalkyl. In some embodiments, the cycloalkyl is a C₅₋₇cycloalkyl. In some embodiments, the cycloalkyl is a C₅₋₆cycloalkyl. In some embodiments, the cycloalkyl is a C₄₋₆ cycloalkyl.

In some embodiments, when X is C—R₁ and Y is C—R₄, at least two of R₁, R₂, and R₄ are not H. In some embodiments, R₁ and R₂ are not H, and R₄ is H. In some embodiments, when X is C—R₁ and Y is C—R₄, R₁ is —C(O)ORₐ, —C(O)N(Rₐ)(Rₐ), or —C(O)NHRₐ; R₂ is optionally substituted phenyl; and R₄ is H, wherein Rₐ is as defined herein. In some embodiments, R₁ is —C(O)ORₐ; R₂ is optionally substituted phenyl; and R₄ is H, wherein Rₐ is as defined herein. In some embodiments, R₁ is —C(O)ORₐ; R₂ is

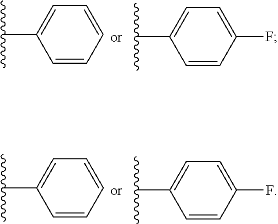

and R₄ is H, wherein Rₐ is as defined herein. In some embodiments, X and Y are N and R₂ is In some embodiments, Z is S. In some embodiments, Z is O. In some embodiments, Z is N—R₅.

In some embodiments, R₅ is H, alkyl, alkylenecycloalkyl, cycloalkyl, aryl, or alkylenearyl. In some embodiments, R₅ is H, alkyl, or alkylenecycloalkyl. In some embodiments, R₅ is H or alkyl. In some embodiments, the alkyl is Me, Et, or iPr. In some embodiments, the aryl is a phenyl. In some embodiments, the cycloalkyl is a C₃₋₆cycloalkyl. In some embodiments, the alkylene is a C₁₋₃alkylene. In some embodiments, the alkylene is a methylene.

In some embodiments, R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —P(O)(OR_{b})₂, or two R₃ taken together with the adjacent carbon atoms to which they are attached form an aryl, cycloalkyl, or heterocyclyl. In some embodiments, R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, or —NHC(O)NR_{b}R_{b}, or two R₃ taken together with the adjacent carbon atoms to which they are attached form an aryl, cycloalkyl, or heterocyclyl. In some embodiments, R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl. In some embodiments, R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN. In some embodiments, R₃ is —S(O)₂NHR_{b}, —S(O)₂NR_{b}R_{b}, —NHS(O)₂R_{b}, —C(O)R_{b}, —C(O)NHR_{b}, —C(O)NR_{b}R_{b}, —NHC(O)R_{b}, —NHC(O)NR_{b}R_{b}, or —CN. In some embodiments, R₃ is —S(O)₂NHR_{b}, —S(O)₂NR_{b}R_{b}, —NHS(O)₂R_{b}, —C(O)NHR_{b}, —C(O)NR_{b}R_{b}, —NHC(O)R_{b}, —NHC(O)NR_{b}R_{b}, or —CN. In some embodiments, R₃ is —N(H)S(O)₂alkyl, —N(H)S(O)₂cycloalkyl, —S(O)₂NH₂, —C(O)alkyl, or —CN. In some embodiments, R₃ is —N(H)S(O)₂(C₁₋₅alkyl), —S(O)₂N(C₁₋₅alkyl)₂, —S(O)₂N(H)(C₁₋₅alkyl), or —S(O)₂NH₂. In some embodiments, R₃ is —N(H)S(O)₂ (C₁₋₅alkyl) or —S(O)₂NH₂. In some embodiments, R₃ is —N(H)S(O)₂CH₃, —S(O)₂NH₂, —C(O)CH₃, or —CN. In some embodiments, R₃ is —N(H)S(O)₂CH₃, —S(O)₂NH₂, or —C(O)CH₃. In some embodiments, R₃ is —N(H)S(O)₂CH₃ or —S(O)₂NH₂.

In some embodiments, m is 0, 1, or 2. In some embodiments, m is 0 or 1. In some embodiments, m is 1 or 2. In some embodiments, m is 0. In some embodiments, m is 1. In some embodiments, m is 2. In some embodiments, m is 3.

In some embodiments, X is C—R₁, Y is C—R₄, R₂ is aryl, heteroaryl; R₃ is —N(H)S(O)₂CH₃, —S(O)₂NH₂, —C(O)CH₃, or —CN; and L is a linker having the structure -A-L₁-B—, wherein: A is —NR_{c}C(O)—; L₁ is alkylene or heterocyclyl; and B is —C(O)— or —OC(O)—, wherein R₁, R₂, and R₄ are as defined above for Formula (I). In some embodiments, X is C—R₁, Y is C—R₄, R₂ is

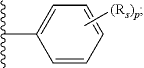

R₃ is —N(H)S(O)₂CH₃, —S(O)₂NH₂, —C(O)CH₃, or —CN; and L is a linker having the structure -A-L₁-B—, wherein: A is —NR_{c}C(O)—; L₁ is alkylene or heterocyclyl; and B is —C(O)— or —OC(O)—, wherein R₁, R₄, R₅, and p are as defined above for Formula (I). In some embodiments, A is —NR_{c}C(O)—; L₁ is C₁₋₃alkylene or 5- to 6-membered heterocyclyl; and B is —C(O)— or —OC(O)—. In some embodiments, A is —NR_{c}C(O)—; L₁ is methylene or pyrrolidine; and B is —C(O)— or —OC(O)—. In some embodiments, A is —NR_{c}C(O)—; L₁ is methylene or

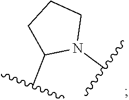 [SMILES: *C1CCCN1*]

and B is —C(O)— or —OC(O)—. In some embodiments, X is C—R₁, Y is C—R₄, R₂ is phenyl or 6-membered heteroaryl; R₃ is —N(H)S(O)₂CH₃, —S(O)₂NH₂, —C(O)CH₃, or —CN; L is a linker having the structure -A-L₁-B, wherein -A-L₁-B— is

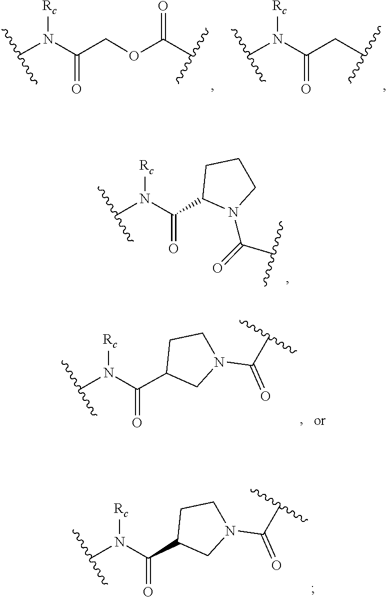

and R₁, R₄, R₅, and p are as defined above for Formula (I). In some embodiments, X is C—R₁, Y is C—R₄, R₂ is

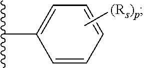

R₃ is —N(H)S(O)₂CH₃, —S(O)₂NH₂, —C(O)CH₃, or —CN; L is a linker having the structure -A-L₁-B, wherein -A-L₁-B— is

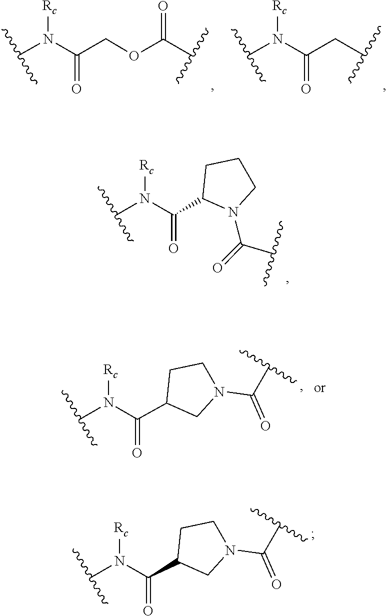

and R₁, R₄, R₅, and p are as defined above for Formula (I).

In some embodiments, the compound of Formula (I) has the structure:

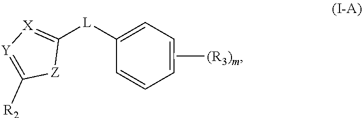

or a pharmaceutically acceptable salt or tautomer thereof, wherein L, X, Y, Z, Z₃, Z₄, Z₅, Z₆, Z₇, R₂, R₃, and m are as defined above in Formula (I).

In some embodiments, the compound of Formula (I) has the structure:

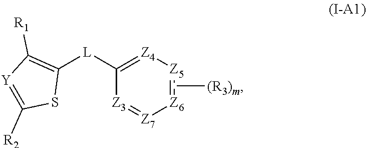

or a pharmaceutically acceptable salt or tautomer thereof, wherein L, Y, Z₃, Z₄, Z₅, Z₆, Z₇, R₁, R₂, R₃, and m are as defined above in Formula (I).

In some embodiments, the compound of Formula (I) has the structure:

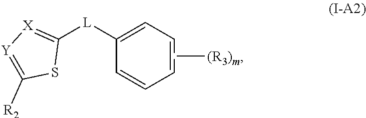

or a pharmaceutically acceptable salt thereof, wherein L, X, Y, R₂, R₃, and m are as defined above in Formula (I).

In some embodiments, the compound of Formula (I) has the structure:

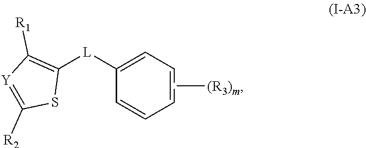

or a pharmaceutically acceptable salt thereof, wherein L, Y, R₁, R₂, R₃, and m are as defined above in Formula (I).

In some embodiments, the compound of Formula (I) has the structure:

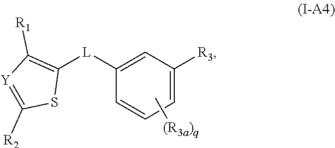

or a pharmaceutically acceptable salt thereof,
wherein:
L, Y, R₁, R₂, and R₃ are as defined above in Formula (I);
R₃ₐ is each independently H, halogen, —OH, —CN, alkyl, haloalkyl, alkoxy, or haloalkoxy; and
q is 0, 1, or 2.

In some embodiments, R₃ₐ is each independently F, —OH, C₁₋₅alkyl, C₁₋₅alkoxy, CF₃, or —OCF₃. In some embodiments, R₃ₐ is each independently F, —OH, Me, Et, iPr, —OMe, OEt, OiPr, CF₃, or —OCF₃. In some embodiments, R₃ₐ is H.

In some embodiments, q is 0 or 1. In some embodiments, q is 0. In some embodiments, q is 1. In some embodiments, q is 2.

In some embodiments, the compound of Formula (I) has the structure:

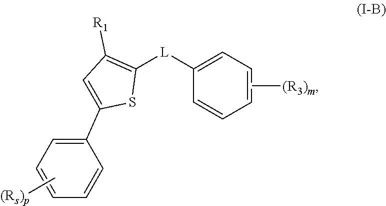

or a pharmaceutically acceptable salt thereof,
wherein:
L, R₁, R₃, and m are as defined herein;
Rₛ is each independently halogen, alkyl, haloalkyl, alkoxy, haloalkoxy, sulfonyl, or two R_{c} taken together with the atoms to which they are attached form an aryl, heteroaryl, cycloalkyl, or heterocycloalkyl; and
p is 0, 1, 2, or 3.

In some embodiments, the compound of Formula (I) has the structure:

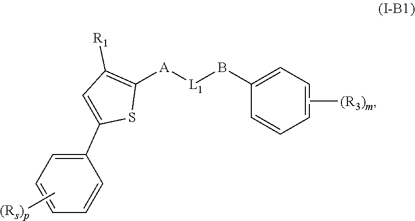

or a pharmaceutically acceptable salt thereof,
wherein:
A, B, L₁, R₁, R₃, and m are as defined herein;
Rₛ is each independently halogen, alkyl, haloalkyl, alkoxy, haloalkoxy, sulfonyl, or two Rₛ taken together with the atoms to which they are attached form an aryl, heteroaryl, cycloalkyl, or heterocycloalkyl; and
p is 0, 1, 2, or 3.

In some embodiments, Rₛ is each independently H, F, C₁₋₅alkyl, —OC₁₋₅alkyl, CF₃, —OCF₃, or —CN. In some embodiments, Rₛ is each independently H or F. In some embodiments, p is 0 or 1. In some embodiments, p is 1 or 2. In some embodiments, p is 0. In some embodiments, p is 1. In some embodiments, p is 2. In some embodiments, p is 1 and R_{c} is in the para position. In some embodiments, p is 1 and R_{c} is in the meta position.

In some embodiments, the compound of Formula (I) has the structure:

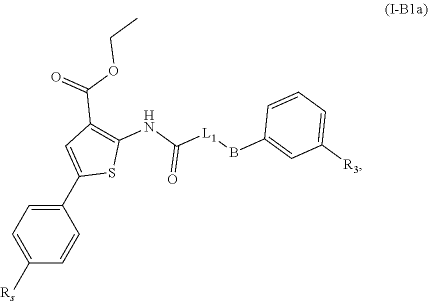

or a pharmaceutically acceptable salt thereof,
wherein:
-L₁-B— is —CH₂—O—C(O)—,

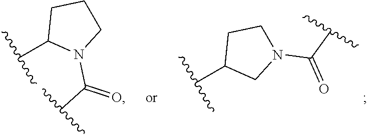 [SMILES: *C(=O)N1CCCC1* ; *C(=O)N1CCC(*)C1]

R₃ is

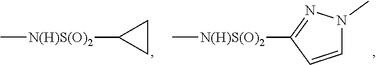

—S(O)₂NH₂, —C(O)CH₃, or —N(H)S(O)₂CH₃; and
Rₛ is H or F.

In some embodiments, -L₁-B— is —CH₂—O—C(O)—. In some embodiments, -L₁-B— is

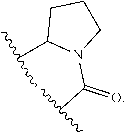 [SMILES: *C(=O)N1CCCC1*]

In some embodiments, -L₁-B— is

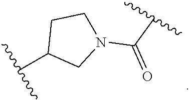 [SMILES: *C(=O)N1CCC(*)C1]

In some embodiments, R₃ is —S(O)₂NH₂ or —N(H)S(O)₂CH₃.

In some embodiments, Rₛ is H. In some embodiments, Rₛ is F.

In some embodiments, the compound of Formula (I) has the structure:

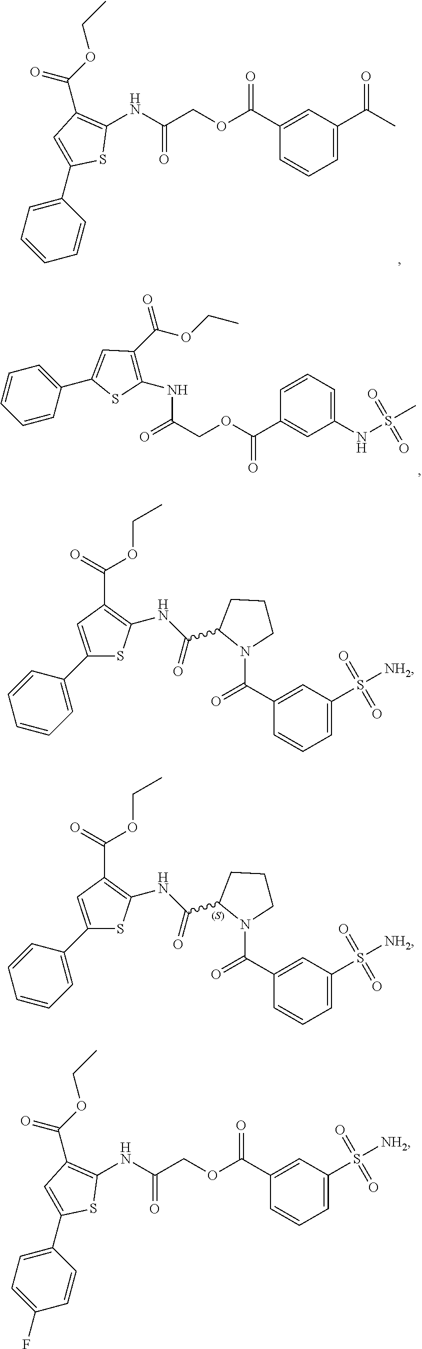

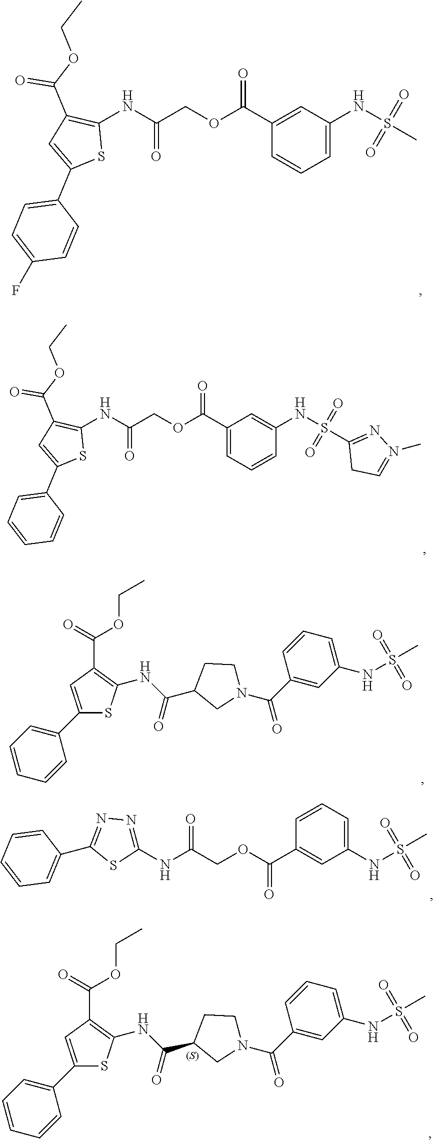

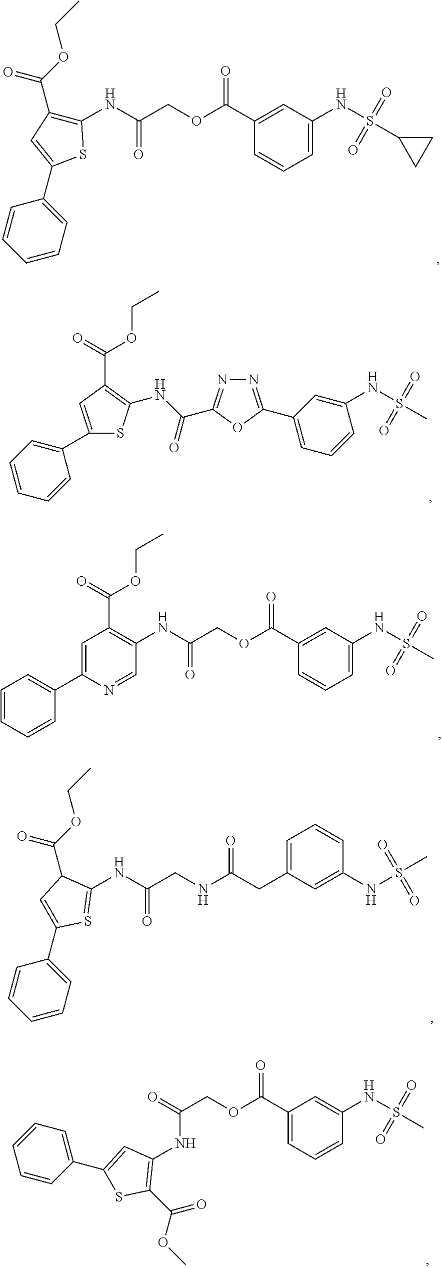

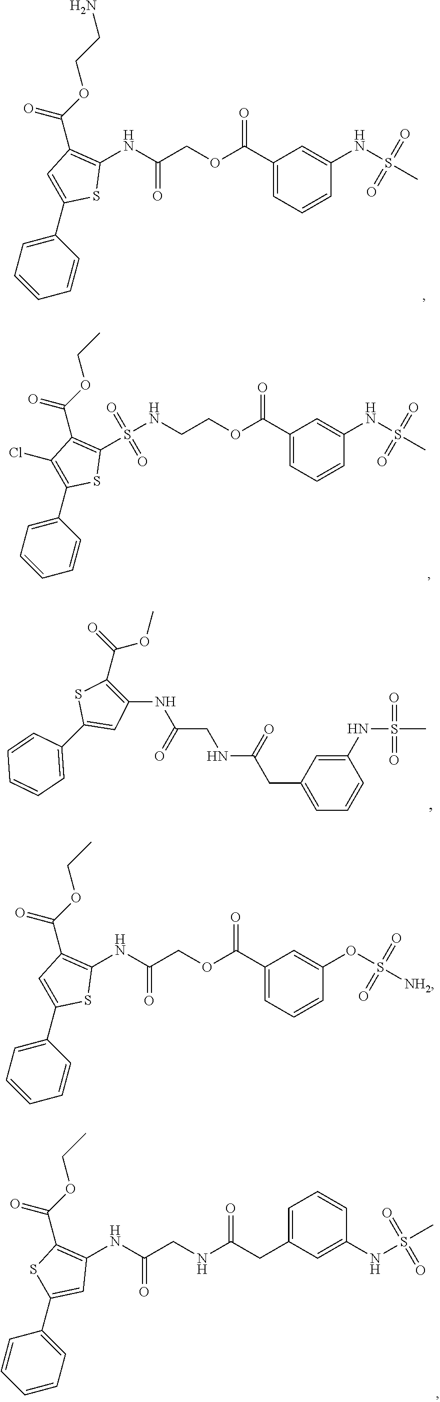

or a pharmaceutically salt or tautomer thereof.

In some embodiments, the compound of Formula (I) has the structure:

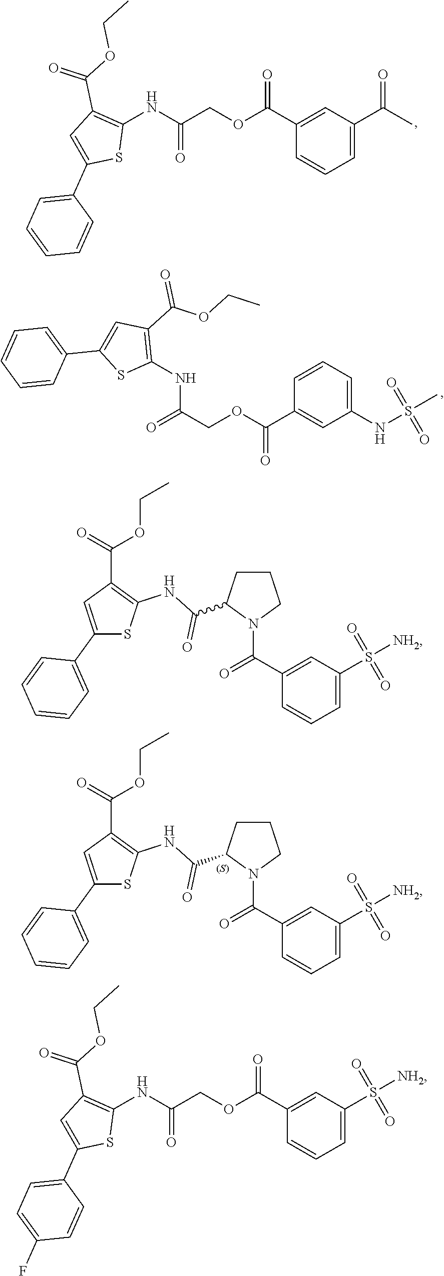

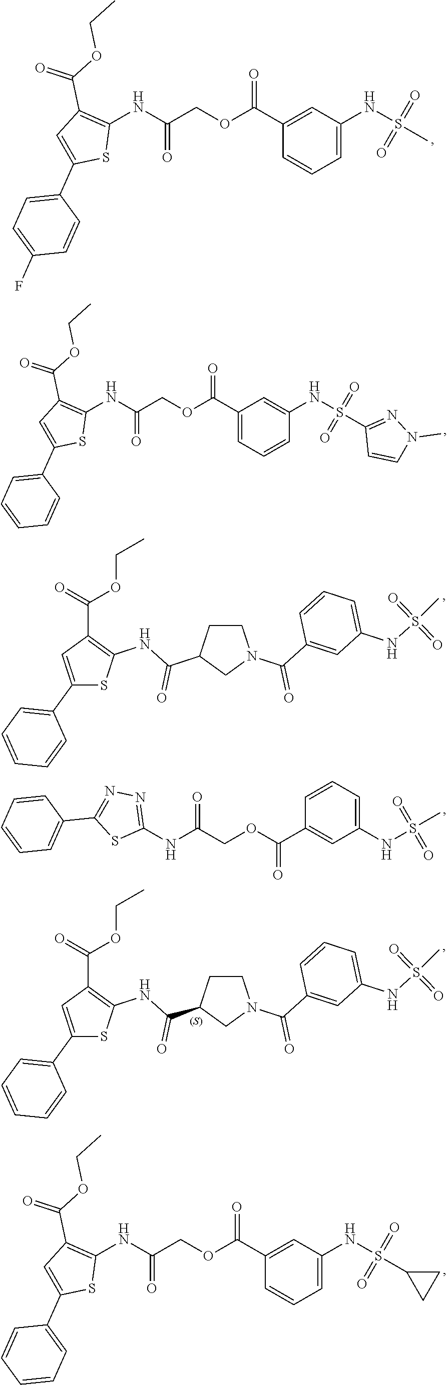

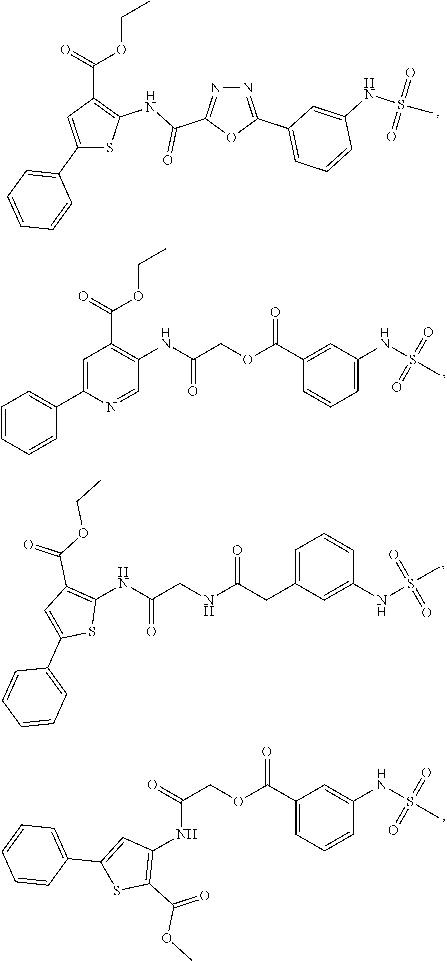 [SMILES: CCOC(=O)c1cc(-c2ccccc2)sc1NC(=O)c1nnc(-c2cccc(NS(C)(=O)=O)c2)o1 ; CCOC(=O)c1cc(-c2ccccc2)ncc1NC(=O)COC(=O)c1cccc(NS(C)(=O)=O)c1 ; CCOC(=O)c1cc(-c2ccccc2)sc1NC(=O)CNC(=O)Cc1cccc(NS(C)(=O)=O)c1 ; COC(=O)c1sc(-c2ccccc2)cc1NC(=O)COC(=O)c1cccc(NS(C)(=O)=O)c1]

or a pharmaceutically salt or tautomer thereof.

In some embodiments, the compound of Formula (I) has the structure:

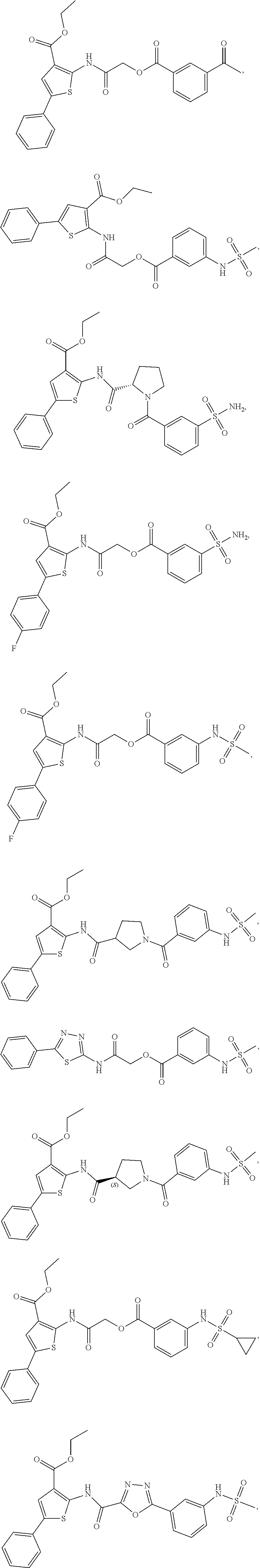

or a pharmaceutically salt or tautomer thereof.

In some embodiments of the present disclosure provides a compound of Formula (II):

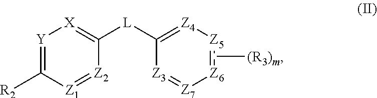

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, or —S(O)₂NR_{c}—;
L₁ is a absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

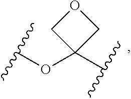 [SMILES: *OC1(*)COC1]

—OC(CF₃)—, —O—, —C(O)-alkylene-, —C(O)—, —NR_{d}S(O₂)—, —S(O₂) NR_{d}—, or heteroaryl; and
R_{c} and R_{d} are each independently H, alkyl, cycloalkyl, heterocyclyl, alkylene-aryl, acyl, or sulfonyl,
   with the proviso that -A-L₁-B— does not contain an —O—O, —NR_{c}—O, —O—NR_{d}—, —NR_{c}—NR_{d}—, or S—S bond;
X is C—R₁ or N;
Y is C—R₄ or N;
Z₁ is N or C—R₅
Z₂ is N or CH;
Z₃, Z₄, Z₅, Z₆, and Z₇ are each independently N or CH, wherein no more than 2 of Y, Z₁, and Z₂ are N and no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl)(Rₐ), —C(O)N(Rₐ)(Rₐ), —NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, halogen, alkyl, alkoxy, aryl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
   wherein at least one of R₁, R₂, and R₄ is not H.

In some embodiments, X is C—R₁. In some embodiments, X is N.

In some embodiments, Y is C—R₄. In some embodiments, Y is N.

In some embodiments, Z₁ is N. In some embodiments, Z₁ is C—R₅.

In some embodiments, Z₂ is N. In some embodiments, Z₂ is CH.

X, Y, Z₁, and Z₂ can be combined in any manner that provides a stable heteroaryl ring, such that no more than 2 of Y, Z₁, and Z₂ are N. In some embodiments, X is C—R₁, Y is C—R₄, Z₁ is N, and Z₂ is CH. In some embodiments, X is C—R₁, Y is N, Z₁ is C—R₅, and Z₂ is CH. In some embodiments, X is N, Y is C—R₄, Z₁ is C—R₅, and Z₂ is CH. In some embodiments, X is C—R₁, Y is C—R₄, Z₁ is C—R₅, and Z₂ is N. In some embodiments, X is C—R₁, Y is N, Z₁ is N, and Z₂ is CH. In some embodiments, X is N, Y is C—R₄, Z₁ is N, and Z₂ is CH. In some embodiments, X is C—R₁, Y is N, Z₁ is C—R₅, and Z₂ is N.

In some embodiments, Z₃, Z₄, Z₅, Z₆, and Z₇ is each H. In some embodiments, no more than one of Z₃, Z₄, Z₅, Z₆, and Z₇ is N. In some embodiments, one of Z₃, Z₄, Z₅, Z₆, and Z₇ is N. In some embodiments, Z₃ is N and Z₄, Z₅, Z₆, and Z₇ are each H. In some embodiments, Z₄ is N and Z₃, Z₅, Z₆, and Z₇ are each H. In some embodiments, Z₅ is N and Z₃, Z₄, Z₆, and Z₇ are each H. In some embodiments, Z₆ is N and Z₃, Z₄, Z₄, and Z₇ are each H. In some embodiments, Z₇ is N and Z₃, Z₄, Z₅, and Z₆ are each H. In some embodiments, no more than two of Z₃, Z₄, Z₅, Z₆, and Z₇ are each N. In some embodiments, two of Z₃, Z₄, Z₅, Z₆, and Z₇ are each N. In some embodiments, Z₃ and Z₄ are each N. In some embodiments, Z₃ and Z₅ are each N. In some embodiments, Z₃ and Z₆ are each N. In some embodiments, Z₄ and Z₆ are each N. In some embodiments, Z₄ and Z₇ are each N. In some embodiments, Z₅ and Z₇ are each N. In some embodiments, Z₃ and Z₇ are each N. In some embodiments, Z₆ and Z₇ are each N. In some embodiments, Z₅ and Z₆ are each N. In some embodiments, Z₄ and Z₅ are each N.

In some embodiments, A is, —NR_{c}—, —NR_{c}C(O)—, —C(O)NR_{c}—, —NR_{c}S(O)₂—, or —S(O)₂NR_{c}—. In some embodiments, A is —NR_{c}C(O)—, —C(O)NR_{c}—, —NR_{c}S(O)₂—, or —S(O)₂NR_{c}—. In some embodiments, A is —NR_{c}C(O)—, —C(O)NR_{c}—, or —C(O)—. In some embodiments, A is —NR_{c}C(O)— or —NR_{c}S(O)₂—. In some embodiments, A is —NR_{c}C(O)—. In some embodiments, R_{c} is H, C₁₋₅alkyl, C₃₋₆cycloalkyl, or —CH₂-phenyl. In some embodiments, R_{c} is H or C₁₋₅alkyl. In some embodiments, the C₁₋₅alkyl is methyl, ethyl, or isopropyl. In some embodiments, the C₁₋₅alkyl is methyl. In some embodiments, the C₃₋₆cycloalkyl is cyclopropyl. In some embodiments, R_{c} is H or methyl. In some embodiments, R_{c} is H.

In some embodiments, L₁ is absent, alkylene, cycloalkyl, heterocyclyl, or heteroaryl. In some embodiments, L₁ is absent, alkylene, cycloalkyl, or heterocyclyl. In some embodiments, L₁ is absent, alkylene, or heterocyclyl. In some embodiments, L₁ is alkylene or heterocyclyl. In some embodiments, the alkylene is a C₁₋₅alkylene. In some embodiments, the alkylene is a C₁₋₃alkylene. In some embodiments, the alkylene is methylene. In some embodiments, L₁ is alkylene, and the alkylene is a C₁₋₃alkylene. In some embodiments, the alkylene is an optionally substituted methylene. In some embodiments, the alkylene comprises the structure:

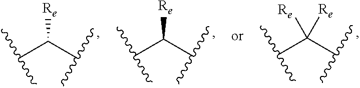

wherein Rₑ is each independently H, F, C₁₋₅ alkyl, —CH₂OH, —CH₂cycloalkyl, — or CH₂aryl, or two Rₑ taken together with the carbon atom to which they are attached form a C₃₋₆ cycloalkyl or a 4- to 6-membered heterocyclyl. In some embodiments, Rₑ is H or C₁₋₅ alkyl.

In some embodiments, L₁ comprises a heterocyclyl. In some embodiments, the heterocyclyl is a 3- to 12-membered heterocyclyl. In some embodiments, the heterocyclyl has the structure:

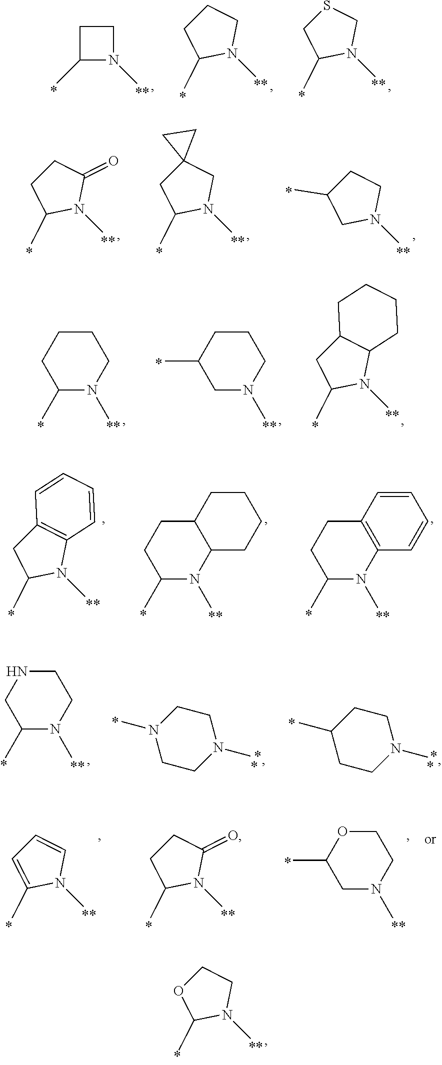

wherein * represents the attachment point to A and ** represents the attachment point to B.

In some embodiments, B is bond, —NR_{b}C(O)—, —NR_{b}S(O)₂—, —C(O)—, —OC(O)—, —C(O)—O—, or a heteroaryl. In some embodiments, B is a bond, —OC(O)—, or —C(O)—. In some embodiments, B is —OC(O)—. In some embodiments, B is —C(O)—. In some embodiments, B is a bond. In some embodiments, B is heteroaryl. In some embodiments, the heteroaryl is a 5- or 6-membered nitrogen-containing heteroaryl. In some embodiments, the heteroaryl is a triazole. In some embodiments, the heteroaryl is a 1,2,3-triazole or 1,2,4-triazole.

In some embodiments, L is a linker having the structure -A-L₁-B—, wherein A is —NR_{c}C(O)— or —C(O)NR_{c}; L₁ is absent, alkylene, cycloalkyl, or heterocyclyl; B is a bond, —NR_{d}—, —N(S(O)₂-alkyl)-, —NR_{d}C(O)—, —OC(O)—, —C(O)-alkylene-, —C(O)—, —NR_{d}S(O₂)—, or —S(O₂) NR_{d}—; and R_{c} and R_{d} are each independently H, alkyl, cycloalkyl, or alkylene-aryl, with the proviso that -A-L₁-B— does not contain an —O—O, —NR_{c}—O, —O—NR_{d}—, —NR_{c}—NR_{d}—, or S—S bond. In some embodiments, R_{c} and R_{d} are each independently H, alkyl, cycloalkyl, alkylene-aryl, acyl, or sulfonyl. In some embodiments, R_{c} and R_{d} are each independently H or alkyl. In some embodiments, R_{c} and R_{d} are each independently H or C₁₋₅alkyl. In some embodiments, R_{c} and Rₐ are each H.

In some embodiments, L is a linker having the structure -A-L₁-B—, wherein: A is —NR_{c}C(O)—; L₁ is alkylene or heterocyclyl; and B is —C(O)— or —OC(O)—. In some embodiments, A is —NR_{c}C(O)—; L₁ is C₁₋₃alkylene or 5- to 6-membered heterocyclyl; and B is —C(O)— or —OC(O)—. In some embodiments, A is —NR_{c}C(O)—; L₁ is methylene or pyrrolidine; and B is —C(O)— or —OC(O)—. In some embodiments, A is —NR_{c}C(O)—; L₁ is methylene or

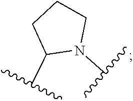 [SMILES: *C1CCCN1*]

and B is —C(O)— or —OC(O)—. In some embodiments, L is a linker having the structure -A-L₁-B—, wherein: A is —NR_{c}C(O)—; L₁ is absent; and B is a heteroaryl. In some embodiments, the heteroaryl is a 5-membered heteroaryl. In some embodiments, the heteroaryl is an oxadiazolyl, thiadiazolyl, triazolyl, oxazolyl, thiazolyl, or imidazolyl. In some embodiments, the heteroaryl has the structure of

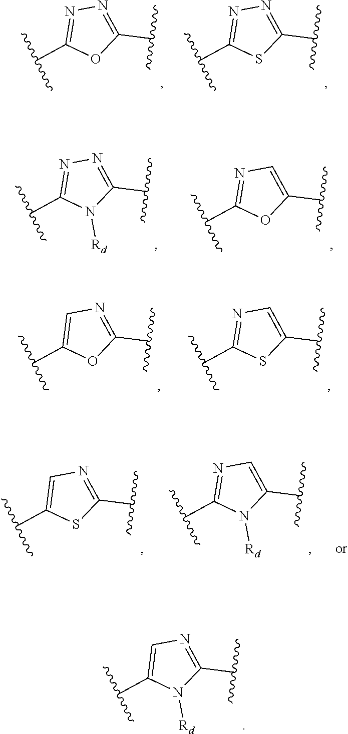

In some embodiments, -A-L₁-B— has the structure:

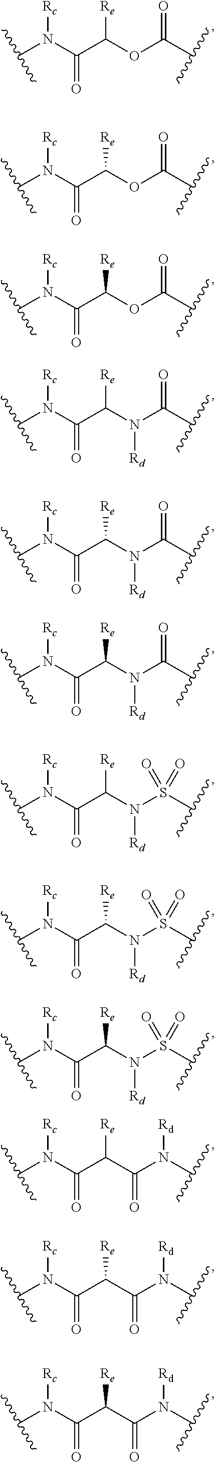

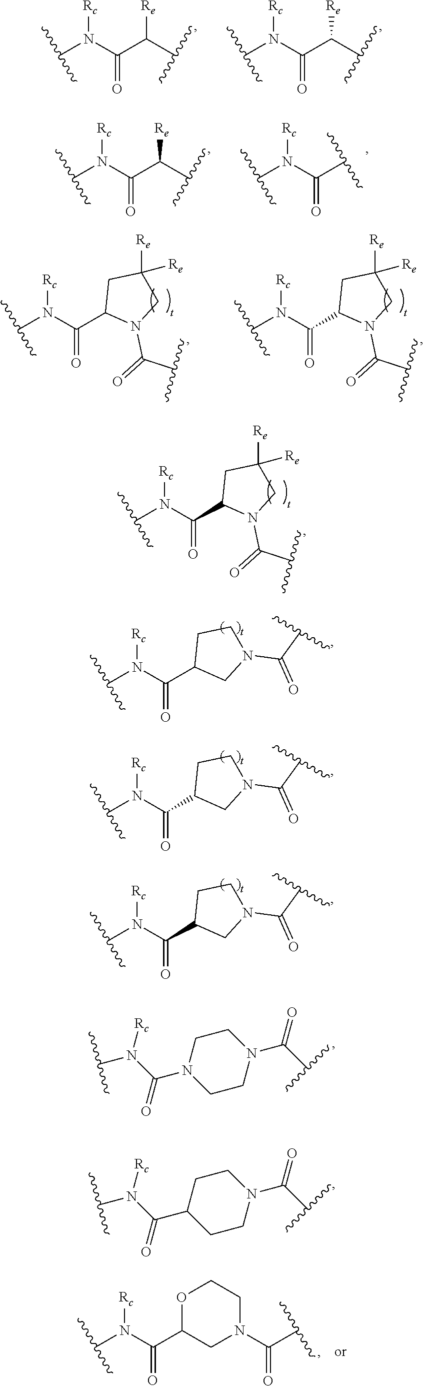

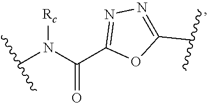

wherein R_{c} and R_{d} are each independently H, alkyl, cycloalkyl, alkylene-aryl, acyl, or sulfonyl; Rₑ is each independently H, F, C₁₋₅ alkyl, —CH₂cycloalkyl, — or CH₂aryl, or two Rₑ taken together with the carbon atom to which they are attached form a C₃₋₆ cycloalkyl or a 4- to 6-membered heterocyclyl; and t is 0, 1 or 2. In some embodiments, R_{c} is H or alkyl. In some embodiments, R_{c} is H or C₁₋₅alkyl. In some embodiments, R_{c} is H. In some embodiments, Rₑ is each independently H, F, or C₁₋₅ alkyl. In some embodiments, Rₑ is each independently H, F, Me, Et, or iPr. In some embodiments, Rₑ is H. In some embodiments, t is 1.

In some embodiments, -A-L₁-B— has the structure:

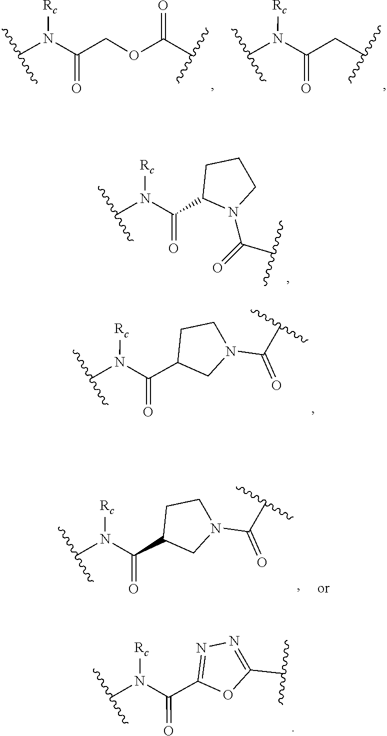

In some embodiments, R_{c} is H or alkyl. In some embodiments, R_{c} is H or C₁₋₅alkyl. In some embodiments, R_{c} is H.

In some embodiments, R₁ is alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ. In some embodiments, R₁ is alkyl, —C(O)ORₐ, —C(O)N(Rₐ)(Rₐ), or —C(O)NHRₐ. In some embodiments, R₁ is —C(O)ORₐ. In some embodiments, each Rₐ is independently H or C₁₋₅ alkyl (e.g., methyl, ethyl, or n-propyl, or i-propyl), or two Rₐ groups taken together with the nitrogen atom to which they are attached form a heterocyclyl. In some embodiments, the heterocyclyl is a 5- or 6-membered nitrogen-containing heterocyclyl. In some embodiments, the heterocyclyl has the structure:

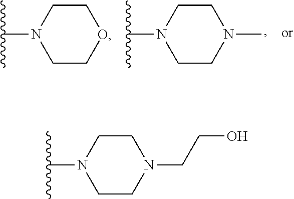

In some embodiments, Rₐ is C₁₋₅ alkyl. In some embodiments, Rₐ is methyl, ethyl, or n-propyl, or i-propyl. In some embodiments, Rₐ is ethyl.

In some embodiments, X is C—R₁, and R₁ and R_{c} taken together with the atoms to which they are attached form a heterocyclyl or heteroaryl.

In some embodiments, R₄ is H, alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ. In some embodiments, R₄ is H, alkyl, aryl, —C(O)ORₐ, or —C(O)NHRₐ. In some embodiments, R₄ is H, alkyl, —C(O)ORₐ, —C(O)N(Rₐ)(Rₐ), or —C(O)NHRₐ. In some embodiments, R₄ is H or —C(O)ORₐ. In some embodiments, R₄ is H. In some embodiments, Rₐ is C₁₋₅ alkyl or —CH₂cycloalkyl. In some embodiments, Rₐ is C₁₋₅ alkyl. In some embodiments, Rₐ is methyl, ethyl, or n-propyl, or i-propyl. In some embodiments, each Rₐ is independently H or C₁₋₅ alkyl (e.g., methyl, ethyl, or n-propyl, or i-propyl), or two Rₐ groups taken together with the nitrogen atom to which they are attached form a heterocyclyl. In some embodiments, the heterocyclyl is a 5- or 6-membered nitrogen-containing heterocyclyl. In some embodiments, the heterocyclyl has the structure:

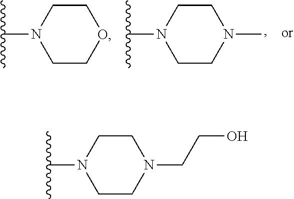

In some embodiments, Rₐ is C₁₋₅ alkyl. In some embodiments, Rₐ is methyl, ethyl, or n-propyl, or i-propyl. In some embodiments, Rₐ is ethyl.

In some embodiments, R₂ is aryl or heteroaryl. In some embodiments, the aryl is phenyl and the heteroaryl is 6-membered nitrogen-containing heteroaryl. In some embodiments, the aryl is phenyl and the heteroaryl is a pyridyl. In some embodiments, R₂ is phenyl or naphthyl. In some embodiments, R₂ is phenyl. In some embodiments, R₂ is

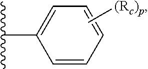

wherein R_{c} is each independently halogen, alkyl, haloalkyl, —OH, alkoxy, haloalkoxy, or amino (e.g., —NH₂, —NHCH₃, —N(CH₃)₂, etc.), or two R_{c} taken together with the atoms to which they are attached form an aryl, heteroaryl, cycloalkyl, or heterocycloalkyl; and p is 0, 1, 2, or 3. In some embodiments, R₂ is

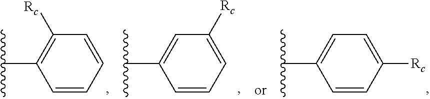

wherein R_{c} is each independently halogen, alkyl, haloalkyl, —OH, alkoxy, haloalkoxy, or amino. In some embodiments, R₂ is

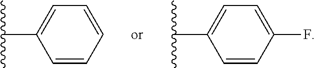 [SMILES: *c1ccccc1 ; *c1ccc(F)cc1]

In some embodiments, R₂ is a 6-membered nitrogen-containing heteroaryl. In some embodiments, R₂ is

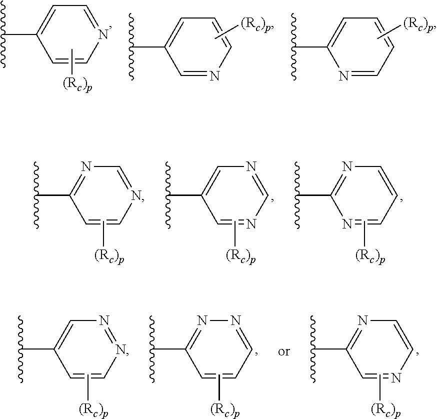

wherein R_{c} is each independently halogen, alkyl, haloalkyl, —OH, alkoxy, haloalkoxy, or amino, or two R_{c} taken together with the atoms to which they are attached form an aryl, heteroaryl, cycloalkyl, or heterocycloalkyl; and p is 0, 1, or 2. In some embodiments, R₂ is

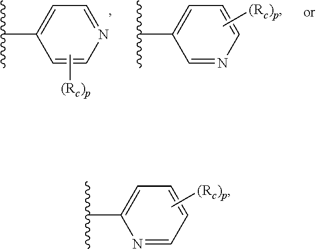

wherein R_{c} is each independently halogen, alkyl, haloalkyl, —OH, alkoxy, haloalkoxy, or amino, or two R_{c} taken together with the atoms to which they are attached form an aryl, heteroaryl, cycloalkyl, or heterocycloalkyl; and p is 0, 1, or 2. In some embodiments, R^{c} is each independently H, F, C₁₋₅alkyl, —OC₁₋₅alkyl, CF₃, —OCF₃, or —CN. In some embodiments, R_{c} is each independently H or F. In some embodiments, p is 0 or 1. In some embodiments, p is 1 or 2. In some embodiments, p is 0. In some embodiments, p is 1. In some embodiments, p is 2.

In some embodiments, R₂ and R₄ taken together with the carbon atoms to which they are attached form a cycloalkyl, heterocyclyl, aryl, or heteroaryl. In some embodiments, R₂ and R₄ taken together with the carbon atoms to which they are attached form a cycloalkyl. In some embodiments, the cycloalkyl is a C₅₋₇cycloalkyl. In some embodiments, the cycloalkyl is a C₅₋₆cycloalkyl. In some embodiments, the cycloalkyl is a C₄₋₆ cycloalkyl.

In some embodiments, when X is C—R₁ and Y is C—R₄, at least two of R₁, R₂, and R₄ are not H. In some embodiments, R¹ and R² are not H, and R₄ is H. In some embodiments, when X is C—R₁ and Y is C—R₄, R₁ is —C(O)ORₐ, —C(O)N(Rₐ)(Rₐ), or —C(O)NHRₐ; R₂ is optionally substituted phenyl; and R₄ is H, wherein Rₐ is as defined herein. In some embodiments, R₁ is —C(O)ORₐ; R₂ is optionally substituted phenyl; and R₄ is H, wherein Rₐ is as defined herein. In some embodiments, R₁ is —C(O)ORₐ; R₂ is

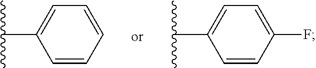 [SMILES: *c1ccccc1 ; *c1ccc(F)cc1]

and R₄ is H, wherein Rₐ is as defined herein. In some embodiments, X and Y are N and R₂ is

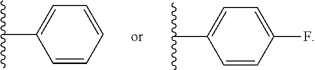 [SMILES: *c1ccccc1 ; *c1ccc(F)cc1]

In some embodiments, Z is S. In some embodiments, Z is O. In some embodiments, Z is N—R₅.

In some embodiments, R₅ is H, halogen, alkyl, alkylenecycloalkyl, cycloalkyl, aryl, or alkylenearyl. In some embodiments, R₅ is H, halogen, alkyl, or alkylenecycloalkyl. In some embodiments, R₅ is H, halogen, or alkyl. In some embodiments, R₅ is H or halogen. In some embodiments, R₅ is H or alkyl. In some embodiments, the alkyl is Me, Et, or iPr. In some embodiments, the aryl is a phenyl. In some embodiments, the cycloalkyl is a C₃₋₆cycloalkyl. In some embodiments, the alkylene is a C₁₋₃alkylene. In some embodiments, the alkylene is a methylene. In some embodiments, the halogen is F, Cl, or Br. In some embodiments, the halogen is F.

In some embodiments, R₃ is —S(O)₂NHR_{b}, —S(O)₂NR_{b}R_{b}, —NHS(O)₂R_{b}, —C(O)R_{b}, —C(O)NHR_{b}, —C(O)NR_{b}R_{b}, —NHC(O)R_{b}, —NHC(O)NR_{b}R_{b}, or —CN. In some embodiments, R₃ is —S(O)₂NHR_{b}, —S(O)₂NR_{b}R_{b}, —NHS(O)₂R_{b}, —C(O)NHR_{b}, —C(O)NR_{b}R_{b}, —NHC(O)R_{b}, —NHC(O)NR_{b}R_{b}, or —CN. In some embodiments, R₃ is —N(H)S(O)₂alkyl, —N(H)S(O)₂cycloalkyl, —S(O)₂NH₂, —C(O)alkyl, or —CN. In some embodiments, R₃ is —N(H)S(O)₂ (C₁₋₅alkyl), —S(O)₂N(C₁₋₅alkyl)₂, —S(O)₂N(H)(C₁₋₅alkyl), or —S(O)₂NH₂. In some embodiments, R₃ is —N(H)S(O)₂ (C₁₋₅alkyl) or —S(O)₂NH₂. In some embodiments, R₃ is —N(H)S(O)₂CH₃, —S(O)₂NH₂, —C(O)CH₃, or —CN. In some embodiments, R₃ is —N(H)S(O)₂CH₃ or —S(O)₂NH₂.

In some embodiments, m is 0, 1, or 2. In some embodiments, m is 0 or 1. In some embodiments, m is 1 or 2. In some embodiments, m is 0. In some embodiments, m is 1. In some embodiments, m is 2. In some embodiments, m is 3.

In some embodiments, X is C—R₁, Y is C—R₄, Z₁ is N, Z₂ is CH, R₂ is aryl, heteroaryl; R₃ is —N(H)S(O)₂CH₃, —S(O)₂NH₂, —C(O)CH₃, or —CN; and L is a linker having the structure -A-L₁-B—, wherein: A is —NR_{c}C(O)—; L₁ is alkylene or heterocyclyl; and B is —C(O)— or —OC(O)—, wherein R₁, R₂, and R₄ are as defined above for Formula (I). In some embodiments, X is C—R₁, Y is C—R₄, Z₁ is N, Z₂ is CH, R₂ is

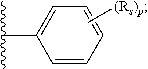

R₃ is —N(H)S(O)₂CH₃, —S(O)₂NH₂, —C(O)CH₃, or —CN; and L is a linker having the structure -A-L₁-B—, wherein: A is —NR_{c}C(O)—; L₁ is alkylene or heterocyclyl; and B is —C(O)— or —OC(O)—, wherein R₁, R₄, R₅, and p are as defined above for Formula (I). In some embodiments, A is —NR_{c}C(O)—; L₁ is C₁₋₃alkylene or 5- to 6-membered heterocyclyl; and B is —C(O)— or —OC(O)—. In some embodiments, A is —NR_{c}C(O)—; L₁ is methylene or pyrrolidine; and B is —C(O)— or —OC(O)—. In some embodiments, A is —NR_{c}C(O)—; L₁ is methylene or

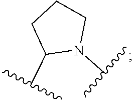 [SMILES: *C1CCCN1*]

and B is —C(O)— or —OC(O)—. In some embodiments, X Is C—R₁, Y is C—R₄, Z₁ is N, Z₂ is CH, R₂ is phenyl or 6-membered heteroaryl; R₃ is —N(H)S(O)₂CH₃, —S(O)₂NH₂, —C(O)CH₃, or —CN; L is a linker having the structure -A-L₁-B, wherein -A-L₁-B— is

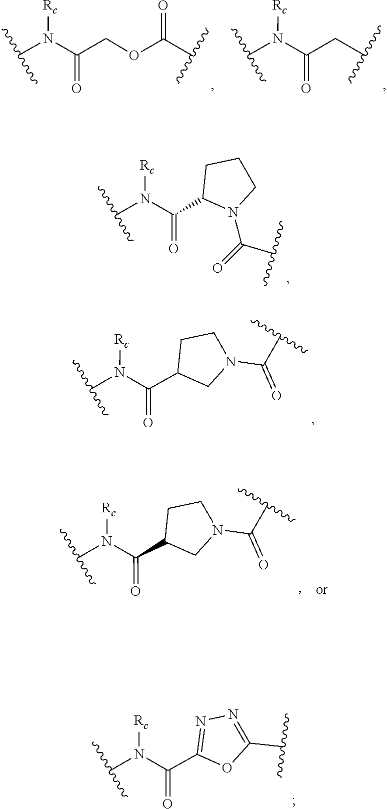

and R₁, R₄, R₅, and p are as defined above for Formula (I). In some embodiments, X is C—R₁, Y is C—R₄, Z₁ is N, Z₂ is CH, R₂ is

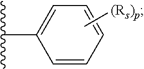

R₃ is —N(H)S(O)₂CH₃, —S(O)₂NH₂, —C(O)CH₃, or —CN; L is a linker having the structure -A-L₁-B, wherein -A-L₁-B— is

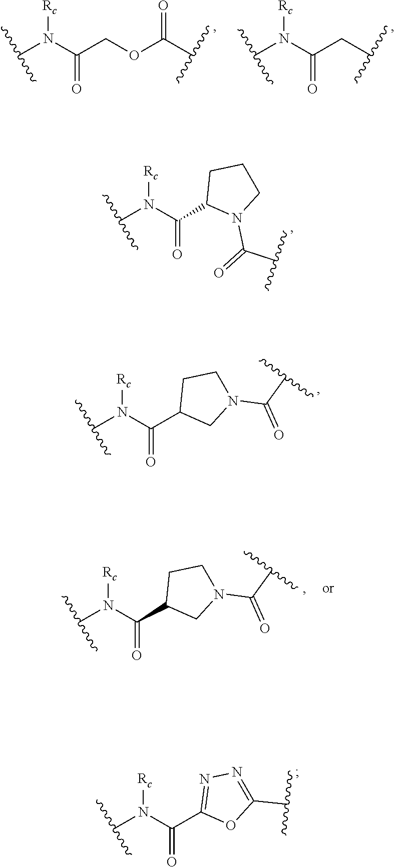

and R₁, R₄, Rₛ, and p are as defined above for Formula (I).

In some embodiments, the compound of Formula (II) has the structure:

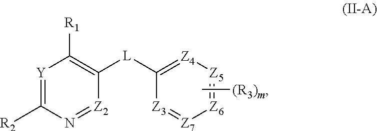

or a pharmaceutically acceptable salt or tautomer thereof, wherein L, Y, Z₂, Z₃, Z₄, Z₅, Z₆, Z₇, R₁, R₂, R₃, and m are as defined above in Formula (II).

In some embodiments, the compound of Formula (II) has the structure:

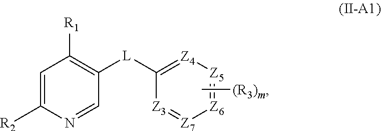

or a pharmaceutically acceptable salt or tautomer thereof, wherein L, Z₃, Z₄, Z₅, Z₆, Z₇, R₁, R₂, R₃, and m are as defined above in Formula (II).

In some embodiments, the compound of Formula (II) has the structure:

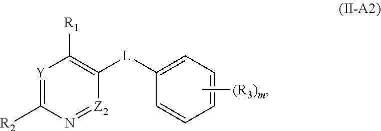

or a pharmaceutically acceptable salt thereof, wherein L, Y, Z₂, Z₃, Z₄, Z₅, Z₆, Z₇, R₁, R₂, R₃, and m are as defined above in Formula (II).

In some embodiments, R₃ₐ is each independently F, —OH, C₁₋₅alkyl, C₁₋₅alkoxy, CF₃, or —OCF₃. In some embodiments, R₃ₐ is each independently F, —OH, Me, Et, iPr, —OMe, OEt, OiPr, CF₃, or —OCF₃. In some embodiments, R₃ₐ is H.

In some embodiments, q is 0 or 1. In some embodiments, q is 0. In some embodiments, q is 1. In some embodiments, q is 2.

In some embodiments, the compound of Formula (II) has the structure:

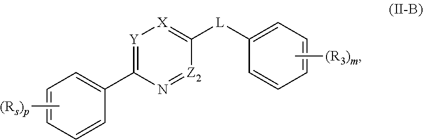

or a pharmaceutically acceptable salt thereof,
wherein:
L, X, Y, Z₂, R₃, and m are as defined herein;
R_{c} is each independently halogen, alkyl, haloalkyl, alkoxy, haloalkoxy, sulfonyl, or two R_{c} taken together with the atoms to which they are attached form an aryl, heteroaryl, cycloalkyl, or heterocycloalkyl; and
p is 0, 1, 2, or 3.

In some embodiments, the compound of Formula (II) has the structure:

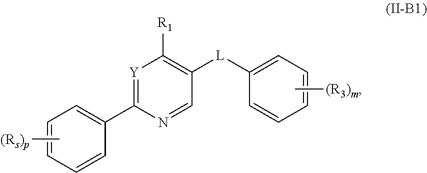

or a pharmaceutically acceptable salt thereof,
wherein:
L, Y, R₁, R₃, and m are as defined herein;
R_{c} is each independently halogen, alkyl, haloalkyl, alkoxy, haloalkoxy, sulfonyl, or two R_{c} taken together with the atoms to which they are attached form an aryl, heteroaryl, cycloalkyl, or heterocycloalkyl; and
p is 0, 1, 2, or 3.

In some embodiments, R_{c} is each independently H, F, C₁₋₅alkyl, —OC₁₋₅alkyl, CF₃, —OCF₃, or —CN. In some embodiments, R_{c} is each independently H or F. In some embodiments, p is 0 or 1. In some embodiments, p is 1 or 2. In some embodiments, p is 0. In some embodiments, p is 1. In some embodiments, p is 2. In some embodiments, p is 1 and R_{c} is in the para position. In some embodiments, p is 1 and R_{c} is in the meta position.

In some embodiments, the compound of Formula (II) has the structure:

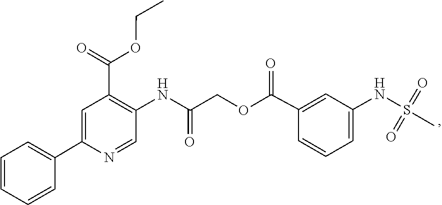 [SMILES: CCOC(=O)c1cc(-c2ccccc2)ncc1NC(=O)COC(=O)c1cccc(NS(C)(=O)=O)c1]

or a pharmaceutically salt or tautomer thereof.

In some aspects, the present disclosure further provides a compound of Formula (III) having the structure:

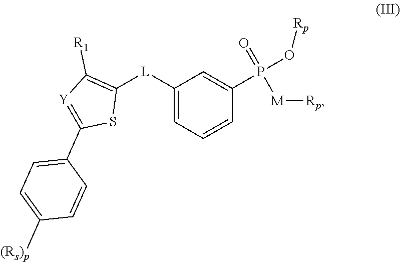

or a pharmaceutically acceptable salt thereof,
wherein:
L, Y, R₁, Rₛ, and p are as defined above in Formula (I);
M is O or —NH; and
Rₚ is each independently -alkylene-O—C(O)-alkyl, -alkylene-O—C(O)—Oalkyl, -alkylene-C(O)—Oalkyl, or aryl; or two Rₚ taken together with the atoms to which they are attached form a heterocyclyl ring.

In some embodiments, Rₚ is each independently -alkylene-O—C(O)-alkyl, -alkylene-O—C(O)—Oalkyl, -alkylene-C(O)—Oalkyl, or aryl, wherein the alkylene is a C₁₋₃alkylene, the alkyl is a C₁₋₅alkyl, and the aryl is a phenyl. In some embodiments, the alkylene is —CH(Me)—. In some embodiments, the C₁₋₅alkyl is methyl, ethyl, isopropyl, or tert-butyl. In some embodiments, Rₚ is each independently —CH₂—O—C(O)-alkyl, —CH₂—O—C(O)—Oalkyl, —CH₂—C(O)—Oalkyl, or phenyl. In some embodiments, Rₚ is each independently —CH₂—O—C(O)—C₁₋₅alkyl, —CH(Me)—O—C(O)—C₁₋₅alkyl, —CH₂—O—C(O)—O(C₁₋₅alkyl), —CH(Me)—O—C(O)—O(C₁₋₅alkyl), —CH₂—C(O)—O(C₁₋₅alkyl), —CH(Me)—C(O)—O(C₁₋₅alkyl), or phenyl. In some embodiments, Rₚ is each independently —CH₂—O—C(O)—C₁₋₅alkyl, —CH₂—O—C(O)—O(C₁₋₅alkyl), —CH₂—C(O)—O(C₁₋₅alkyl), or phenyl. In some embodiments, the C₁₋₅alkyl is methyl, ethyl, isopropyl, or tert-butyl. In some embodiments, the C₁₋₅alkyl is isopropyl or tert-butyl.

In some embodiments, two Rₚ taken together with the atoms to which they are attached form a 5- to 10-membered heterocyclyl ring. In some embodiments, the heterocyclyl ring comprises a disulfide bond. In some embodiments, the heterocyclyl ring has the structure:

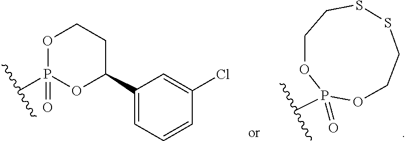 [SMILES: *P1(=O)OCC[C@@H](c2cccc(Cl)c2)O1 ; *P1(=O)OCCSSCCO1]

In some embodiments, M is O. In some embodiments, M is —NH.

In some embodiments, the compound of Formula (III) has the structure:

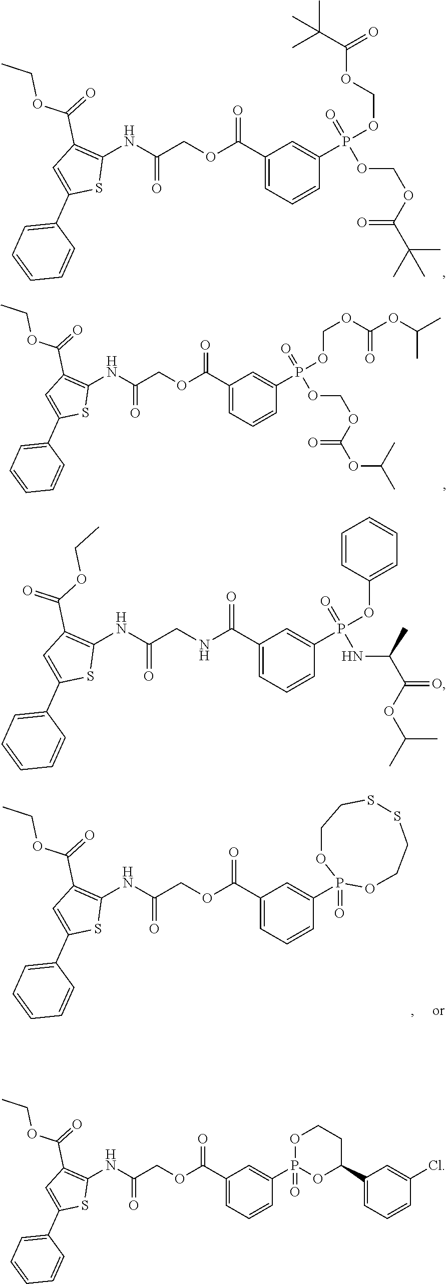

In some aspects, the present disclosure further provides a compound of Formula A having the structure:

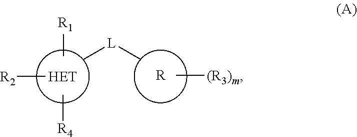

or a pharmaceutically acceptable salt thereof,
wherein:

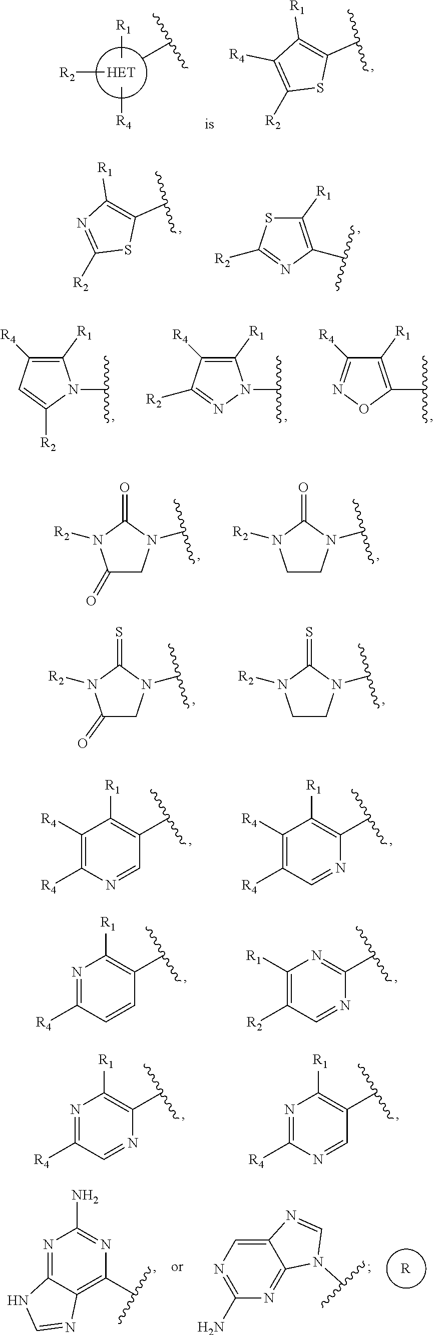

is aryl, heteroaryl, cycloalkyl, or heterocyclyl;
L is a linker having structure -A-L₁-B—, wherein A, L₁, B, R₁, R₂, R₃, R₄, and m are as defined above for Formula (I) and Formula (II),
wherein at least one of R₁, R₂, and R₄ is not H.

In some embodiments,

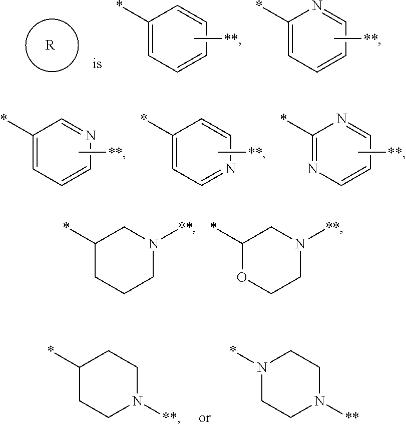

wherein * represents the attachment point to L and ** represents the attachment point to the one or more R₃. In some embodiments,

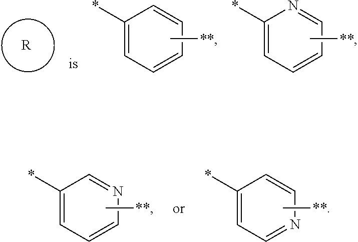

In some embodiments,

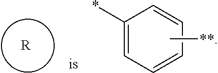

In some embodiments, the compound disclosed herein has one of the following structures:

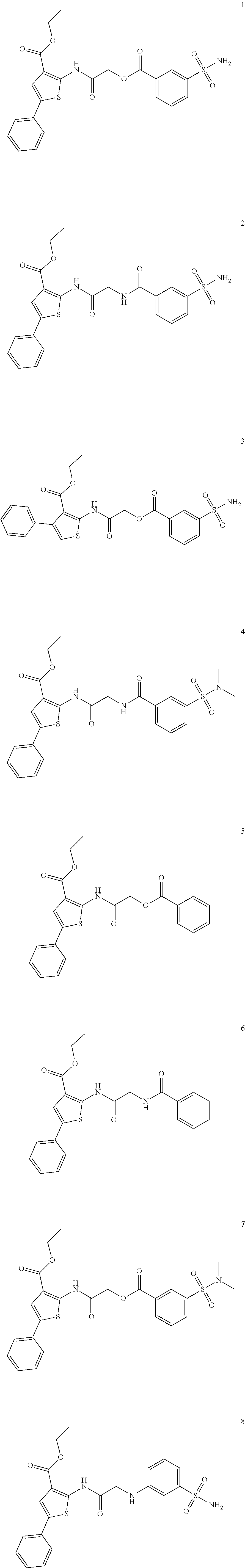

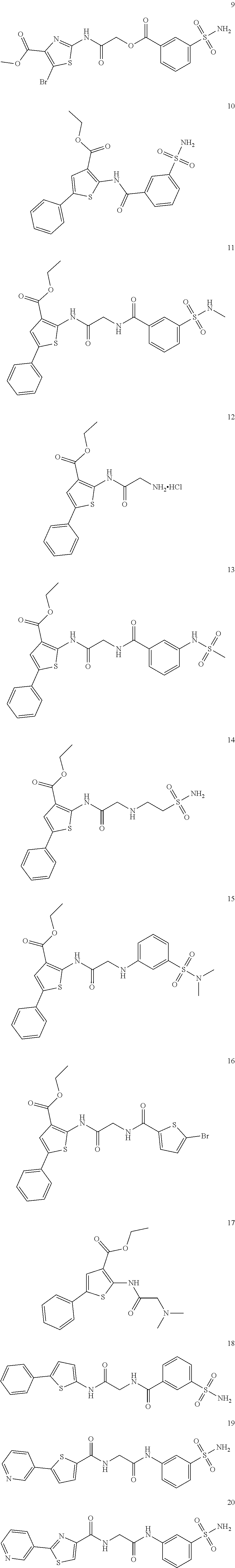

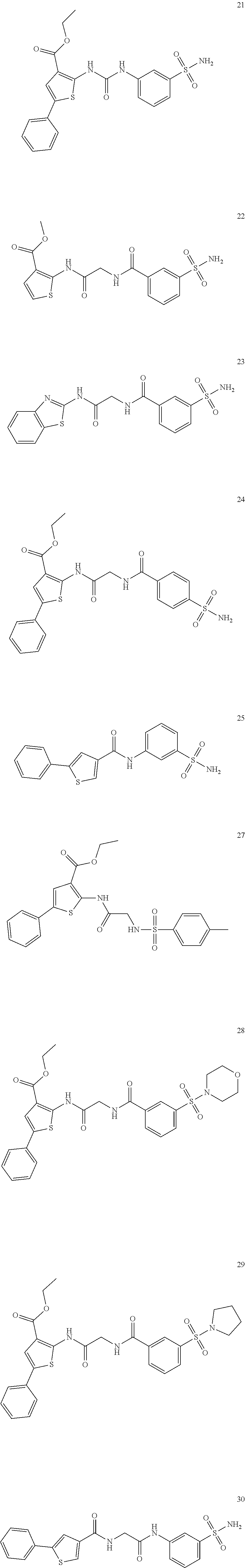

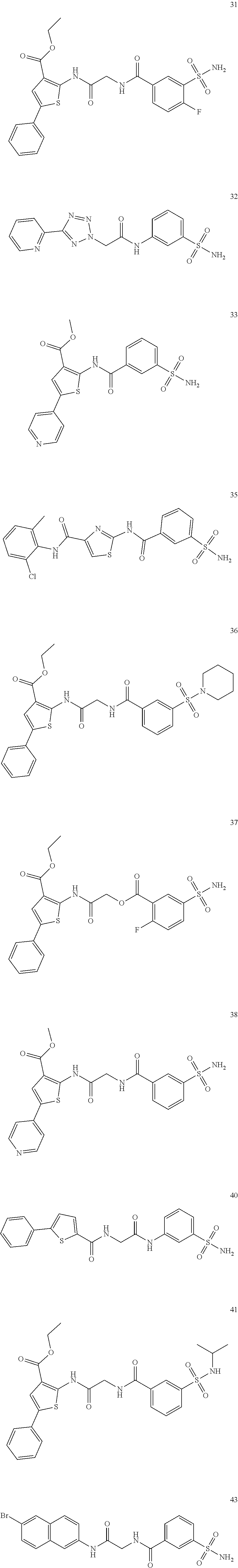

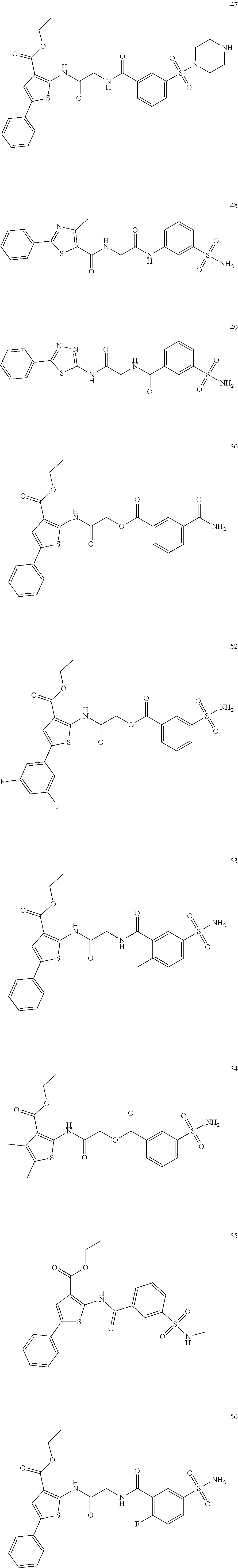

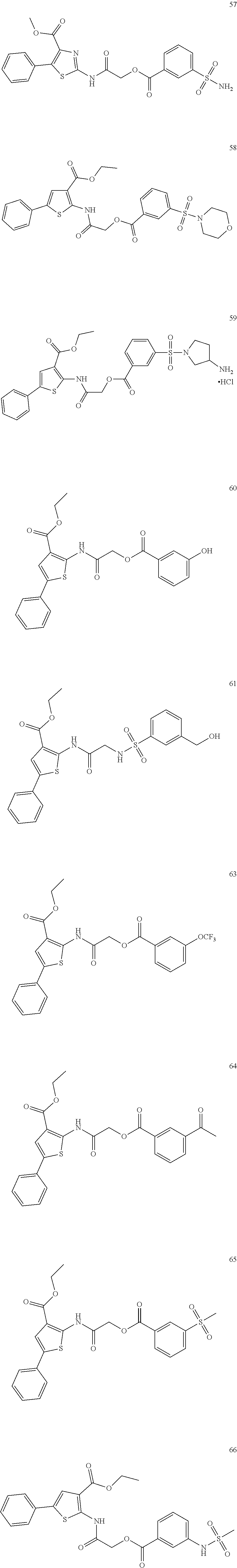

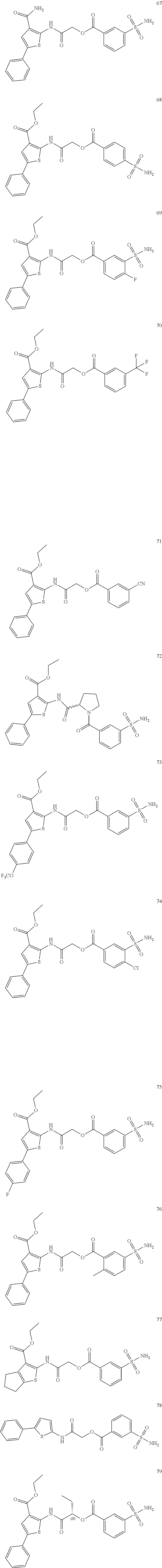

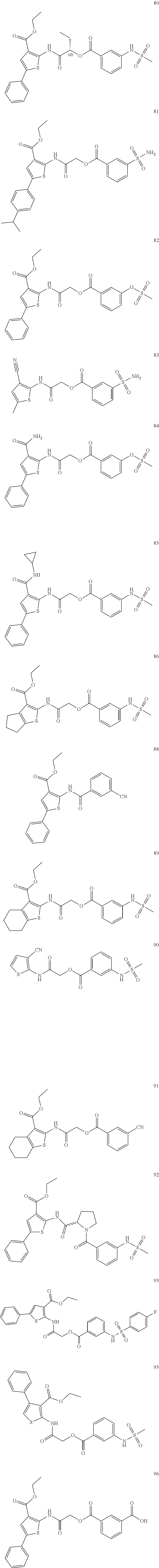

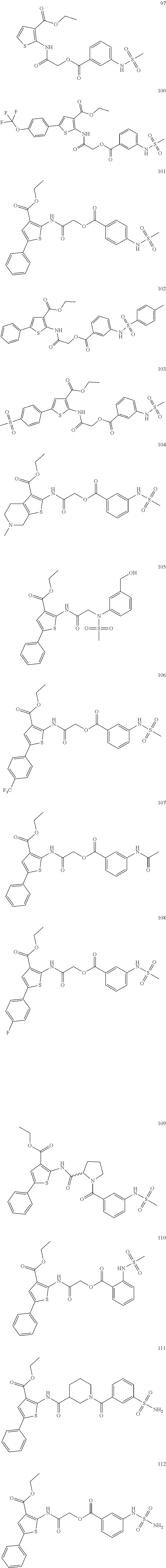

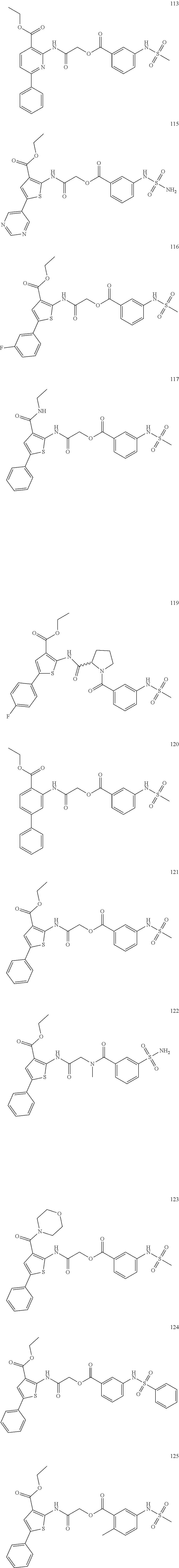

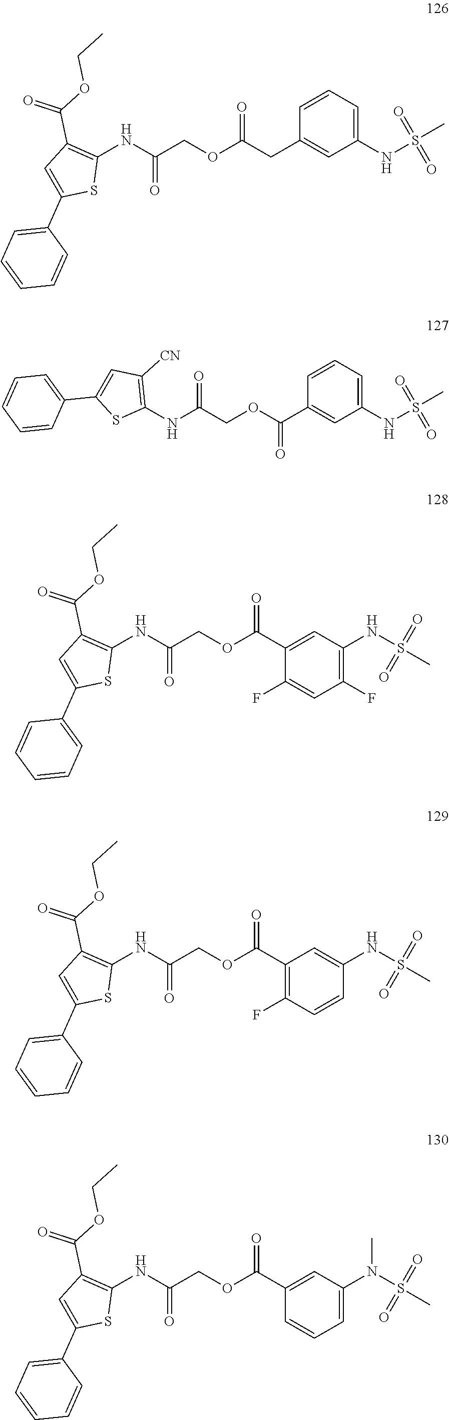 [SMILES: CCOC(=O)c1cc(-c2ccccc2)sc1NC(=O)COC(=O)Cc1cccc(NS(C)(=O)=O)c1 ; CS(=O)(=O)Nc1cccc(C(=O)OCC(=O)Nc2sc(-c3ccccc3)cc2C#N)c1 ; CCOC(=O)c1cc(-c2ccccc2)sc1NC(=O)COC(=O)c1cc(NS(C)(=O)=O)c(F)cc1F ; CCOC(=O)c1cc(-c2ccccc2)sc1NC(=O)COC(=O)c1cc(NS(C)(=O)=O)ccc1F ; CCOC(=O)c1cc(-c2ccccc2)sc1NC(=O)COC(=O)c1cccc(N(C)S(C)(=O)=O)c1]

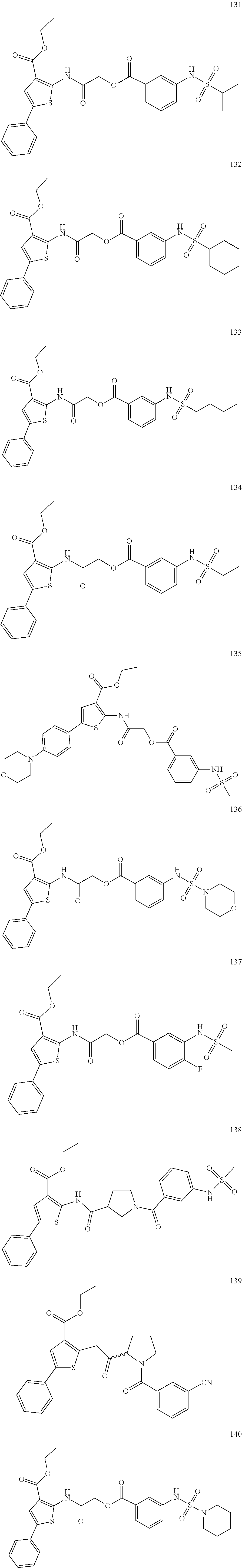

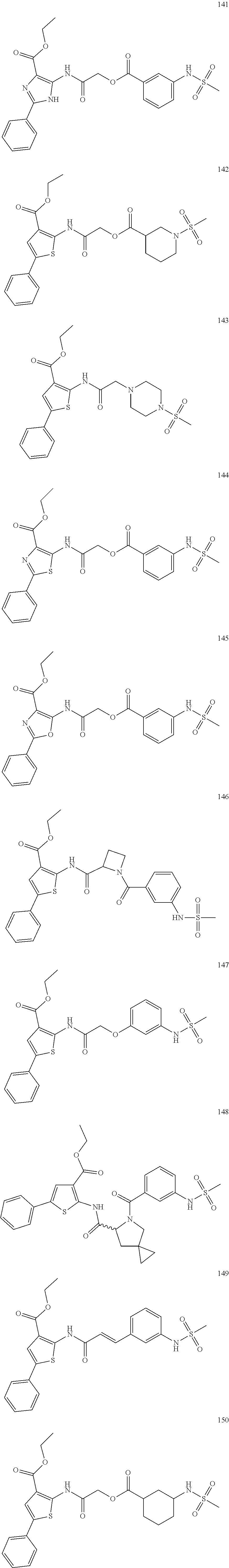

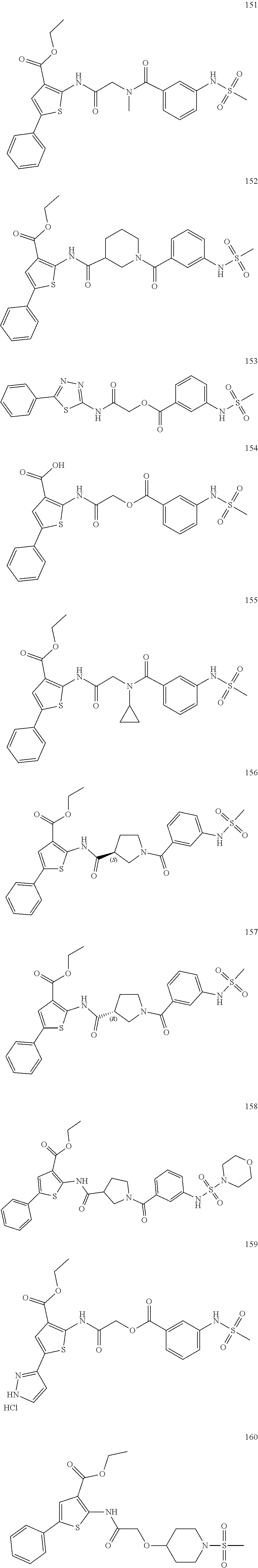

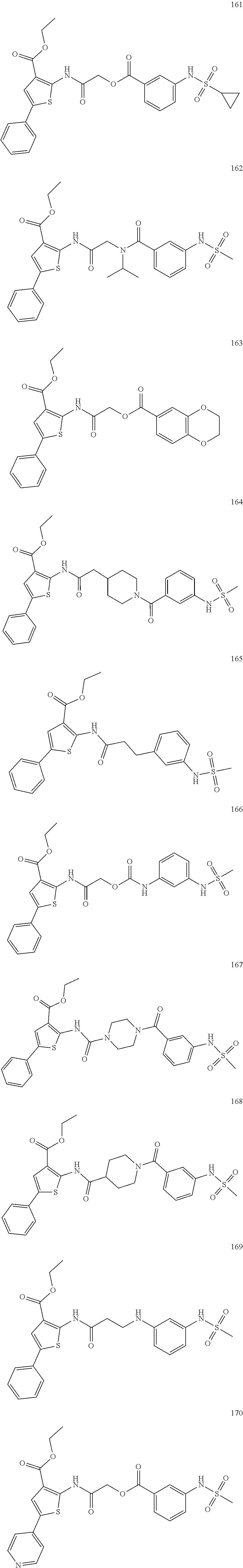

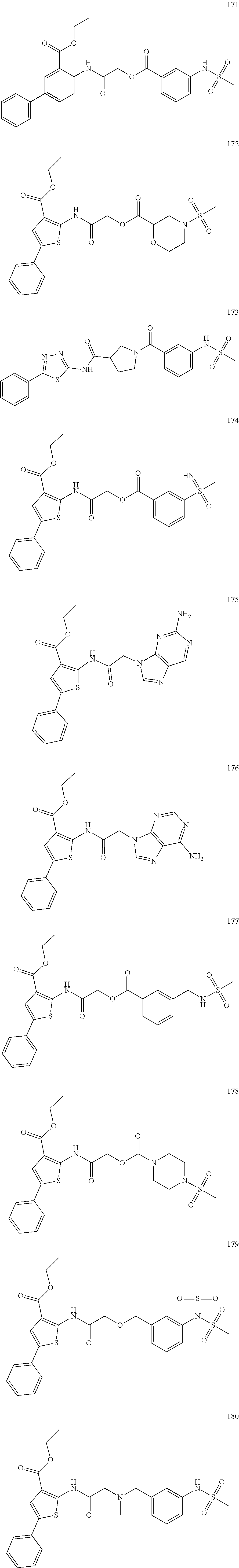

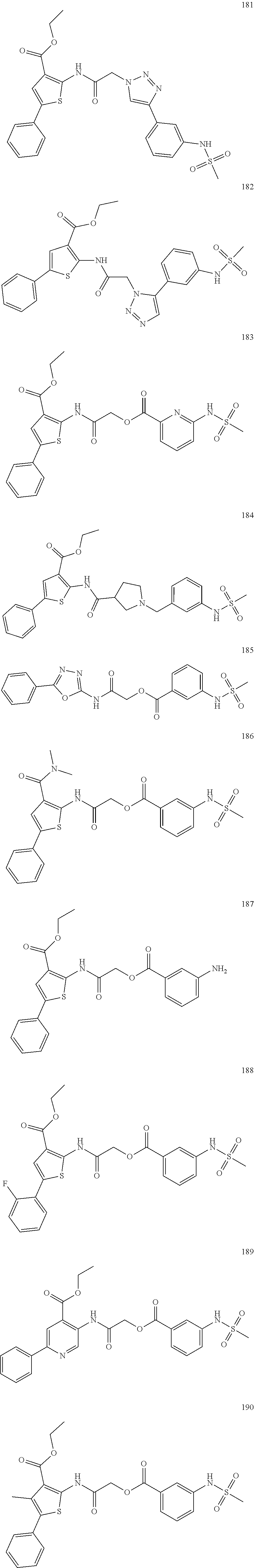

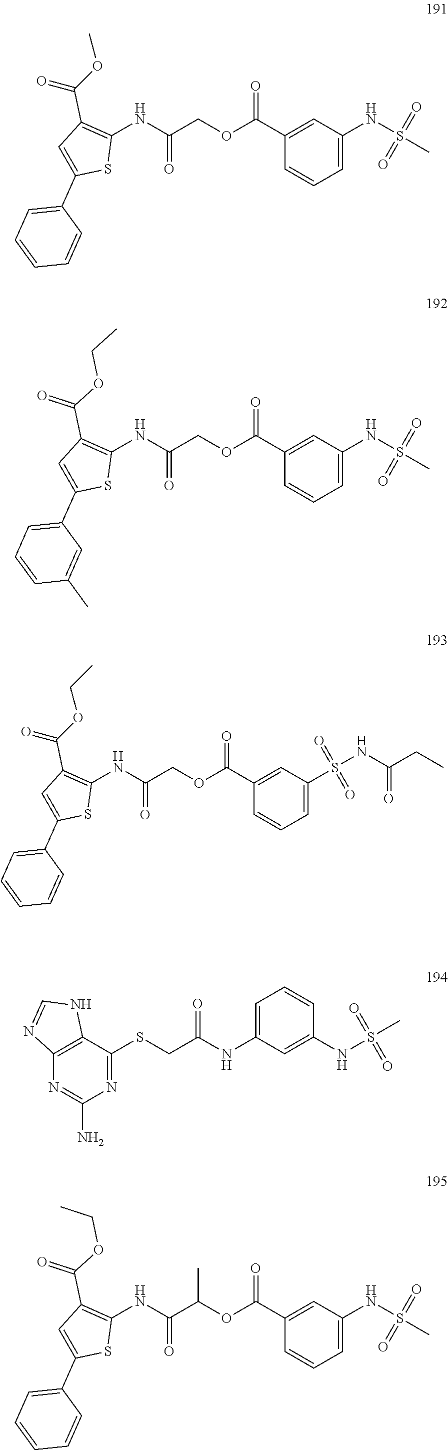 [SMILES: COC(=O)c1cc(-c2ccccc2)sc1NC(=O)COC(=O)c1cccc(NS(C)(=O)=O)c1 ; CCOC(=O)c1cc(-c2cccc(C)c2)sc1NC(=O)COC(=O)c1cccc(NS(C)(=O)=O)c1 ; CCOC(=O)c1cc(-c2ccccc2)sc1NC(=O)COC(=O)c1cccc(S(=O)(=O)NC(=O)CC)c1 ; CS(=O)(=O)Nc1cccc(NC(=O)CSc2nc(N)nc3nc[nH]c23)c1 ; CCOC(=O)c1cc(-c2ccccc2)sc1NC(=O)C(C)OC(=O)c1cccc(NS(C)(=O)=O)c1]

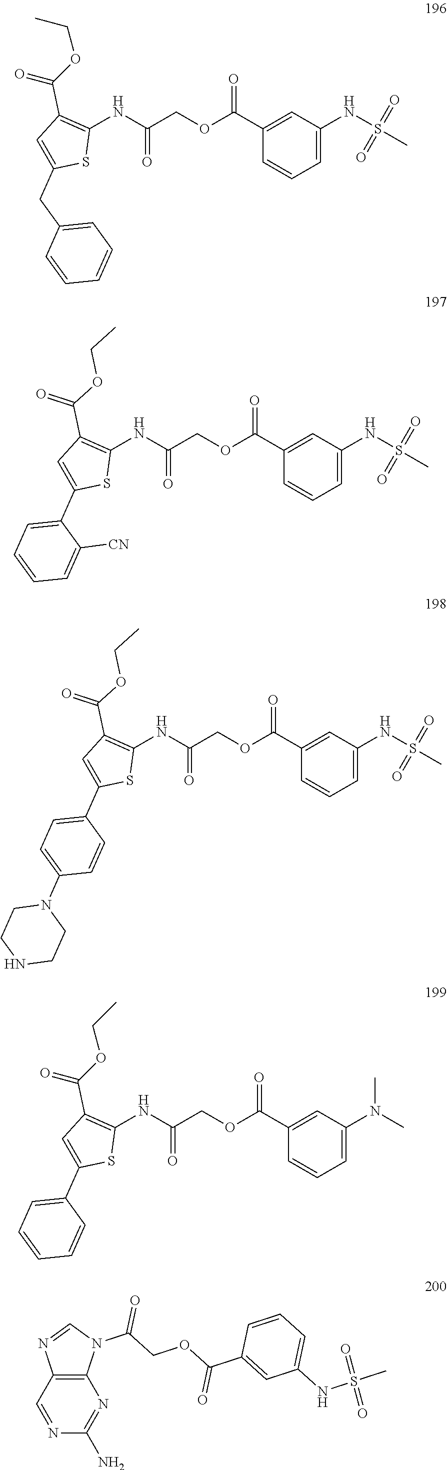 [SMILES: CCOC(=O)c1cc(Cc2ccccc2)sc1NC(=O)COC(=O)c1cccc(NS(C)(=O)=O)c1 ; CCOC(=O)c1cc(-c2ccccc2C#N)sc1NC(=O)COC(=O)c1cccc(NS(C)(=O)=O)c1 ; CCOC(=O)c1cc(-c2ccc(N3CCNCC3)cc2)sc1NC(=O)COC(=O)c1cccc(NS(C)(=O)=O)c1 ; CCOC(=O)c1cc(-c2ccccc2)sc1NC(=O)COC(=O)c1cccc(N(C)C)c1 ; CS(=O)(=O)Nc1cccc(C(=O)OCC(=O)n2cnc3cnc(N)nc32)c1]

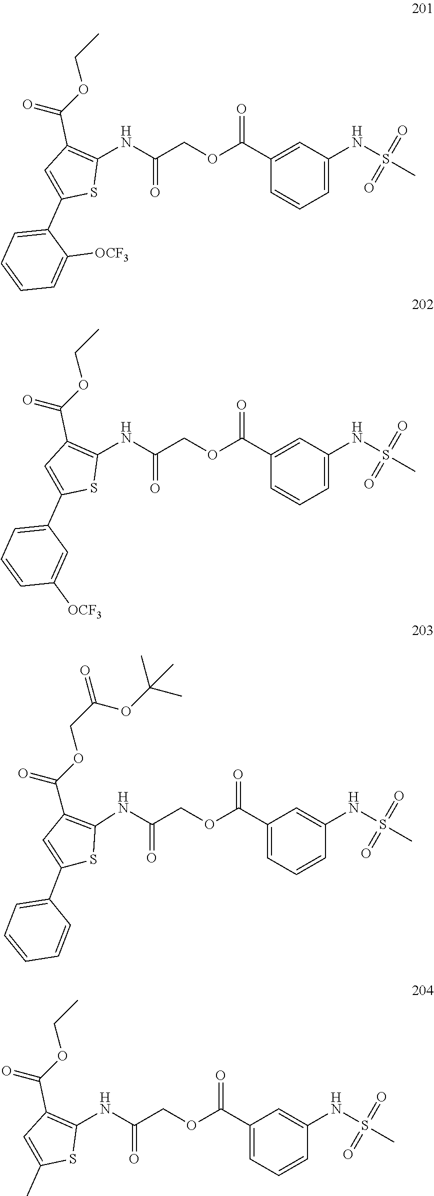 [SMILES: CCOC(=O)c1cc(-c2ccccc2OC(F)(F)F)sc1NC(=O)COC(=O)c1cccc(NS(C)(=O)=O)c1 ; CCOC(=O)c1cc(-c2cccc(OC(F)(F)F)c2)sc1NC(=O)COC(=O)c1cccc(NS(C)(=O)=O)c1 ; CC(C)(C)OC(=O)COC(=O)c1cc(-c2ccccc2)sc1NC(=O)COC(=O)c1cccc(NS(C)(=O)=O)c1 ; CCOC(=O)c1cc(C)sc1NC(=O)COC(=O)c1cccc(NS(C)(=O)=O)c1]

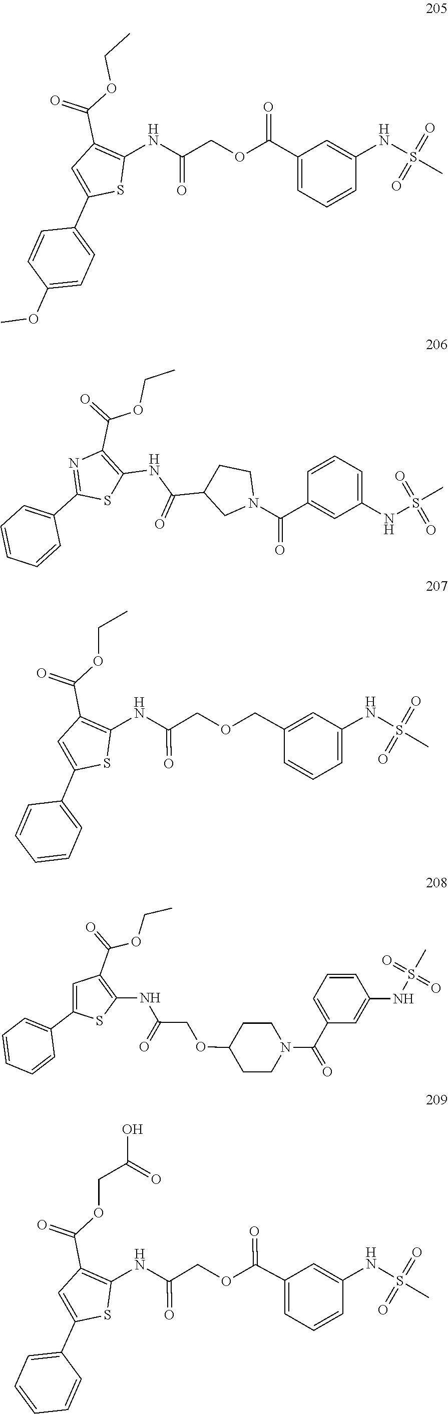

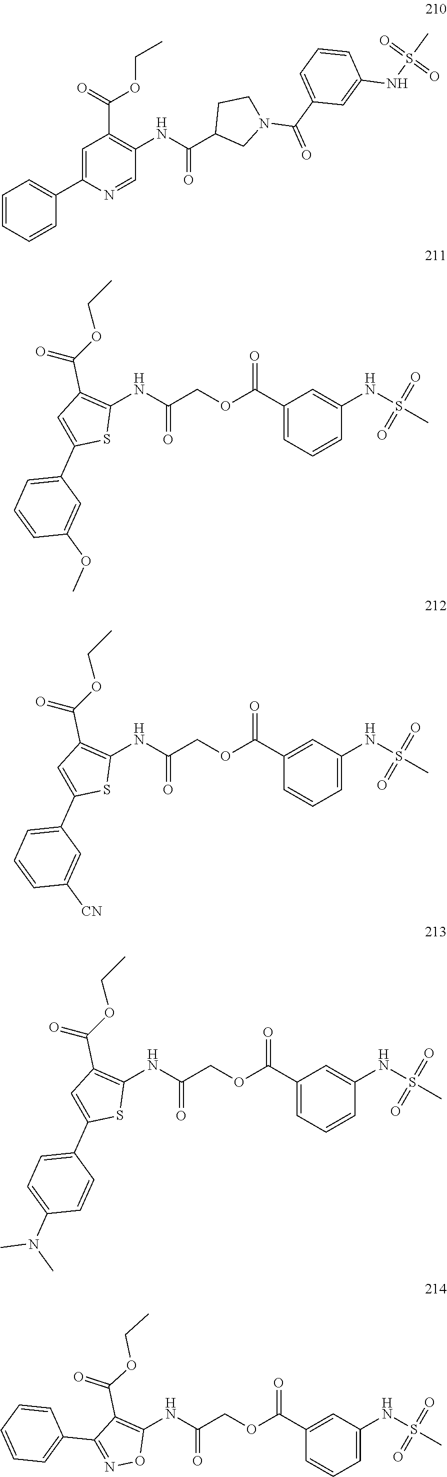

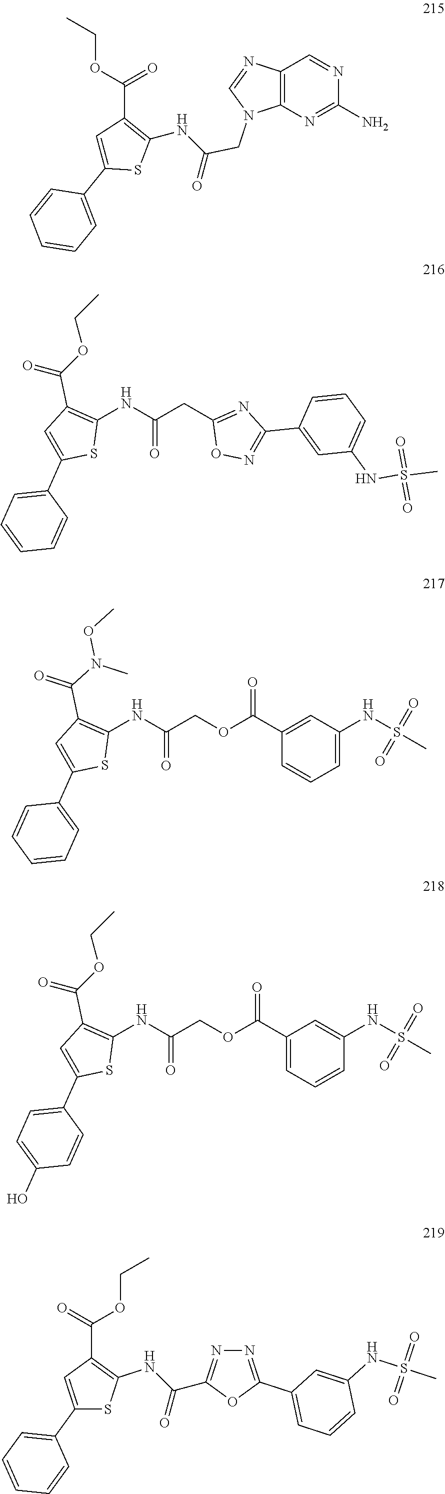 [SMILES: CCOC(=O)c1cc(-c2ccccc2)sc1NC(=O)Cn1cnc2cnc(N)nc21 ; CCOC(=O)c1cc(-c2ccccc2)sc1NC(=O)Cc1nc(-c2cccc(NS(C)(=O)=O)c2)no1 ; CON(C)C(=O)c1cc(-c2ccccc2)sc1NC(=O)COC(=O)c1cccc(NS(C)(=O)=O)c1 ; CCOC(=O)c1cc(-c2ccc(O)cc2)sc1NC(=O)COC(=O)c1cccc(NS(C)(=O)=O)c1 ; CCOC(=O)c1cc(-c2ccccc2)sc1NC(=O)c1nnc(-c2cccc(NS(C)(=O)=O)c2)o1]

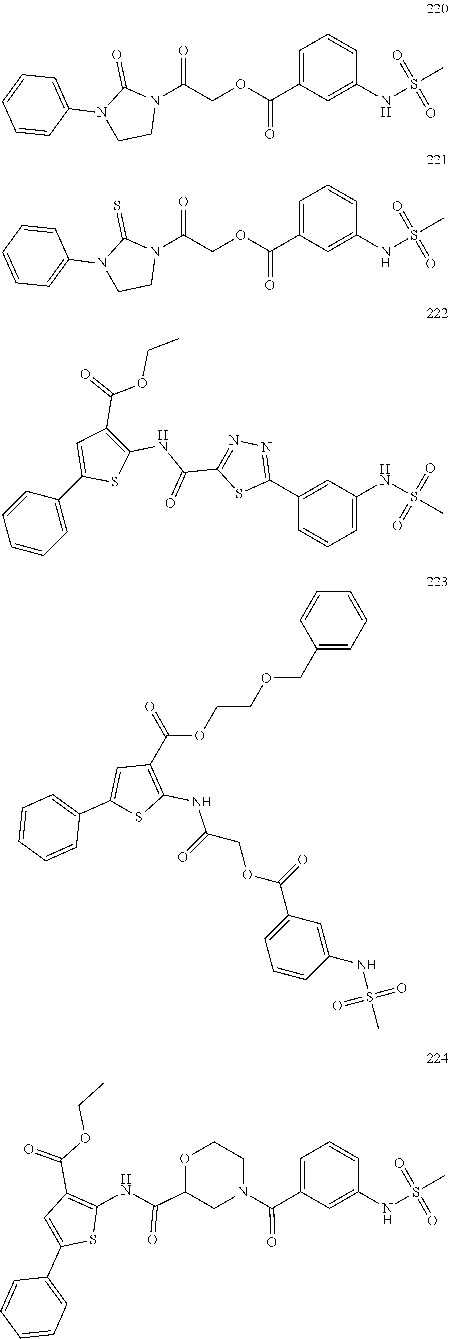

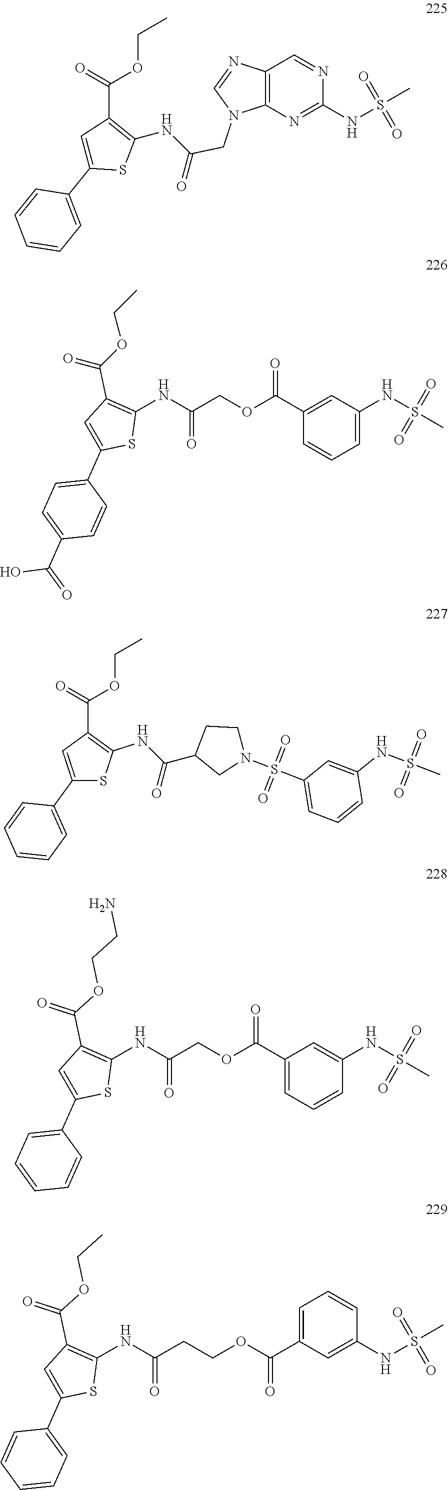 [SMILES: CCOC(=O)c1cc(-c2ccccc2)sc1NC(=O)Cn1cnc2cnc(NS(C)(=O)=O)nc21 ; CCOC(=O)c1cc(-c2ccc(C(=O)O)cc2)sc1NC(=O)COC(=O)c1cccc(NS(C)(=O)=O)c1 ; CCOC(=O)c1cc(-c2ccccc2)sc1NC(=O)C1CCN(S(=O)(=O)c2cccc(NS(C)(=O)=O)c2)C1 ; CS(=O)(=O)Nc1cccc(C(=O)OCC(=O)Nc2sc(-c3ccccc3)cc2C(=O)OCCN)c1 ; CCOC(=O)c1cc(-c2ccccc2)sc1NC(=O)CCOC(=O)c1cccc(NS(C)(=O)=O)c1]

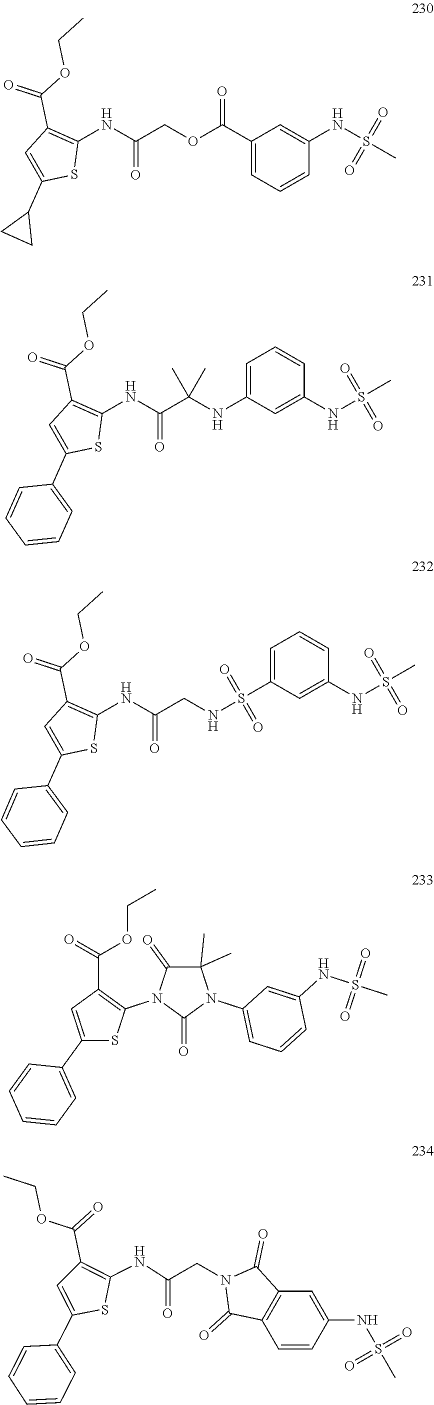

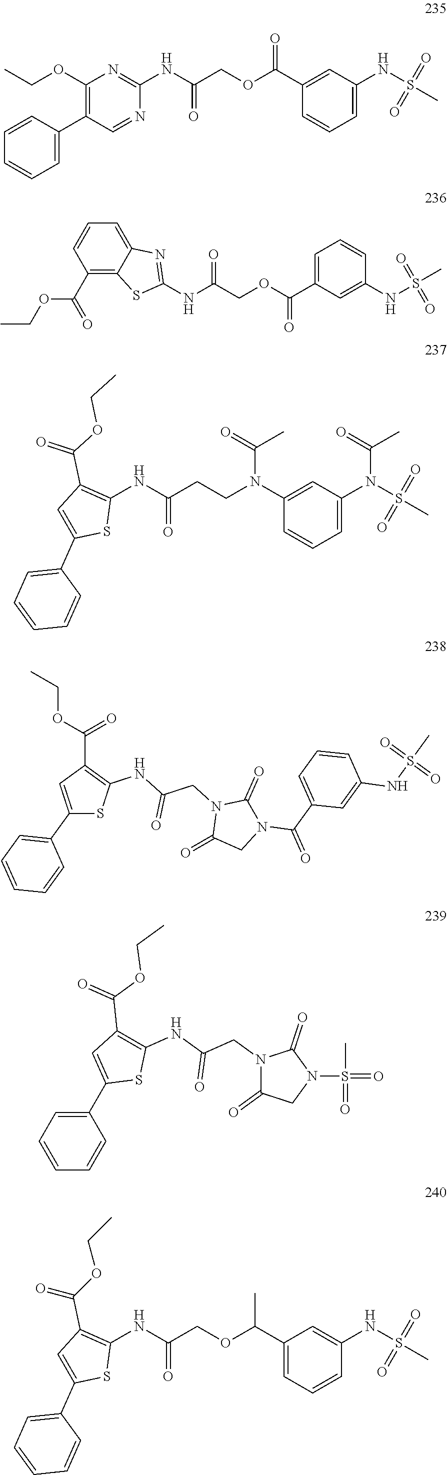

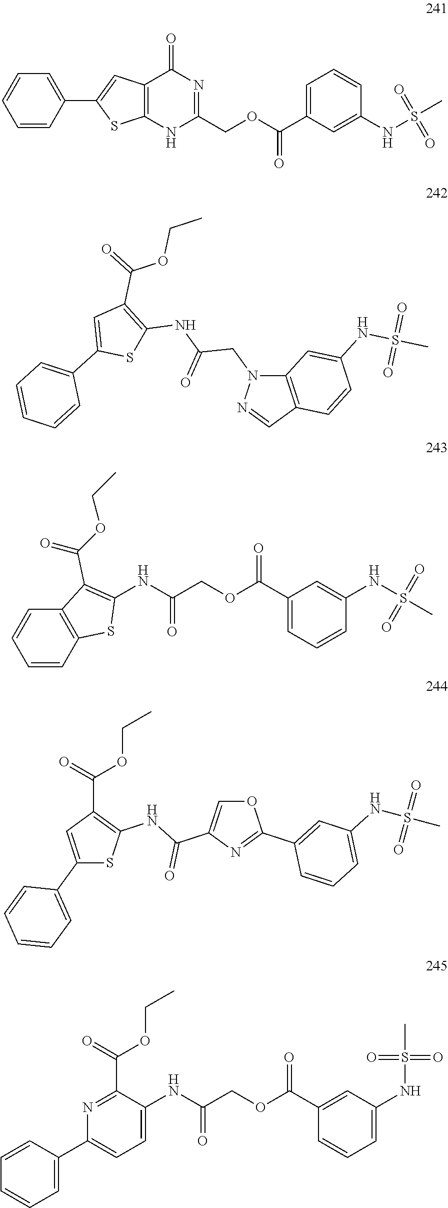 [SMILES: CS(=O)(=O)Nc1cccc(C(=O)OCc2nc(=O)c3cc(-c4ccccc4)sc3[nH]2)c1 ; CCOC(=O)c1cc(-c2ccccc2)sc1NC(=O)Cn1ncc2ccc(NS(C)(=O)=O)cc21 ; CCOC(=O)c1c(NC(=O)COC(=O)c2cccc(NS(C)(=O)=O)c2)sc2ccccc12 ; CCOC(=O)c1cc(-c2ccccc2)sc1NC(=O)c1coc(-c2cccc(NS(C)(=O)=O)c2)n1 ; CCOC(=O)c1nc(-c2ccccc2)ccc1NC(=O)COC(=O)c1cccc(NS(C)(=O)=O)c1]

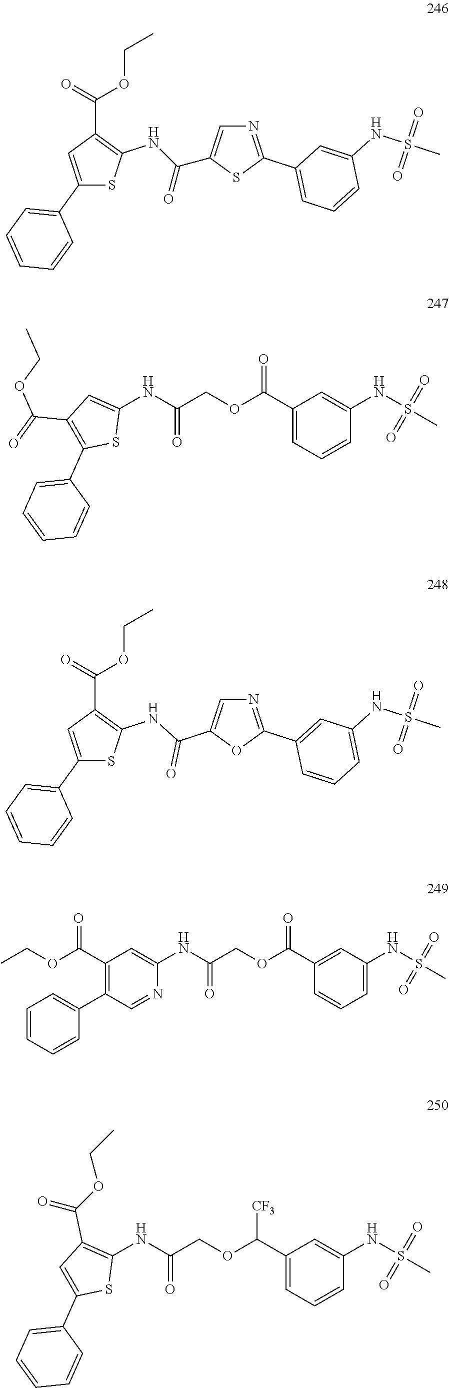

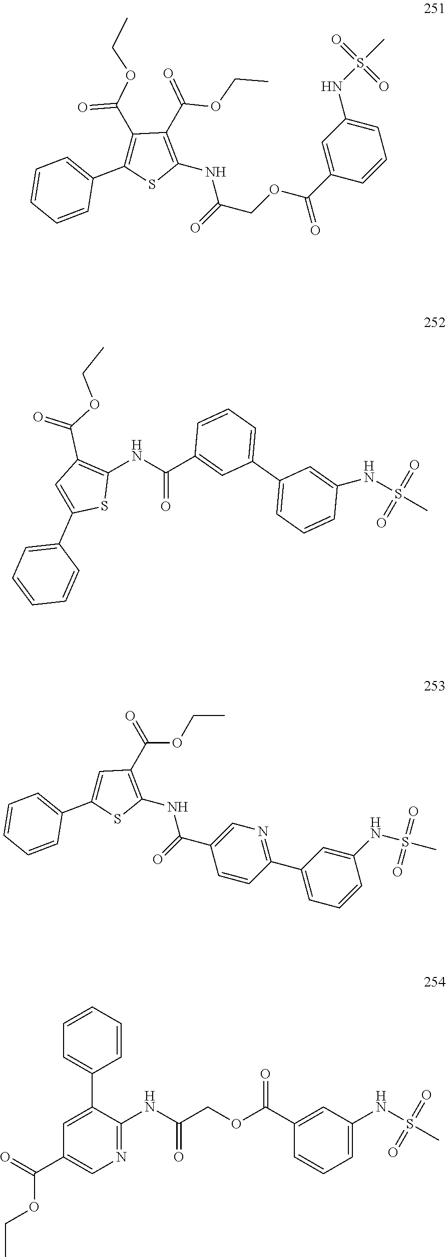 [SMILES: CCOC(=O)c1c(NC(=O)COC(=O)c2cccc(NS(C)(=O)=O)c2)sc(-c2ccccc2)c1C(=O)OCC ; CCOC(=O)c1cc(-c2ccccc2)sc1NC(=O)c1cccc(-c2cccc(NS(C)(=O)=O)c2)c1 ; CCOC(=O)c1cc(-c2ccccc2)sc1NC(=O)c1ccc(-c2cccc(NS(C)(=O)=O)c2)nc1 ; CCOC(=O)c1cnc(NC(=O)COC(=O)c2cccc(NS(C)(=O)=O)c2)c(-c2ccccc2)c1]

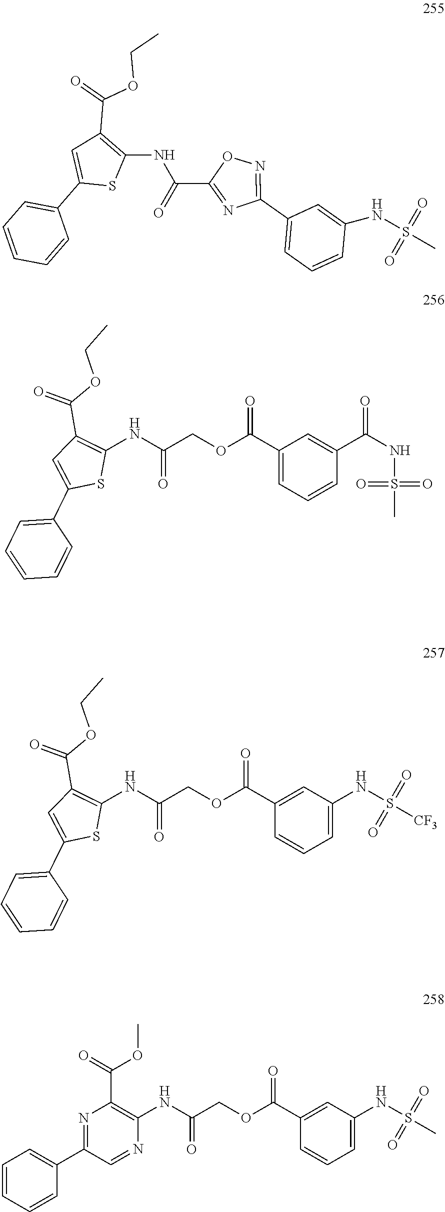 [SMILES: CCOC(=O)c1cc(-c2ccccc2)sc1NC(=O)c1nc(-c2cccc(NS(C)(=O)=O)c2)no1 ; CCOC(=O)c1cc(-c2ccccc2)sc1NC(=O)COC(=O)c1cccc(C(=O)NS(C)(=O)=O)c1 ; CCOC(=O)c1cc(-c2ccccc2)sc1NC(=O)COC(=O)c1cccc(NS(=O)(=O)C(F)(F)F)c1 ; COC(=O)c1nc(-c2ccccc2)cnc1NC(=O)COC(=O)c1cccc(NS(C)(=O)=O)c1]

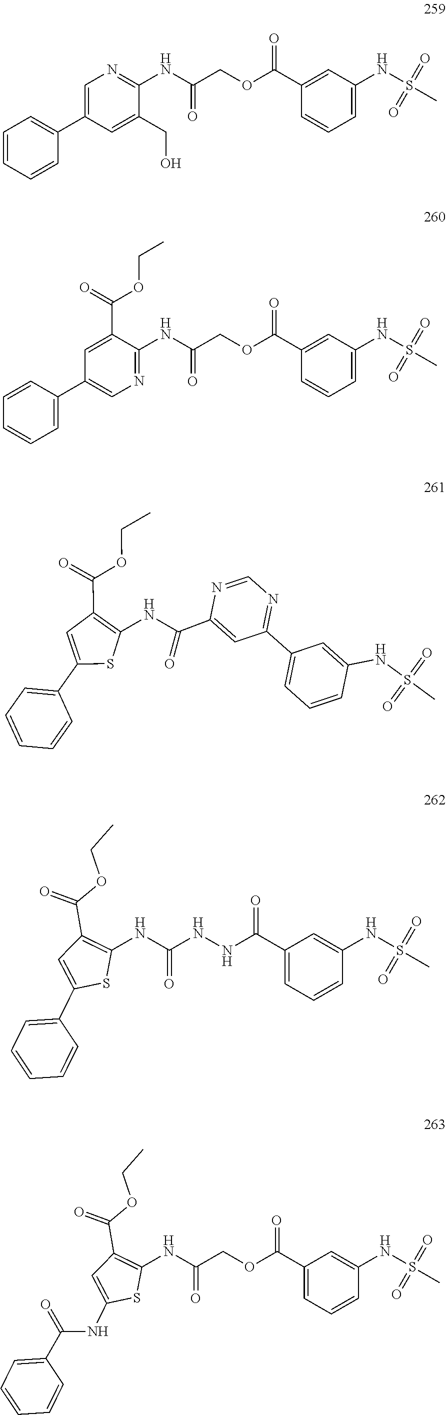 [SMILES: CS(=O)(=O)Nc1cccc(C(=O)OCC(=O)Nc2ncc(-c3ccccc3)cc2CO)c1 ; CCOC(=O)c1cc(-c2ccccc2)cnc1NC(=O)COC(=O)c1cccc(NS(C)(=O)=O)c1 ; CCOC(=O)c1cc(-c2ccccc2)sc1NC(=O)c1cc(-c2cccc(NS(C)(=O)=O)c2)ncn1 ; CCOC(=O)c1cc(-c2ccccc2)sc1NC(=O)NNC(=O)c1cccc(NS(C)(=O)=O)c1 ; CCOC(=O)c1cc(NC(=O)c2ccccc2)sc1NC(=O)COC(=O)c1cccc(NS(C)(=O)=O)c1]

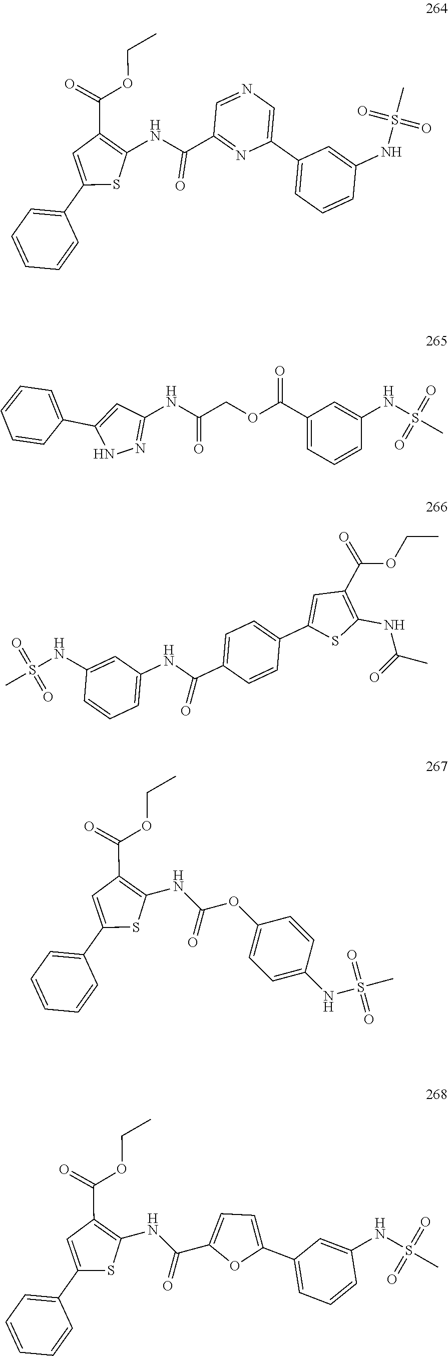

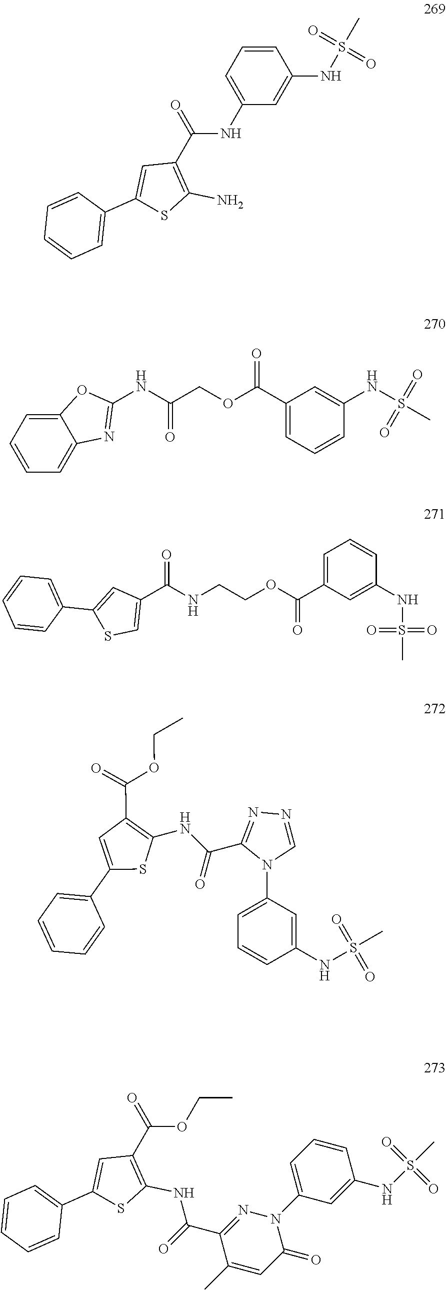 [SMILES: CS(=O)(=O)Nc1cccc(NC(=O)c2cc(-c3ccccc3)sc2N)c1 ; CS(=O)(=O)Nc1cccc(C(=O)OCC(=O)Nc2nc3ccccc3o2)c1 ; CS(=O)(=O)Nc1cccc(C(=O)OCCNC(=O)c2csc(-c3ccccc3)c2)c1 ; CCOC(=O)c1cc(-c2ccccc2)sc1NC(=O)c1nncn1-c1cccc(NS(C)(=O)=O)c1 ; CCOC(=O)c1cc(-c2ccccc2)sc1NC(=O)c1nn(-c2cccc(NS(C)(=O)=O)c2)c(=O)cc1C]

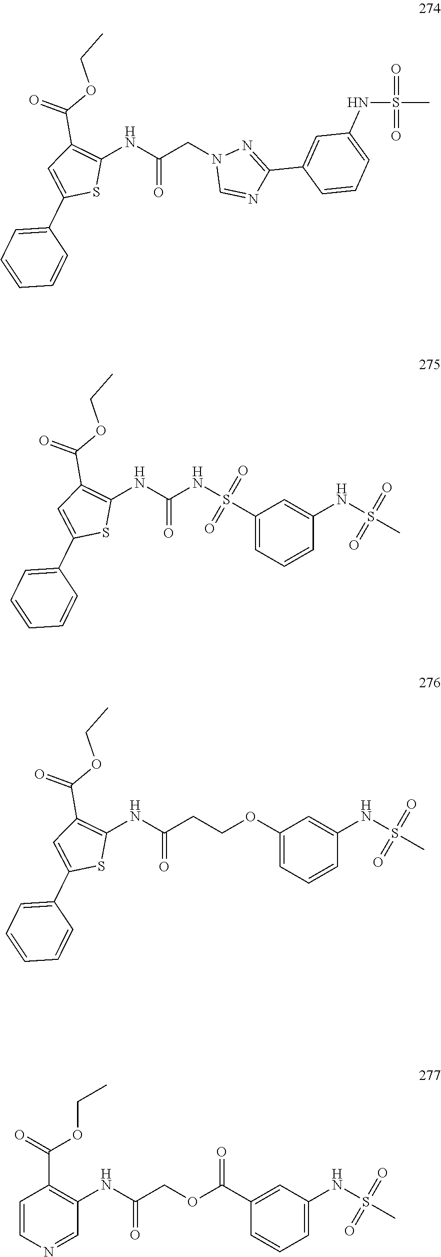 [SMILES: CCOC(=O)c1cc(-c2ccccc2)sc1NC(=O)Cn1cnc(-c2cccc(NS(C)(=O)=O)c2)n1 ; CCOC(=O)c1cc(-c2ccccc2)sc1NC(=O)NS(=O)(=O)c1cccc(NS(C)(=O)=O)c1 ; CCOC(=O)c1cc(-c2ccccc2)sc1NC(=O)CCOc1cccc(NS(C)(=O)=O)c1 ; CCOC(=O)c1ccncc1NC(=O)COC(=O)c1cccc(NS(C)(=O)=O)c1]

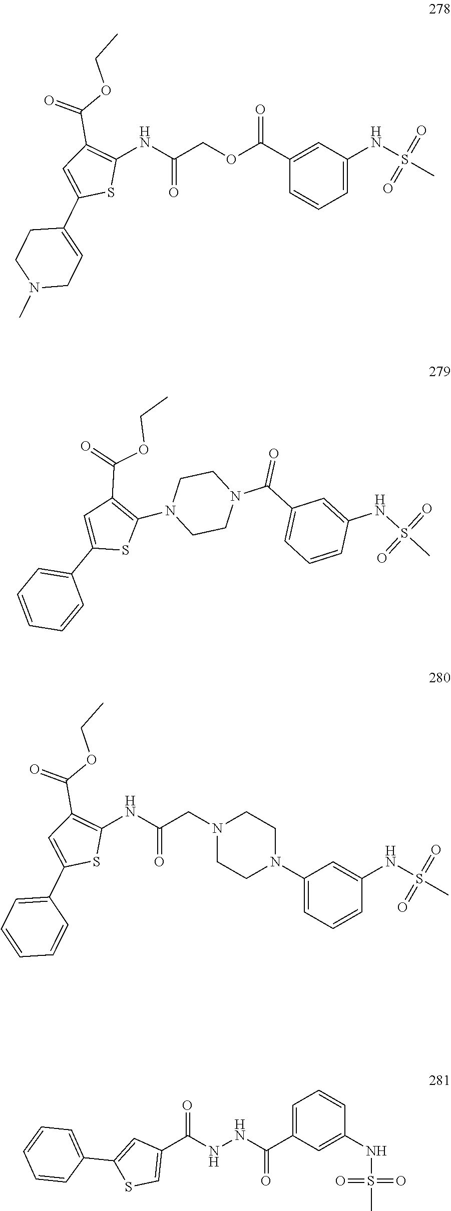 [SMILES: CCOC(=O)c1cc(C2=CCN(C)CC2)sc1NC(=O)COC(=O)c1cccc(NS(C)(=O)=O)c1 ; CCOC(=O)c1cc(-c2ccccc2)sc1N1CCN(C(=O)c2cccc(NS(C)(=O)=O)c2)CC1 ; CCOC(=O)c1cc(-c2ccccc2)sc1NC(=O)CN1CCN(c2cccc(NS(C)(=O)=O)c2)CC1 ; CS(=O)(=O)Nc1cccc(C(=O)NNC(=O)c2csc(-c3ccccc3)c2)c1]

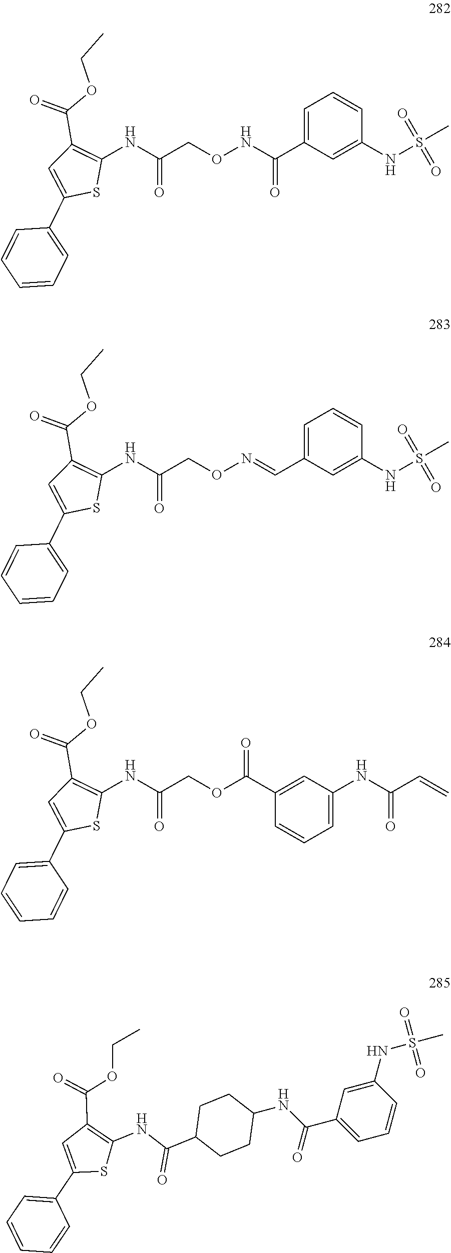 [SMILES: CCOC(=O)c1cc(-c2ccccc2)sc1NC(=O)CONC(=O)c1cccc(NS(C)(=O)=O)c1 ; CCOC(=O)c1cc(-c2ccccc2)sc1NC(=O)CO/N=C/c1cccc(NS(C)(=O)=O)c1 ; C=CC(=O)Nc1cccc(C(=O)OCC(=O)Nc2sc(-c3ccccc3)cc2C(=O)OCC)c1 ; CCOC(=O)c1cc(-c2ccccc2)sc1NC(=O)C1CCC(NC(=O)c2cccc(NS(C)(=O)=O)c2)CC1]

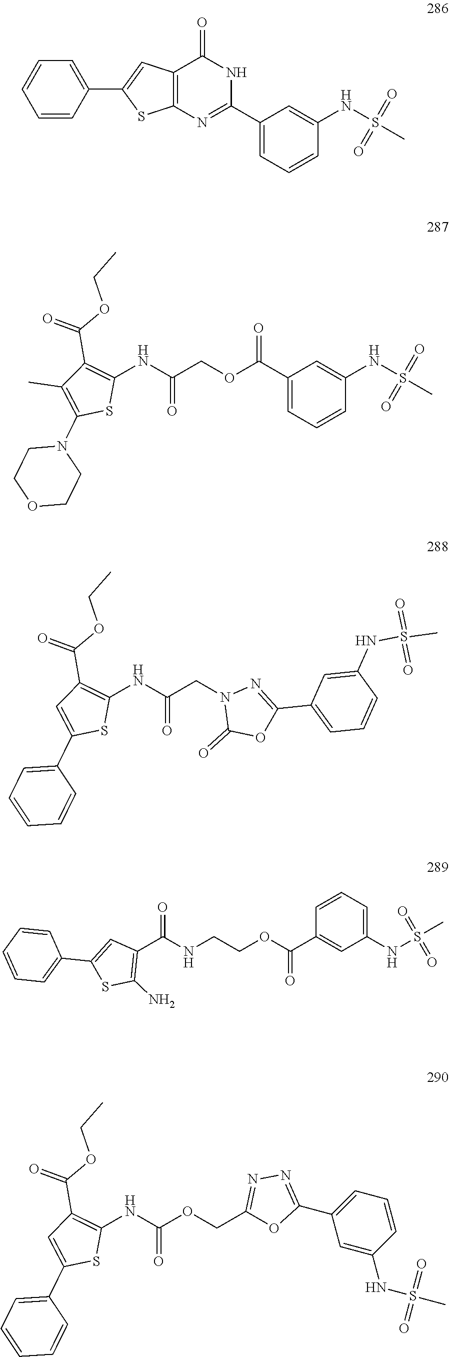 [SMILES: CS(=O)(=O)Nc1cccc(-c2nc3sc(-c4ccccc4)cc3c(=O)[nH]2)c1 ; CCOC(=O)c1c(NC(=O)COC(=O)c2cccc(NS(C)(=O)=O)c2)sc(N2CCOCC2)c1C ; CCOC(=O)c1cc(-c2ccccc2)sc1NC(=O)Cn1nc(-c2cccc(NS(C)(=O)=O)c2)oc1=O ; CS(=O)(=O)Nc1cccc(C(=O)OCCNC(=O)c2cc(-c3ccccc3)sc2N)c1 ; CCOC(=O)c1cc(-c2ccccc2)sc1NC(=O)OCc1nnc(-c2cccc(NS(C)(=O)=O)c2)o1]

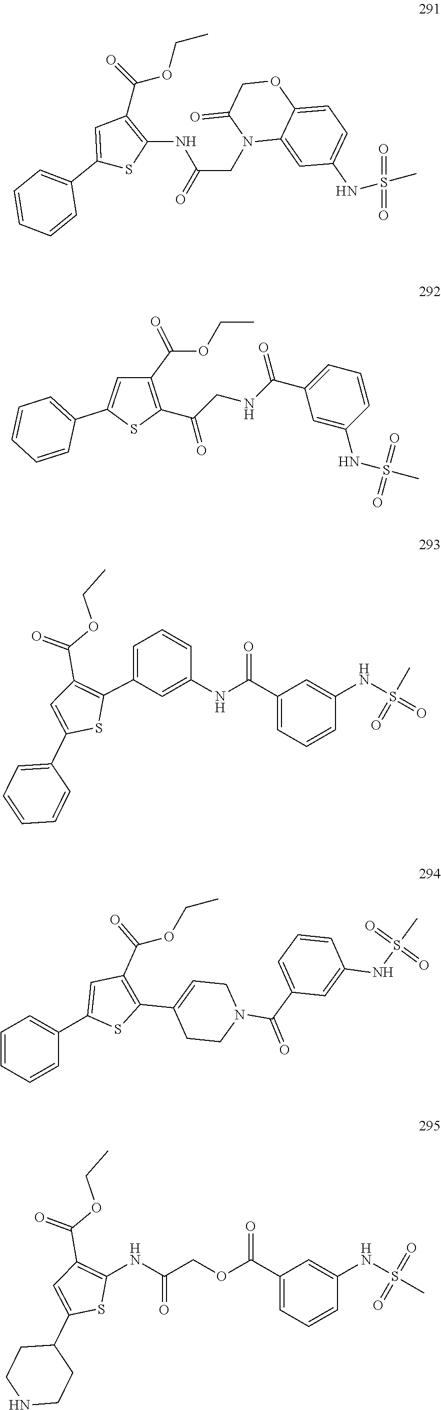 [SMILES: CCOC(=O)c1cc(-c2ccccc2)sc1NC(=O)CN1C(=O)COc2ccc(NS(C)(=O)=O)cc21 ; CCOC(=O)c1cc(-c2ccccc2)sc1C(=O)CNC(=O)c1cccc(NS(C)(=O)=O)c1 ; CCOC(=O)c1cc(-c2ccccc2)sc1-c1cccc(NC(=O)c2cccc(NS(C)(=O)=O)c2)c1 ; CCOC(=O)c1cc(-c2ccccc2)sc1C1=CCN(C(=O)c2cccc(NS(C)(=O)=O)c2)CC1 ; CCOC(=O)c1cc(C2CCNCC2)sc1NC(=O)COC(=O)c1cccc(NS(C)(=O)=O)c1]

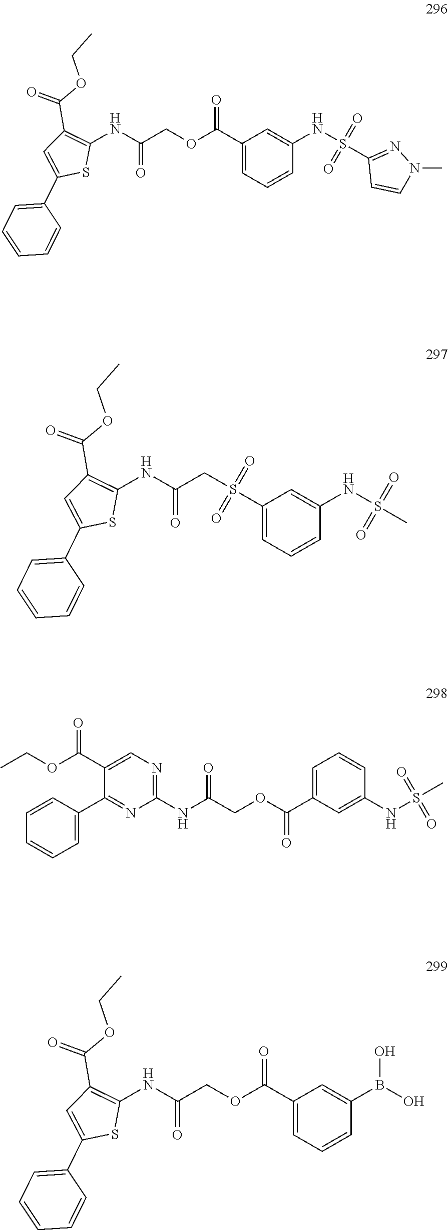

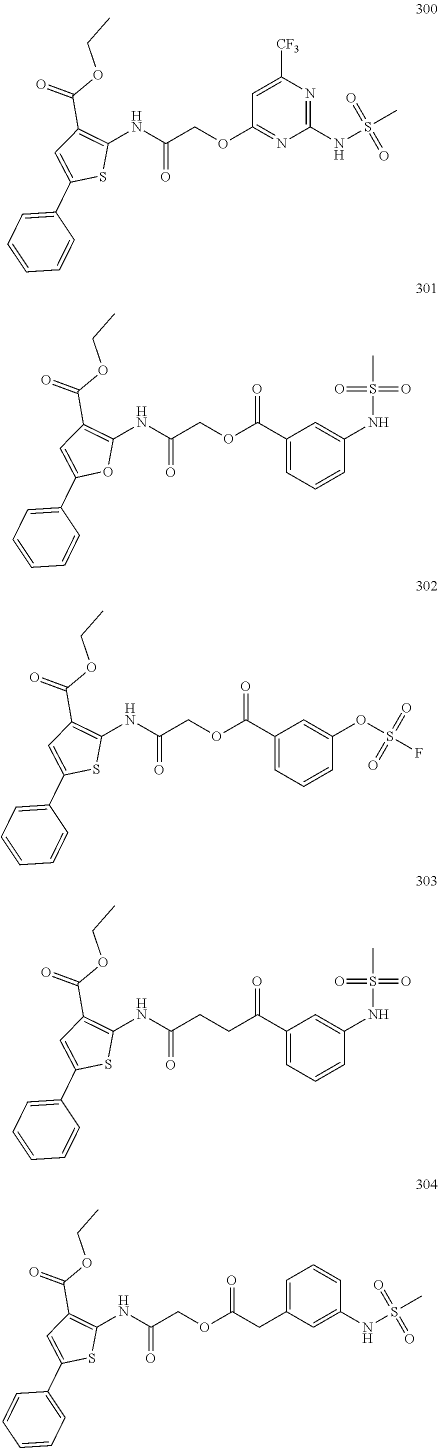 [SMILES: CCOC(=O)c1cc(-c2ccccc2)sc1NC(=O)COc1cc(C(F)(F)F)nc(NS(C)(=O)=O)n1 ; CCOC(=O)c1cc(-c2ccccc2)oc1NC(=O)COC(=O)c1cccc(NS(C)(=O)=O)c1 ; CCOC(=O)c1cc(-c2ccccc2)sc1NC(=O)COC(=O)c1cccc(OS(=O)(=O)F)c1 ; CCOC(=O)c1cc(-c2ccccc2)sc1NC(=O)CCC(=O)c1cccc(NS(C)(=O)=O)c1 ; CCOC(=O)c1cc(-c2ccccc2)sc1NC(=O)COC(=O)Cc1cccc(NS(C)(=O)=O)c1]

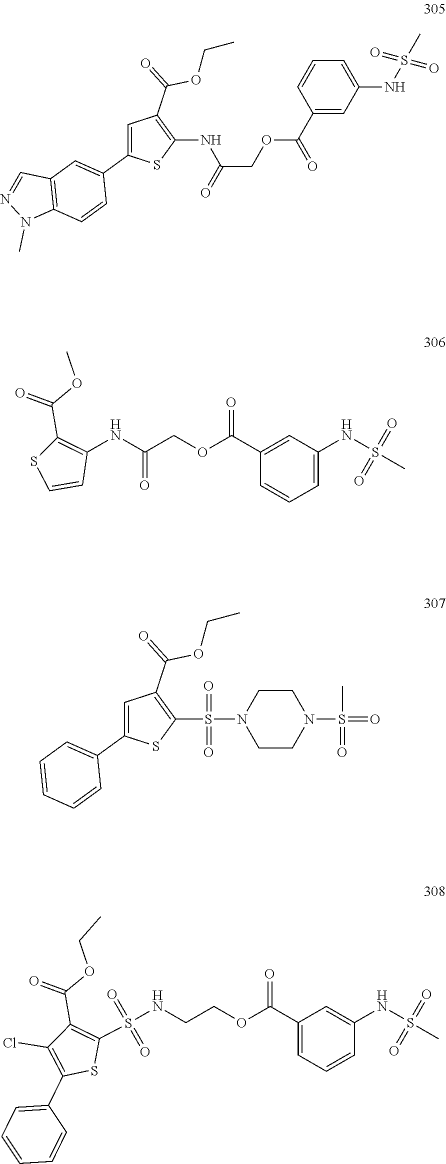 [SMILES: CCOC(=O)c1cc(-c2ccc3c(cnn3C)c2)sc1NC(=O)COC(=O)c1cccc(NS(C)(=O)=O)c1 ; COC(=O)c1sccc1NC(=O)COC(=O)c1cccc(NS(C)(=O)=O)c1 ; CCOC(=O)c1cc(-c2ccccc2)sc1S(=O)(=O)N1CCN(S(C)(=O)=O)CC1 ; CCOC(=O)c1c(S(=O)(=O)NCCOC(=O)c2cccc(NS(C)(=O)=O)c2)sc(-c2ccccc2)c1Cl]

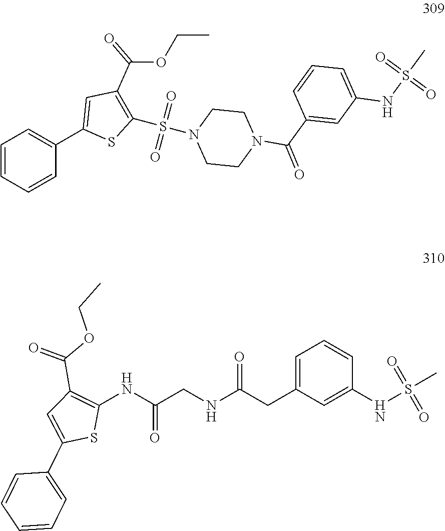 [SMILES: CCOC(=O)c1cc(-c2ccccc2)sc1S(=O)(=O)N1CCN(C(=O)c2cccc(NS(C)(=O)=O)c2)CC1 ; CCOC(=O)c1cc(-c2ccccc2)sc1NC(=O)CNC(=O)Cc1cccc(NS(C)(=O)=O)c1]

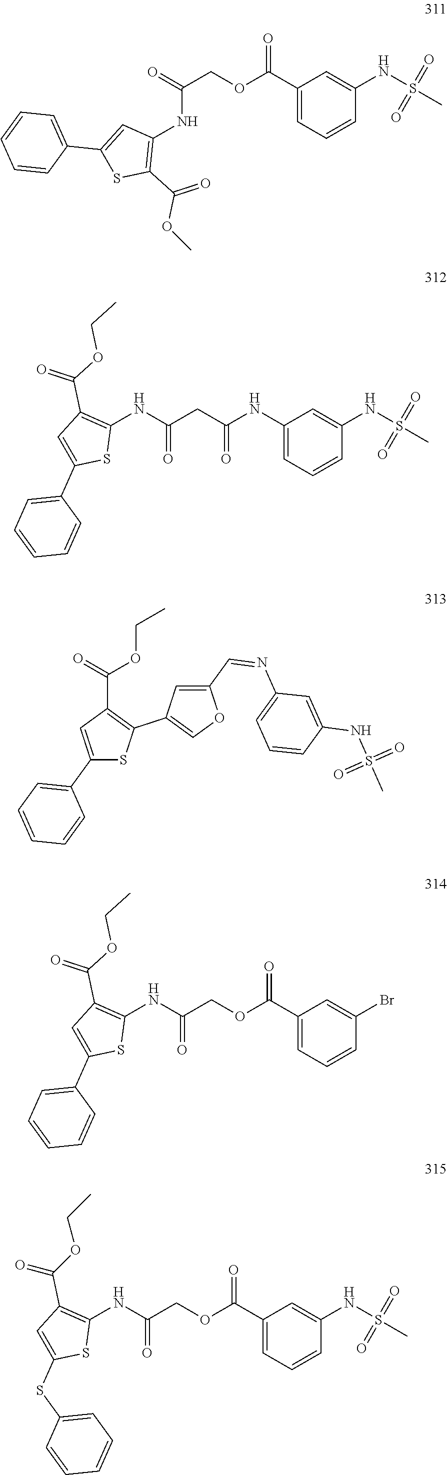 [SMILES: COC(=O)c1sc(-c2ccccc2)cc1NC(=O)COC(=O)c1cccc(NS(C)(=O)=O)c1 ; CCOC(=O)c1cc(-c2ccccc2)sc1NC(=O)CC(=O)Nc1cccc(NS(C)(=O)=O)c1 ; CCOC(=O)c1cc(-c2ccccc2)sc1-c1coc(/C=N\c2cccc(NS(C)(=O)=O)c2)c1 ; CCOC(=O)c1cc(-c2ccccc2)sc1NC(=O)COC(=O)c1cccc(Br)c1 ; CCOC(=O)c1cc(Sc2ccccc2)sc1NC(=O)COC(=O)c1cccc(NS(C)(=O)=O)c1]

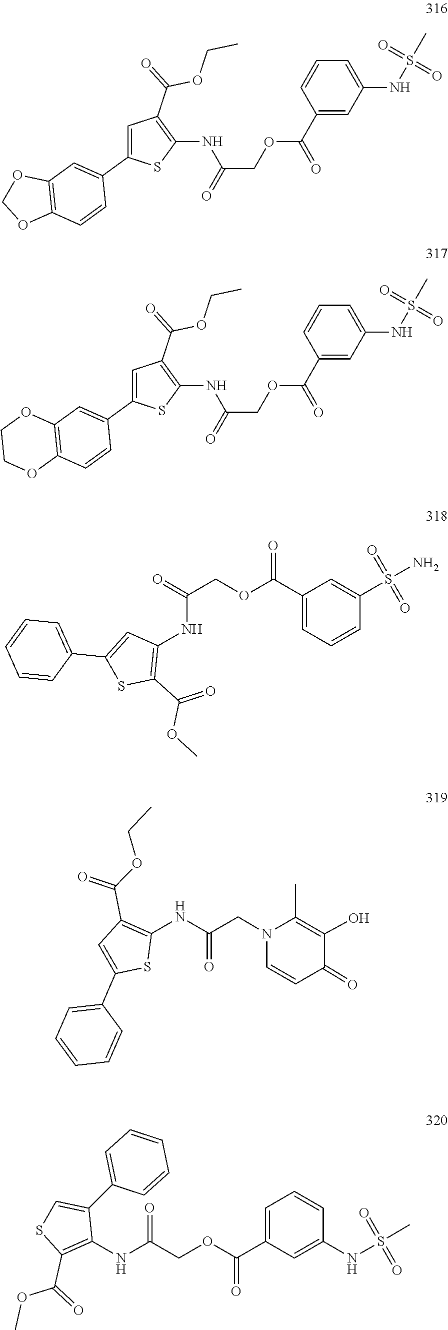

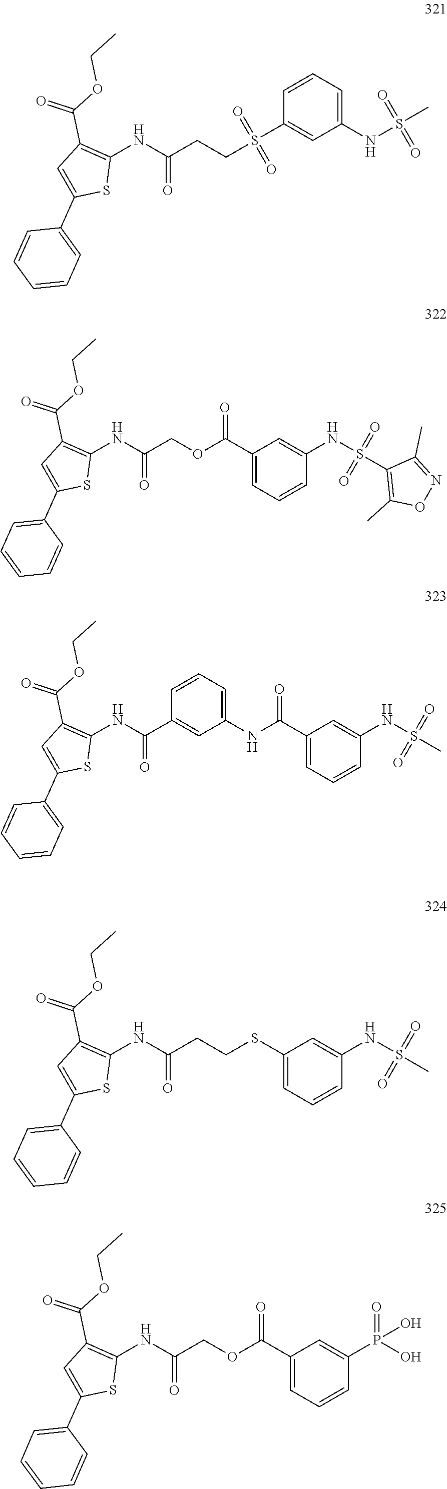 [SMILES: CCOC(=O)c1cc(-c2ccccc2)sc1NC(=O)CCS(=O)(=O)c1cccc(NS(C)(=O)=O)c1 ; CCOC(=O)c1cc(-c2ccccc2)sc1NC(=O)COC(=O)c1cccc(NS(=O)(=O)c2c(C)noc2C)c1 ; CCOC(=O)c1cc(-c2ccccc2)sc1NC(=O)c1cccc(NC(=O)c2cccc(NS(C)(=O)=O)c2)c1 ; CCOC(=O)c1cc(-c2ccccc2)sc1NC(=O)CCSc1cccc(NS(C)(=O)=O)c1 ; CCOC(=O)c1cc(-c2ccccc2)sc1NC(=O)COC(=O)c1cccc(P(=O)(O)O)c1]

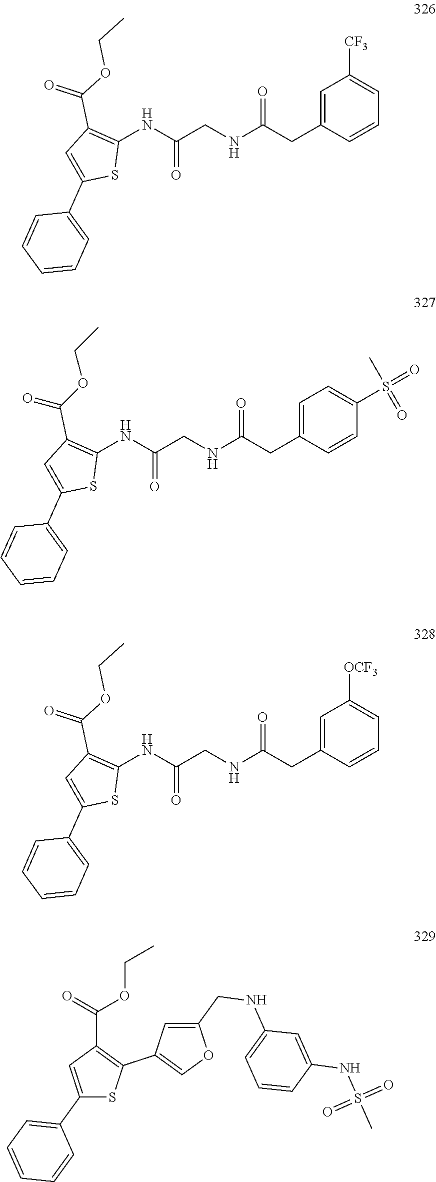 [SMILES: CCOC(=O)c1cc(-c2ccccc2)sc1NC(=O)CNC(=O)Cc1cccc(C(F)(F)F)c1 ; CCOC(=O)c1cc(-c2ccccc2)sc1NC(=O)CNC(=O)Cc1ccc(S(C)(=O)=O)cc1 ; CCOC(=O)c1cc(-c2ccccc2)sc1NC(=O)CNC(=O)Cc1cccc(OC(F)(F)F)c1 ; CCOC(=O)c1cc(-c2ccccc2)sc1-c1coc(CNc2cccc(NS(C)(=O)=O)c2)c1]

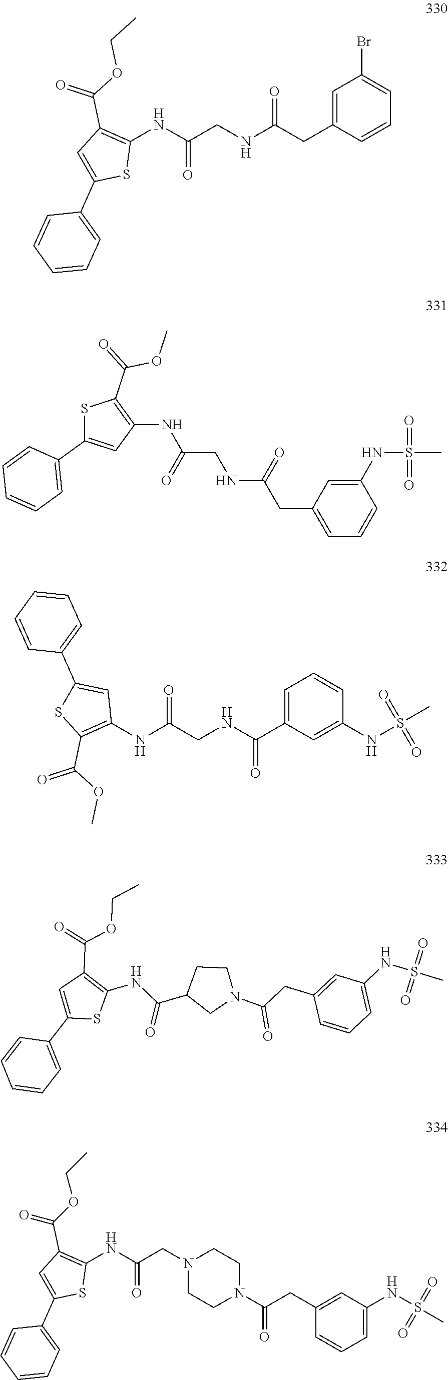 [SMILES: CCOC(=O)c1cc(-c2ccccc2)sc1NC(=O)CNC(=O)Cc1cccc(Br)c1 ; COC(=O)c1sc(-c2ccccc2)cc1NC(=O)CNC(=O)Cc1cccc(NS(C)(=O)=O)c1 ; COC(=O)c1sc(-c2ccccc2)cc1NC(=O)CNC(=O)c1cccc(NS(C)(=O)=O)c1 ; CCOC(=O)c1cc(-c2ccccc2)sc1NC(=O)C1CCN(C(=O)Cc2cccc(NS(C)(=O)=O)c2)C1 ; CCOC(=O)c1cc(-c2ccccc2)sc1NC(=O)CN1CCN(C(=O)Cc2cccc(NS(C)(=O)=O)c2)CC1]

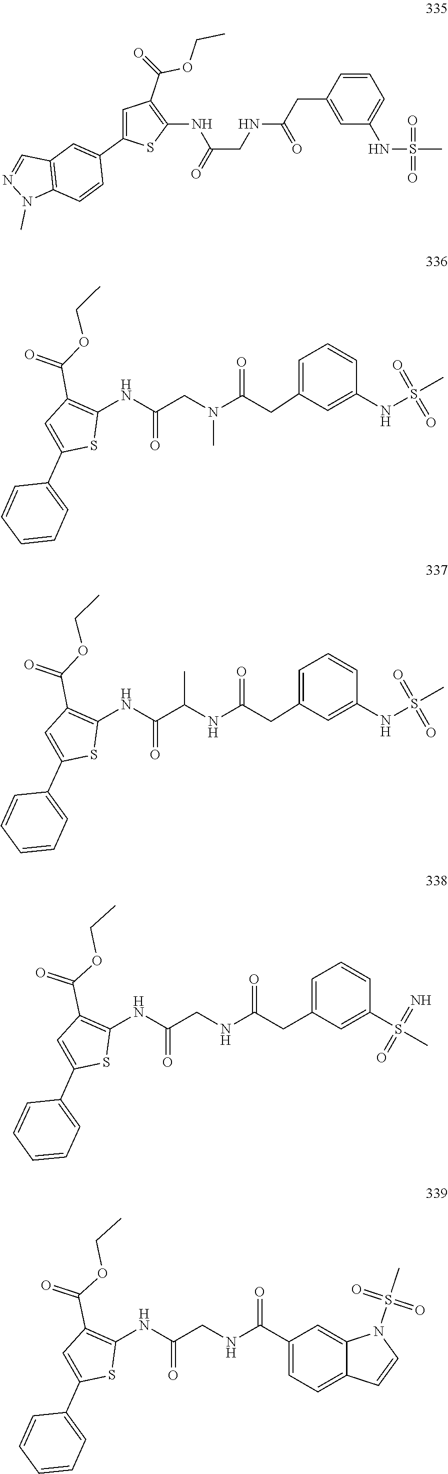 [SMILES: CCOC(=O)c1cc(-c2ccc3c(cnn3C)c2)sc1NC(=O)CNC(=O)Cc1cccc(NS(C)(=O)=O)c1 ; CCOC(=O)c1cc(-c2ccccc2)sc1NC(=O)CN(C)C(=O)Cc1cccc(NS(C)(=O)=O)c1 ; CCOC(=O)c1cc(-c2ccccc2)sc1NC(=O)C(C)NC(=O)Cc1cccc(NS(C)(=O)=O)c1 ; CCOC(=O)c1cc(-c2ccccc2)sc1NC(=O)CNC(=O)Cc1cccc(S(C)(=N)=O)c1 ; CCOC(=O)c1cc(-c2ccccc2)sc1NC(=O)CNC(=O)c1ccc2ccn(S(C)(=O)=O)c2c1]

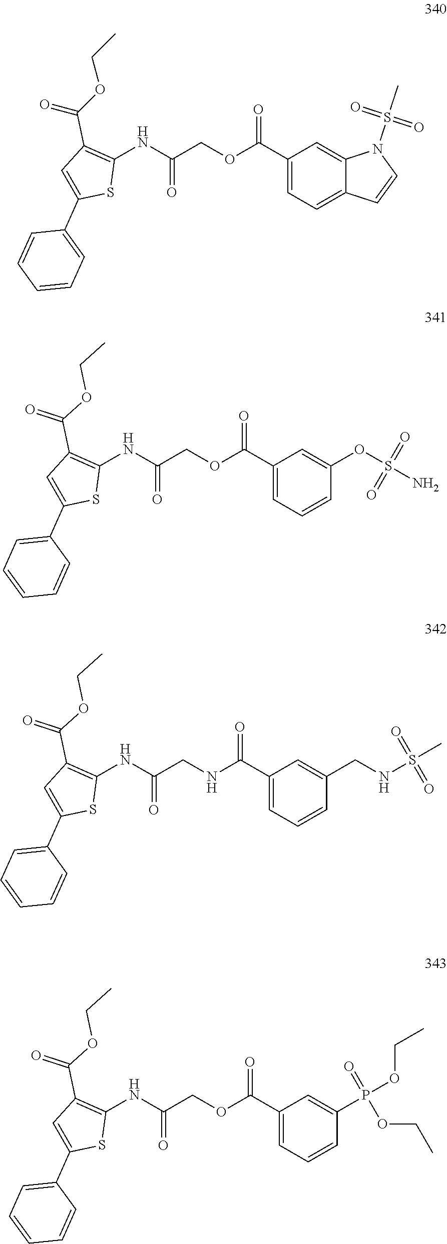 [SMILES: CCOC(=O)c1cc(-c2ccccc2)sc1NC(=O)COC(=O)c1ccc2ccn(S(C)(=O)=O)c2c1 ; CCOC(=O)c1cc(-c2ccccc2)sc1NC(=O)COC(=O)c1cccc(OS(N)(=O)=O)c1 ; CCOC(=O)c1cc(-c2ccccc2)sc1NC(=O)CNC(=O)c1cccc(CNS(C)(=O)=O)c1 ; CCOC(=O)c1cc(-c2ccccc2)sc1NC(=O)COC(=O)c1cccc(P(=O)(OCC)OCC)c1]

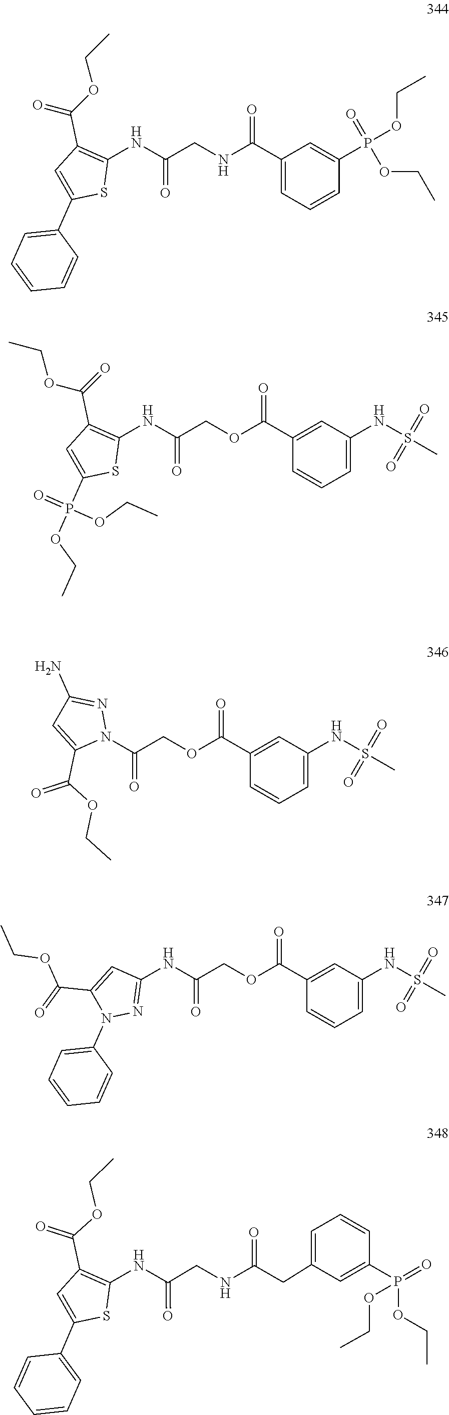

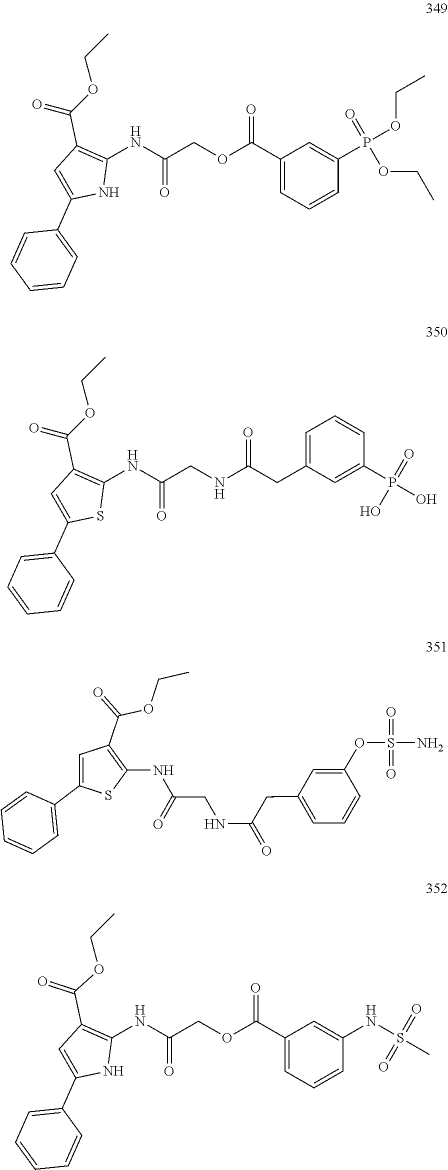 [SMILES: CCOC(=O)c1cc(-c2ccccc2)[nH]c1NC(=O)COC(=O)c1cccc(P(=O)(OCC)OCC)c1 ; CCOC(=O)c1cc(-c2ccccc2)sc1NC(=O)CNC(=O)Cc1cccc(P(=O)(O)O)c1 ; CCOC(=O)c1cc(-c2ccccc2)sc1NC(=O)CNC(=O)Cc1cccc(OS(N)(=O)=O)c1 ; CCOC(=O)c1cc(-c2ccccc2)[nH]c1NC(=O)COC(=O)c1cccc(NS(C)(=O)=O)c1]

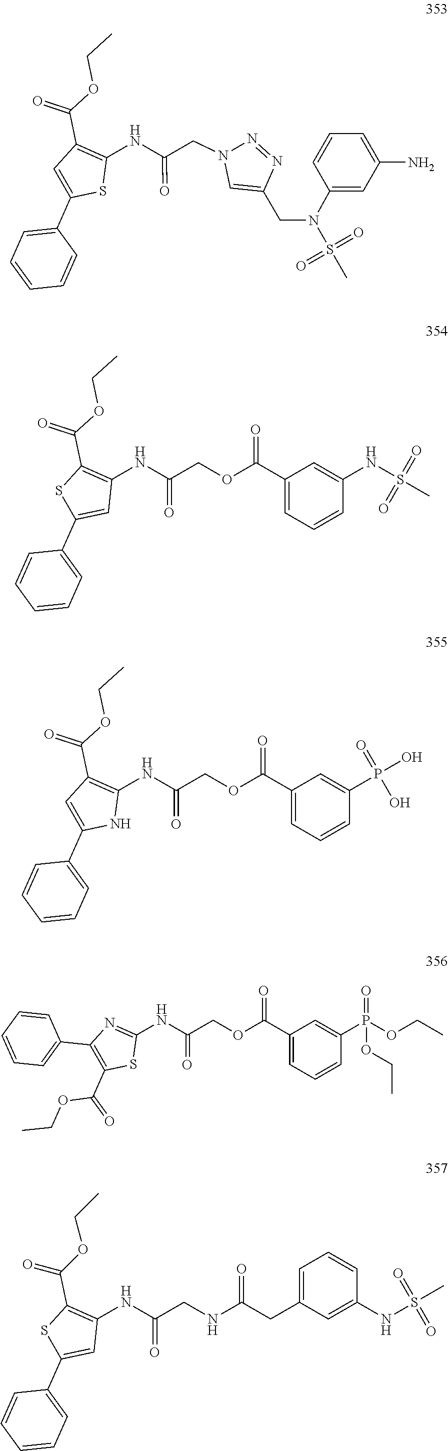

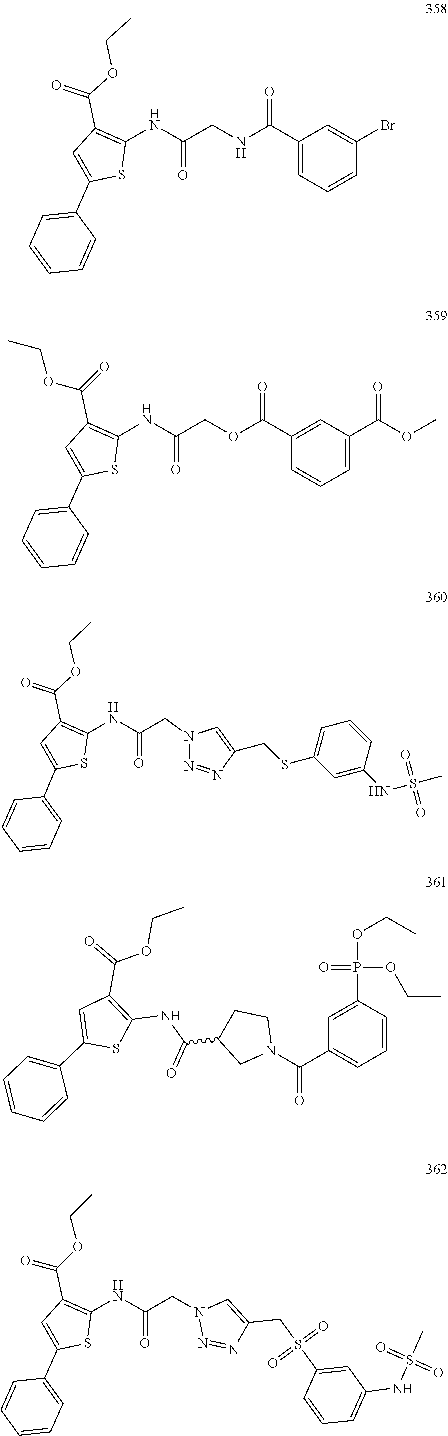 [SMILES: CCOC(=O)c1cc(-c2ccccc2)sc1NC(=O)CNC(=O)c1cccc(Br)c1 ; CCOC(=O)c1cc(-c2ccccc2)sc1NC(=O)COC(=O)c1cccc(C(=O)OC)c1 ; CCOC(=O)c1cc(-c2ccccc2)sc1NC(=O)Cn1cc(CSc2cccc(NS(C)(=O)=O)c2)nn1 ; CCOC(=O)c1cc(-c2ccccc2)sc1NC(=O)C1CCN(C(=O)c2cccc(P(=O)(OCC)OCC)c2)C1 ; CCOC(=O)c1cc(-c2ccccc2)sc1NC(=O)Cn1cc(CS(=O)(=O)c2cccc(NS(C)(=O)=O)c2)nn1]

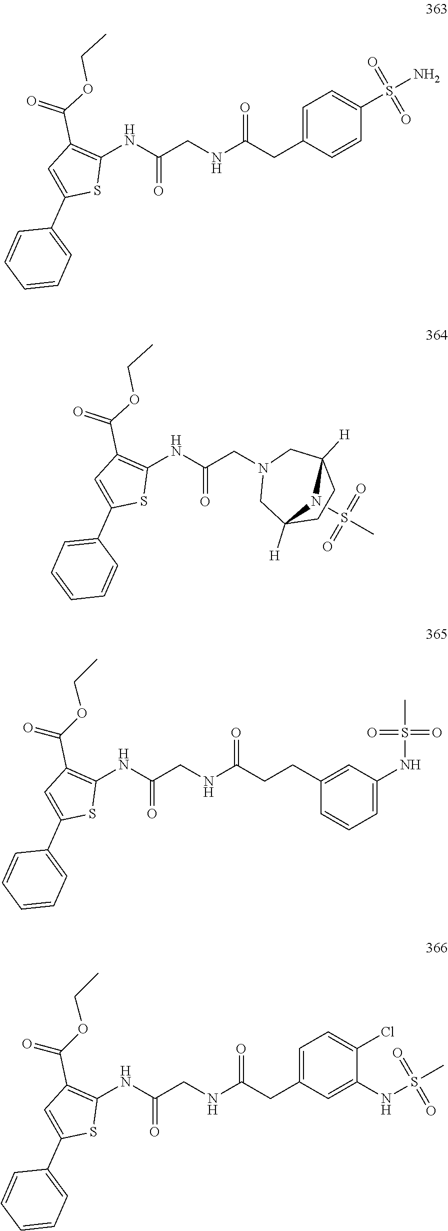 [SMILES: CCOC(=O)c1cc(-c2ccccc2)sc1NC(=O)CNC(=O)Cc1ccc(S(N)(=O)=O)cc1 ; CCOC(=O)c1cc(-c2ccccc2)sc1NC(=O)CN1C[C@H]2CC[C@@H](C1)N2S(C)(=O)=O ; CCOC(=O)c1cc(-c2ccccc2)sc1NC(=O)CNC(=O)CCc1cccc(NS(C)(=O)=O)c1 ; CCOC(=O)c1cc(-c2ccccc2)sc1NC(=O)CNC(=O)Cc1ccc(Cl)c(NS(C)(=O)=O)c1]

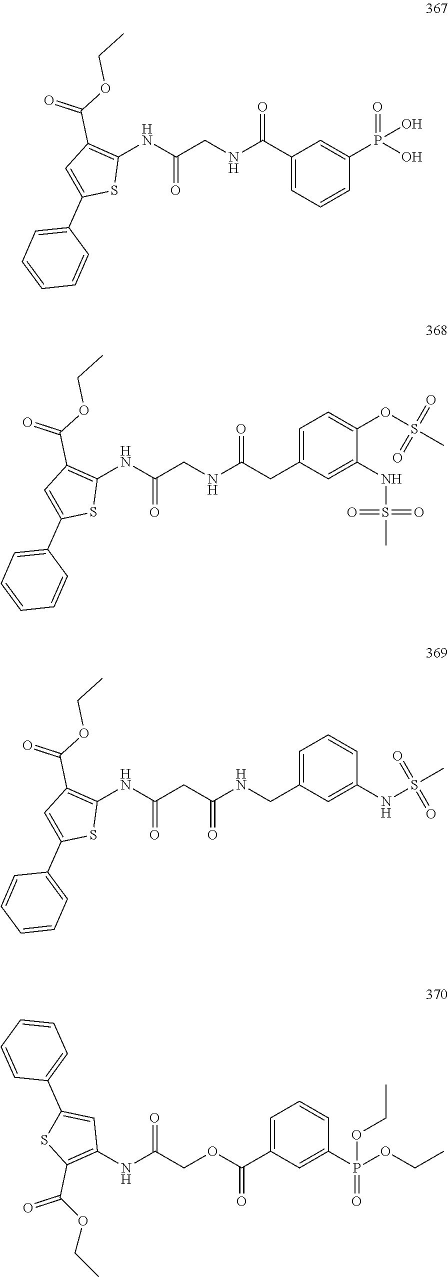 [SMILES: CCOC(=O)c1cc(-c2ccccc2)sc1NC(=O)CNC(=O)c1cccc(P(=O)(O)O)c1 ; CCOC(=O)c1cc(-c2ccccc2)sc1NC(=O)CNC(=O)Cc1ccc(OS(C)(=O)=O)c(NS(C)(=O)=O)c1 ; CCOC(=O)c1cc(-c2ccccc2)sc1NC(=O)CC(=O)NCc1cccc(NS(C)(=O)=O)c1 ; CCOC(=O)c1sc(-c2ccccc2)cc1NC(=O)COC(=O)c1cccc(P(=O)(OCC)OCC)c1]

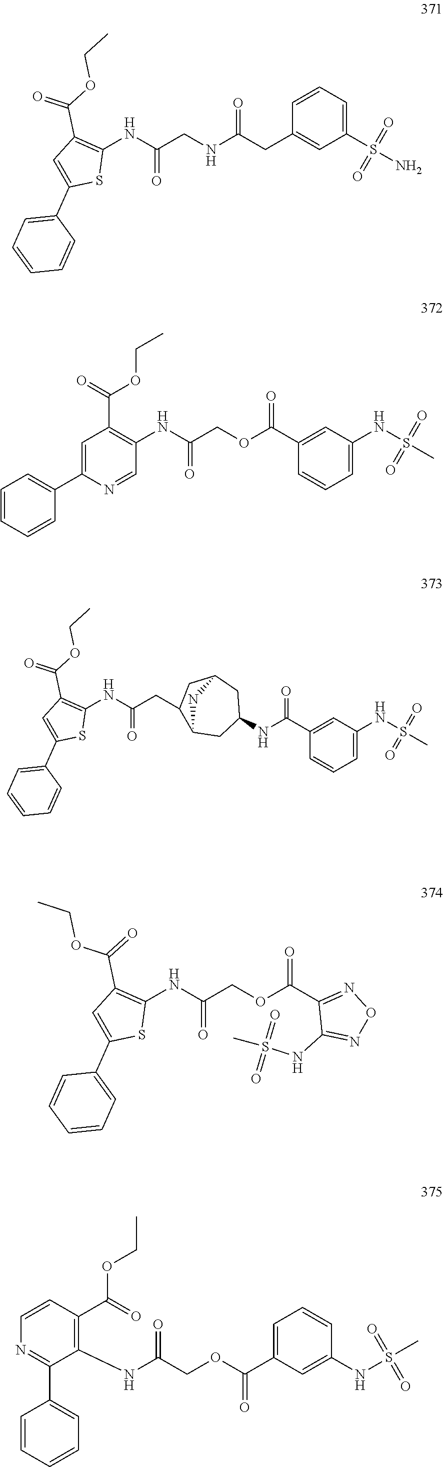

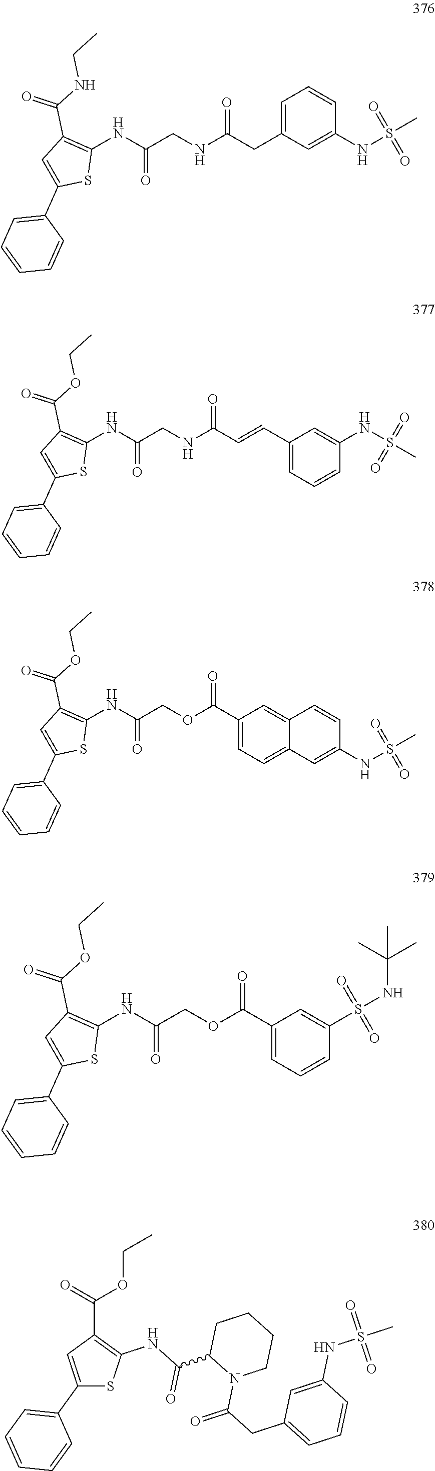 [SMILES: CCNC(=O)c1cc(-c2ccccc2)sc1NC(=O)CNC(=O)Cc1cccc(NS(C)(=O)=O)c1 ; CCOC(=O)c1cc(-c2ccccc2)sc1NC(=O)CNC(=O)/C=C/c1cccc(NS(C)(=O)=O)c1 ; CCOC(=O)c1cc(-c2ccccc2)sc1NC(=O)COC(=O)c1ccc2cc(NS(C)(=O)=O)ccc2c1 ; CCOC(=O)c1cc(-c2ccccc2)sc1NC(=O)COC(=O)c1cccc(S(=O)(=O)NC(C)(C)C)c1 ; CCOC(=O)c1cc(-c2ccccc2)sc1NC(=O)C1CCCCN1C(=O)Cc1cccc(NS(C)(=O)=O)c1]

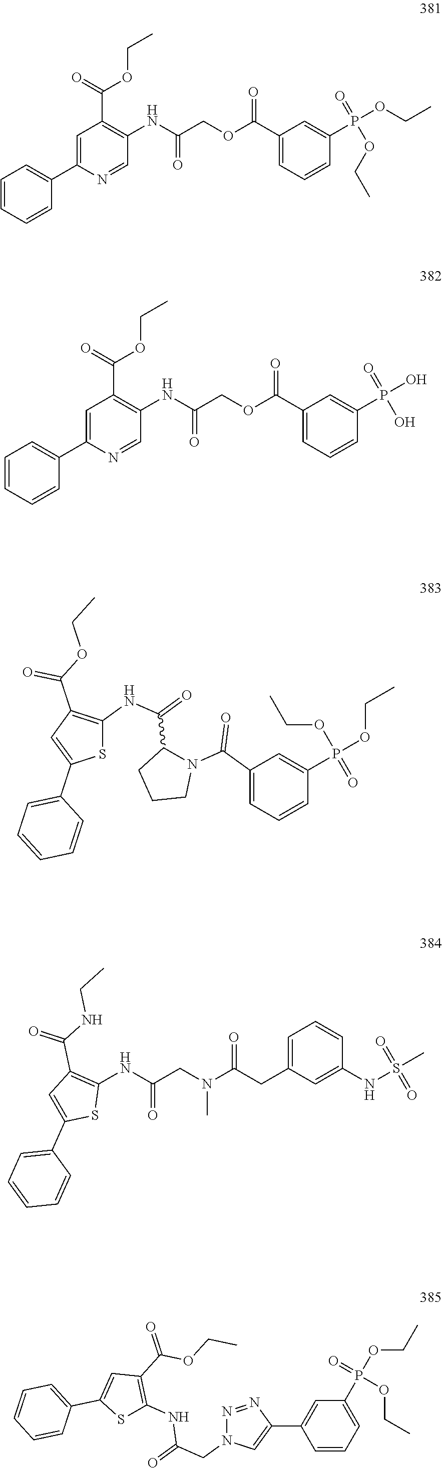 [SMILES: CCOC(=O)c1cc(-c2ccccc2)ncc1NC(=O)COC(=O)c1cccc(P(=O)(OCC)OCC)c1 ; CCOC(=O)c1cc(-c2ccccc2)ncc1NC(=O)COC(=O)c1cccc(P(=O)(O)O)c1 ; CCOC(=O)c1cc(-c2ccccc2)sc1NC(=O)C1CCCN1C(=O)c1cccc(P(=O)(OCC)OCC)c1 ; CCNC(=O)c1cc(-c2ccccc2)sc1NC(=O)CN(C)C(=O)Cc1cccc(NS(C)(=O)=O)c1 ; CCOC(=O)c1cc(-c2ccccc2)sc1NC(=O)Cn1cc(-c2cccc(P(=O)(OCC)OCC)c2)nn1]

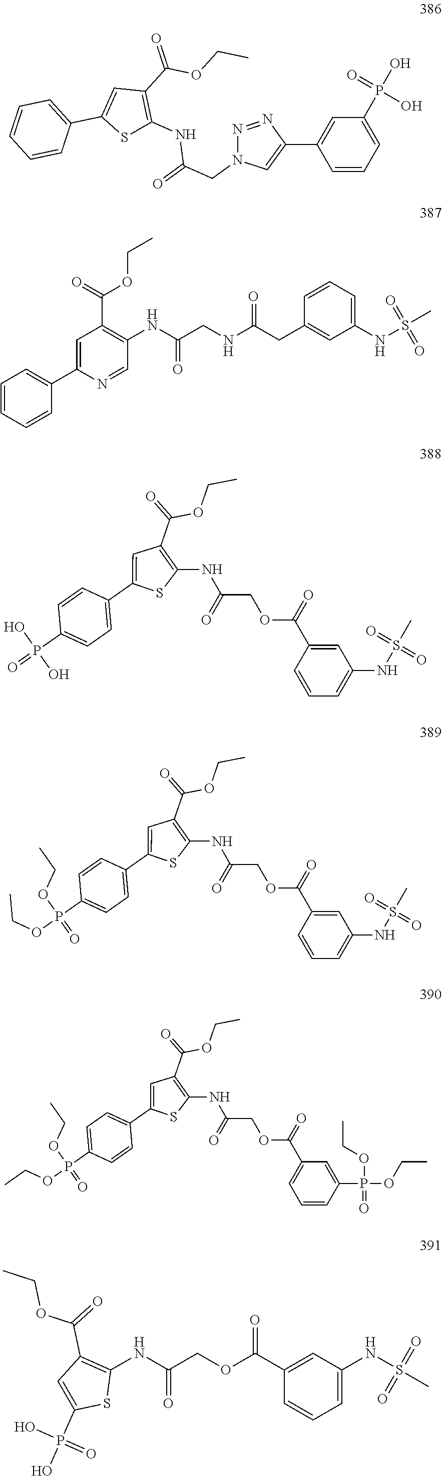

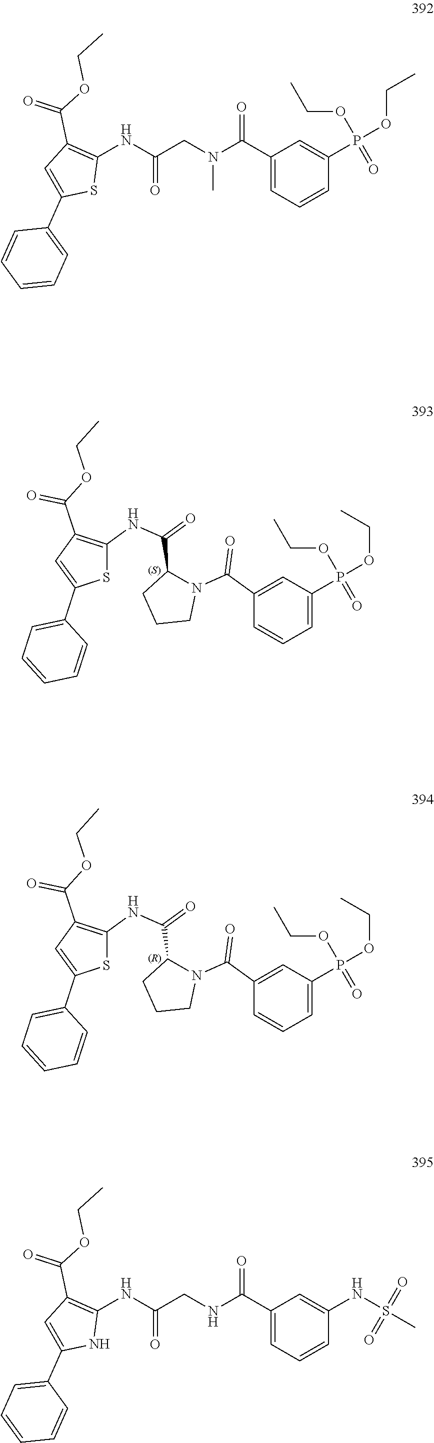

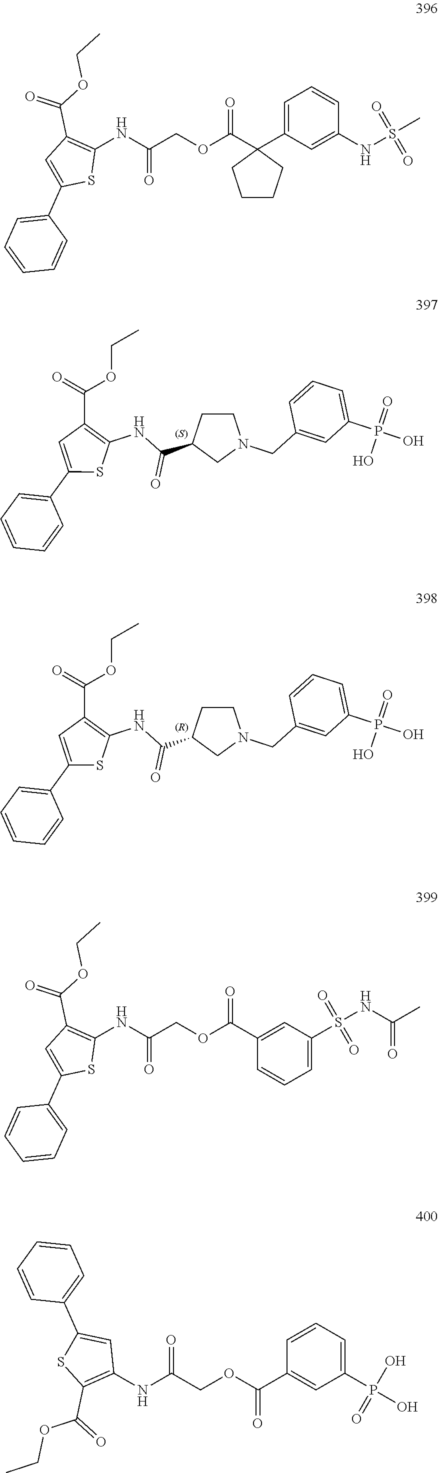

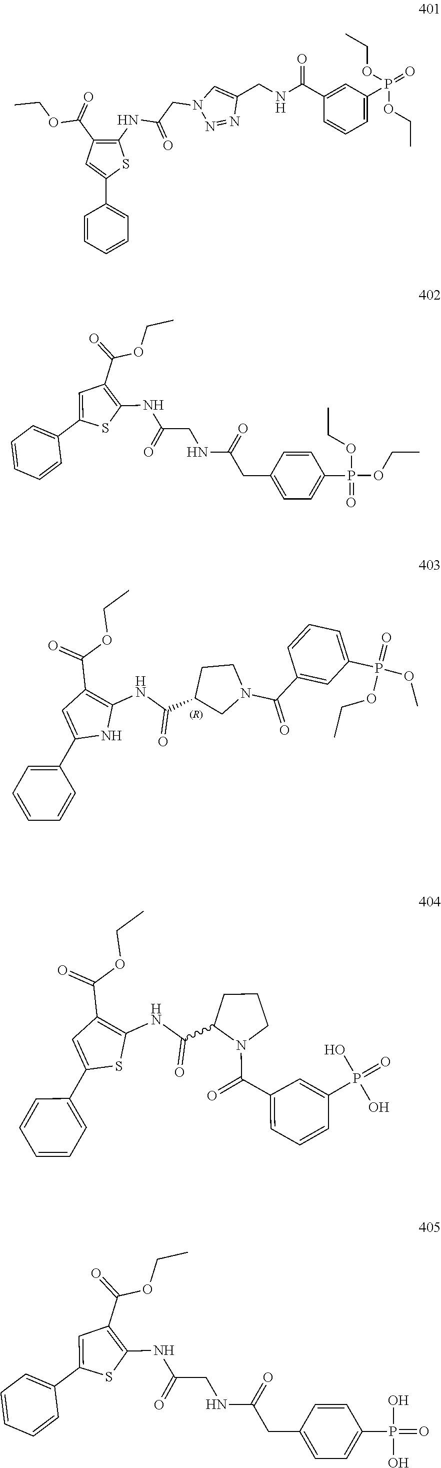

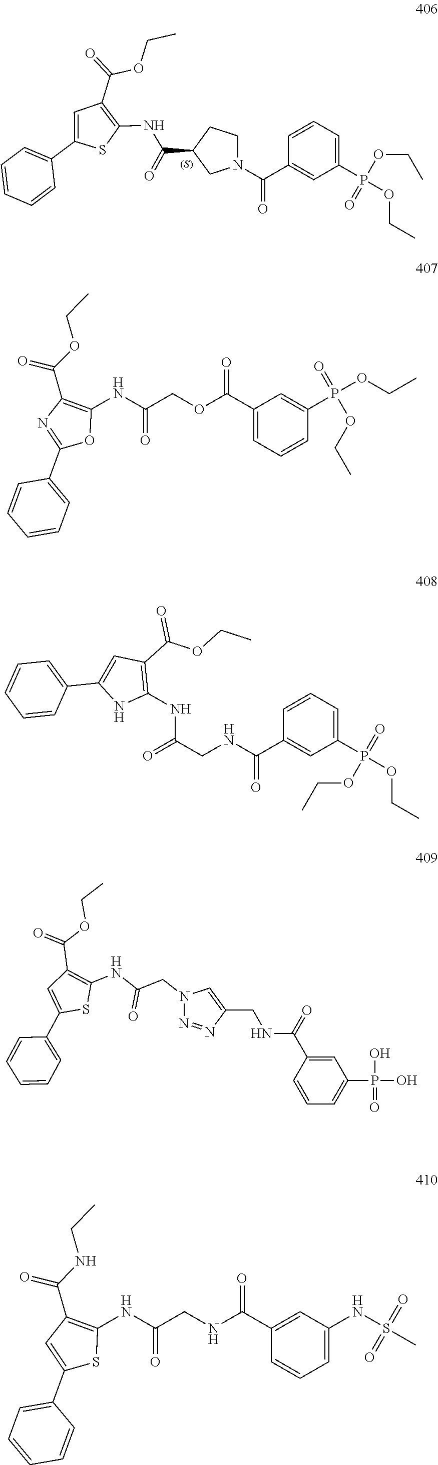

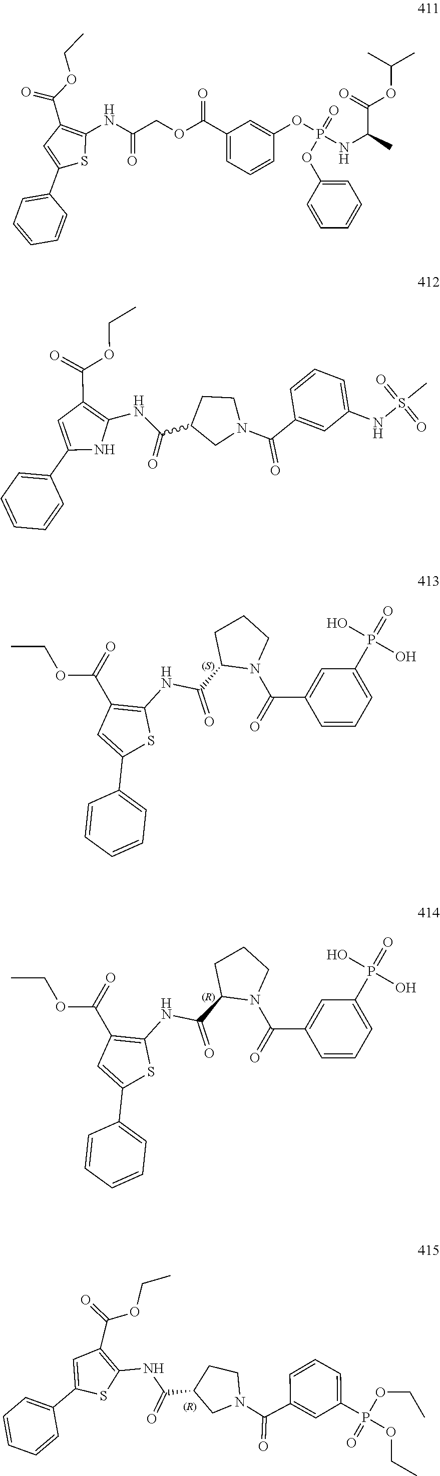

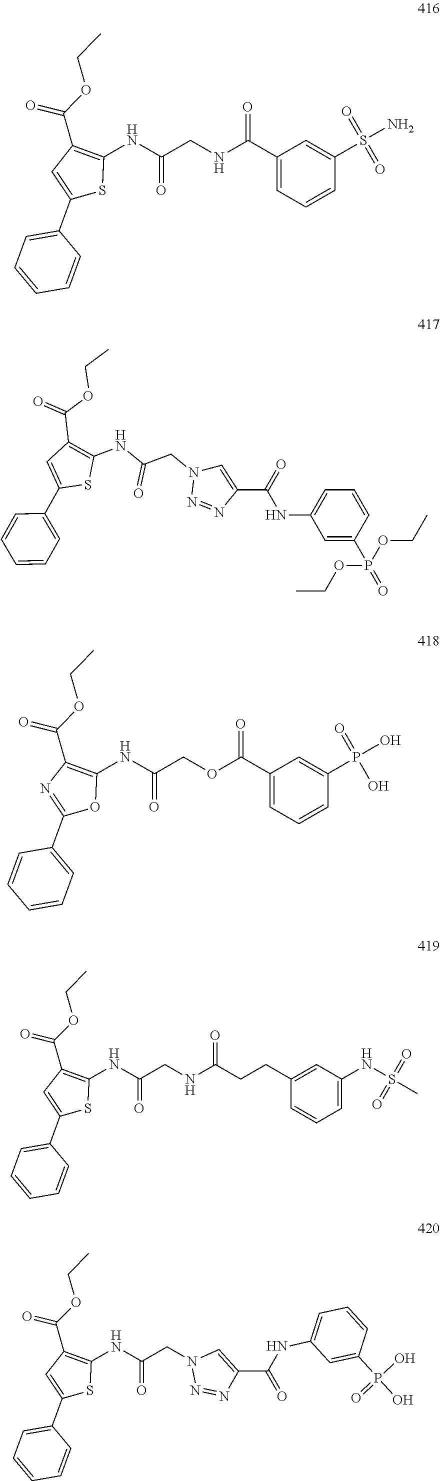 [SMILES: CCOC(=O)c1cc(-c2ccccc2)sc1NC(=O)CNC(=O)c1cccc(S(N)(=O)=O)c1 ; CCOC(=O)c1cc(-c2ccccc2)sc1NC(=O)Cn1cc(C(=O)Nc2cccc(P(=O)(OCC)OCC)c2)nn1 ; CCOC(=O)c1nc(-c2ccccc2)oc1NC(=O)COC(=O)c1cccc(P(=O)(O)O)c1 ; CCOC(=O)c1cc(-c2ccccc2)sc1NC(=O)CNC(=O)CCc1cccc(NS(C)(=O)=O)c1 ; CCOC(=O)c1cc(-c2ccccc2)sc1NC(=O)Cn1cc(C(=O)Nc2cccc(P(=O)(O)O)c2)nn1]

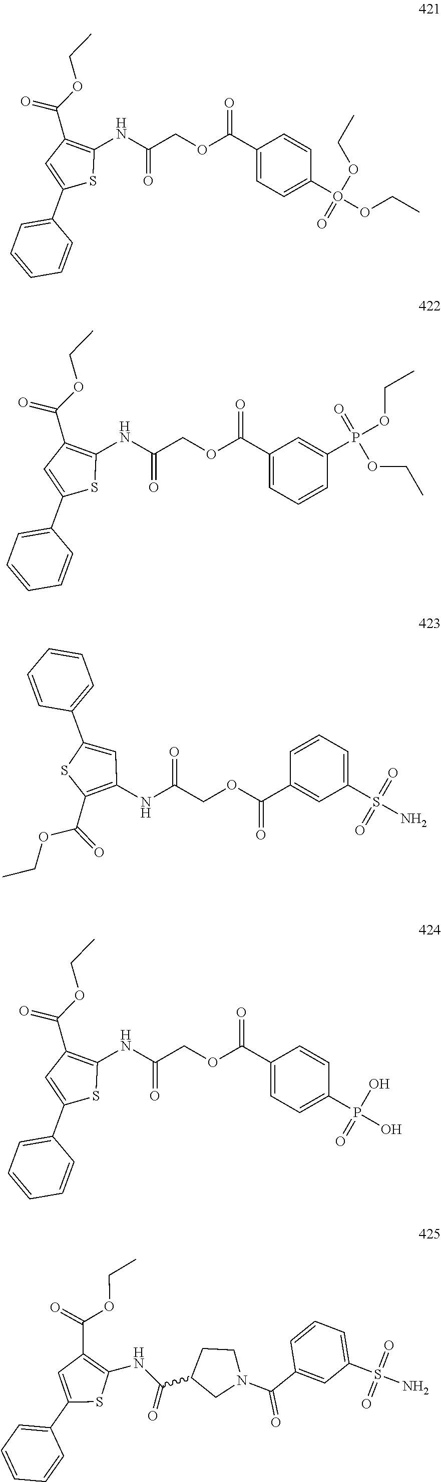

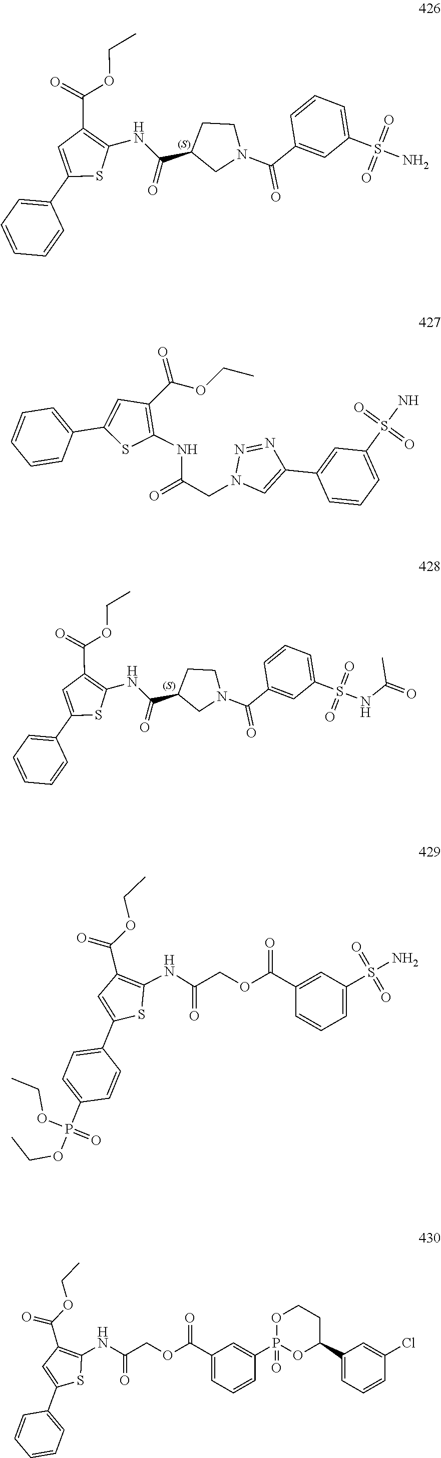

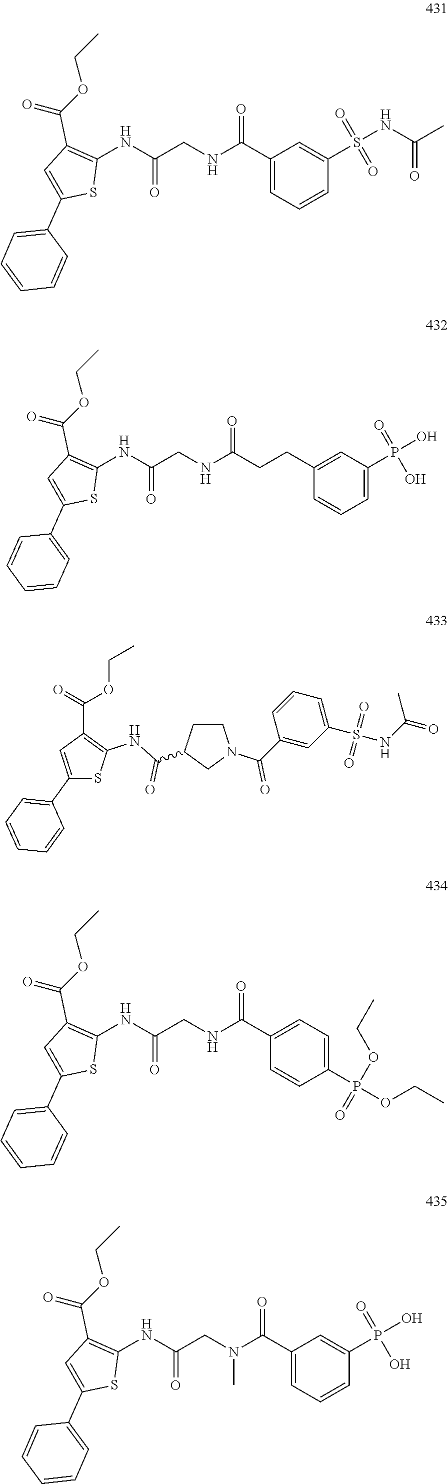 [SMILES: CCOC(=O)c1cc(-c2ccccc2)sc1NC(=O)CNC(=O)c1cccc(S(=O)(=O)NC(C)=O)c1 ; CCOC(=O)c1cc(-c2ccccc2)sc1NC(=O)CNC(=O)CCc1cccc(P(=O)(O)O)c1 ; CCOC(=O)c1cc(-c2ccccc2)sc1NC(=O)C1CCN(C(=O)c2cccc(S(=O)(=O)NC(C)=O)c2)C1 ; CCOC(=O)c1cc(-c2ccccc2)sc1NC(=O)CNC(=O)c1ccc(P(=O)(OCC)OCC)cc1 ; CCOC(=O)c1cc(-c2ccccc2)sc1NC(=O)CN(C)C(=O)c1cccc(P(=O)(O)O)c1]

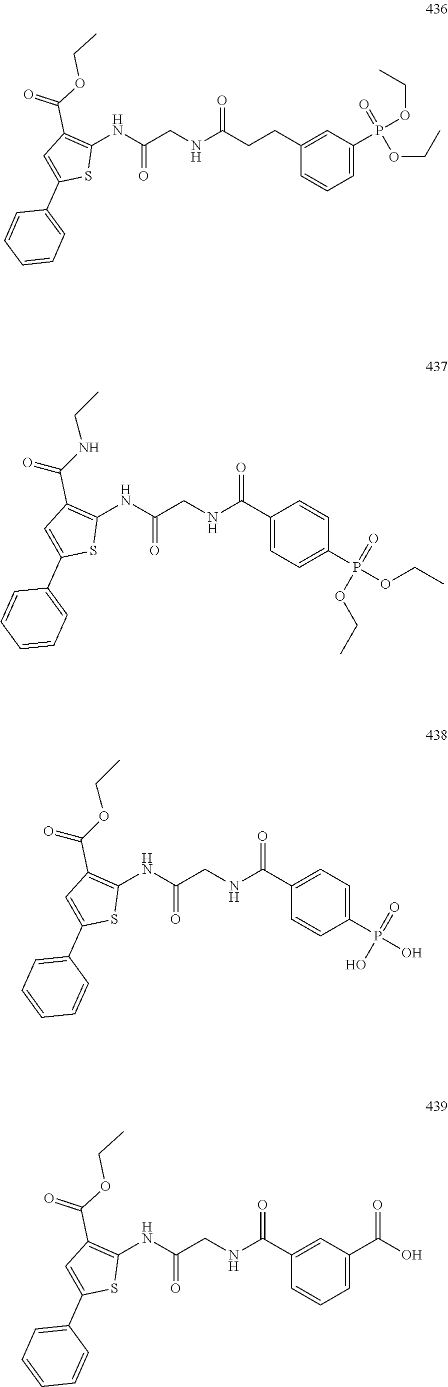 [SMILES: CCOC(=O)c1cc(-c2ccccc2)sc1NC(=O)CNC(=O)CCc1cccc(P(=O)(OCC)OCC)c1 ; CCNC(=O)c1cc(-c2ccccc2)sc1NC(=O)CNC(=O)c1ccc(P(=O)(OCC)OCC)cc1 ; CCOC(=O)c1cc(-c2ccccc2)sc1NC(=O)CNC(=O)c1ccc(P(=O)(O)O)cc1 ; CCOC(=O)c1cc(-c2ccccc2)sc1NC(=O)CNC(=O)c1cccc(C(=O)O)c1]

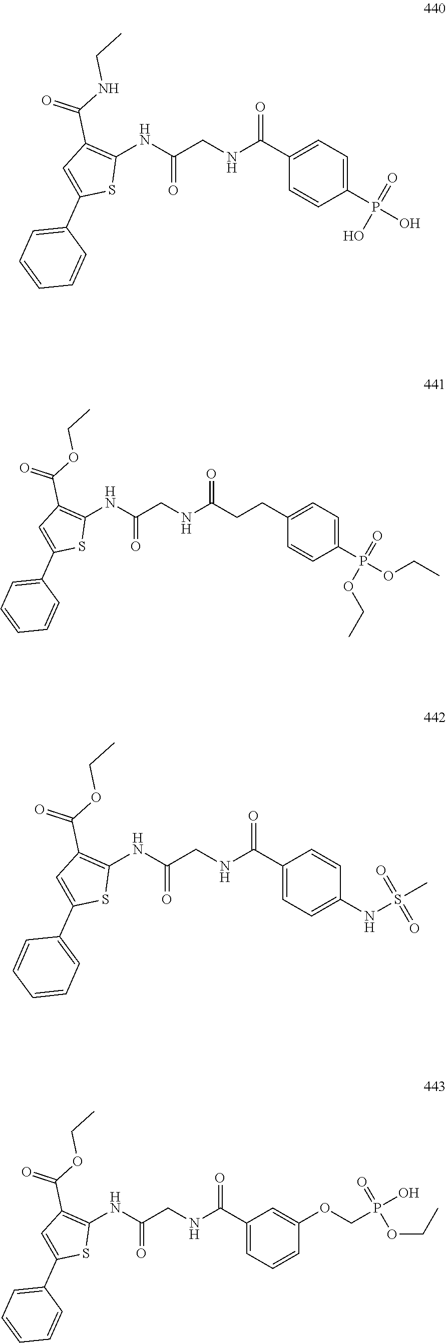 [SMILES: CCNC(=O)c1cc(-c2ccccc2)sc1NC(=O)CNC(=O)c1ccc(P(=O)(O)O)cc1 ; CCOC(=O)c1cc(-c2ccccc2)sc1NC(=O)CNC(=O)CCc1ccc(P(=O)(OCC)OCC)cc1 ; CCOC(=O)c1cc(-c2ccccc2)sc1NC(=O)CNC(=O)c1ccc(NS(C)(=O)=O)cc1 ; CCOC(=O)c1cc(-c2ccccc2)sc1NC(=O)CNC(=O)c1cccc(OCP(=O)(O)OCC)c1]

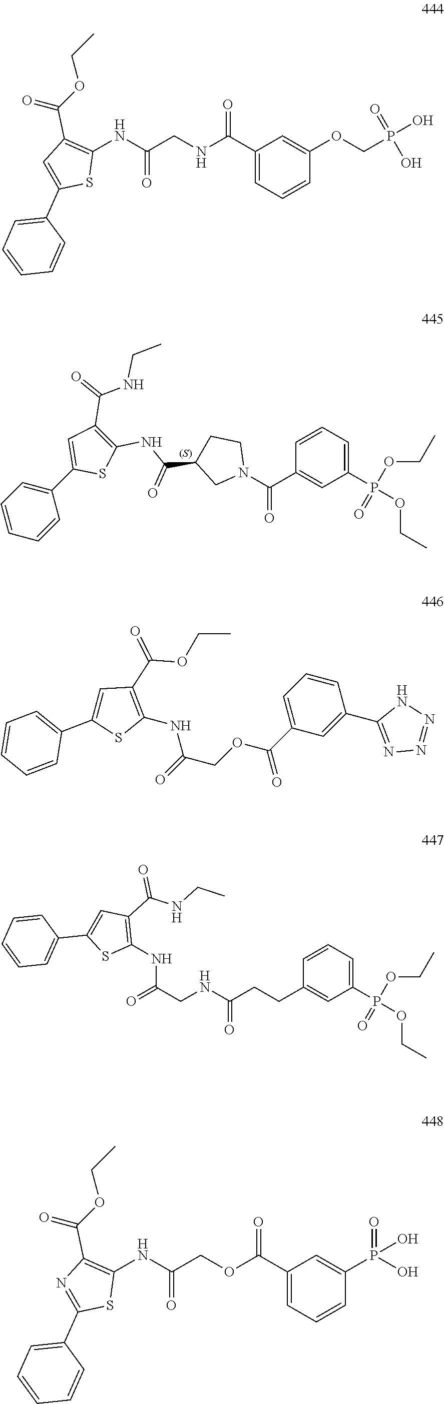

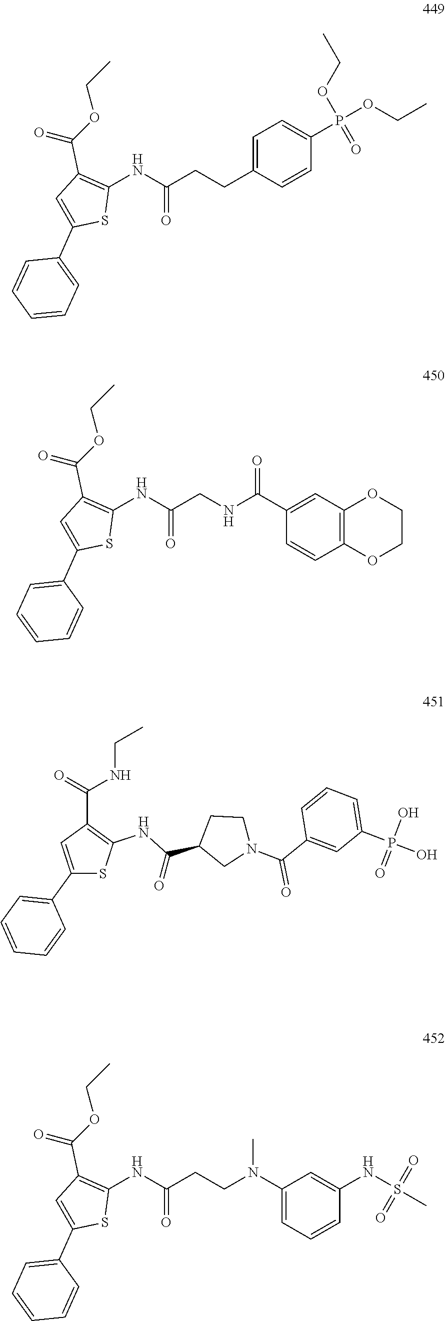 [SMILES: CCOC(=O)c1cc(-c2ccccc2)sc1NC(=O)CCc1ccc(P(=O)(OCC)OCC)cc1 ; CCOC(=O)c1cc(-c2ccccc2)sc1NC(=O)CNC(=O)c1ccc2c(c1)OCCO2 ; CCNC(=O)c1cc(-c2ccccc2)sc1NC(=O)[C@H]1CCN(C(=O)c2cccc(P(=O)(O)O)c2)C1 ; CCOC(=O)c1cc(-c2ccccc2)sc1NC(=O)CCN(C)c1cccc(NS(C)(=O)=O)c1]

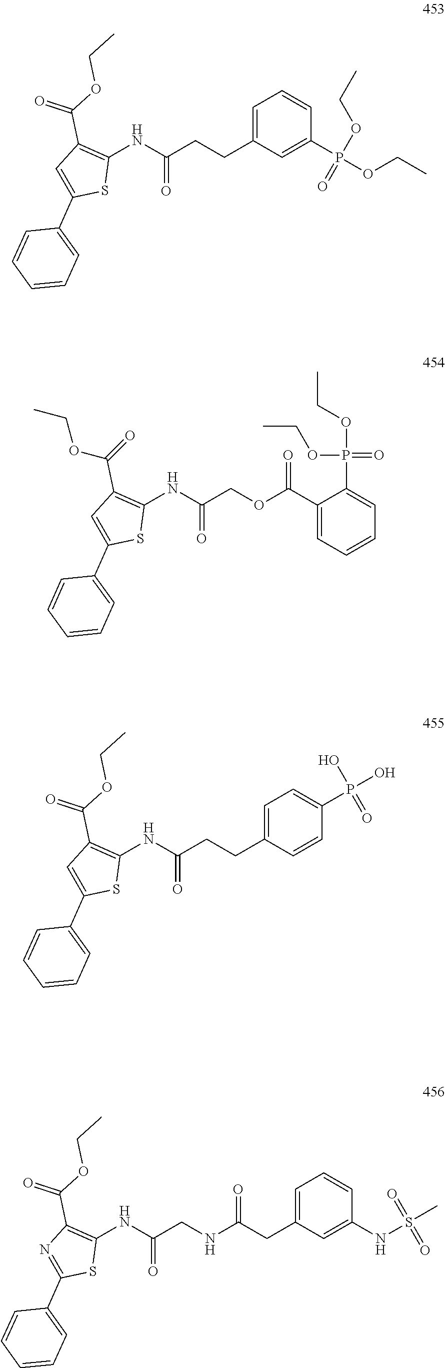

and a pharmaceutically acceptable salt or tautomer thereof.

In some embodiments, the compound of the present disclosure is a compound provided in Table 1, or a pharmaceutically acceptable salt or tautomer thereof.

In some embodiments, the compound of the present disclosure is a compound provided in Table 2, or a pharmaceutically acceptable salt or tautomer thereof. In some embodiments, the compound of the present disclosure is a compound provided in Table 2 having “A” activity, or a pharmaceutically acceptable salt or tautomer thereof.

The compounds described herein for Formula (I), Formula (I-A), Formula (I-A1), Formula (I-A2), Formula (I-A3), Formula (I-A4), Formula (I-B), Formula (I-B1), Formula (I-B1a), Formula (II), Formula (II-A), Formula (II-A1), Formula (II-A2), Formula (II-B), Formula (II-B1), and Formula (A) are meant to include all racemic mixtures, all individual enantiomers or combinations thereof, as well as all diastereomers or combinations thereof when two or more stereocenters are present, regardless of whether or not they are specifically depicted herein.

### Pharmaceutical Compositions

In some embodiments, the present disclosure provides pharmaceutical compositions comprising (i) an effective amount of a compound disclosed herein, or a pharmaceutically acceptable salt or tautomer thereof; and (ii) one or more pharmaceutically acceptable carriers or excipients.

In some embodiments, the present disclosure provides pharmaceutical compositions comprising (i) a compound disclosed herein, or a pharmaceutically acceptable salt or tautomer thereof; (ii) an immune checkpoint inhibitor (ICI); and (iii) one or more pharmaceutically acceptable carriers or excipients.

In various embodiments, the pharmaceutical compositions of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The effective amount of a compound of the present disclosure, including pharmaceutically acceptable salts, esters, prodrugs, hydrates, solvates and isomers thereof, or pharmaceutical compositions thereof may be determined by one skilled in the art based on known methods.

In one embodiment, a pharmaceutical composition or a pharmaceutical formulation comprises a compound of the present disclosure, or a pharmaceutically acceptable salt thereof, and a pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M, for example 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, for example sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets for example contain up to 99% of the active compound. Suitable solid carriers include, for example, calcium phosphate, magnesium stearate, talc, sugars, lactose, dextrin, starch, gelatin, cellulose, polyvinylpyrrolidone, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGITI), potassium chloride, powdered cellulose, sodium chloride, sorbitol, and talc.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington's Pharmaceutical Sciences). In particular embodiments, the composition is administered topically, instead of enterally. For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, such as in physiologically compatible buffers like as Hank's solution, Ringer's solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

Dragee cores are provided with suitable coatings. For this purpose, concentrated sugar solutions may be used, which may optionally contain talc, polyvinylpyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds doses.

### Methods of Treatment

The present disclosure provides compounds and compositions that are useful in treating cancers and other conditions associated with abnormal levels of ADAR1 expression. In some embodiments, the cancers and other conditions are associated with increased ADAR1 expression. In some embodiments, the compounds disclosed herein are selective inhibitors of ADAR1. In some embodiments, the compounds disclosed herein are selective inhibitors of ADAR1 p150.

In some embodiments, the present methods are useful in treating disorders of uncontrolled cellular proliferation in a subject in need thereof comprising administering to the subject a therapeutic amount of a compound disclosed herein e.g., a compound of Formula (I), Formula (I-A), Formula (I-A1), Formula (I-A2), Formula (I-A3), Formula (I-A4), Formula (I-B), Formula (I-B1), Formula (I-B1a), Formula (II), Formula (II-A), Formula (II-A1), Formula (II-A2), Formula (II-B), Formula (II-B1), or Formula (A)), a pharmaceutically acceptable salt or tautomer thereof, or a composition thereof. In some embodiments, the disorder of uncontrolled cellular proliferation is a cancer or a tumor. In some embodiments, the disorder or uncontrolled cellular proliferation is associated with elevated levels of ADAR1 expression. In some embodiments, the disorder or uncontrolled cellular proliferation is associated with elevated levels of the ADAR1 p150 isoform.

In some embodiments, the present disclosure provides methods of treating cancer in a subject in need thereof, comprising administering to the subject an effective amount of a compound disclosed herein (e.g., a compound of Formula (I), Formula (I-A), Formula (I-A1), Formula (I-A2), Formula (I-A3), Formula (I-A4), Formula (I-B), Formula (I-B1), Formula (I-B1a), Formula (II), Formula (II-A), Formula (II-A1), Formula (II-A2), Formula (II-B), Formula (II-B1), or Formula (A)), or a pharmaceutically acceptable salt or tautomer thereof, or a pharmaceutical composition thereof.

In some embodiments, the present disclosure provides methods of treating cancer in a subject in need thereof, comprising administering to the subject (i) a compound disclosed herein (e.g., a compound of Formula (I), Formula (I-A), Formula (I-A1), Formula (I-A2), Formula (I-A3), Formula (I-A4), Formula (I-B), Formula (I-B1), Formula (I-B1a), Formula (II), Formula (II-A), Formula (II-A1), Formula (II-A2), Formula (II-B), Formula (II-B1), or Formula (A)), or a pharmaceutically acceptable salt or tautomer thereof, or a pharmaceutical composition thereof; and (ii) an immune checkpoint inhibitor, in an amount effective to treat the subject.

In some embodiments, the present methods comprise administering to the subject a compound of Table 2. In some embodiments, the present methods comprise administering to the subject a compound of Table 2 having “A” activity.

In some embodiments, the present methods further comprise administering to the subject an immune checkpoint inhibitor in combination with a compound of the present disclosure (e.g., a compound of Formula (I), Formula (I-A), Formula (I-A1), Formula (I-A2), Formula (I-A3), Formula (I-A4), Formula (I-B), Formula (I-B1), Formula (I-B1a), Formula (II), Formula (II-A), Formula (II-A1), Formula (II-A2), Formula (II-B), Formula (II-B1), or Formula (A)). In some embodiments, the immune checkpoint inhibitor is an anti-programmed cell death protein-1 (anti-PD-1) or anti-programmed cell death protein-1/ligand-1 (anti-PD-L1) antibody. In some embodiments, the anti-PD-1 antibody is pembrolizumab, nivolumab, cemiplimab, dostarlimab, or retifanlimab. In some embodiments, the anti-PD-L1 antibody is durvalumab, atezolizumab, or avelumab.

In some embodiments of the present methods, administering the compound disclosed herein and an immune checkpoint inhibitor provides a synergistic effect in treating cancer.

In some embodiments, the cancer is a cancer characterized by overexpression of ADAR1 relative to normal cells. In some embodiments, the cancer is breast cancer, lung cancer, pancreatic cancer, melanoma, multiple myeloma, colon carcinoma, colorectal cancer, or glioblastoma.

The present disclosure further provides methods of treating an RNA virus infection in a subject in need thereof, comprising administering to the subject an effective amount of a compound disclosed herein or the pharmaceutical composition thereof.

In some embodiments, the RNA viral infection is HIV-1, HTLV-1, or SARS-CoV-2.

### NUMBERED EMBODIMENTS

1. A compound of Formula (I) having the structure:

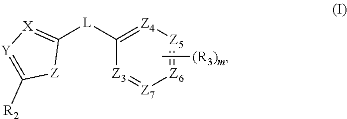

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, or —S(O)₂NR_{c}—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

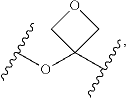 [SMILES: *OC1(*)COC1]

—OC(CF₃)—, —O—, —C(O)-alkylene-, —C(O)—, —NR_{d}S(O₂)—, —S(O₂)NR_{d}—, or heteroaryl;
R_{c} and R_{d} are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}—NR_{d}—, or S—S bond;
X is C—R₁ or N;
Y is C—R₄ or N;
Z is O, S, N—R₅, or N(R₅)—CH₂—;
Z₃, Z₄, Z₅, Z₆, and Z₇ are each independently N or CH, wherein no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, —CN, or —P(O)(OR_{b})₂, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, alkyl, alkylenecycloalkyl, cycloalkyl, aryl, alkylenearyl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
   wherein the compound is not:

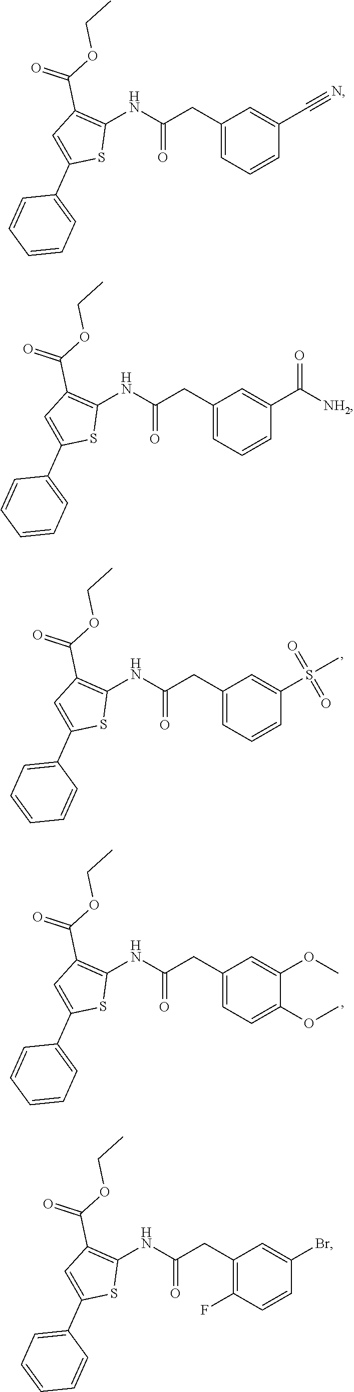 [SMILES: CCOC(=O)c1cc(-c2ccccc2)sc1NC(=O)Cc1cccc(C#N)c1 ; CCOC(=O)c1cc(-c2ccccc2)sc1NC(=O)Cc1cccc(C(N)=O)c1 ; CCOC(=O)c1cc(-c2ccccc2)sc1NC(=O)Cc1cccc(S(C)(=O)=O)c1 ; CCOC(=O)c1cc(-c2ccccc2)sc1NC(=O)Cc1ccc(OC)c(OC)c1 ; CCOC(=O)c1cc(-c2ccccc2)sc1NC(=O)Cc1cc(Br)ccc1F]

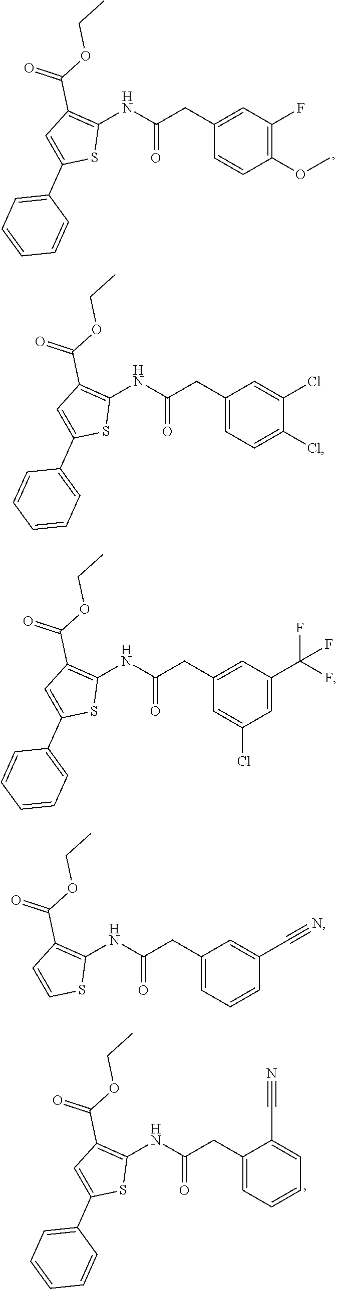

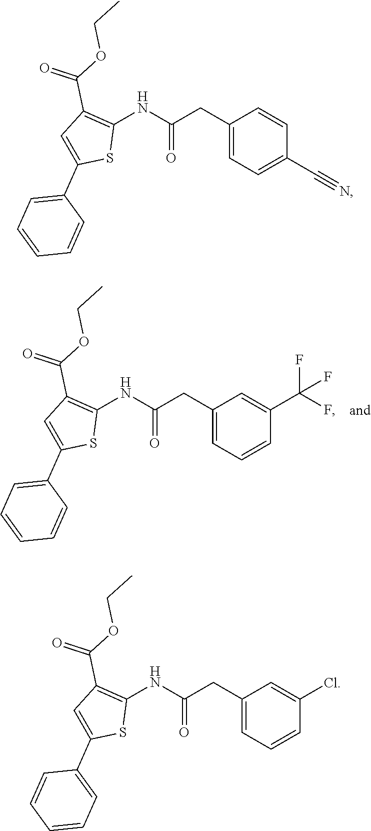 [SMILES: CCOC(=O)c1cc(-c2ccccc2)sc1NC(=O)Cc1ccc(C#N)cc1 ; CCOC(=O)c1cc(-c2ccccc2)sc1NC(=O)Cc1cccc(C(F)(F)F)c1 ; "and" ; CCOC(=O)c1cc(-c2ccccc2)sc1NC(=O)Cc1cccc(Cl)c1]

2. The compound of embodiment 1, wherein at least two of R₁, R₂, and R₄ are not H.
3. The compound of embodiment 1 or 2, wherein no more than one of Z₃, Z₄, Z₅, Z₆, and Z₇ is N.
4. The compound of embodiment 1 or 2, wherein Z₃, Z₄, Z₅, Z₆, and Z₇ is each H.
5. The compound of any one of embodiments 1-4 having the structure:

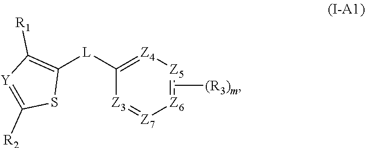

or a pharmaceutically acceptable salt or tautomer thereof.
6. The compound of any one of embodiments 1-4, wherein the compound has the structure:

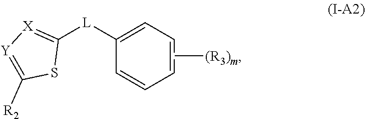

or a pharmaceutically acceptable salt thereof.
7. The compound of any one of embodiments 1-6, wherein the compound has the structure:

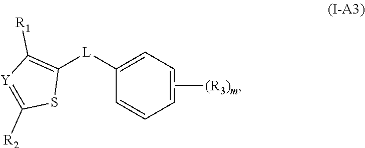

or a pharmaceutically acceptable salt thereof.
8. The compound of any one of embodiments 1-7, wherein the compound has the structure:

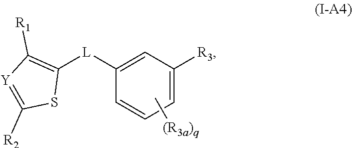

or a pharmaceutically acceptable salt thereof,
wherein:
R₃ₐ is each independently halogen, —OH, alkyl, haloalkyl, alkoxy, or haloalkoxy; and
q is 0, 1, or 2.
   9. The compound of any one of embodiments 1-8, wherein L is a linker having the structure -A-L₁-B—, wherein:
A is —NR_{c}C(O)— or —C(O)NR_{c};
L₁ is absent, alkylene, cycloalkyl, or heterocyclyl;
B is a bond, —NR_{d}—, —N(S(O)₂-alkyl)-, —NR_{d}C(O)—, —OC(O)—, —C(O)-alkylene-, —C(O)—, —NR_{d}S(O₂)—, or —S(O₂)NR_{d}—; and
R_{c} and R_{d} are each independently H, alkyl, cycloalkyl, or alkylene-aryl, with the proviso that -A-L₁-B— does not contain an —O—O, —NR_{c}—O, —O—NR_{d}—, —NR_{c}—NR_{d}—, or S—S bond.
   10. The compound of any one of embodiments 1-9, wherein A is —NR_{c}C(O)—, —C(O)NR_{c}—, or —C(O)—.
   11. The compound of any one of embodiments 1-10, wherein A is —NR_{c}C(O)—.
   12. The compound of any one of embodiments 1-11, wherein R_{c} is H, C₁₋₅alkyl, C₃₋₆cycloalkyl, or —CH₂-phenyl.
   13. The compound of any one of embodiments 1-12, wherein L₁ is absent, alkylene, or heterocyclyl.
   14. The compound of any one of embodiments 1-13, wherein L₁ is alkylene, and the alkylene is a C₁₋₃ alkylene.
   15. The compound of any one of embodiments 1-14, wherein the alkylene of L₁ is an optionally substituted methylene.
   16. The compound of any one of embodiments 1-15, wherein the alkylene of L₁ comprises the structure:

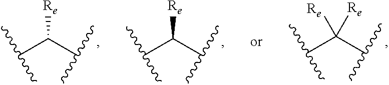

wherein Rₑ is each independently H, F, C₁₋₅ alkyl, —CH₂OH, —CH₂cycloalkyl, — or CH₂aryl, or two Rₑ taken together with the carbon atom to which they are attached form a C₃₋₆ cycloalkyl or a 4- to 6-membered heterocyclyl.
17. The compound of embodiment 16, wherein Rₑ is H or C₁₋₅ alkyl.
18. The compound of any one of embodiments 1-13, wherein L₁ is a heterocyclyl.
19. The compound of any one of embodiments 1-13 and 18, wherein the heterocyclyl of L₁ is a 3- to 12-membered heterocyclyl.
20. The compound of any one of embodiments 1-13, 18, and 19, wherein the heterocyclyl of L₁ has the structure:

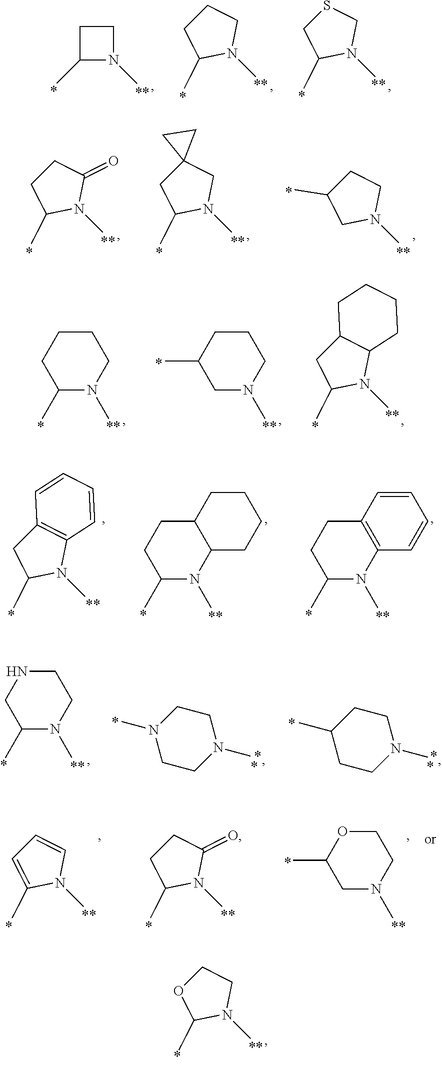

wherein * represents the attachment point to A and ** represents the attachment point to B.
21. The compound of any one of embodiments 1-20, wherein B is bond, —NR_{b}C(O)—, —NR_{b}S(O)₂—, —C(O)—, —OC(O)—, —C(O)—O—, or heteroaryl.
22. The compound of any one of embodiments 1-21, wherein B is a bond, —OC(O)—, or —C(O)—.
23. The compound of embodiment 21, wherein the heteroaryl is a 5- or 6-membered nitrogen-containing heteroaryl.
24. The compound of any one of embodiments 1-22, wherein -A-L₁-B— has the structure:

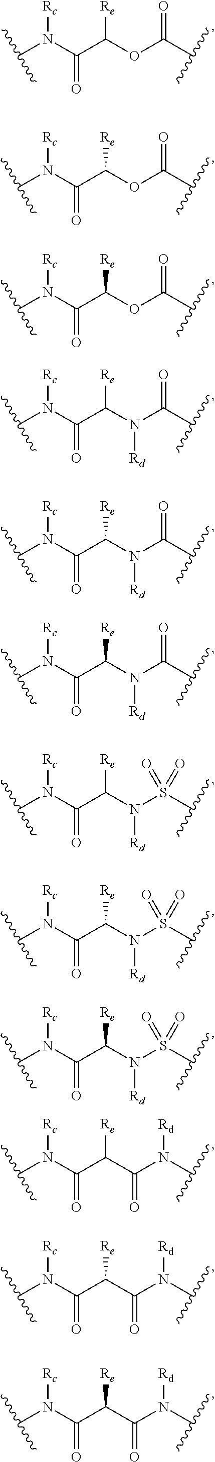

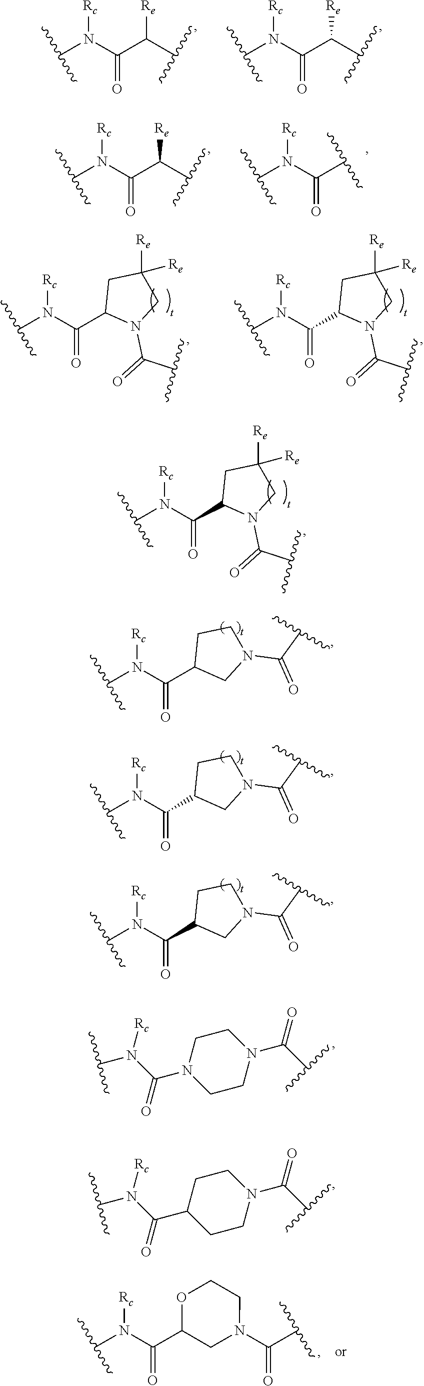

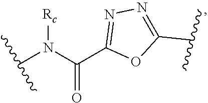

wherein:
   R_{c} and R_{d} are each independently H, alkyl, cycloalkyl, alkylene-aryl, acyl, or sulfonyl;
   Rₑ is each independently H, F, C₁₋₅ alkyl, —CH₂cycloalkyl, — or CH₂aryl, or two Rₑ taken together with the carbon atom to which they are attached form a C₃₋₆ cycloalkyl or a 4- to 6-membered heterocyclyl; and
   t is 0, 1 or 2.
      25. The compound of any one of embodiments 1-24, wherein A-L₁-B has the structure:

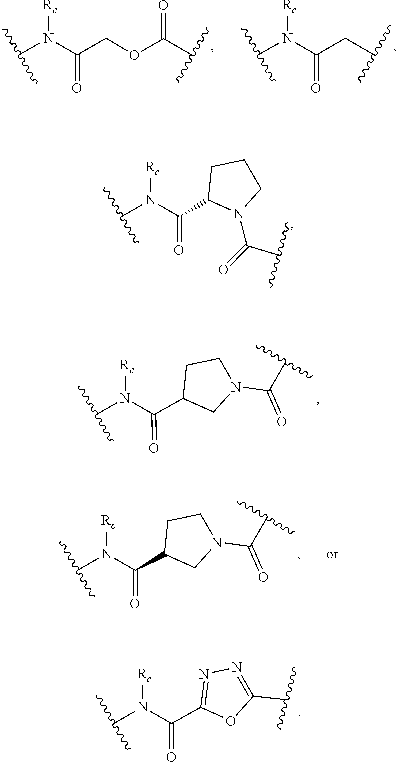

26. The compound of embodiment 24 or 25, wherein R_{c} is H.
27. The compound of any one of embodiments 1-26, wherein X is C—R₁.
28 The compound of any one of embodiments 1-27, wherein R₁ is halogen, alkyl, —C(O)ORₐ, or —C(O)NHRₐ.
29 The compound of any one of embodiments 1-28, wherein R₁ is —C(O)ORₐ.
30. The compound of any one of embodiments 1-26, wherein X is N.
31. The compound of any one of embodiments 1-30, wherein Y is C—R₄.
32. The compound of any one of embodiments 1-31, wherein R₄ is H, alkyl, aryl, —C(O)ORₐ, or —C(O)NHRₐ.
33. The compound of any one of embodiments 1-32, wherein R₄ is H.
34. The compound of any one of embodiments 1-33, wherein Rₐ is C₁₋₅ alkyl or —CH₂cycloalkyl.
35. The compound of any one of embodiments 1-34, wherein Rₐ is C₁₋₅ alkyl.
36. The compound of any one of embodiments 1-35, wherein Rₐ is methyl, ethyl, or n-propyl, or i-propyl.
37. The compound of any one of embodiments 1-36, wherein R₂ is aryl or heteroaryl.
38. The compound of any one of embodiments 1-37, wherein R₂ is phenyl or naphthyl.
39 The compound of any one of embodiments 1-38, wherein R₂ is

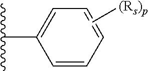

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, 2, or 3.
40. The compound of anyone of embodiments 1-39, wherein R₂ is

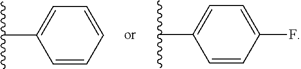 [SMILES: *c1ccccc1 ; *c1ccc(F)cc1]

41. The compound of any one of embodiments 1-37, wherein R₂ is a 5- or 6-membered nitrogen-containing heteroaryl.
42. The compound of any one of embodiments 1-37 and 41, wherein R₂ is

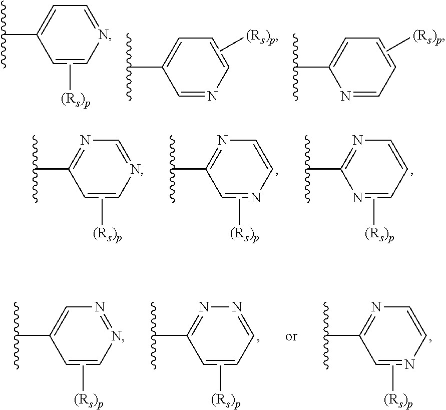

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, or 2.
43. The compound of any one of embodiments 1-31, wherein R₂ and R₄ taken together with the carbon atoms to which they are attached form a cycloalkyl, heterocyclyl, aryl, or heteroaryl.
44. The compound of embodiment 43, wherein R₂ and R₄ taken together with the carbon atoms to which they are attached form a cycloalkyl.
45. The compound of embodiment 43 or 44, wherein the cycloalkyl is a C₄₋₆ cycloalkyl.
46. The compound of any one of embodiments 1-40, having the structure:

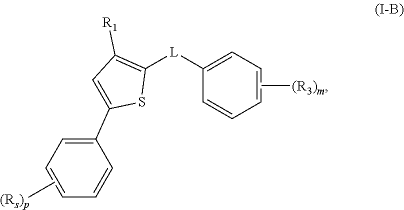

or a pharmaceutically acceptable salt thereof,
wherein:
R_{c} is each independently halogen, alkyl, haloalkyl, alkoxy, haloalkoxy, sulfonyl, or two R_{c} taken together with the atoms to which they are attached form an aryl, heteroaryl, cycloalkyl, or heterocycloalkyl; and
p is 0, 1, 2, or 3.
   47. The compound of any one of embodiments 1-40 and 46, having the structure:

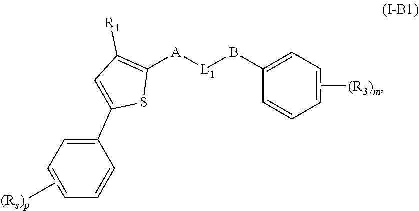

or a pharmaceutically acceptable salt thereof,
wherein:
A is —NR_{d}C(O)—, —C(O)NR_{d}—, —NR_{d}S(O)₂— or —S(O)₂NR_{d}—;
B is —NR_{d}—, —NR_{d}C(O)—, —OC(O)—, —C(O)-alkylene-, —C(O), —NR_{d}S(O₂)—, or —S(O₂)NR_{d}—; and
L₁ is a bond, C₁₋₅ alkylene, C₁₋₅ alkenylene, C₁₋₅ alkynylene, or heterocyclyl, each of which is optionally substituted.
   48. The compound of any one of embodiments 1-47, wherein R₃ is —S(O)₂NHR_{b}, —S(O)₂NR_{b}R_{b}, —NHS(O)₂R_{b}, —C(O)R_{b}, —C(O)NHR_{b}, —C(O)NR_{b}R_{b}, —NHC(O)R_{b}, —NHC(O)NR_{b}R_{b}, or —CN.
   49. The compound of any one of embodiments 1-48, wherein R₃ is —N(H)S(O)₂alkyl, —N(H)S(O)₂cycloalkyl, —S(O)₂NH₂, —C(O)alkyl, or —CN.
   50. The compound of any one of embodiments 1-49, wherein R₃ is —N(H)S(O)₂CH₃, —S(O)₂NH₂, —C(O)CH₃, or —CN.
   51. The compound of any one of embodiments 1-40 and 46-50, having the structure:

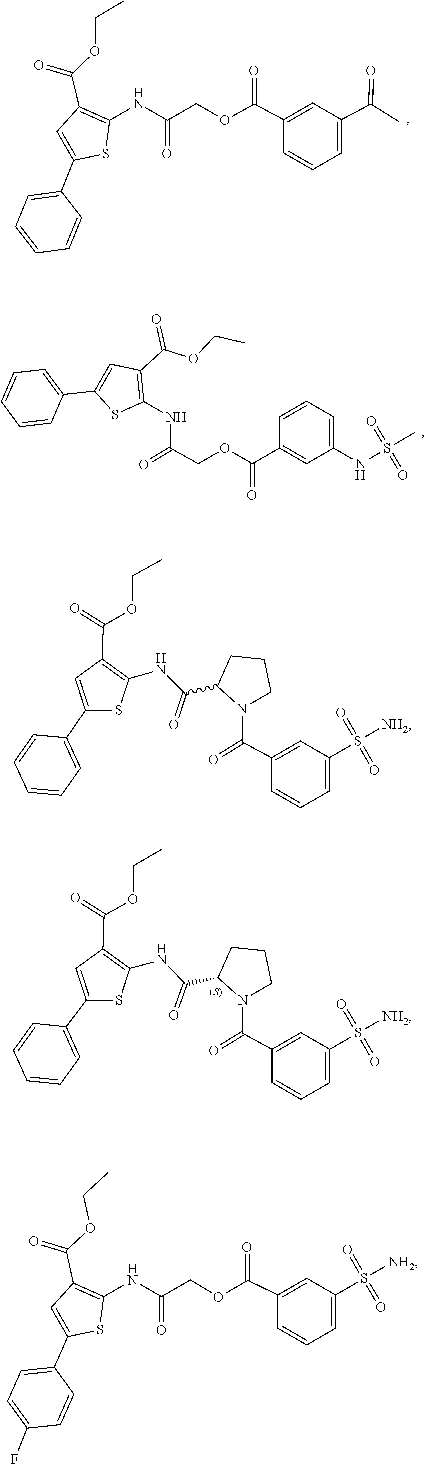

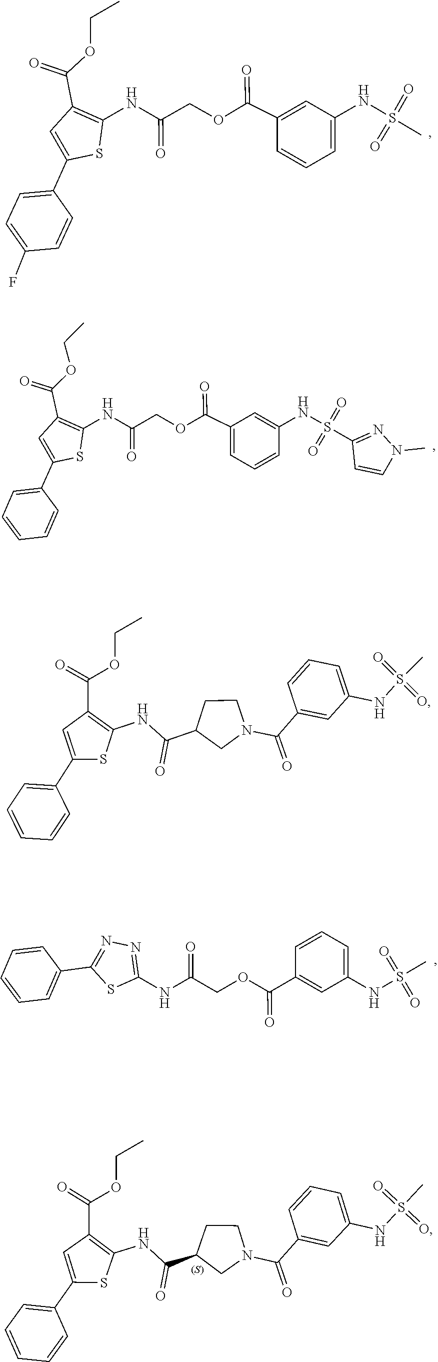

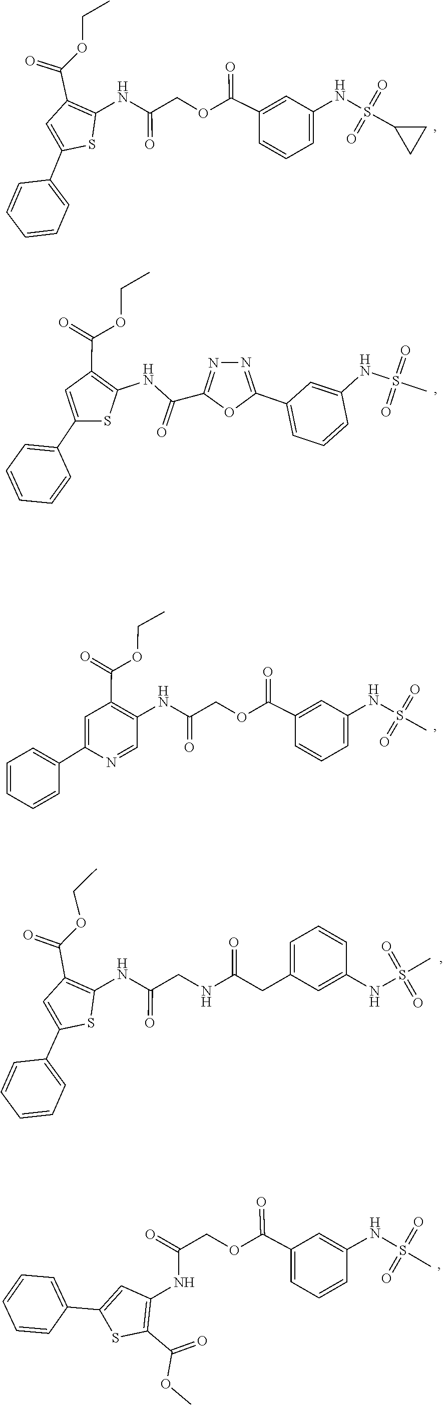 [SMILES: CCOC(=O)c1cc(-c2ccccc2)sc1NC(=O)COC(=O)c1cccc(NS(=O)(=O)C2CC2)c1 ; CCOC(=O)c1cc(-c2ccccc2)sc1NC(=O)c1nnc(-c2cccc(NS(C)(=O)=O)c2)o1 ; CCOC(=O)c1cc(-c2ccccc2)ncc1NC(=O)COC(=O)c1cccc(NS(C)(=O)=O)c1 ; CCOC(=O)c1cc(-c2ccccc2)sc1NC(=O)CNC(=O)Cc1cccc(NS(C)(=O)=O)c1 ; COC(=O)c1sc(-c2ccccc2)cc1NC(=O)COC(=O)c1cccc(NS(C)(=O)=O)c1]

or a pharmaceutically salt or tautomer thereof.
52. A compound of Formula (II) having the structure:

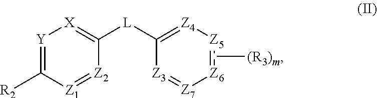

or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, or —S(O)₂NR_{c}—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

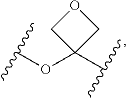 [SMILES: *OC1(*)COC1]

—OC(CF₃)—, —O—, —C(O)-alkylene-, —C(O)—, —NR_{d}S(O₂)—, —S(O₂) NR_{d}—, or heteroaryl; and
R_{c} and R_{d} are each independently H, alkyl, cycloalkyl, heterocyclyl, alkylene-aryl, acyl, or sulfonyl,
   with the proviso that -A-L₁-B— does not contain an —O—O, —NR_{c}—O, —O—NR_{d}—, —NR_{c}—NR_{d}—, or S—S bond;
X is C—R₁;
Y is C—R₄ or N;
Z₁ is N or C—R₅
Z₂ is N or CH;
Z₃, Z₄, Z₅, Z₆, and Z₇ are each independently N or CH, wherein no more than 2 of Y, Z₁, and Z₂ are N and no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl) (Rₐ), —C(O)N(Rₐ)(Rₐ), —C(O)NRₐRₐ, —NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, halogen, alkyl, alkoxy, alkylenecycloalkyl, cycloalkyl, aryl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
wherein at least one of R₁, R₂, and R₄ is not H.
   53. The compound of embodiment 52, having the structure:

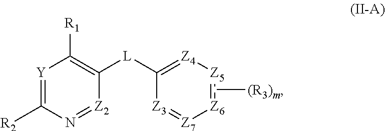

or a pharmaceutically acceptable salt or tautomer thereof.
54. The compound of embodiment 52 or 53, having the structure:

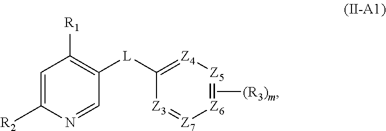

or a pharmaceutically acceptable salt or tautomer thereof.
55. The compound of embodiment 52 or 53, having the structure:

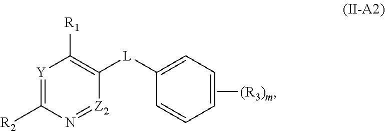

or a pharmaceutically acceptable salt or tautomer thereof.
56. The compound of any one of embodiments 52-55, wherein L is a linker having the structure -A-L₁-B—, wherein:
A is —NR_{c}C(O)— or —C(O)NR_{c};
L₁ is absent, alkylene, cycloalkyl, or heterocyclyl;
B is a bond, —NR_{d}—, —N(S(O)₂-alkyl)-, —NR_{d}C(O)—, —OC(O)—, —C(O)-alkylene-, —C(O)—, —NR_{d}S(O₂)—, or —S(O₂) NR_{d}—; and
R_{c} and R_{d} are each independently H, alkyl, cycloalkyl, or alkylene-aryl, with the proviso that -A-L₁-B— does not contain an —O—O, —NR_{c}—O, —O—NR_{d}—, —NR_{c}—NR_{d}—, or S—S bond.
   57. The compound of any one of embodiments 52-56, wherein A is —NR_{c}C(O)—, —C(O)NR_{c}—, or —C(O)—.
   58 The compound of any one of embodiments 52-57, wherein A is —NR_{c}C(O)—.
   59. The compound of any one of embodiments 52-58, wherein R^{c} is H, C₁₋₅alkyl, C₃₋₆cycloalkyl, or —CH₂-phenyl.
   60. The compound of any one of embodiments 52-59, wherein L₁ is absent, alkylene, or heterocyclyl.
   61. The compound of any one of embodiments 52-60, wherein L₁ is alkylene, and the alkylene is a C₁₋₃ alkylene.
   62. The compound of any one of embodiments 52-61, wherein the alkylene of L₁ is an optionally substituted methylene.
   63. The compound of any one of embodiments 52-62, wherein the alkylene of L₁ comprises the structure:

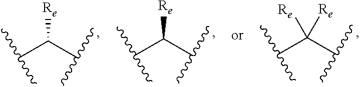

wherein Rₑ is each independently H, F, C₁₋₅ alkyl, —CH₂OH, —CH₂cycloalkyl, — or CH₂aryl, or two Rₑ taken together with the carbon atom to which they are attached form a C₃₋₆ cycloalkyl or a 4- to 6-membered heterocyclyl.
64 The compound of embodiment 63, wherein Rₑ is H or C₁₋₅ alkyl.
65. The compound of any one of embodiments 52-60, wherein L₁ is a heterocyclyl.
66. The compound of any one of embodiments 52-60 and 65, wherein the heterocyclyl of L₁ is a 3- to 12-membered heterocyclyl.
67. The compound of any one of embodiments 52-60, 65, and 66, wherein the heterocyclyl of L₁ has the structure:

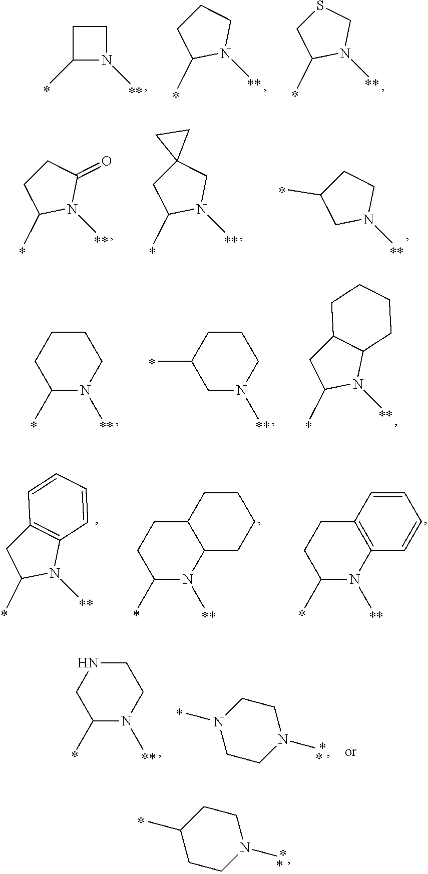

wherein * represents the attachment point to A and ** represents the attachment point to B.
68. The compound of any one of embodiments 52-67, wherein B is bond, —NR_{b}C(O)—, —NR_{b}S(O)₂—, —C(O)—, —OC(O)—, —C(O)—O—, or heteroaryl;
69. The compound of any one of embodiments 52-68, wherein B is a bond, —OC(O)—, or —C(O)—.
70. The compound of embodiment 68, wherein the heteroaryl is a 5- or 6-membered nitrogen-containing heteroaryl.
71. The compound of any one of embodiments 52-70, wherein -A-L₁-B— has the structure:

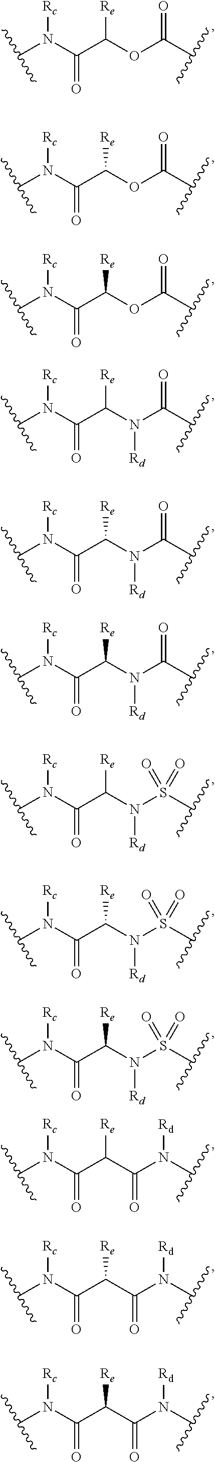

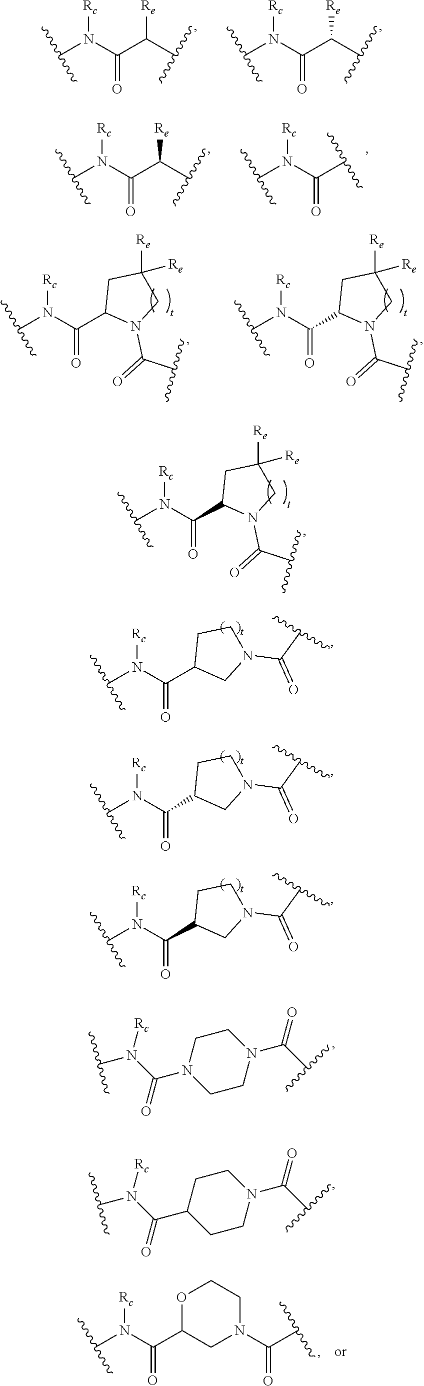

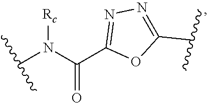

wherein:
   R_{c} and R_{d} are each independently H, alkyl, cycloalkyl, alkylene-aryl, acyl, or sulfonyl;
   Rₑ is each independently H, F, C₁₋₅ alkyl, —CH₂cycloalkyl, — or CH₂aryl, or two Rₑ taken together with the carbon atom to which they are attached form a C₃₋₆ cycloalkyl or a 4- to 6-membered heterocyclyl; and
   t is 1 or 2.
      72. The compound of any one of embodiments 52-71, wherein A-L₁-B has the structure:

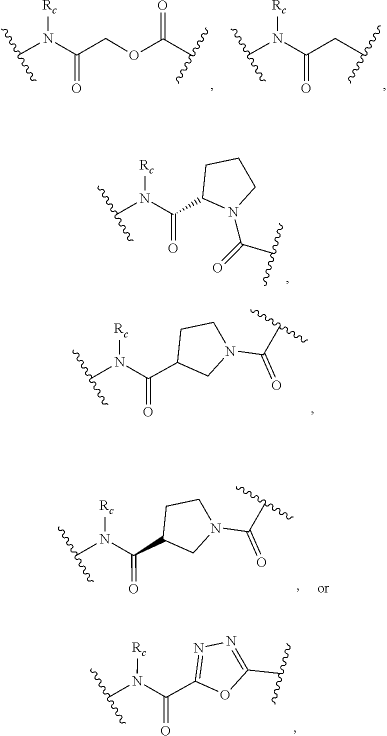

73. The compound of embodiment 71 or 72, wherein R_{c} is H.
74. The compound of any one of embodiments 52-73, wherein X is C—R₁.
75. The compound of any one of embodiments 52-74, wherein R₁ is H, halogen, alkyl, —C(O)ORₐ, or —C(O)NHRₐ.
76. The compound of any one of embodiments 52-75, wherein R₁ is halogen, alkyl, —C(O)ORₐ, or —C(O)NHRₐ.
77 The compound of any one of embodiments 52-76, wherein R₁ is —C(O)ORₐ.
78 The compound of any one of embodiments 52-77, wherein Y is C—R₄.
79. The compound of any one of embodiments 52-78, wherein R₄ is H, alkyl, aryl, —C(O)ORₐ, or —C(O)NHRₐ.
80. The compound of any one of embodiments 52-79, wherein R₄ is H.
81. The compound of any one of embodiments 52-80, wherein Rₐ is C₁₋₅ alkyl or —CH₂cycloalkyl.
82. The compound of any one of embodiments 52-81, wherein Rₐ is C₁₋₅ alkyl.
83. The compound of any one of embodiments 52-82, wherein Rₐ is methyl, ethyl, or n-propyl, or i-propyl.
84. The compound of any one of embodiments 52-83, wherein R₂ is aryl or heteroaryl.
85. The compound of any one of embodiments 52-84, wherein R₂ is phenyl or naphthyl.
86. The compound of any one of embodiments 52-85, wherein R₂ is

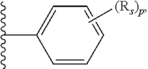

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, 2, or 3.
87. The compound of anyone of embodiments 52-86, wherein R₂ is

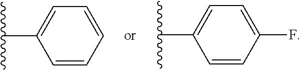 [SMILES: *c1ccccc1 ; *c1ccc(F)cc1]

88. The compound of any one of embodiments 52-84, wherein R₂ is a 5- or 6-membered nitrogen-containing heteroaryl.
89. The compound of any one of embodiments 52-84 and 88, wherein R₂ is

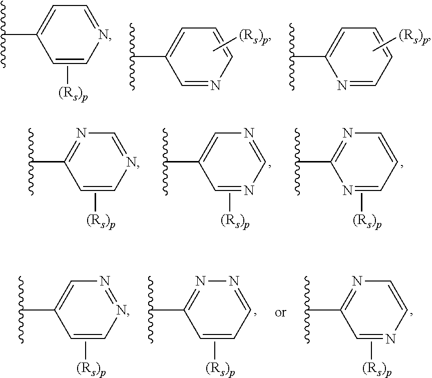

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, or 2.
90. The compound of any one of embodiments 52-78, wherein R₂ and R₄ taken together with the carbon atoms to which they are attached form a cycloalkyl, heterocyclyl, aryl, or heteroaryl.
91. The compound of embodiment 90, wherein R₂ and R₄ taken together with the carbon atoms to which they are attached form a cycloalkyl.
92. The compound of embodiment 90 or 91, wherein the cycloalkyl is a C₄₋₆ cycloalkyl.
93. The compound of any one of embodiments 52-92, wherein R₃ is —S(O)₂NHR_{b}, —S(O)₂NR_{b}R_{b}, —NHS(O)₂R_{b}, —C(O)R_{b}, —C(O)NHR_{b}, —C(O)NR_{b}R_{b}, —NHC(O)R_{b}, —NHC(O)NR_{b}R_{b}, or —CN.
94. The compound of any one of embodiments 52-93, wherein R₃ is —N(H)S(O)₂alkyl, —N(H)S(O)₂cycloalkyl, —S(O)₂NH₂, —C(O)alkyl, or —CN.
95. The compound of any one of embodiments 1-94, wherein R₃ is —N(H)S(O)₂CH₃, —S(O)₂NH₂, —C(O)CH₃, or —CN.
96 The compound of embodiment 52, wherein the compound has the structure:

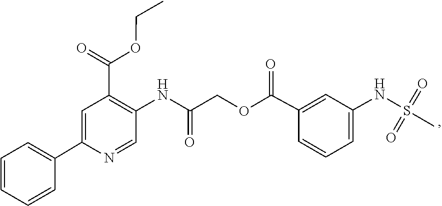 [SMILES: CCOC(=O)c1cc(-c2ccccc2)ncc1NC(=O)COC(=O)c1cccc(NS(C)(=O)=O)c1]

or a pharmaceutically acceptable salt thereof.
97. A pharmaceutical composition comprising (i) a compound of any one of embodiments 1-96; and (ii) one of more pharmaceutically acceptable carriers or excipients.
98. A method of treating cancer in a subject in need thereof, comprising administering to the subject an effective amount of a compound of any one of embodiments 1-96 or the pharmaceutical composition of embodiment 97.
99 The method of embodiment 98, wherein the cancer is breast cancer, lung cancer, pancreatic cancer, melanoma, multiple myeloma, colon carcinoma, colorectal cancer, or glioblastoma.
100. The method of embodiment 98 or 99, further comprising administering an anti-PD-1 antibody.
101. The method of embodiment 100, wherein administering the compound and the anti-PD-1 antibody to the subject provides a synergistic effect in treating cancer.
102. A compound of Formula (I) having the structure:

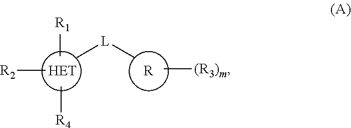

or a pharmaceutically acceptable salt thereof,
wherein:

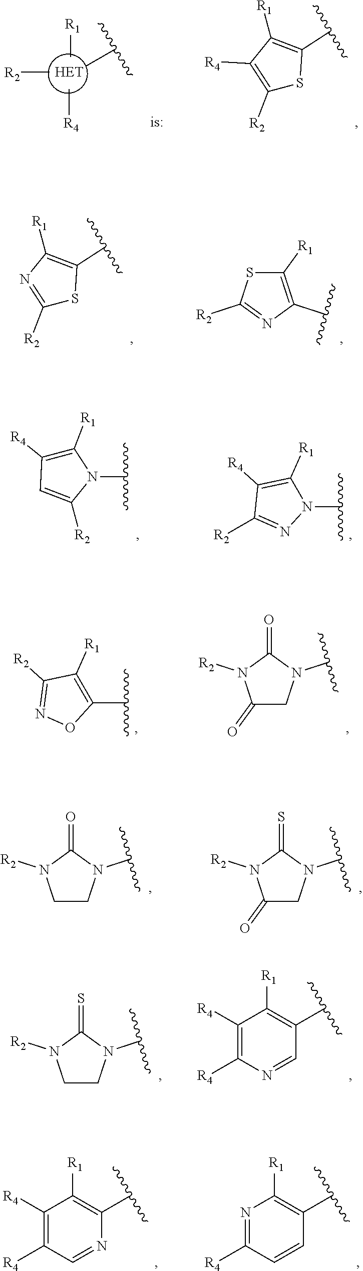

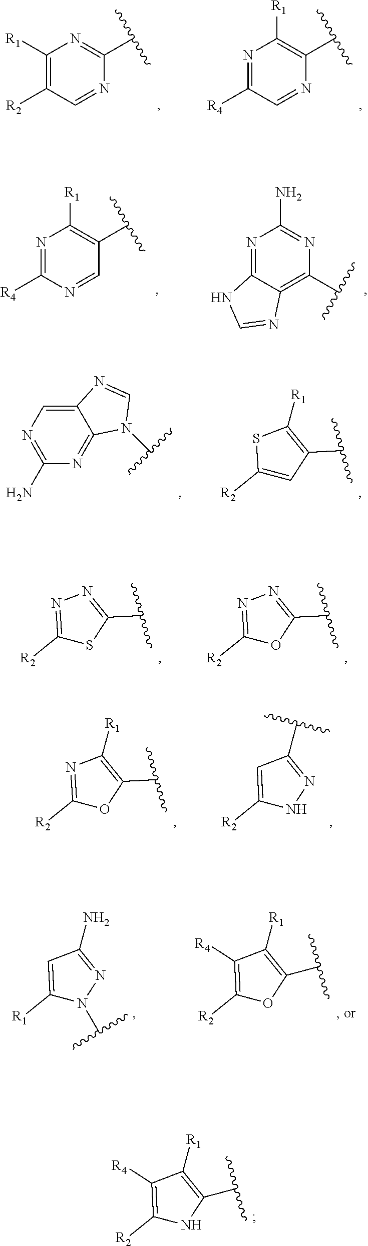

is aryl, heteroaryl, cycloalkyl, or heterocyclyl;
L is a linker having structure -A-L₁-B—,
wherein:
A is, —NR_{c}—, —NR_{c}C(O)—, —C(O)NR_{c}—, —NR_{c}S(O)₂—, or —S(O)₂NR_{c}—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—,

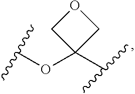 [SMILES: *OC1(*)COC1]

—OC(CF₃)—, —O—, —C(O)-alkylene-, —C(O)—, —NR_{d}S(O₂)—, —S(O₂) NR_{d}—, or heteroaryl; and
R_{c} and R_{d} are each independently H, alkyl, cycloalkyl, heterocyclyl, alkylene-aryl, acyl, or sulfonyl,
   with the proviso that -A-L₁-B— does not contain an —O—O, —NR_{c}—O, —O—NR_{d}—, —NR_{c}—NR_{d}—, or S—S bond;
R₁ is H, halogen, alkyl, —CN, —C(O)Rₐ, —C(O)ORₐ, —C(O)NHRₐ, —C(O)N(alkyl) (Rₐ), —C(O)NRₐRₐ, —NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, or —CN, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
wherein at least one of R₁, R₂, and R₄ is not H.
   103. The compound of embodiment 102, wherein

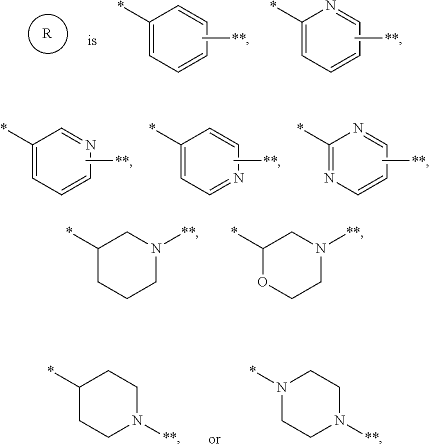

wherein * represents the attachment point to L and ** represents the attachment point to the one or more R₃.
104. The compound of embodiment 102 and 103, wherein at least two of R₁, R₂, and R₄ are not H.
105. The compound of any one of embodiments 102-104, wherein L is a linker having the structure -A-L₁-B—, wherein:
A is —NR_{c}C(O)— or —C(O)NR_{c};
L₁ is absent, alkylene, cycloalkyl, or heterocyclyl;
B is a bond, —NR_{d}—, —N(S(O)₂-alkyl)-, —NR_{d}C(O)—, —OC(O)—, —C(O)-alkylene-, —C(O)—, —NR_{d}S(O₂)—, or —S(O₂) NR_{d}—; and
R_{c} and R_{d} are each independently H, alkyl, cycloalkyl, or alkylene-aryl, with the proviso that -A-L₁-B— does not contain an —O—O, —NR_{c}—O, —O—NR_{d}—, —NR_{c}—NR_{d}—, or S—S bond.
   106. The compound of any one of embodiments 102-105, wherein A is —NR_{c}C(O)—, —C(O)NR_{c}—, or —C(O)—.
   107. The compound of any one of embodiments 102-106, wherein A is —NR_{c}C(O)—.
   108. The compound of any one of embodiments 102-107, wherein R_{c} is H, C₁₀₂₋₅alkyl, C₃₋₆cycloalkyl, or —CH₂-phenyl.
   109. The compound of any one of embodiments 102-108, wherein L₁ is absent, alkylene, or heterocyclyl.
   110. The compound of any one of embodiments 102-109, wherein L₁ is alkylene, and the alkylene is a C₁₀₂₋₃ alkylene.
   111. The compound of any one of embodiments 102-110, wherein the alkylene of L₁ is an optionally substituted methylene.
   112. The compound of any one of embodiments 102-111, wherein the alkylene of L₁ comprises the structure:

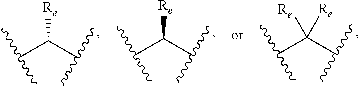

wherein Rₑ is each independently H, F, C₁₀₂₋₅ alkyl, —CH₂OH, —CH₂cycloalkyl, — or CH₂aryl, or two Rₑ taken together with the carbon atom to which they are attached form a C₃₋₆ cycloalkyl or a 4- to 6-membered heterocyclyl.
113. The compound of embodiment 16, wherein Rₑ is H or C₁₀₂₋₅ alkyl.
114. The compound of any one of embodiments 102-109, wherein L₁ is a 3- to 12-membered heterocyclyl.
115. The compound of any one of embodiments 102-109, and 114, wherein the heterocyclyl of L₁ has the structure:

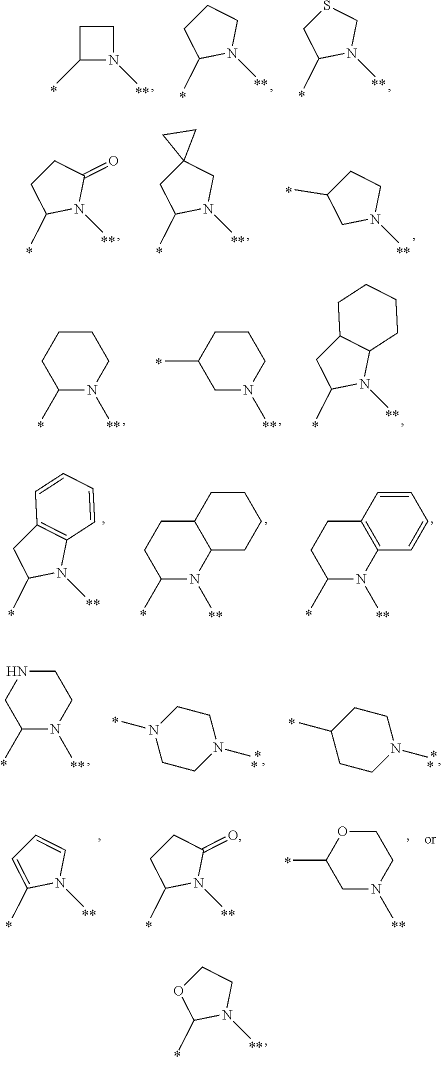

wherein * represents the attachment point to A and ** represents the attachment point to B.
116. The compound of any one of embodiments 102-115, wherein B is bond, —NR_{b}C(O)—, —NR_{b}S(O)₂—, —C(O)—, —OC(O)—, —C(O)—O—, or heteroaryl.
117. The compound of any one of embodiments 102-116, wherein B is a bond, —OC(O)—, or —C(O)—.
118. The compound of embodiment 116, wherein the heteroaryl is a 5- or 6-membered nitrogen-containing heteroaryl.
119. The compound of any one of embodiments 102-117, wherein-A-L₁-B— has the structure:

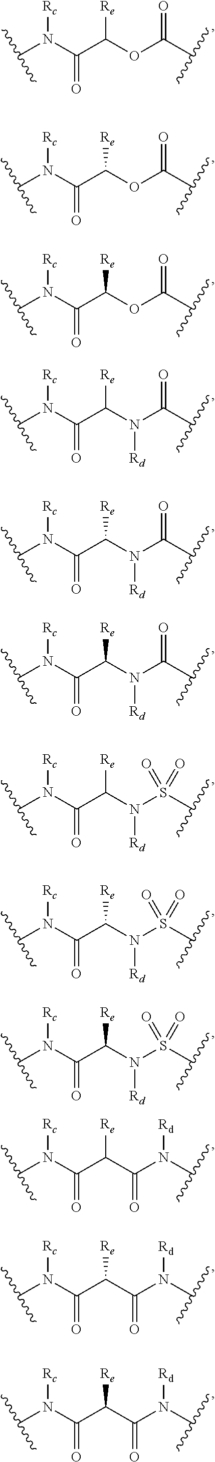

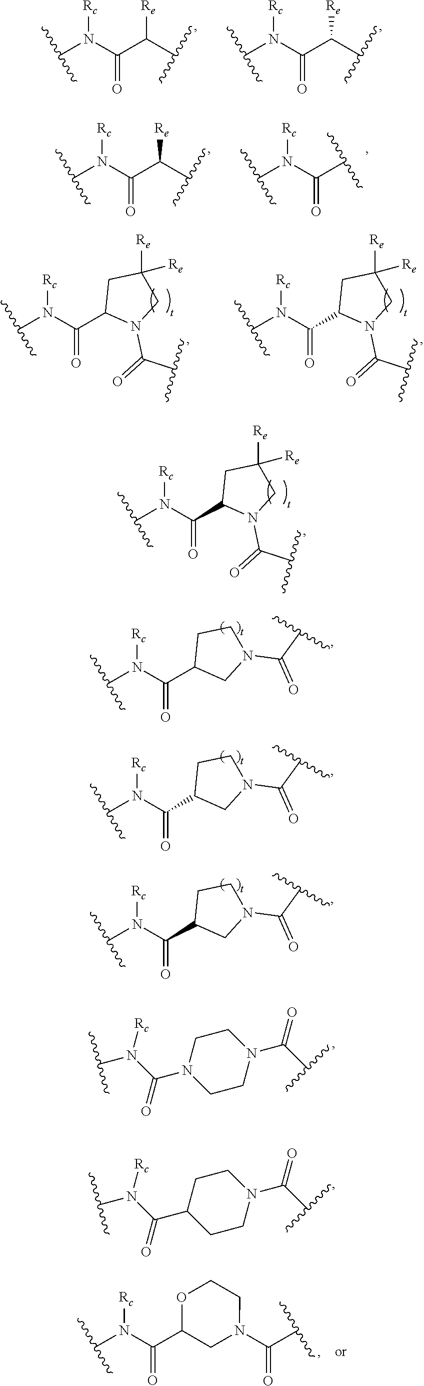

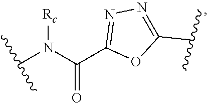

wherein:
   R_{c} and R_{d} are each independently H, alkyl, cycloalkyl, alkylene-aryl, acyl, or sulfonyl;
   Rₑ is each independently H, F, C₁₀₂₋₅ alkyl, —CH₂cycloalkyl, — or CH₂aryl, or two Rₑ taken together with the carbon atom to which they are attached form a C₃₋₆ cycloalkyl or a 4- to 6-membered heterocyclyl; and
t is 1 or 2.
   120. The compound of any one of embodiments 102-119, wherein A-L₁-B has the structure:

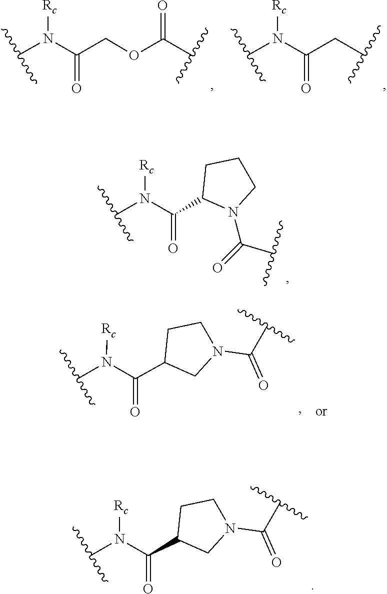

121. The compound of embodiment 119 or 120, wherein R_{c} is H.
122. The compound of any one of embodiments 102-121, wherein R₁ is halogen, alkyl, —C(O)ORₐ, or —C(O)NHRₐ.
123. The compound of any one of embodiments 102-122, wherein R₁ is —C(O)ORₐ.
124. The compound of any one of embodiments 102-123, wherein R₄ is H, alkyl, aryl, —C(O)ORₐ, or —C(O)NHRₐ.
125. The compound of any one of embodiments 102-124, wherein R₄ is H.
126. The compound of any one of embodiments 102-125, wherein Rₐ is C₁₋₅ alkyl or —CH₂cycloalkyl.
127. The compound of any one of embodiments 102-126, wherein R_{d} is C₁₋₅ alkyl.
128. The compound of any one of embodiments 102-127, wherein R_{d} is methyl, ethyl, or n-propyl, or i-propyl.
129. The compound of any one of embodiments 102-128, wherein R₂ is aryl or heteroaryl.
130. The compound of any one of embodiments 102-129, wherein R₂ is phenyl or naphthyl.
131. The compound of any one of embodiments 102-130, wherein R₂ is

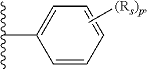

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, 2, or 3.
132. The compound of anyone of embodiments 102-131, wherein R₂ is

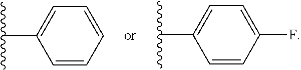 [SMILES: *c1ccccc1 ; *c1ccc(F)cc1]

133. The compound of any one of embodiments 102-129, wherein R₂ is a 5- or 6-membered nitrogen-containing heteroaryl.
134. The compound of any one of embodiments 102-129 and 133, wherein R₂ is

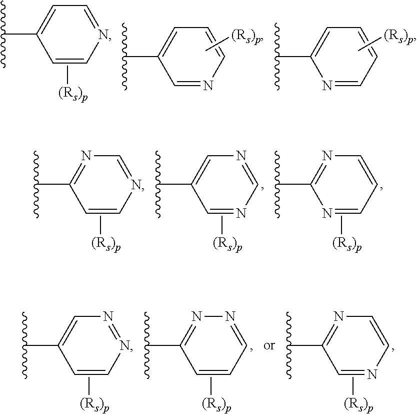

wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, or 2.
135. The compound of any one of embodiments 102-123, wherein R₂ and R₄ taken together with the carbon atoms to which they are attached form a cycloalkyl, heterocyclyl, aryl, or heteroaryl.
136. The compound of embodiment 135, wherein R₂ and R₄ taken together with the carbon atoms to which they are attached form a cycloalkyl.
137. The compound of embodiment 135 or 136, wherein the cycloalkyl is a C₄₋₆ cycloalkyl.
138. The compound of any one of embodiments 102-137, wherein R₃ is —S(O)₂NHR_{b}, —S(O)₂NR_{b}R_{b}, —NHS(O)₂R_{b}, —C(O)R_{b}, —C(O)NHR_{b}, —C(O)NR_{b}R_{b}, —NHC(O)R_{b}, —NHC(O)NR_{b}R_{b}, or —CN.
139. The compound of any one of embodiments 102-138, wherein R₃ is —N(H)S(O)₂alkyl, —N(H)S(O)₂cycloalkyl, —S(O)₂NH₂, —C(O)alkyl, or —CN.
140. The compound of any one of embodiments 102-139, wherein R₃ is —N(H)S(O)₂CH₃, —S(O)₂NH₂, —C(O)CH₃, or —CN.
141. A pharmaceutical composition comprising (i) a compound of any one of embodiments 102-140; and (ii) one of more pharmaceutically acceptable carriers or excipients.
142. A method of treating cancer in a subject in need thereof, comprising administering to the subject an effective amount of a compound of any one of embodiments 102-140 or the pharmaceutical composition of embodiment 141.
143. The method of embodiment 142, wherein the cancer is breast cancer, lung cancer, pancreatic cancer, melanoma, multiple myeloma, colon carcinoma, colorectal cancer, or glioblastoma.
144. The method of embodiment 142 or 143, further comprising administering an anti-PD-1 antibody.
145. The method of embodiment 144, wherein administering the compound and the anti-PD-1 antibody to the subject provides a synergistic effect in treating cancer.
146. The method of any one of embodiments 98-101 and 142-145, wherein the compound has the structure:

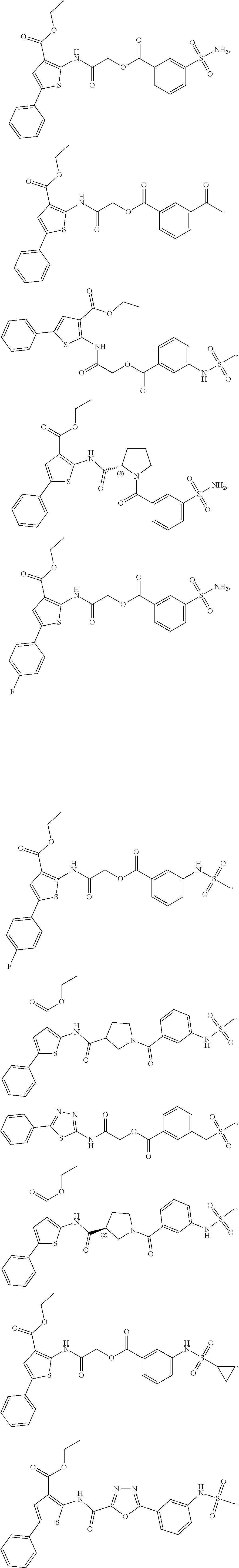

or a pharmaceutically salt or tautomer thereof.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All cited documents are herein incorporated by reference in their entirety for all purposes.

### EXAMPLES

### Example 1

### Representative Syntheses of Compounds Disclosed Herein

### Synthesis of Compound 1

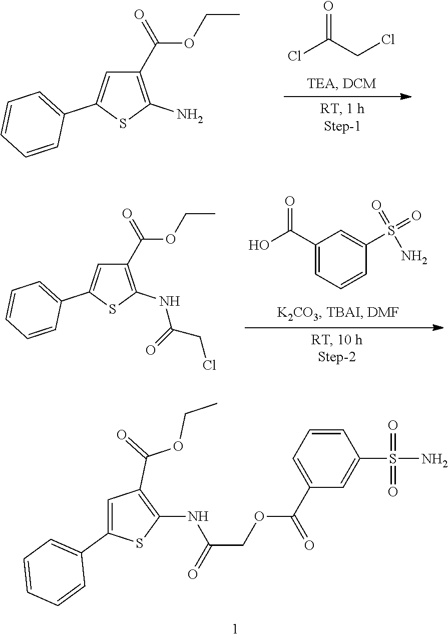

### Step 1: Ethyl 2-(2-chloroacetamido)-5-phenylthiophene-3-carboxylate

Procedure: Triethylamine (0.84 mL, 6.06 mmol) was added to a solution of ethyl 2-amino-5-phenylthiophene-3-carboxylate (1.0 g, 4.40 mmol) in anhydrous dichloromethane (30 mL). The resulting reaction mixture was cooled to 0° C. and chloro acetyl chloride (0.38 mL, 4.80 mmol) was added dropwise. The reaction mixture was then stirred at room temperature. The progress of the reaction was monitored by TLC. After completion of the reaction, water (50 mL) was added and the organic layer was separated. The aqueous layer was further extracted with dichloromethane. The organics were combined, and dried over sodium sulphate and concentrated under reduced pressure to get crude compound. The crude compound was purified by flash chromatography using ethyl acetate:hexane as the eluent system. Yield: 0.9 g (85%).

### Step 2: Ethyl 5-phenyl-2-(2-((3-sulfamoylbenzoyl)oxy) acetamido)thiophene-3-carboxylate

Procedure: Potassium carbonate (412.1 mg, 2.98 mmol) was added to a solution of ethyl 2-(2-chloroacetamido)-5-phenylthiophene-3-carboxylate (529 mg, 1.64 mmol) and 3-sulfamoyl benzoic acid (300 mg, 1.49 mmol) in anhydrous dimethylformamide (15 mL) and resulting reaction mixture was stirred at room temperature for 10 min. TBAI (55.07 mg, 0.149 mmol) was then added and reaction mixture was stirred at room temperature. The progress of the reaction was monitored by TLC. After completion of reaction, water (50 mL) was added to the reaction mixture and was extracted with ethyl acetate. The organics were combined, and dried over sodium sulphate and concentrated under reduced pressure to give crude residue. The crude compound was triturated with pentane and dichloromethane, solvent was decanted. Obtained compound was dissolved in 10% Methanol: Dichloromethane, and was filtered and dried. White solid; Yield: 237 mg (30%).

### Synthesis of Compound 66

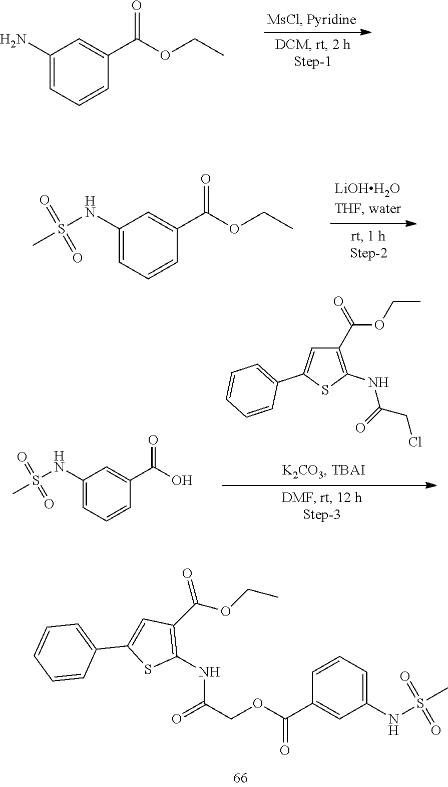 [SMILES: CCOC(=O)c1cccc(N)c1 ; CCOC(=O)c1cccc(NS(C)(=O)=O)c1 ; CS(=O)(=O)Nc1cccc(C(=O)O)c1 ; CCOC(=O)c1cc(-c2ccccc2)sc1NC(=O)CCl ; CCOC(=O)c1cc(-c2ccccc2)sc1NC(=O)COC(=O)c1cccc(NS(C)(=O)=O)c1]

### Step 1: Ethyl 3-(methylsulfonamido)benzoate

Procedure: Pyridine (10.0 mL, 121.06 mmol) was added to a stirred solution of ethyl 3-aminobenzoate (10.0 g, 60.53 mmol) in dichloromethane (100.0 mL), followed by methanesulfonyl chloride (10 mL, 127.12 mmol) at 0° C. Reaction mixture was allowed to stir at room temperature. The progress of the reaction was monitored by TLC. After completion of reaction, reaction mixture was evaporated to dryness. The crude residue was taken in water and extracted with ethyl acetate. The organics were combined, and dried over sodium sulphate and concentrated under reduced pressure to get compound. Yield: 14.0 g (95%).

### Step 2:3-(Methylsulfonamido)benzoic acid

Procedure: LiOH·H₂O (7.2 g, 172.7 mmol) was added to a stirred solution of ethyl 3-(methylsulfonamido)benzoate (14.0 g, 57.58 mmol) in a mixture of THF:H₂O (1:1, 140.0 mL) and reaction mixture was allowed to stir at room temperature. The progress of the reaction was monitored by TLC. After completion of reaction, reaction mixture was evaporated to dryness and was quenched with 1N HCl to get precipitates. The precipitate was filtered and dried over vacuum to get off white solid. Yield: 11.7 g, (95%).

### Step 3: Ethyl 2-(2-((3-(methylsulfonamido) benzoyl)oxy) acetamido)-5-phenylthiophene-3-carboxylate

Procedure: K₂CO₃ (188 mg, 1.38 mmol) was added to a stirred solution of ethyl 2-(2-chloroacetamido)-5-phenylthiophene-3-carboxylate (200 mg, 0.62 mmol) and 3-(methylsulfonamido)benzoic acid (137 mg, 0.68 mmol) in DMF (2.0 mL) and resulting reaction mixture was stirred at room temperature for 10 min. TBAI (23 mg, 0.062 mmol) was then added to the reaction mixture and was allowed to stir at room temperature. The progress of the reaction was monitored by TLC. After completion of reaction, water (10 mL) was added to the reaction mixture and was extracted with ethyl acetate. The organics were combined, and dried over sodium sulphate and concentrated under reduced pressure to give crude residue. The crude compound was purified by flash chromatography using methanol:dichloromethane as the eluent system. Off white Solid; Yield: 100 mg (32%).

### Synthesis of Compound 138

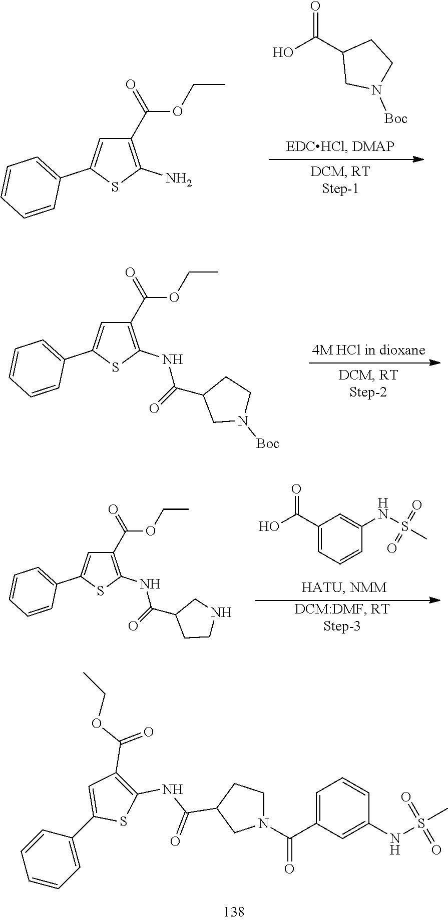

### Step 1: tert-butyl 3-((3-(ethoxycarbonyl)-5-phenylthiophen-2-yl)carbamoyl)pyrrolidine-1-carboxylate

Procedure: EDC.HCl (309 mg, 1.61 mmol) followed by DMAP (98.8 mg, 0.81 mmol) were added to a stirred solution of ethyl 2-amino-5-phenylthiophene-3-carboxylate (200 mg, 0.81 mmol) and 1-(tert-butoxycarbonyl) pyrrolidine-3-carboxylic acid (174.0 mg, 0.81 mmol) in DCM (10 mL) and reaction mixture was stirred at room temperature. The progress of the reaction was monitored by TLC. After completion of reaction, water (10 mL) was added to the reaction mixture and was extracted with ethyl acetate. The organics were combined, and dried over sodium sulphate and concentrated under reduced pressure to give crude residue. The crude compound was purified by flash chromatography using ethyl acetate:hexane as the eluent system. Yield: 150 mg (42%).

### Step 2: Ethyl 5-phenyl-2-(pyrrolidine-3-carboxamido)thiophene-3-carboxylate hydrochloride

Procedure: 4M HCl in Dioxane (5 mL) was added to a stirred solution of tert-butyl 3-((3-(ethoxycarbonyl)-5-phenylthiophen-2-yl) carbamoyl) pyrrolidine-1-carboxylate (150 mg, 0.34 mmol) in DCM (5 mL) and reaction mixture was stirred at room temperature. The progress of the reaction was monitored by TLC. After Completion of reaction, solvent was removed to get crude compound, obtained crude was washed with diethyl ether to get compound as white solid. Yield: 120 mg (94%).

### Step 3 Ethyl 2-(1-(3-(methylsulfonamido)benzoyl)pyrrolidine-3-carboxamido)-5-phenylthiophene-3-carboxylate

Procedure: HATU (220.4 mg, 0.58 mmol) and N-methylmorpholine (58.58 mg, 0.58 mmol) were added to a solution of ethyl 5-phenyl-2-(pyrrolidine-3-carboxamido) thiophene-3-carboxylate hydrochloride (100 mg, 0.29 mmol) and 3-(methylsulfonamido) benzoic acid (62.5 mg, 0.29 mmol) in DMF:DCM (3:3 ml). The resulting reaction mixture was stirred at room temperature. The progress of the reaction was monitored by TLC. After completion of reaction, water (10 mL) was added to the reaction mixture and was extracted with ethyl acetate. The organics were combined, and dried over sodium sulphate and concentrated under reduced pressure to give crude residue. The crude compound was purified by flash chromatography using ethyl acetate:hexane as the eluent system. Yield: 10 mg (7%).

TABLE 1
Characterization Data of Exemplified Compounds Synthesized Herein

| Compd. | | |
|---|---|---|
| No. | Structure | Characterization Data |
| 1 | 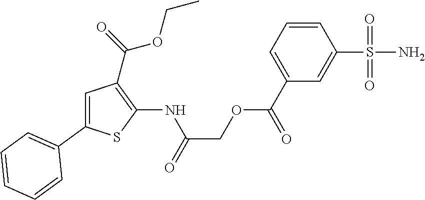 [SMILES: CCOC(=O)c1cc(-c2ccccc2)sc1NC(=O)COC(=O)c1cccc(S(N)(=O)=O)c1] | White solid; Yield: 30%; ¹H NMR (400 MHz, DMSO-d₆): δ 11.46 (s, 1H), 8.52 (s, 1H), 8.51- 8.35 (m, 1H), 8.10-8.15 (m, 1H), 7.83 (t, J = 8.00 Hz, 1H), 7.68 (d, J = 4.00 Hz, 2H), 7.58 (s, 3H), 7.42 (t, J = 8.00 Hz, 2H), 7.32 (t, J = 8.00 Hz, 1H), 5.25 (s, 2H), 4.35 (q, J = 8.00 Hz, 2H), 1.33 (t, J = 8.00 Hz, 3H); HPLC-Purity: 97.3%; MS (m/z): 488.07 [M]⁺, calcd. For C₂₂H₂₀N₂O₇S₂ m/z = 488.07. |
| 3 | 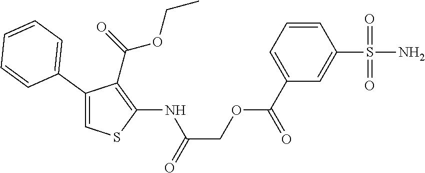 [SMILES: CCOC(=O)c1c(-c2ccccc2)csc1NC(=O)COC(=O)c1cccc(S(N)(=O)=O)c1] | White solid; Yield: 11%; ¹H NMR (400 MHz, DMSO-d₆): δ 11.55 (s, 1H), 8.51 (t, J = 1.7 Hz, 1H), 8.36 (dt, J = 7.8, 1.3 Hz, 1H), 8.15 (d, J = 7.9 Hz, 1H), 7.82 (t, J = 7.8 Hz, 1H), 7.57 (s, 2H), 7.41-7.26 (m, 5H), 6.99 (s, 1H), 5.20 (s, 2H), 4.05 (q, J = 7.1 Hz, 2H), 0.89 (t, J = 7.1 Hz, 3H); HPLC-Purity: 99.2%; LC-MS (m/z): 488.37 [M]⁺, calcd. For C₂₂H₂₀N₂O₇S₂ m/z = 488.07. |
| 13 | 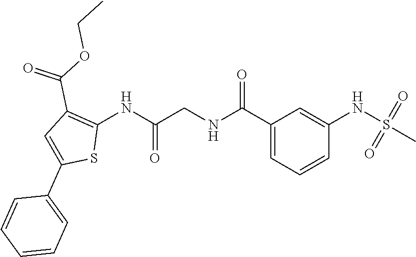 [SMILES: CCOC(=O)c1cc(-c2ccccc2)sc1NC(=O)CNC(=O)c1cccc(NS(C)(=O)=O)c1] | Off-white Solid; Yield: 8%; ¹H NMR (400 MHz, DMSO-d₆): δ 11.26 (s, 1H), 9.98 (s, 1H), 9.31 (t, J = 4 Hz, 1H), 7.75 (s, 1H), 7.67-7.69 (m, 3H), 7.51 (s, 1H), 7.49 (t, J = 8 Hz, 1H), 7.42-7.46 (m, 3H), 7.22-7.26 (m, 1H), 4.27 (q, J = 4 Hz, 2H), 4.24 (t, J = 4 Hz, 2H), 3.04 (s, 3H), 1.27 (t, J = 4 Hz, 3H); HPLC-Purity: 94.73% MS (m/z) 501.1 [M]⁺, calcd for C₂₃H₂₃N₃O₆S₂ m/z = 501.0. |
| 36 | 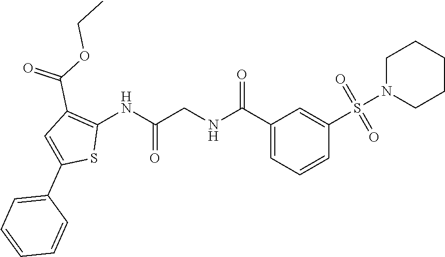 [SMILES: CCOC(=O)c1cc(-c2ccccc2)sc1NC(=O)CNC(=O)c1cccc(S(=O)(=O)N2CCCCC2)c1] | White solid; Yield: 70%; ¹H NMR (400 MHz, DMSO-d₆): δ 11.31 (s, 1H), 9.61 (t, J = 8.0 Hz, 1H), 8.27 (q, J = 8.0 Hz, 2H), 7.95 (s, 1H), 7.82 (t, J = 8.0 Hz, 1H), 7.66 (d, J = 8.0 Hz, 2H), 7.55 (s, 1H), 7.42 (t, J = 8.0 Hz, 2H), 7.31 (t, J = 8.0 Hz, 1H), 4.26 (q, J = 4.0 Hz, 4H), 2.94 (t, J = 8.0 Hz, 4H), 2.89 (s, 1H), 2.71 (s, 1H), 1.55 (s, 5H), 1.26 (t, J = 4.0 Hz, 2H); HPLC-Purity: 99.4%; MS (m/z): 555.15 [M]⁺, calcd. For C₂₇H₂₉N₃O₆S₂ m/z = 555.6. |
| 64 | 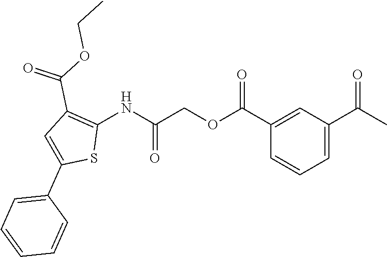 [SMILES: CCOC(=O)c1cc(-c2ccccc2)sc1NC(=O)COC(=O)c1cccc(C(C)=O)c1] | White Solid; Yield: 30%; ¹H-NMR (400 MHz, DMSO-d₆): δ 11.05 (s, 1H), 8.63 (t, J = 8.00 Hz, 1H), 8.40-8.37 (m, 1H), 8.31-8.29 (m, 1H), 7.78 (t, J = 8.00 Hz, 1H), 7.67 (d, J = 8.00 Hz, 2H), 7.58 (s, 1H), 7.42 (t, J = 8.00 Hz, 2H), 7.32 (t, J = 8.00 Hz, 1H), 5.21 (s, 2H), 4.33 (q, J = 8.00 Hz, 2H), 2.70 (s, 3H), 1.32 (t, J = 8.00 Hz, 3H); HPLC-Purity: 98.0%; MS (m/z): 451 [M]⁺, calcd for C₂₄H₂₁NO₆S m/z = 451 |
| 66 | 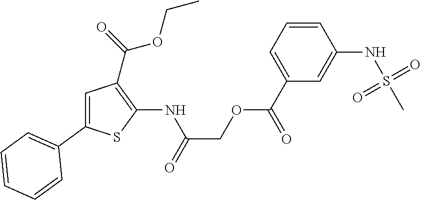 [SMILES: CCOC(=O)c1cc(-c2ccccc2)sc1NC(=O)COC(=O)c1cccc(NS(C)(=O)=O)c1] | Off-white Solid; Yield: 32%; ¹H NMR (400 MHz, DMSO-d₆): δ 11.0 (s, 1H), 7.88 (s, 1H), 7.82 (d, J = 8 Hz, 1H), 7.66 (d, J = 8 Hz, 2H), 7.57 (s, 1H), 7.50 (q, J = 8 Hz 2H), 7.42 (t, J = 8 Hz 2H), 7.31 (t, J = 8 Hz, 1H), 5.16 (s, 2H), 4.31 (q, J = 8 Hz 2H), 2.99 (s, 3H), 1.31 (t, , J = 8 Hz, 3H); HPLC-Purity: 96%; MS (m/z): 541.0 [M + 39]⁺, calcd for C₂₃H₂₂N₂O₇S₂ m/z = 502.0 |
| 67 | 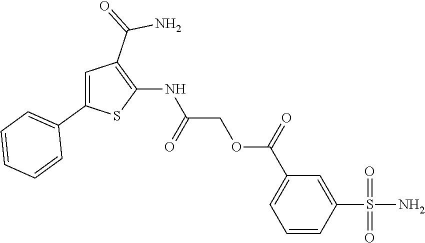 [SMILES: NC(=O)c1cc(-c2ccccc2)sc1NC(=O)COC(=O)c1cccc(S(N)(=O)=O)c1] | White solid; Yield: 6%; ¹H NMR (400 MHz, DMSO-d₆): δ 12.76 (s, 1H), 10.07 (s, 1H), 7.93- 7.96 (m, 3H), 7.89 (s, 1H), 7.68 (s, 1H), 7.55- 7.52 (m, 4H), 7.44 (t, J = 8.00 Hz, 2H), 7.31 (t, J = 8.00 Hz, 1H), 5.10 (s, 2H); 3.07 (s, 3H); HPLC-Purity: 92.9%; MS (m/z): 473.3 [M]⁺, calcd. For C₂₁H₁₉N₃O₆S₂ m/z = 473.07. |
| 72 | 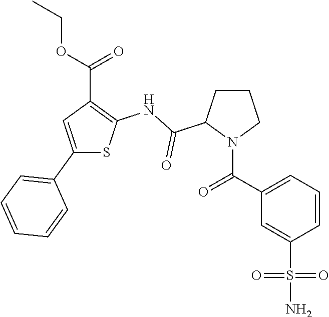 [SMILES: CCOC(=O)c1cc(-c2ccccc2)sc1NC(=O)C1CCCN1C(=O)c1cccc(S(N)(=O)=O)c1] | White Solid; Yield: 12%; ¹H NMR (400 MHz, DMSO-d₆): δ 11.50 (s, 1H), 8.10 (s, 1H), 7.96 (d, J = 8.00 Hz, 1H), 7.87 (d, J = 8.0 Hz, 1H), 7.73-7.66 (m, 3H), 7.56 (d, J = 4.00 Hz, 1H), 7.48 (s, 2H), 7.42 (t, J = 8.00 Hz, 2H), 7.32 (t, J = 8.00 Hz, 1H), 4.87-4.84 (m, 1H), 4.38 (q, J = 8.00 Hz, 2H), 4.06-3.68 (m, 1H), 2.40-2.33 (m, 1H), 2.20-2.07 (m, 1H), 1.99-1.35 (m, 3H), 1.23 (t, J = 8.00 Hz, 3H); HPLC-Purity: 98.53%; MS (m/z): 527.12 [M]⁺, calcd. For C₂₅H₂₅N₃O₆S₂ m/z = 527 |
| 75 | 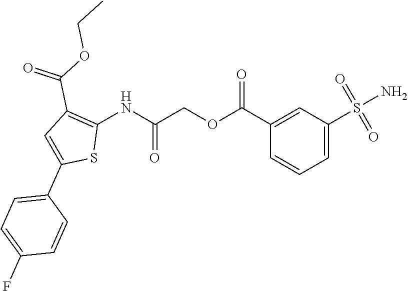 [SMILES: CCOC(=O)c1cc(-c2ccc(F)cc2)sc1NC(=O)COC(=O)c1cccc(S(N)(=O)=O)c1] | White solid; Yield: 58%; ¹H NMR (400 MHz, DMSO-d₆): δ 11.46 (s, 1H), 8.51 (s, 1H), 8.36 (d, J = 8.0 Hz, 1H), 8.16 (d, J = 8.0 Hz, 1H), 7.83 (t, J = 16.0 Hz, 1H), 7.71 (t, J = 12.0 Hz, 2H), 7.56 (d, J = 4.0 Hz, 3H), 7.25 (t, J = 12.0 Hz, 2H), 5.24 (s, 2H), 4.34 (q, J = 12.0 Hz, 2H), 1.33 (t, J = 12.0 Hz, 3H); HPLC-Purity: 98.8%; MS (m/z): 506.1 [M]⁺, calcd. For C₂₂H₁₉FN₂O₇S₂ m/z = 506.3. |
| 80 | 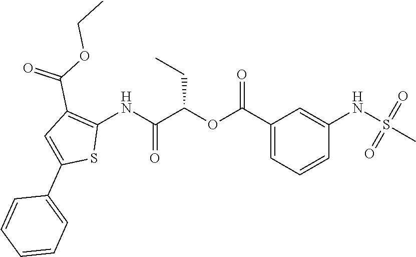 | Off-white solid; Yield: 35%; ¹H NMR (400 MHz, DMSO-d₆): δ 11.5 (s, 1H), 10.05 (s, 1H), 7.95 (s, 1H), 7.89 (m, 1H), 7.67 (m, 2H), 7.60 (m, 3H), 7.43 (m, 2H), 7.30 (m, 1H), 5.60 (m, 1H), 4.30 (q, J = 8.00 Hz, 2H), 2.07 (m, 2H), 3.04 (s, 3H), 1.30 (t, J = 8.00 Hz, 3H), 0.89 (t, J = 8.00 Hz, 3H); HPLC-Purity: 98.6%; MS (m/z): 516.5, calcd for C₂₄H₂₄N₂O₇S₂ m/z = 516.12. |
| 108 | 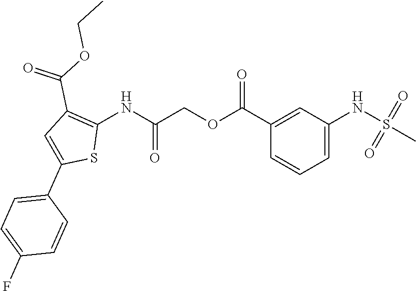 [SMILES: CCOC(=O)c1cc(-c2ccc(F)cc2)sc1NC(=O)COC(=O)c1cccc(NS(C)(=O)=O)c1] | White solid; Yield: 58%; ¹H NMR (400 MHz, DMSO-d₆): δ 11.45 (s, 1H), 10.01 (s, 1H), 7.93- 7.89 (m, 2H), 7.71 (t, J = 12.0 Hz, 2H), 7.56 (d, J = 8.0 Hz, 3H), 7.25 (t, J = 12.0 Hz, 2H), 7.18 (s, 2H), 5.24 (s, 2H), 4.33 (q, J = 12.0 Hz, 2H), 3.04 (s, 3H), 1.32 (t, J = 12.0 Hz, 3H); HPLC- Purity: 97.42%; MS (m/z): 520.08 [M]⁺, calcd. For C₂₃H₂₁FN₂O₇S₂ m/z = 520.3. |
| 134 | 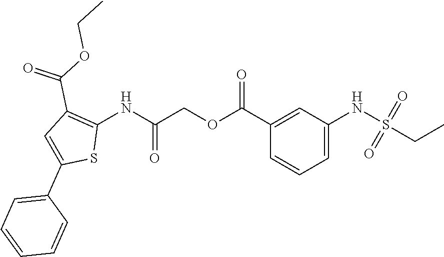 [SMILES: CCOC(=O)c1cc(-c2ccccc2)sc1NC(=O)COC(=O)c1cccc(NS(=O)(=O)CC)c1] | White solid; Yield: 52%; ¹H NMR (400 MHz, DMSO-d₆): δ 11.42 (s, 1H), 10.11 (s, 1H), 7.94- 7.91 (m, 1H), 7.89-7.89 (m, 1H), 7.67 (t, J = 8.0 Hz, 2H), 7.58-7.55 (m, 3H), 7.42 (t, J = 8.0 Hz, 2H), 7.33 (t, J = 8.0 Hz, 1H), 5.18 (s, 2H), 4.33 (q, J = 4.0 Hz, 2H), 3.15 (q, J = 8.0 Hz, 2H), 1.32 (t, J = 4.0 Hz, 3H), 1.22 (t, J = 8.0 Hz, 3H); HPLC-Purity: 95.7%; MS (m/z): 516.5 [M]⁺, calcd. For C₂₄H₂₄N₂O₇S₂ m/z = 516.1. |
| 138 | 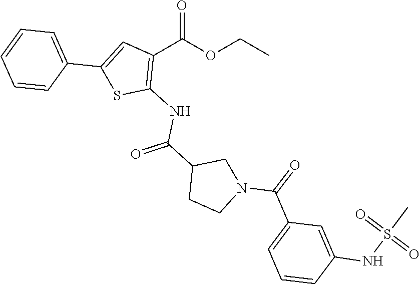 [SMILES: CCOC(=O)c1cc(-c2ccccc2)sc1NC(=O)C1CCN(C(=O)c2cccc(NS(C)(=O)=O)c2)C1] | White Solid; Yield: 7%; ¹H NMR (400 MHz, DMSO-d₆): δ 11.08 (s, 1H), 9.92 (s, 1H), 7.63 (q, J = 8.00 Hz, 2H), 7.53 (s, 1H), 7.44-7.39 (m, 3H), 7.35-7.31 (m, 2H), 7.31-7.27 (m, 2H), 4.36 (q, J = 8.00 Hz, 2H), 3.82 (d, J = 8.00 Hz, 1H), 3.74-3.64 (m, 1H), 3.61-3.53 (m, 3H), 3.02 (s, 3H), 2.33-2.29 (m, 1H), 2.25-2.10 (m, 1H), 1.31 (t, J = 4.00 Hz, 3H); HPLC-Purity: 97%; MS (m/z): 541.0 [M]⁺, calcd for C₂₆H₂₇N₃O₆S₂ m/z = 541.13. |
| 144 | 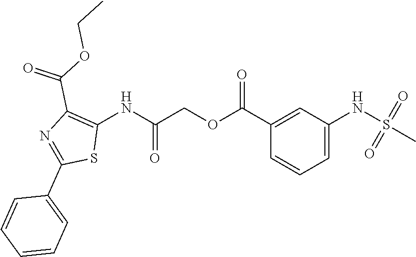 [SMILES: CCOC(=O)c1nc(-c2ccccc2)sc1NC(=O)COC(=O)c1cccc(NS(C)(=O)=O)c1] | White solid; Yield: 87%; ¹H NMR (400 MHz, DMSO-d₆): δ 11.38 (s, 1H), 10.06 (s, 1H), 7.89- 7.87 (m, 4H), 7.52-7.50 (m, 5H), 5.24 (s, 2H), 4.40 (q, J = 4.00 Hz, 2H), 3.05 (s, 3H), 1.35 (t, J = 4.00 Hz, 3H); HPLC-Purity: 91.54%; MS (m/z): 503.3 [M]⁺, calcd. For C₂₂H₂₁N₃O₇S₂ m/z = 503.8. |
| 153 | 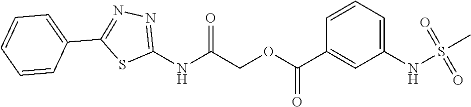 [SMILES: CS(=O)(=O)Nc1cccc(C(=O)OCC(=O)Nc2nnc(-c3ccccc3)s2)c1] | Off-white Solid; Yield: 12%; ¹H NMR (400 MHz, DMSO-d₆): δ 13.08 (s, 1H), 10.07 (s, 1H), 7.93-7.95 (m, 2H), 7.76-7.79 (m, 1H), 7.75-7.78 (m, 1H), 7.52-7.54 (m, 5H), 5.14 (s, 2H), 3.04 (s, 3H); HPLC-Purity: 94%; MS (m/z) 432.0 [M]⁺, calcd for C₁₈H₁₆N₄O₅S₂ m/z = 432.1. |
| 157 | 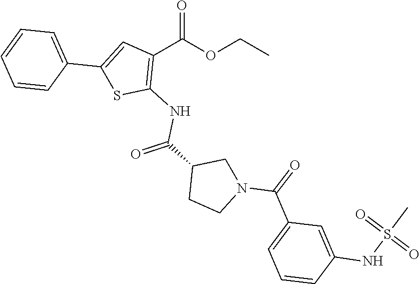 [SMILES: CCOC(=O)c1cc(-c2ccccc2)sc1NC(=O)[C@H]1CCN(C(=O)c2cccc(NS(C)(=O)=O)c2)C1] | White Solid; Yield: 17%; ¹H NMR (400 MHz, DMSO-d₆): δ 10.99 (s, 1H), 9.93 (s, 1H), 7.65 (t, J = 8.00 Hz, 3H), 7.56 (d, J = 4.00 Hz, 1H), 7.43-7.35 (m, 3H), 7.33 (t, J = 4.00 Hz, 2H), 7.31-7.28 (m, 3H), 4.38-4.29 (m, 2H), 3.74-3.68 (m, 1H), 3.60-3.55 (m, 1H), 3.02 (s, 3H), 2.16- 2.08 (m, 2H), 1.34 (t, J = 8.00 Hz, 3H); HPLC- Purity: 97.75%; Chiral HPLC-Purity 99%; MS (m/z): 541.13 [M]⁺, calcd. For C₂₆H₂₇N₃O₆S₂ m/z = 541. |
| 161 | 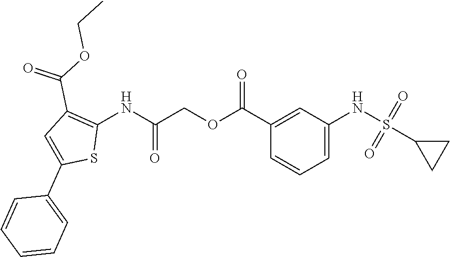 [SMILES: CCOC(=O)c1cc(-c2ccccc2)sc1NC(=O)COC(=O)c1cccc(NS(=O)(=O)C2CC2)c1] | White solid; Yield: 52%; ¹H NMR (400 MHz, DMSO-d₆): ¹H NMR (400 MHz, DMSO-d₆): δ 11.43 (s, 1H), 10.05 (s, 1H), 7.93 (d, J = 8.0 Hz, 1H), 7.67 (d, J = 4.0 Hz, 1H), 7.57 (d, J = 8.0 Hz, 2H), 7.43 (d, J = 8.0 Hz, 3H), 7.42 (t, J = 8.0 Hz, 2H), 7.33 (d, J = 8.0 Hz, 1H), 5.19 (s, 2H), 4.34 (q, J = 8.0 Hz, 2H), 1.33 (t, J = 8.0 Hz, 3H), 1.24 (s, 1H), 0.95 (d, J = 4.0 Hz, 4H); HPLC-Purity: 99.8%; MS (m/z): 528.5 [M]⁺, calcd. For C₂₅H₂₄N₂O₇S₂ m/z = 528.5. |
| 166 | 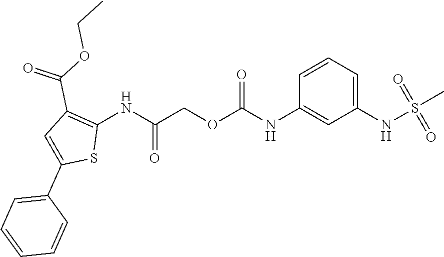 [SMILES: CCOC(=O)c1cc(-c2ccccc2)sc1NC(=O)COC(=O)Nc1cccc(NS(C)(=O)=O)c1] | Off-white solid; Yield: 40%; ¹H NMR (400 MHz, DMSO-d₆): δ 11.23 (s, 1H), 10.20 (s, 1H), 11.23 (s, 1H), 7.66 (d, J = 8.0 Hz, 2H), 7.57 (s, 1H), 7.49 (s, 1H), 7.42 (t, J = 8.0 Hz, 2H), 7.33-7.32 (m, 1H), 7.25-7.21 (m, 2H), 6.89 (d, J = 8.0 Hz, 1H), 4.97 (s, 2H), 4.33 (q, J = 8.0 Hz, 2H), 2.98 (s, 3H), 1.32 (t, J = 8.0 Hz, 3H); HPLC-Purity: 96%; MS (m/z): 517.57 [M]⁺, calcd. For C₂₃H₂₃N₃O₇S₂ m/z = 517.10. |
| 189 | 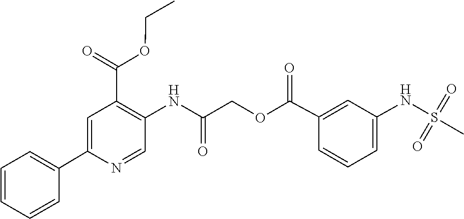 [SMILES: CCOC(=O)c1cc(-c2ccccc2)ncc1NC(=O)COC(=O)c1cccc(NS(C)(=O)=O)c1] | White solid; Yield 42%; ¹H NMR (400 MHz, DMSO-d₆): δ 10.86 (s, 1H), 10.05 (s, 1H), 9.38 (t, J = 16.0 Hz, 1H), 8.23 (t, J = 4.0 Hz, 1H), 8.10-8.09 (m, 2H), 7.94 (t, J = 4.0 Hz, 1H), 7.90- 7.88 (m, 1H), 7.47-7.67 (m, 5H), 5.03 (s, 2H), 4.33 (q, J = 8.0 Hz, 2H), 3.04 (s, 3H), 1.34 (q, J = 3.0 Hz, 3H); HPLC-Purity: 98.61%; MS (m/z): 497 [M]⁺, calcd. For C₂₄H₂₃N₃O₇S m/z = 497. |
| 219 | 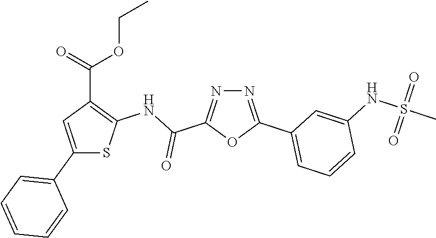 [SMILES: CCOC(=O)c1cc(-c2ccccc2)sc1NC(=O)c1nnc(-c2cccc(NS(C)(=O)=O)c2)o1] | Off-white solid; Yield: 28%; ¹H NMR (400 MHz, DMSO-d₆): δ 12.25 (s, 1H), 10.21 (s, 1H), 7.97 (t, J = 4.0 Hz, 1H), 7.86-7.84 (m, 1H), 7.75 (d, J = 8.0 Hz, 2H), 7.73 (s, 1H), 7.65 (t, J = 8.0 Hz, 1H), 7.69-7.63 (m, 1H), 7.46 (t, J = 8.0 Hz, 2H), 7.36 (t, J = 8.0 Hz, 1H), 4.43 (q, J = 8.0 Hz, 2H), 3.10 (s, 3H), 1.40 (t, J = 4.0 Hz, 3H); HPLC-Purity: 98.50%; MS (m/z): 512.56 [M]⁺, calcd. For C₂₃H₂₀N₄O₆S₂ m/z: 512.08. |

### Example 2

### Compounds as Inhibitors of ADAR1

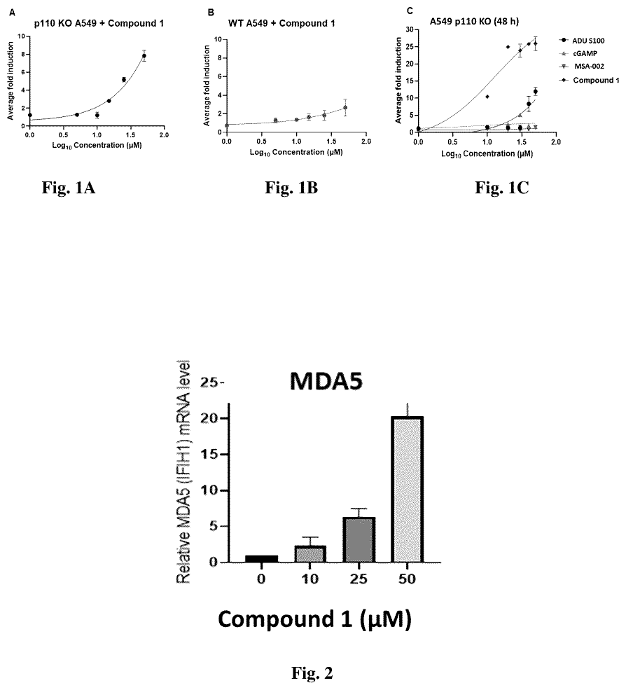

Treatment of A549 cells with Compound 1 induced IFN-β via inhibition of ADAR1 (FIGS. 1A-1C). Specifically, treatment of A549 p110 KP cells demonstrated dose-dependent IFN-β induction in A549 p110 KO cells. Lower IFN-β induction was observed in WT A549 cells treated with Compound 1 due to the presence of ADAR1 p110 activity. Compound 1 demonstrated superior IFN-β induction in A549 p110 KO cells compared to STING agonists cGAMP, ADU-S100, and MSA-002.

### Example 3

### Evaluation of ADAR1 Inhibition and Binding Affinity

6× his tag Za domain of ADAR1 p150 was purified using bacterial purification system. Protein was isolated from supernatant of cell lysate using Ni-NTA resin. For binding assay using SPR, 10 μg/ml of Zα domain of ADAR1 was immobilized on CM5 chip using anti-HIS antibody. Binding was quantified using the following condition below:
Analyte: 25, 12.5, 6.25, 3.125, 1.56, 0.78, 0 μM
Contact time—60 seconds,
Dissociation time—320 seconds
Flow rate—30 μL/minute Running buffer-PBSP+5% DMSO
After curve fitting, Kd values for synthesized compounds was quantified using the instrument software.

An ADAR1 Inhibition Assay was used to determine the biological activity (EC50) of compounds disclosed herein. 100 μL of p110 KO A549 dual cell suspension in supplemented DMEM (20,000 cells per well) was added into a flat bottom 96 well plate. Next day, post media change, 180 μL of supplemented DMEM was added. 20 μL of test compound or positive control was added to the respective wells (Final concentration of DMSO is 1%). The plate was incubated for 24 and 48 hours at 37° C. in a 5% CO₂ incubator. After 24 and 48 h, 20 μL of cell suspension was taken in black well plates and 50 μL of Quanti-Luc was added to each well and luminescence was measured using a microplate reader (Spectra Max). IFN fold-induction was plotted considering DMSO as control using the relative luminescence unit (RLU) values. (Fold induction: Sample RLU/Average DMSO RLU).

TABLE 2
Biological Activity of Disclosed Compounds

| | EC₅₀ | K_{d} | | EC₅₀ | K_{d} |
|---|---|---|---|---|---|
| Compound | (μM) | (μM) | Compound | (μM) | (μM) |
| 1 | A | X | 210 | D | ND |
| 64 | A | ND | 211 | D | ND |
| 66 | A | X | 212 | D | ND |
| 72 | A | Y | 213 | D | ND |
| 75 | A | ND | 214 | D | ND |
| 108 | A | ND | 215 | D | ND |
| 138 | A | ND | 216 | D | ND |
| 153 | A | ND | 217 | D | ND |
| 156 | A | ND | 218 | D | ND |
| 161 | A | NB | 220 | D | X |
| 189 | A | ND | 221 | D | ND |
| 219 | A | ND | 222 | D | ND |
| 296 | A | ND | 223 | D | ND |
| 310 | A | X | 224 | D | ND |
| 311 | A | ND | 225 | D | ND |
| 228 | B | ND | 226 | D | ND |
| 308 | B | ND | 227 | D | ND |
| 331 | B | ND | 229 | D | ND |
| 341 | B | ND | 230 | D | ND |
| 357 | B | Y | 231 | D | ND |
| 301 | C | ND | 232 | D | ND |
| 303 | C | ND | 233 | D | ND |
| 305 | C | ND | 234 | D | ND |
| 309 | C | ND | 235 | D | ND |
| 312 | C | ND | 236 | D | ND |
| 314 | C | ND | 237 | D | ND |
| 318 | C | ND | 238 | D | ND |
| 333 | C | ND | 239 | D | ND |
| 361 | C | NB | 240 | D | ND |
| 392 | C | ND | 241 | D | X |
| 402 | C | X | 242 | D | ND |
| 403 | C | NB | 243 | D | ND |
| 404 | C | ND | 244 | D | ND |
| 405 | C | X | 245 | D | ND |
| 406 | C | X | 246 | D | ND |
| 407 | C | ND | 247 | D | ND |
| 408 | C | ND | 248 | D | ND |
| 410 | C | ND | 249 | D | ND |
| 2 | D | X | 250 | D | ND |
| 3 | D | ND | 251 | D | ND |
| 4 | D | ND | 252 | D | NE |
| 5 | D | ND | 253 | D | X |
| 6 | D | ND | 254 | D | ND |
| 7 | D | ND | 255 | D | ND |
| 8 | D | ND | 256 | D | ND |
| 9 | D | ND | 257 | D | ND |
| 10 | D | ND | 258 | D | ND |
| 11 | D | ND | 259 | D | ND |
| 12 | D | ND | 260 | D | ND |
| 13 | D | X | 261 | D | ND |
| 14 | D | ND | 262 | D | ND |
| 15 | D | ND | 263 | D | ND |
| 16 | D | ND | 264 | D | ND |
| 17 | D | ND | 265 | D | ND |
| 18 | D | ND | 266 | D | ND |
| 19 | D | ND | 267 | D | ND |
| 20 | D | ND | 268 | D | ND |
| 21 | D | ND | 269 | D | ND |
| 22 | D | ND | 270 | D | X |
| 23 | D | ND | 271 | D | ND |
| 24 | D | ND | 272 | D | ND |
| 25 | D | ND | 273 | D | ND |
| 27 | D | ND | 274 | D | X |
| 28 | D | ND | 275 | D | ND |
| 29 | D | ND | 276 | D | ND |
| 30 | D | ND | 277 | D | ND |
| 31 | D | ND | 278 | D | ND |
| 32 | D | ND | 279 | D | ND |
| 33 | D | ND | 280 | D | ND |
| 35 | D | ND | 281 | D | ND |
| 36 | D | ND | 282 | D | ND |
| 37 | D | ND | 283 | D | ND |
| 38 | D | ND | 284 | D | ND |
| 40 | D | ND | 285 | D | ND |
| 41 | D | ND | 286 | D | ND |
| 43 | D | ND | 287 | D | ND |
| 47 | D | ND | 288 | D | ND |
| 48 | D | ND | 289 | D | ND |
| 49 | D | ND | 290 | D | ND |
| 50 | D | ND | 291 | D | ND |
| 52 | D | ND | 292 | D | X |
| 53 | D | ND | 293 | D | NB |
| 54 | D | ND | 294 | D | X |
| 55 | D | ND | 295 | D | ND |
| 56 | D | ND | 297 | D | ND |
| 57 | D | ND | 298 | D | ND |
| 58 | D | ND | 299 | D | ND |
| 59 | D | ND | 300 | D | ND |
| 60 | D | ND | 304 | D | ND |
| 61 | D | ND | 306 | D | ND |
| 63 | D | ND | 307 | D | ND |
| 65 | D | ND | 313 | D | ND |
| 67 | D | ND | 315 | D | ND |
| 68 | D | ND | 316 | D | ND |
| 69 | D | ND | 317 | D | ND |
| 70 | D | NB | 319 | D | ND |
| 71 | D | X | 320 | D | ND |
| 73 | D | ND | 321 | D | X |
| 74 | D | ND | 322 | D | ND |
| 76 | D | ND | 323 | D | ND |
| 77 | D | ND | 324 | D | ND |
| 78 | D | ND | 325 | D | X |
| 79 | D | ND | 326 | D | X |
| 80 | D | ND | 327 | D | X |
| 81 | D | ND | 328 | D | X |
| 82 | D | ND | 329 | D | Y |
| 83 | D | ND | 330 | D | X |
| 84 | D | ND | 332 | D | ND |
| 85 | D | ND | 334 | D | ND |
| 86 | D | ND | 335 | D | ND |
| 88 | D | ND | 336 | D | ND |
| 89 | D | ND | 337 | D | ND |
| 90 | D | ND | 338 | D | ND |
| 91 | D | ND | 339 | D | ND |
| 92 | D | ND | 340 | D | ND |
| 93 | D | ND | 342 | D | ND |
| 95 | D | ND | 343 | D | X |
| 96 | D | ND | 344 | D | NB |
| 97 | D | ND | 345 | D | ND |
| 100 | D | ND | 346 | D | X |
| 101 | D | ND | 347 | D | ND |
| 102 | D | ND | 348 | D | ND |
| 103 | D | ND | 349 | D | NB |
| 104 | D | ND | 350 | D | X |
| 105 | D | ND | 351 | D | X |
| 106 | D | ND | 352 | D | ND |
| 107 | D | X | 353 | D | ND |
| 109 | D | ND | 354 | D | ND |
| 110 | D | ND | 355 | D | X |
| 111 | D | ND | 356 | D | NB |
| 112 | D | ND | 358 | D | X |
| 113 | D | ND | 359 | D | X |
| 115 | D | ND | 360 | D | ND |
| 116 | D | ND | 362 | D | ND |
| 117 | D | ND | 363 | D | X |
| 119 | D | ND | 364 | D | NB |
| 120 | D | ND | 365 | D | X |
| 121 | D | X | 366 | D | X |
| 122 | D | ND | 367 | D | X |
| 123 | D | ND | 368 | D | ND |
| 124 | D | ND | 369 | D | NB |
| 125 | D | ND | 370 | D | ND |
| 126 | D | ND | 371 | D | ND |
| 127 | D | X | 372 | D | NB |
| 128 | D | ND | 373 | D | ND |
| 129 | D | ND | 374 | D | ND |
| 130 | D | X | 375 | D | ND |
| 131 | D | ND | 376 | D | X |
| 132 | D | X | 377 | D | ND |
| 133 | D | ND | 378 | D | ND |
| 134 | D | ND | 379 | D | ND |
| 135 | D | ND | 380 | D | X |
| 136 | D | ND | 381 | D | ND |
| 137 | D | NB | 382 | D | ND |
| 139 | D | ND | 383 | D | ND |
| 140 | D | ND | 384 | D | Y |
| 141 | D | ND | 385 | D | X |
| 142 | D | ND | 386 | D | NB |
| 143 | D | ND | 387 | D | X |
| 144 | D | X | 388 | D | X |
| 145 | D | ND | 389 | D | NB |
| 146 | D | ND | 390 | D | ND |
| 147 | D | X | 391 | D | ND |
| 148 | D | ND | 393 | D | ND |
| 149 | D | ND | 394 | D | ND |
| 150 | D | NB | 395 | D | NB |
| 151 | D | ND | 396 | D | X |
| 152 | D | ND | 397 | D | X |
| 154 | D | ND | 398 | D | NB |
| 155 | D | ND | 399 | D | X |
| 157 | D | ND | 400 | D | Y |
| 158 | D | ND | 401 | D | ND |
| 159 | D | ND | 409 | D | X |
| 160 | D | ND | 411 | D | ND |
| 162 | D | ND | 412 | D | NB |
| 163 | D | X | 413 | D | ND |
| 164 | D | ND | 414 | D | X |
| 165 | D | ND | 415 | D | X |
| 166 | D | ND | 416 | D | X |
| 167 | D | ND | 417 | D | ND |
| 168 | D | ND | 418 | D | Z |
| 169 | D | ND | 419 | D | X |
| 170 | D | ND | 420 | D | X |
| 171 | D | NB | 421 | D | X |
| 172 | D | X | 422 | D | ND |
| 173 | D | ND | 423 | D | ND |
| 174 | D | ND | 424 | D | X |
| 175 | D | ND | 425 | D | ND |
| 176 | D | ND | 426 | D | X |
| 177 | D | ND | 427 | D | X |
| 178 | D | ND | 428 | D | X |
| 179 | D | ND | 429 | D | ND |
| 180 | D | X | 302 | ND | X |
| 181 | D | ND | 430 | ND | ND |
| 182 | D | ND | 431 | ND | ND |
| 183 | D | ND | 432 | ND | X |
| 184 | D | ND | 433 | ND | ND |
| 185 | D | ND | 434 | ND | ND |
| 186 | D | ND | 435 | ND | X |
| 187 | D | ND | 436 | ND | X |
| 188 | D | ND | 437 | ND | X |
| 190 | D | ND | 438 | ND | ND |
| 191 | D | ND | 439 | ND | X |
| 192 | D | ND | 440 | ND | ND |
| 193 | D | ND | 441 | ND | ND |
| 194 | D | ND | 442 | ND | ND |
| 195 | D | ND | 443 | ND | ND |
| 196 | D | ND | 444 | ND | ND |
| 197 | D | ND | 445 | ND | ND |
| 198 | D | ND | 446 | ND | ND |
| 199 | D | ND | 447 | ND | ND |
| 200 | D | ND | 448 | ND | ND |
| 201 | D | ND | 449 | ND | ND |
| 202 | D | ND | 450 | ND | ND |
| 203 | D | ND | 451 | ND | ND |
| 204 | D | ND | 452 | ND | ND |
| 205 | D | ND | 453 | ND | ND |
| 206 | D | ND | 454 | ND | ND |
| 207 | D | ND | 455 | ND | ND |
| 208 | D | ND | 456 | ND | ND |
| 209 | D | ND | | | |

A ≤ 25 MM; 25 pM < B ≤ 55 uM; 50 AM < C ≤ 100 AM
SPR (Kd): X ≤1 uM; 1 uM < Y ≤ 10 UM; Z > 10 uM; NB: No binding; ND: Not determined

### Example 4

### Compound 1 Treatment Induces IFN-β via ADAR 1 Inhibition

Compound 1 demonstrated dose-dependent IFN-induction (via ADAR1 inhibition) in A549 p110 KO cells (FIG. 1A).

Compound 1 treatment of WT A549 cells has lower IFN-induction-due to presence of ADAR1 p110 activity (FIG. 1B).

Compound 1 demonstrated superior IFN-β induction in A549 p110 KO cells compared to STING agonists cGAMP, ADU-S100, MSA-002 (FIG. 1C).

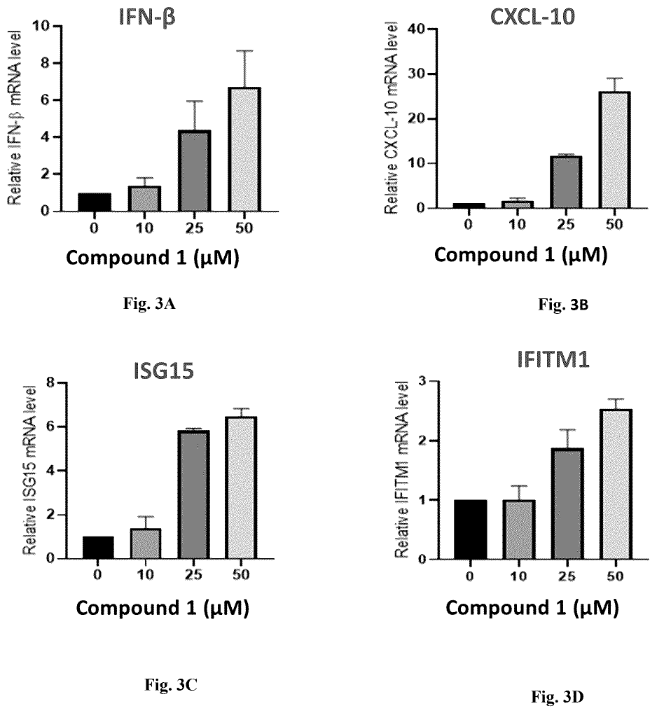

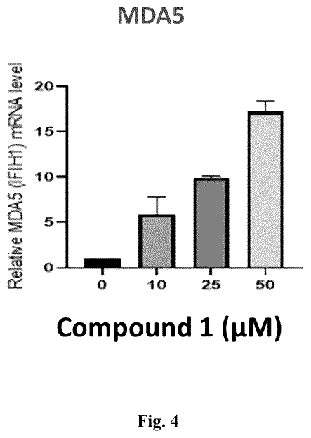

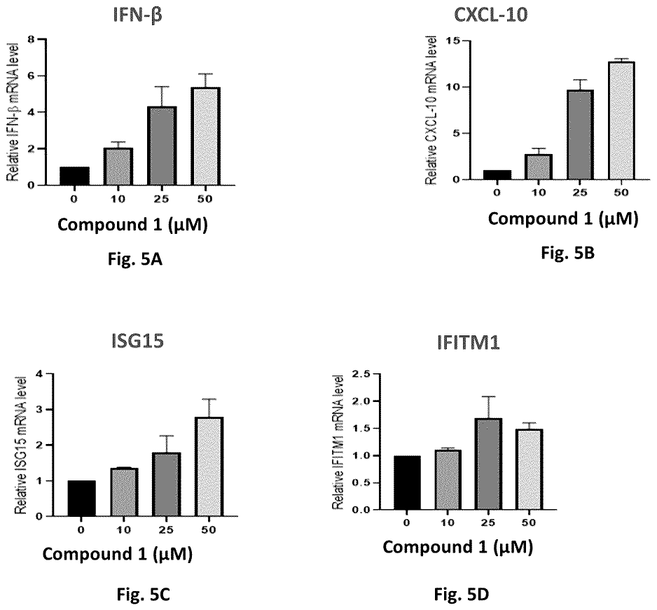

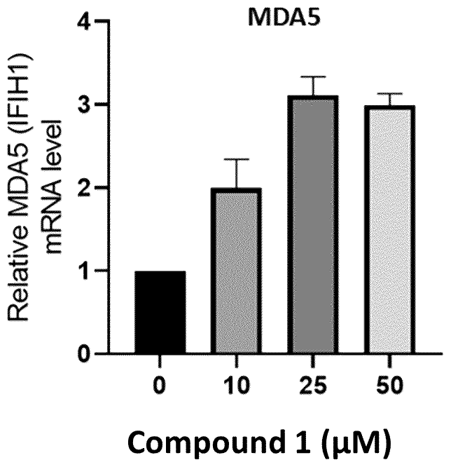

Compound 1 treatment in human cancer cell lines A549 (p110 KO) and HCT 116 induced dose-dependent expression of several interferon-stimulated genes (ISGs). See FIG. 2, FIGS. 3A-3D, FIG. 4, and FIGS. 5A-5D.

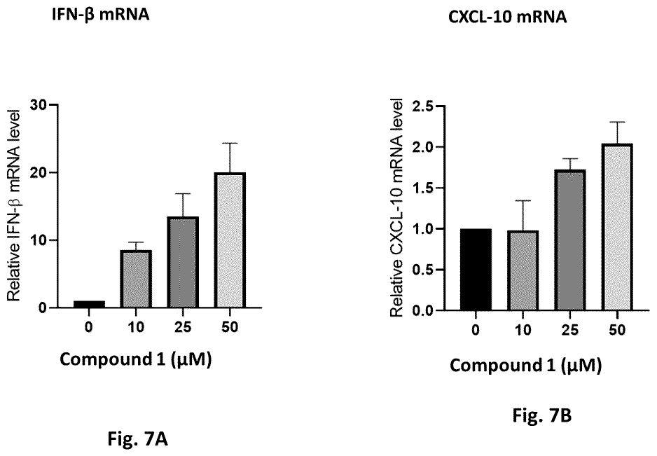

Compound 1 treatment in murine cancer cell line B16F10 induced dose-dependent upregulation of MDA5, IFN-β, and CXCL₁₀. See FIG. 6 and FIGS. 7A-7B.

### Example 5

### Evaluation of Anti-Tumor Efficacy of Disclosed Compounds in a B16F10 Mouse Melanoma Model

Study: A B16F10 mouse melanoma model was used to evaluate the anti-tumor efficacy of Compound 1, alone or in combination with anti-PD-1 therapy.

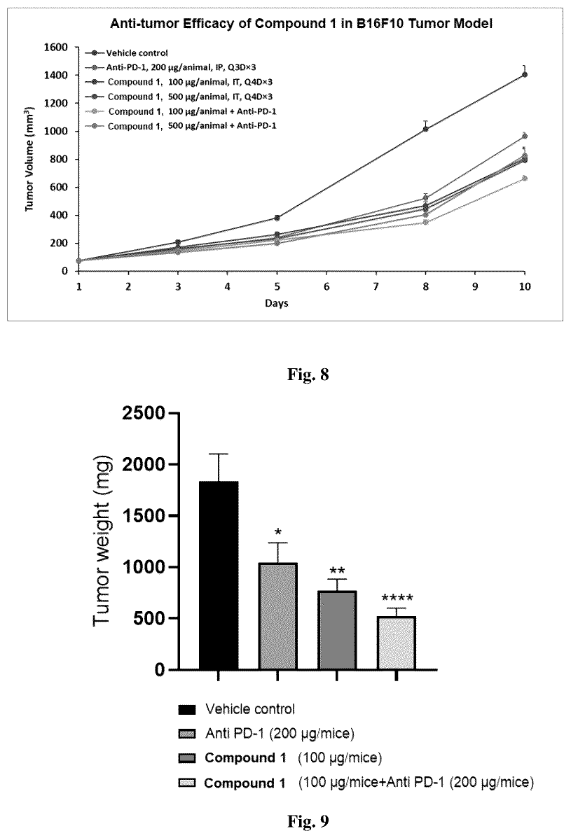

Results: As shown in FIG. 8, Compound 1 demonstrated monotherapy efficacy by providing 45% tumor growth inhibition (TGI) compared to about 30% TGI when anti-PD-1 treatment was used alone in the B16F10 mouse melanoma model.

The combination of Compound 1 and anti-PD-1 therapy provided synergistic efficacy of 55% TGI. This synergistic effect was about 2-times greater than anti-PD-1 treatment alone.

Tumor weight reduction was also evaluated in B16F10 melanoma mice (FIG. 9). As shown in FIG. 9, mice treated with anti-PD-1 therapy provided a 40% tumor weight reduction compared to vehicle control, while treatment of mice with 100 μg of Compound 1 reduced tumor weight by about 60%. Combining Compound 1 with anti-PD-1 provided a synergistic effect by reducing tumor weight by about 70% compared to control.

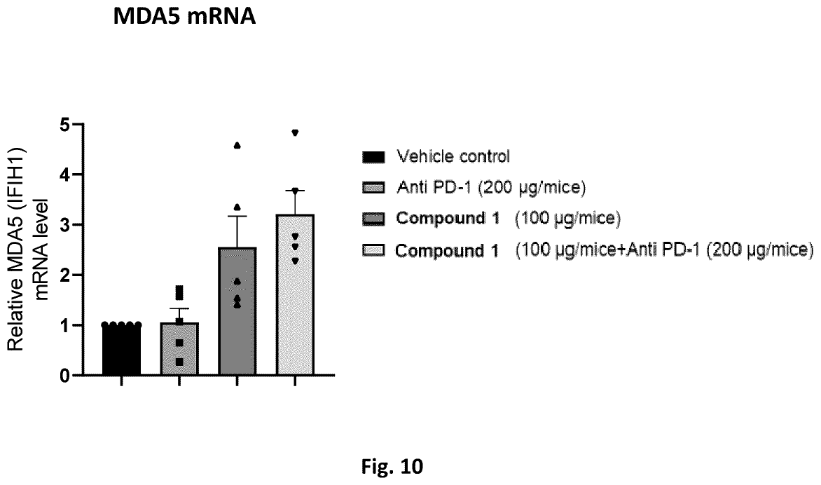

In the melanoma model, Compound 1 treatment demonstrated significant increase in tumor MDA5 mRNA levels confirming induction of downstream cytokines takes place via ADAR1-MDA5 pathway (FIG. 10).

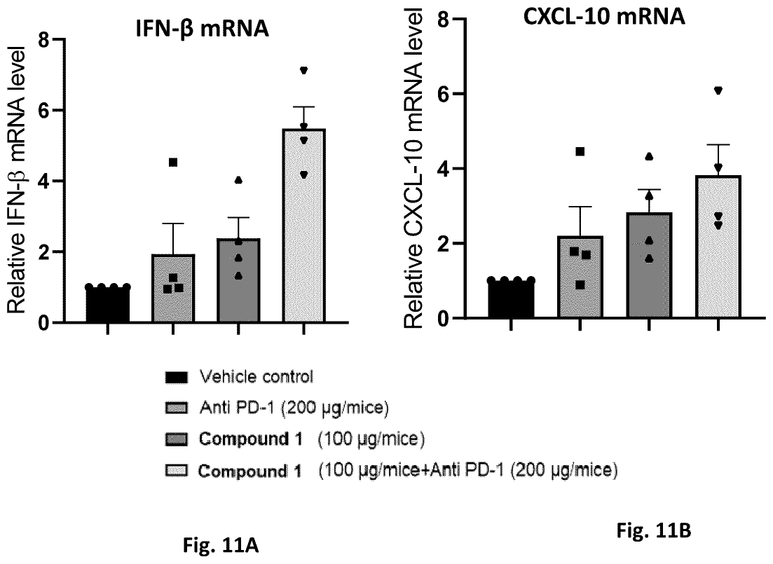

Compound 1 treatment also demonstrated increased induction of IFN-β and CXCL₁₀ in mice both as monotherapy and combination with anti-PD-1 in B16F10 tumors (FIG. 11A and FIG. 11B).

Conclusion: Treatment of B16F10 melanoma mice with compound 1 demonstrated superior efficacy to anti-PD-1 monotherapy and synergistic efficacy with anti-PD-1.

What is claimed is:

1. A compound of Formula (I) having the structure: 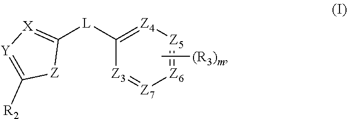
or a pharmaceutically acceptable salt or tautomer thereof,
wherein:
L is a linker having structure -A-L₁-B—,
wherein:
A is, —NR_{c}—, —NR_{c}C(O)—, —C(O)NR_{c}—, —NR_{c}S(O)₂—, or —S(O)₂NR_{c}—;
L₁ is absent, alkylene, alkenylene, alkynylene, cycloalkyl, heterocyclyl, aryl, or heteroaryl;
B is a bond, —NR_{d}—, —N(S(O)₂-alkyl)-, —NR_{d}C(O)—, —OC(O)—, 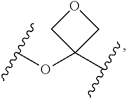 [SMILES: *OC1(*)COC1]
—OC(CF₃)—, —O—, —C(O)-alkylene-, —C(O)—, —NR_{d}S(O₂)—, —S(O₂) NR_{d}—, or heteroaryl;
R_{c} and R_{d} are each independently H, alkyl, cycloalkyl, heterocyclyl, alkylene-aryl, acyl, or sulfonyl,
with the proviso that -A-L₁-B— does not contain an —O—O, —NR_{c}—O, —O—NR_{d}—, —NR_{c}—NR_{d}—, or S—S bond;
X is C—R₁ or N;
Y is C—R₄ or N;
Z is O, S, N—R₅, or N(R₅)—CH₂—;
Z₃, Z₄, Z₅, Z₆, and Z₇ are each independently N or CH, wherein no more than 2 of Z₃, Z₄, Z₅, Z₆, and Z₇ are N;
R₁ is H, halogen, alkyl, —CN, —C(O)Rₐ, —C(O)ORₐ, —C(O)NHRₐ, —C(O)N(alkyl)(Rₐ), —C(O)N(Rₐ)(Rₐ), —C(O)NRₐRₐ, or —S(O)₂Rₐ, or —S(O)₂ORₐ, or R₁ and R_{c} taken together with the atoms to which they are attached form a heterocyclyl or heteroaryl;
R₂ is H, halogen, alkyl, aryl, heteroaryl, cycloalkyl, cycloalkenyl, heterocyclyl, or heterocycloalkenyl;
R₃ is each independently halogen, —OH, —CH₂OH, alkyl, haloalkyl, alkoxy, haloalkoxy, —S(O)₂NHR_{b}, —S(O)₂NR_{b}R_{b}, —NHS(O)₂R_{b}, —C(O)R_{b}, —C(O)OR_{b}, —C(O)NHR_{b}, —C(O)NR_{b}R_{b}, —NHC(O)R_{b}, —NHC(O)NR_{b}R_{b}, —CN, or —P(O)(OR_{b})₂, or two R₃ taken together with the adjacent carbon atoms to which they are attached form an aryl, cycloalkyl, or heterocyclyl;
R₄ is H, halogen, alkyl, —C(O)ORₐ, —C(O)NHRₐ, or aryl, or R₂ and R₄ taken together with the carbon atoms to which they are attached form a cycloalkyl, heterocyclyl, aryl, or heteroaryl;
R₅ is H, alkyl, alkylenecycloalkyl, cycloalkyl, aryl, alkylenearyl, or heteroaryl, or R₂ and R₅ taken together with the adjacent carbon atoms to which they are attached form an aryl or heteroaryl;
Rₐ is each independently H, alkyl, cycloalkyl, alkylene-cycloalkyl, aryl, heteroaryl, alkylene-aryl, or alkylene-heteroaryl, or two Rₐ groups taken together with the nitrogen atom to which they are attached form a heterocyclyl;
R_{b} is H, alkyl, cycloalkyl, alkylene-cycloalkyl, heterocyclyl, or aryl, or two R_{b} groups taken together with the nitrogen atom to which they are attached form a heterocyclyl; and
m is 0, 1, 2, or 3,
wherein the compound is not:

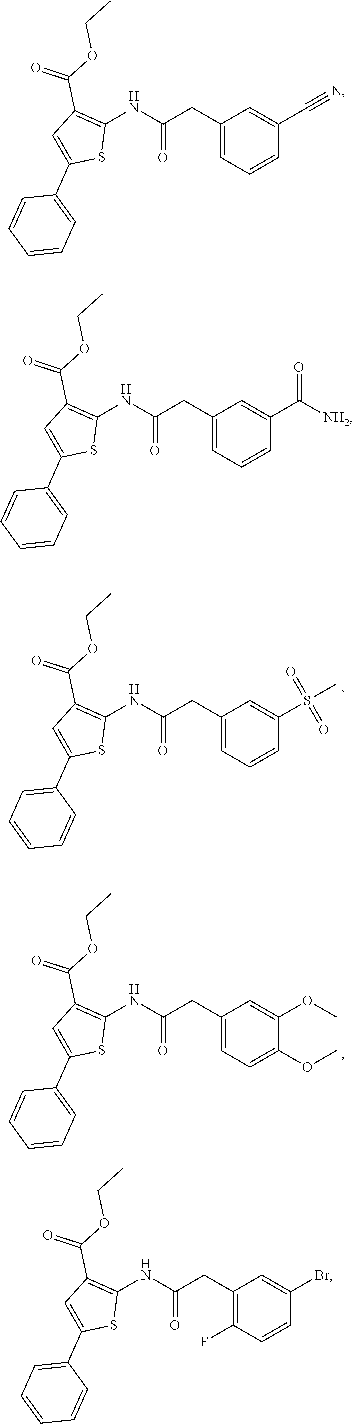 [SMILES: CCOC(=O)c1cc(-c2ccccc2)sc1NC(=O)Cc1cccc(C#N)c1 ; CCOC(=O)c1cc(-c2ccccc2)sc1NC(=O)Cc1cccc(C(N)=O)c1 ; CCOC(=O)c1cc(-c2ccccc2)sc1NC(=O)Cc1cccc(S(C)(=O)=O)c1 ; CCOC(=O)c1cc(-c2ccccc2)sc1NC(=O)Cc1ccc(OC)c(OC)c1 ; CCOC(=O)c1cc(-c2ccccc2)sc1NC(=O)Cc1cc(Br)ccc1F]

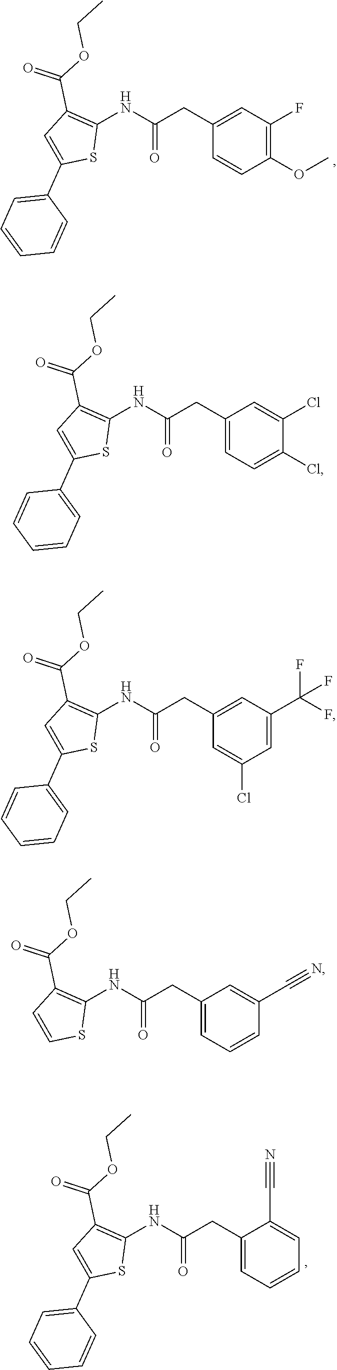 [SMILES: CCOC(=O)c1cc(-c2ccccc2)sc1NC(=O)Cc1ccc(OC)c(F)c1 ; CCOC(=O)c1cc(-c2ccccc2)sc1NC(=O)Cc1ccc(Cl)c(Cl)c1 ; CCOC(=O)c1cc(-c2ccccc2)sc1NC(=O)Cc1cc(Cl)cc(C(F)(F)F)c1 ; CCOC(=O)c1ccsc1NC(=O)Cc1cccc(C#N)c1 ; CCOC(=O)c1cc(-c2ccccc2)sc1NC(=O)Cc1ccccc1C#N]

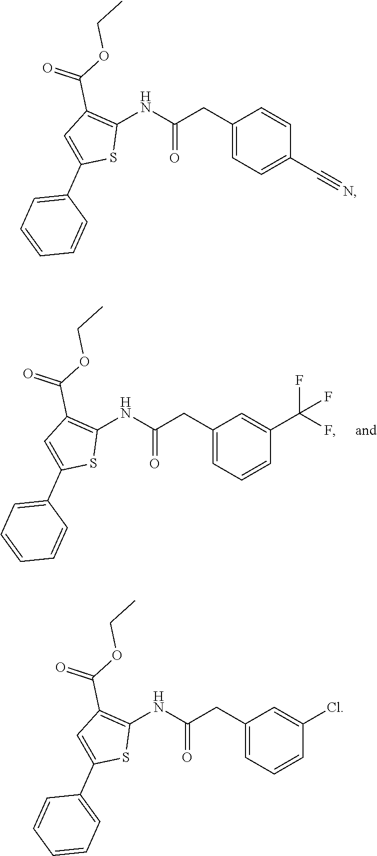 [SMILES: CCOC(=O)c1cc(-c2ccccc2)sc1NC(=O)Cc1ccc(C#N)cc1 ; CCOC(=O)c1cc(-c2ccccc2)sc1NC(=O)Cc1cccc(C(F)(F)F)c1 ; CCOC(=O)c1cc(-c2ccccc2)sc1NC(=O)Cc1cccc(Cl)c1]

2. The compound of claim 1, wherein at least two of R₁, R₂, and R₄ are not H.

3. The compound of claim 1 or 2, wherein no more than one of Z₃, Z₄, Z₅, Z₆, and Z₇ is N.

4. The compound of claim 1 or 2, wherein Z₃, Z₄, Z₅, Z₆, and Z₇ is each H.

5. The compound of any one of claims 1-4 having the structure: 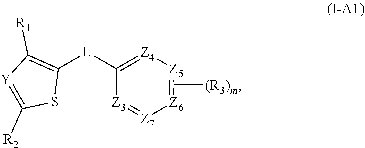
or a pharmaceutically acceptable salt or tautomer thereof.

6. The compound of any one of claims 1-4, wherein the compound has the structure: 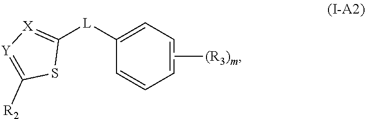
or a pharmaceutically acceptable salt thereof.

7. The compound of any one of claims 1-6, wherein the compound has the structure: 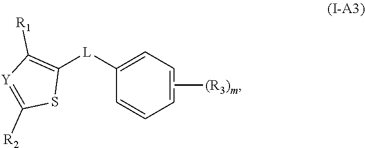
or a pharmaceutically acceptable salt thereof.

8. The compound of any one of claims 1-7, wherein the compound has the structure: 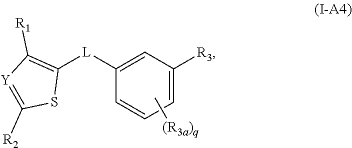
or a pharmaceutically acceptable salt thereof,
wherein:
R₃ₐ is each independently halogen, —OH, alkyl, haloalkyl, alkoxy, or haloalkoxy; and
q is 0, 1, or 2.

9. The compound of any one of claims 1-8, wherein L is a linker having the structure -A-L₁-B—, wherein:
A is —NR_{c}C(O)— or —C(O)NR_{c};
L₁ is absent, alkylene, cycloalkyl, or heterocyclyl;
B is a bond, —NR_{d}—, —N(S(O)₂-alkyl)-, —NR_{d}C(O)—, —OC(O)—, —C(O)-alkylene-, —C(O)—, —NR_{d}S(O₂)—, or —S(O₂) NR_{d}—; and
R_{c} and R_{d} are each independently H, alkyl, cycloalkyl, or alkylene-aryl, with the proviso that -A-L₁-B— does not contain an —O—O, —NR_{c}—O, —O—NR_{d}—, —NR_{c}—NR_{d}—, or S—S bond.

10. The compound of any one of claims 1-9, wherein A is —NR_{c}C(O)—, —C(O)NR_{c}—, or —C(O)—.

11. The compound of any one of claims 1-10, wherein A is —NR_{c}C(O)—.

12. The compound of any one of claims 1-11, wherein R_{c} is H, C₁₋₅alkyl, C₃₋₆cycloalkyl, or —CH₂-phenyl.

13. The compound of any one of claims 1-12, wherein L₁ is absent, alkylene, or heterocyclyl.

14. The compound of any one of claims 1-13, wherein L₁ is alkylene, and the alkylene is a C₁₋₃ alkylene.

15. The compound of any one of claims 1-14, wherein the alkylene of L₁ is an optionally substituted methylene.

16. The compound of any one of claims 1-15, wherein the alkylene of L₁ comprises the structure: 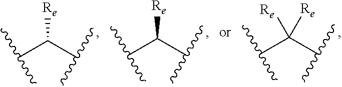
wherein Rₑ is each independently H, F, C₁₋₅ alkyl, —CH₂OH, —CH₂cycloalkyl, — or CH₂aryl, or two Rₑ taken together with the carbon atom to which they are attached form a C₃₋₆ cycloalkyl or a 4- to 6-membered heterocyclyl.

17. The compound of claim 16, wherein Rₑ is H or C₁₋₅ alkyl.

18. The compound of any one of claims 1-13, wherein L₁ is a heterocyclyl.

19. The compound of any one of claims 1-13 and 18, wherein the heterocyclyl of L₁ is a 3- to 12-membered heterocyclyl.

20. The compound of any one of claims 1-13, 18, and 19, wherein the heterocyclyl of L₁ has the structure: 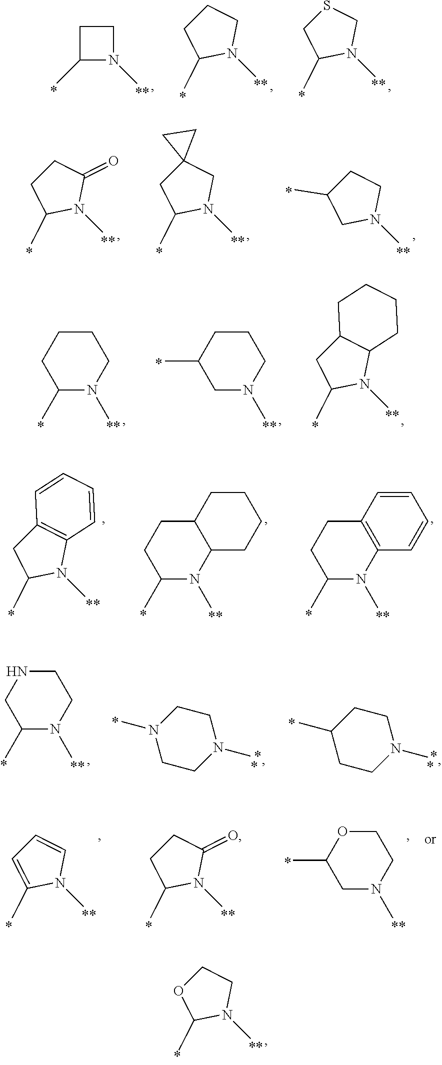
wherein * represents the attachment point to A and ** represents the attachment point to B.

21. The compound of any one of claims 1-20, wherein B is bond, —NR_{b}C(O)—, —NR_{b}S(O)₂—, —C(O)—, —OC(O)—, —C(O)—O—, or heteroaryl.

22. The compound of any one of claims 1-21, wherein B is a bond, —OC(O)—, or —C(O)—.

23. The compound of claim 21, wherein the heteroaryl is a 5- or 6-membered nitrogen-containing heteroaryl.

24. The compound of any one of claims 1-22, wherein-A-L₁-B— has the structure: 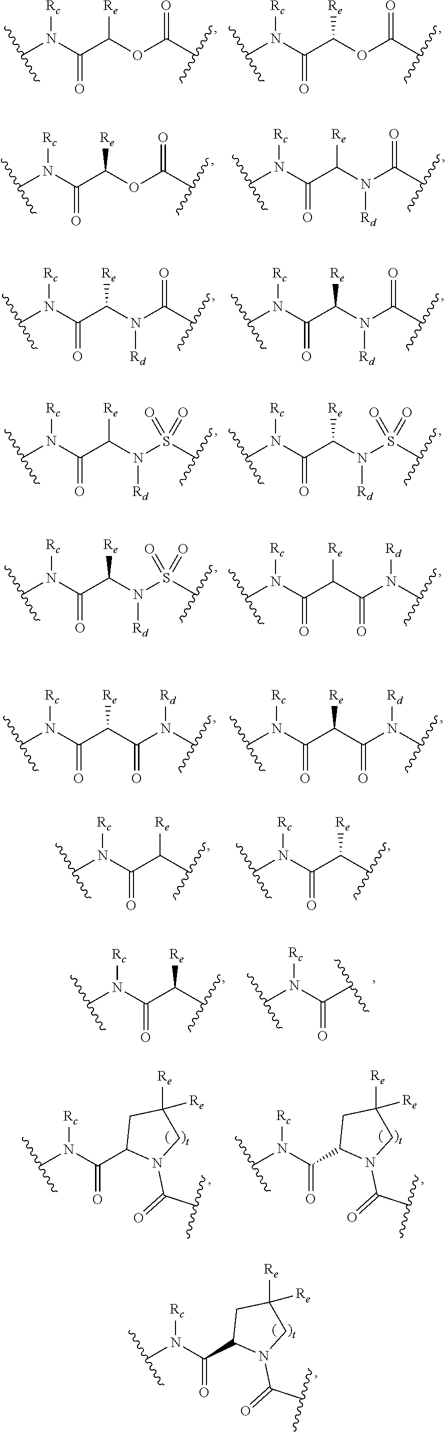 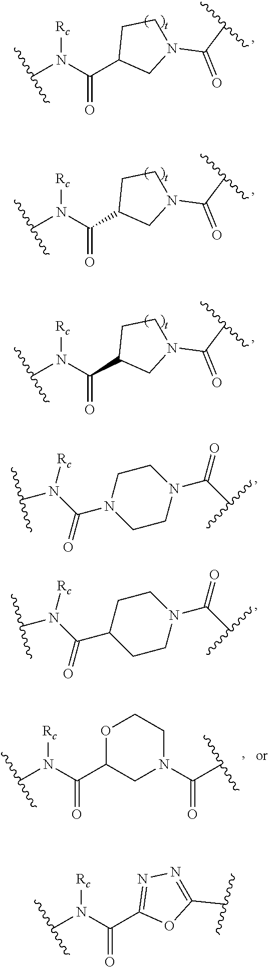
wherein:
R_{c} and R_{d} are each independently H, alkyl, cycloalkyl, alkylene-aryl, acyl, or sulfonyl;
Rₑ is each independently H, F, C₁₋₅ alkyl, —CH₂cycloalkyl, — or CH₂aryl, or two Rₑ taken together with the carbon atom to which they are attached form a C₃₋₆ cycloalkyl or a 4- to 6-membered heterocyclyl; and
t is 0, 1 or 2.

25. The compound of any one of claims 1-24, wherein A-L₁-B has the structure:

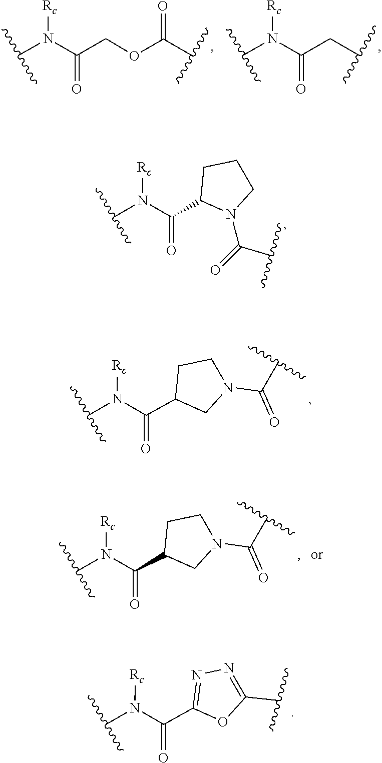

26. The compound of claim 24 or 25, wherein R_{c} is H.

27. The compound of any one of claims 1-26, wherein X is C—R₁.

28. The compound of any one of claims 1-27, wherein R₁ is halogen, alkyl, —C(O)ORₐ, or —C(O)NHRₐ.

29. The compound of any one of claims 1-28, wherein R₁ is —C(O)ORₐ.

30. The compound of any one of claims 1-26, wherein X is N.

31. The compound of any one of claims 1-30, wherein Y is C—R₄.

32. The compound of any one of claims 1-31, wherein R₄ is H, alkyl, aryl, —C(O)ORₐ, or —C(O)NHRₐ.

33. The compound of any one of claims 1-32, wherein R₄ is H.

34. The compound of any one of claims 1-33, wherein R_{d} is C₁₋₅ alkyl or —CH₂cycloalkyl.

35. The compound of any one of claims 1-34, wherein R_{d} is C₁₋₅ alkyl.

36. The compound of any one of claims 1-35, wherein R_{d} is methyl, ethyl, or n-propyl, or i-propyl.

37. The compound of any one of claims 1-36, wherein R₂ is aryl or heteroaryl.

38. The compound of any one of claims 1-37, wherein R₂ is phenyl or naphthyl.

39. The compound of any one of claims 1-38, wherein R₂ is 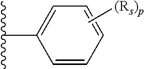
wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, 2, or 3.

40. The compound of anyone of claims 1-39, wherein R₂ is

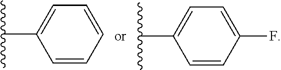

41. The compound of any one of claims 1-37, wherein R₂ is a 5- or 6-membered nitrogen-containing heteroaryl.

42. The compound of any one of claims 1-37 and 41, wherein R₂ is 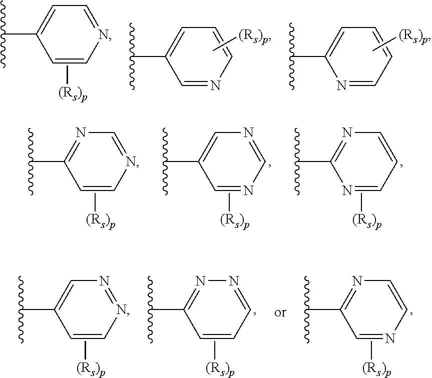
wherein Rₛ is each independently halogen, alkyl, haloalkyl, —OH, alkoxy, haloalkoxy, or amino, or two Rₛ taken together with the atoms to which they are attached form an aryl, heteroaryl, cycloalkyl, or heterocycloalkyl; and p is 0, 1, or 2.

43. The compound of any one of claims 1-31, wherein R₂ and R₄ taken together with the carbon atoms to which they are attached form a cycloalkyl, heterocyclyl, aryl, or heteroaryl.

44. The compound of claim 43, wherein R₂ and R₄ taken together with the carbon atoms to which they are attached form a cycloalkyl.

45. The compound of claim 43 or 44, wherein the cycloalkyl is a C₄₋₆ cycloalkyl.

46. The compound of any one of claims 1-40, having the structure: 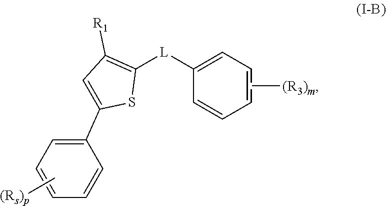
or a pharmaceutically acceptable salt thereof,
wherein:
R_{c} is each independently halogen, alkyl, haloalkyl, alkoxy, haloalkoxy, sulfonyl, or two R_{c} taken together with the atoms to which they are attached form an aryl, heteroaryl, cycloalkyl, or heterocycloalkyl; and
p is 0, 1, 2, or 3.

47. The compound of any one of claims 1-40 and 46, having the structure: 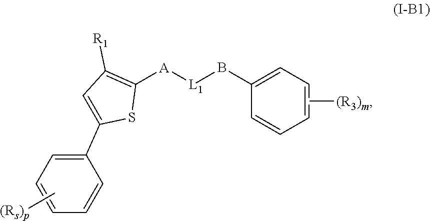
or a pharmaceutically acceptable salt thereof,
wherein:
A is —NR_{d}C(O)—, —C(O)NR_{d}—, —NR_{d}S(O)₂— or —S(O)₂NR_{d}—;
B is —NR_{d}—, —NR_{d}C(O)—, —OC(O)—, —C(O)-alkylene-, —C(O), —NR_{d}S(O₂)—, or —S(O₂)NR_{d}—; and
L₁ is a bond, C₁₋₅ alkylene, C₁₋₅ alkenylene, C₁₋₅ alkynylene, or heterocyclyl, each of which is optionally substituted.

48. The compound of any one of claims 1-47, wherein R₃ is —S(O)₂NHR_{b}, —S(O)₂NR_{b}R_{b}, —NHS(O)₂R_{b}, —C(O)R_{b}, —C(O)NHR_{b}, —C(O)NR_{b}R_{b}, —NHC(O)R_{b}, —NHC(O)NR_{b}R_{b}, or —CN.

49. The compound of any one of claims 1-48, wherein R₃ is —N(H)S(O)₂alkyl, —N(H)S(O)₂cycloalkyl, —S(O)₂NH₂, —C(O)alkyl, or —CN.

50. The compound of any one of claims 1-49, wherein R₃ is —N(H)S(O)₂CH₃, —S(O)₂NH₂, —C(O)CH₃, or —CN.

51. The compound of any one of claims 1-40 and 46-50, having the structure: 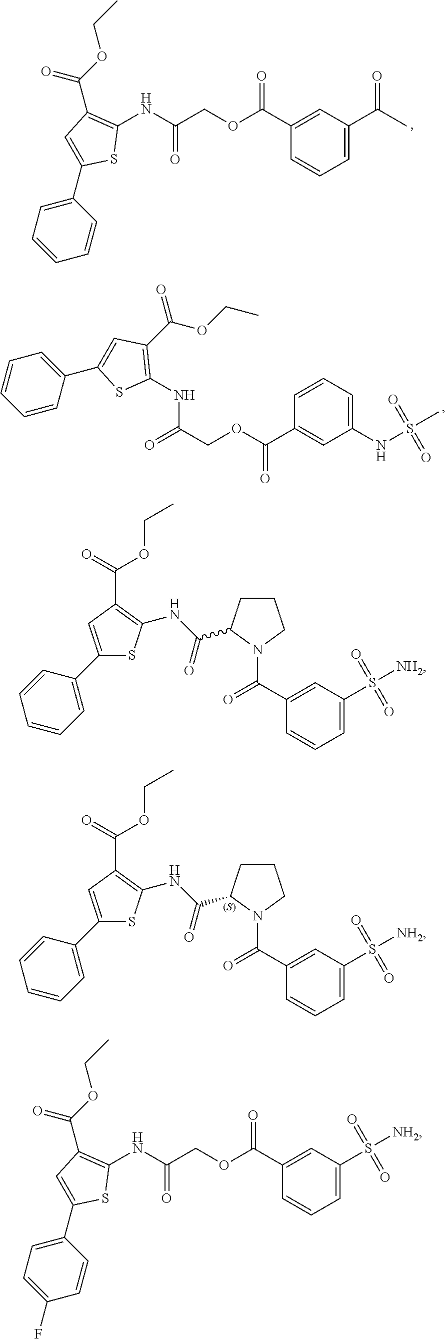 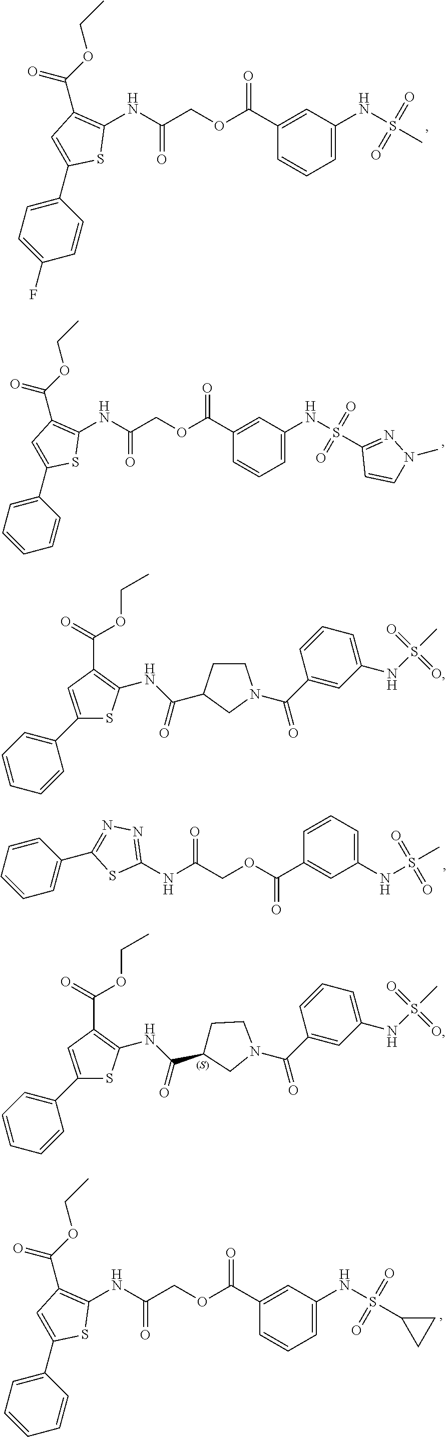 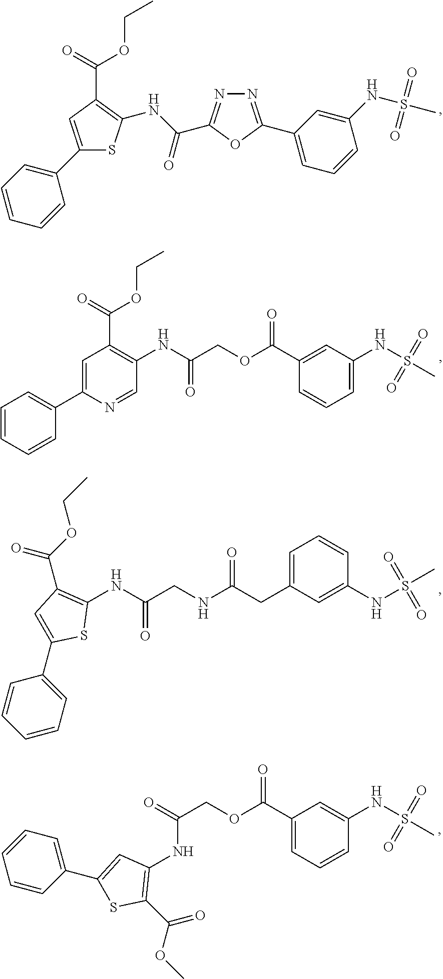 [SMILES: CCOC(=O)c1cc(-c2ccccc2)sc1NC(=O)c1nnc(-c2cccc(NS(C)(=O)=O)c2)o1 ; CCOC(=O)c1cc(-c2ccccc2)ncc1NC(=O)COC(=O)c1cccc(NS(C)(=O)=O)c1 ; CCOC(=O)c1cc(-c2ccccc2)sc1NC(=O)CNC(=O)Cc1cccc(NS(C)(=O)=O)c1 ; COC(=O)c1sc(-c2ccccc2)cc1NC(=O)COC(=O)c1cccc(NS(C)(=O)=O)c1]
or a pharmaceutically salt or tautomer thereof.

52. A pharmaceutical composition comprising a compound of any one of claims 1-51 and one or more pharmaceutically acceptable excipients.

53. A method of treating cancer in a subject in need thereof, comprising administering to the subject an effective amount of a compound of any one of claims 1-51.

54. The method of claim 53, wherein the cancer is breast cancer, lung cancer, pancreatic cancer, melanoma, multiple myeloma, colon carcinoma, colorectal cancer, or glioblastoma.

55. The method of claim 53 or 54, further comprising administering an anti-PD-1 antibody.

56. The method of claim 55, wherein administering the compound and the anti-PD-1 antibody to the subject provides a synergistic effect in treating cancer.
